SEQUENCE LISTING (73 sequences)

SEQ ID NO: 1 - RNA, 23 nt, Artificial
  Synthetic primer
uggcagugucuuagcugguuguu

SEQ ID NO: 2 - RNA, 23 nt, Artificial
  Synthetic primer
uaggcagugucauuagcugauug

SEQ ID NO: 3 - RNA, 23 nt, Artificial
  Synthetic primer
aggcaguguaguuagcugauugc

SEQ ID NO: 4 - RNA, 22 nt, Artificial
  Synthetic primer
aggcagugugguuagcugguug

SEQ ID NO: 5 - RNA, 23 nt, Artificial
  Synthetic primer
uaggcaguguaauuagcugauug

SEQ ID NO: 6 - RNA, 22 nt, Artificial
  Synthetic primer
uggcagugugguuagcugguug

SEQ ID NO: 7 - RNA, 22 nt, Artificial
  Synthetic primer
aggcagugugguuagcugguug

SEQ ID NO: 8 - RNA, 22 nt, Artificial
  Synthetic primer
uggcagugucuuagcugguugu

SEQ ID NO: 9 - RNA, 23 nt, Artificial
  Synthetic primer
uaggcaguguaauuagcugauug

SEQ ID NO: 10 - RNA, 22 nt, Artificial
  Synthetic primer
uggcagugucuuagcugguugu

SEQ ID NO: 11 - RNA, 22 nt, Artificial
  Synthetic primer
uggcagugucuuagcugguugu

SEQ ID NO: 12 - RNA, 22 nt, Artificial
  Synthetic primer
aggcaguguaguuagcugauug

SEQ ID NO: 13 - RNA, 23 nt, Artificial
  Synthetic primer
aggcagugcaguuaguugauuac

SEQ ID NO: 14 - RNA, 23 nt, Artificial
  Synthetic primer
uggcagugucuuagcugguuguu

SEQ ID NO: 15 - RNA, 23 nt, Artificial
  Synthetic primer
uggcagugucuuagcugguuguu

SEQ ID NO: 16 - RNA, 22 nt, Artificial
  Synthetic primer
aggcaguguaauuagcugauug

SEQ ID NO: 17 - RNA, 22 nt, Artificial
  Synthetic primer
uggcagugugguuagcugguug

SEQ ID NO: 18 - RNA, 22 nt, Artificial
  Synthetic primer
uggcagugucuuagcugguugu

SEQ ID NO: 19 - RNA, 23 nt, Artificial
  Synthetic primer
uggcagugucuuagcugguuguu

SEQ ID NO: 20 - RNA, 23 nt, Artificial
  Synthetic primer
uggcagugucuuagcugguuguu

SEQ ID NO: 21 - RNA, 22 nt, Artificial
  Synthetic primer
uggcagugucuuagcugguugu

SEQ ID NO: 22 - RNA, 23 nt, Artificial
  Synthetic primer
caggcaguguaguuagcugauug

SEQ ID NO: 23 - RNA, 22 nt, Artificial
  Synthetic primer
uggcagugucuuagcugguugu

SEQ ID NO: 24 - RNA, 23 nt, Artificial
  Synthetic primer
aggcaguguaguuagcugauugc

SEQ ID NO: 25 - RNA, 23 nt, Artificial
  Synthetic primer
caggcaguguaguuagcugauug

SEQ ID NO: 26 - RNA, 22 nt, Artificial
  Synthetic primer
uggcagugucuuagcugguugu

SEQ ID NO: 27 - RNA, 23 nt, Artificial
  Synthetic primer
aggcaguguaguuagcugauugc

SEQ ID NO: 28 - RNA, 22 nt, Artificial
  Synthetic primer
uggcagugucuuagcugguugu

SEQ ID NO: 29 - RNA, 23 nt, Artificial
  Synthetic primer
uggcagugucuuagcugguuguu

SEQ ID NO: 30 - RNA, 22 nt, Artificial
  Synthetic primer
uggcagugugguuagcugguug

SEQ ID NO: 31 - RNA, 23 nt, Artificial
  Synthetic primer
uaggcaguguuguuagcugauug

SEQ ID NO: 32 - RNA, 23 nt, Artificial
  Synthetic primer
aggcaguguaguuagcugauugc

SEQ ID NO: 33 - RNA, 22 nt, Artificial
  Synthetic primer
uggcagugucuuagcugguugu

SEQ ID NO: 34 - RNA, 22 nt, Artificial
  Synthetic primer
uggcagugucuuagcugguugu

SEQ ID NO: 35 - RNA, 110 nt, Artificial
  Synthetic primer
ggccagcugugaguguuucuuuggcagugucuuagcugguuguugugagcaauaguaaggaagcaaucagcaaguauacugcccuagaagugcugcacguuguggggccc

SEQ ID NO: 36 - RNA, 84 nt, Artificial
  Synthetic primer
gugcucgguuuguaggcagugucauuagcugauuguacuguggugguuacaaucacuaacuccacugccaucaaaacaaggcac

SEQ ID NO: 37 - RNA, 77 nt, Artificial
  Synthetic primer
agucuaguuacuaggcaguguaguuagcugauugcuaauaguaccaaucacuaaccacacggccagguaaaaagauu

SEQ ID NO: 38 - RNA, 74 nt, Artificial
  Synthetic primer
agccugguuaccaggcaguguaguuagcugauugccaccaggaccaaucacuaaccacacagccagguaaaaag

SEQ ID NO: 39 - RNA, 67 nt, Artificial
  Synthetic primer
uucaggcaguguaguuagcugauuguguuauaucaaauuugcaaucacuagcuaaacuaccauaaaa

SEQ ID NO: 40 - RNA, 111 nt, Artificial
  Synthetic primer
ggccagcugugaguguuucuuuggcagugucuuagcugguuguugugugcaauagugaaggaagcaaucagcaaguauacugcccuagaagugcugcacguuguggggccc

SEQ ID NO: 41 - RNA, 110 nt, Artificial; identical to SEQ ID NO: 35
  Synthetic primer

SEQ ID NO: 42 - RNA, 84 nt, Artificial
  Synthetic primer
gugcucgguuuguaggcaguguaauuagcugauuguacucucaugcuuacaaucacuaguuccacugccaucaaaacaaggcac

SEQ ID NO: 43 - RNA, 110 nt, Artificial
  Synthetic primer
ggccagcugugaguguuucuuuggcagugucuuagcugguuguugugagcaauaguaaggaagcaaucagcaaguauacugcccuagaagugcuacacauuguggggccu

SEQ ID NO: 44 - RNA, 84 nt, Artificial
  Synthetic primer
gugcuugguuugcaggcaguguaguuagcugauuguacccagcgccccacaaucacuaaauucacugccaucaaaacaaggcac

SEQ ID NO: 45 - RNA, 77 nt, Artificial
  Synthetic primer
agucuaguuacuaggcaguguaguuagcugauugcuaauaguaccaaucacuaaccacacagccagguaaaaagacu

SEQ ID NO: 46 - RNA, 67 nt, Artificial; identical to SEQ ID NO: 39
  Synthetic primer

SEQ ID NO: 47 - RNA, 96 nt, Artificial
  Synthetic primer
cugugaguguuucuuuggcagugucuuagcugguuguuguggcacguuauagaaguagcaaucagcaaauauacugcccuagaaguucugcacauu

SEQ ID NO: 48 - RNA, 77 nt, Artificial; identical to SEQ ID NO: 45
  Synthetic primer

SEQ ID NO: 49 - RNA, 111 nt, Artificial
  Synthetic primer
ggccagcugugaguguuucuuuggcagugucuuagcugguuguugugagcaauagugaaggaagcaaucagcaaguauacugcccuagaagugcugcacguuguggggccc

SEQ ID NO: 50 - RNA, 98 nt, Artificial
  Synthetic primer
agaaucaggguagaccgcguuggcagugugguuagcugguuguguauggaaaugacaacagccacuaacgacacugcuccugcgugcacccuaaauca

SEQ ID NO: 51 - RNA, 111 nt, Artificial
  Synthetic primer
ggccggcugugaguguuucuuuggcagugucuuagcugguuguugugagcaauagugaaggaagcaaucagcaaguauacugcccuagaagugcugcacguuguggggccc

SEQ ID NO: 52 - RNA, 84 nt, Artificial
  Synthetic primer
ugcuguguggucaccaggcagugcaguuaguugauuacaauccauaaaguaaucacuaaccucacuaccaggugaaggcuagua

SEQ ID NO: 53 - RNA, 84 nt, Artificial
  Synthetic primer
gugcucgguuuguaggcaguguaauuagcugauuguagugcggugcugacaaucacuaacuccacugccaucaaaacaaggcac

SEQ ID NO: 54 - RNA, 111 nt, Artificial
  Synthetic primer
ggccagcugugaguguuucuuuggcagugucuuagcugguuguugugaguaauagauaaggaagcaaucagcaaguauacugcccuagaagugcugcacguuguuaggccc

SEQ ID NO: 55 - RNA, 110 nt, Artificial; identical to SEQ ID NO: 35
  Synthetic primer

SEQ ID NO: 56 - RNA, 110 nt, Artificial; identical to SEQ ID NO: 43
  Synthetic primer

SEQ ID NO: 57 - RNA, 85 nt, Artificial
  Synthetic primer
gggguuggucuguaggcaguguuguuagcugauuguuucauaugaacuauaaucacuaaccauacugccaacacaacaaccuaca

SEQ ID NO: 58 - RNA, 98 nt, Artificial
  Synthetic primer
cugcugugagugguucucuggcagugucuuagcugguuguuguguggagugagaacgaagcaaucagcaaguauacugccgcagaaacucgucaccuu

SEQ ID NO: 59 - RNA, 102 nt, Artificial
  Synthetic primer
ccagcugugaguaauucuuuggcagugucuuagcugguuguugugaguauuagcuaaggaagcaaucagcaaguauacugcccuagaagugcugcacauugu

SEQ ID NO: 60 - RNA, 110 nt, Artificial
  Synthetic primer
ggccagcugugaguguuucuuuggcagugucuuagcugguuguugugagcaauaguaaggaagcaaucagcaaguauacugcccuagaagugcugcacguuguggccccc

SEQ ID NO: 61 - RNA, 102 nt, Artificial
  Synthetic primer
ccggcugugaguaauucuuuggcagugucuuagcugguuguugugaguauuagcuaaggaagcaaucagcaaguauacugcccuagaagugcugcacguugu

SEQ ID NO: 62 - RNA, 85 nt, Artificial
  Synthetic primer
uguuggguuuucaggcaguguaguuagcugauuguguuaacauaagacuugcaaucacuagcuaaacuaccagcaaaacuaaaca

SEQ ID NO: 63 - RNA, 110 nt, Artificial; identical to SEQ ID NO: 35
  Synthetic primer

SEQ ID NO: 64 - RNA, 85 nt, Artificial; identical to SEQ ID NO: 62
  Synthetic primer

SEQ ID NO: 65 - RNA, 93 nt, Artificial
  Synthetic primer
aagcacucauggucgugaggcagugugguuagcugguugcauacacagguugacaacggcuaccuucacugccaccccgaacauguaguccuc

SEQ ID NO: 66 - RNA, 109 nt, Artificial
  Synthetic primer
gccagcugugaguguuucuuuggcagugucuuagcugguuguugugagcaauaguuaaggaagcaaucagcaaguauacugcccuagaagugcuacacauuguugggcc

SEQ ID NO: 67 - RNA, 77 nt, Artificial
  Synthetic primer
agucuaguuacuaggcaguguaguuagcugauugcuaauaauaccaaucacuaaccacacggccagguaaaaagauu

SEQ ID NO: 68 - RNA, 100 nt, Artificial
  Synthetic primer
aauuggcuaugcgcuuuggcagugugguuagcugguuguguagccaaaauauugccuuugaccauucacagccacuaucuucacugccgccgcgacaagc

SEQ ID NO: 69 - RNA, 99 nt, Artificial
  Synthetic primer
aauuggcuaugcgcuuuggcagugugguuagcugguuguguagccaauuauugccguugacaauucacagccacuaucuucacugccgccgcgacaagc

SEQ ID NO: 70 - RNA, 84 nt, Artificial; identical to SEQ ID NO: 53
  Synthetic primer

SEQ ID NO: 71 - RNA, 97 nt, Artificial
  Synthetic primer
cggacaaugcucgagaggcagugugguuagcugguugcauauuuccuugacaacggcuaccuucacugccaccccgaacaugucguccaucuuugaa

SEQ ID NO: 72 - RNA, 22 nt, Artificial
  Synthetic primer
wggcaguguvruuaggugruur

SEQ ID NO: 73 - RNA, 22 nt, Artificial
  Synthetic primer
wggcaguguvruuaggugruug

(19) United States
(12) Patent Application Publication
(10) Pub. No.: US 2009/0227533 A1
(43) Pub. Date: Sep. 10, 2009

(54) miR-34 Regulated Genes and Pathways as Targets for Therapeutic Intervention

(76) Inventors: Andreas G. Bader, Austin, TX (US); Lubna Patrawala, Austin, TX (US); Jason F. Wiggins, Austin, TX (US); Mike W. Byrom, Austin, TX (US); Charles D. Johnson, Austin, TX (US); David Brown, Austin, TX (US)

Correspondence Address: Fullbright & Jaworski L.L.P., 600 Congress Avenue, Suite 2400, Austin, TX, 78701, US

(21) Appl. No.: 12/134,932

(22) Filed: Jun. 6, 2008

Related U.S. Application Data
(60) Provisional application No. 60/942,971, filed on Jun. 8, 2007.

(51) Int. Cl. A61K 31/7105; C12N 5/06; G01N 33/50; C12Q 1/68; A61P 35/00

(52) U.S. Cl. 514/44; 435/375; 436/94; 435/6

(57) ABSTRACT

The present invention concerns methods and compositions for identifying genes or genetic pathways modulated by miR-34, using miR-34 to modulate a gene or gene pathway, using this profile in assessing the condition of a patient and/or treating the patient with an appropriate miRNA.

This application claims priority to U.S. Provisional Application Ser. No. 60/942,971 filed Jun. 8, 2007, which is incorporated herein by reference in its entirety.

### BACKGROUND OF THE INVENTION

I. Field of the Invention

The present invention relates to the fields of molecular biology and medicine. More specifically, the invention relates to methods and compositions for the treatment of diseases or conditions that are affected by miR-34 microRNAs, microRNA expression, and genes and cellular pathways directly and indirectly modulated by such.

II. Background

In 2001, several groups used a cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are distinct.

miRNAs thus far observed have been approximately 21-22 nucleotides in length, and they arise from longer precursors, which are transcribed from non-protein-encoding genes. See review of Carrington and Ambros (2003). The precursors form structures that fold back on themselves in self-complementary regions; they are then processed by the nuclease Dicer (in animals) or DCL1 (in plants) to generate the short double-stranded miRNA. One of the miRNA strands is incorporated into a complex of proteins and miRNA called the RNA-induced silencing complex. The miRNA guides the RISC complex to a target mRNA, which is then cleaved or translationally silenced, depending on the degree of sequence complementarity of the miRNA to its target mRNA. Currently, it is believed that perfect or nearly perfect complementarity leads to mRNA degradation, as is most commonly observed in plants. In contrast, imperfect base pairing, as is primarily found in animals, leads to translational silencing. However, recent data suggest additional complexity (Bagga et al., 2005; Lim et al., 2005), and mechanisms of gene silencing by miRNAs remain under intense study.

Recent studies have shown that changes in the expression levels of numerous miRNAs are associated with various cancers (reviewed in Esquela-Kerscher and Slack, 2006; Calin and Croce, 2006). miRNAs have also been implicated in regulating cell growth and cell and tissue differentiation—cellular processes that are associated with the development of cancer.

The inventors previously demonstrated that hsa-miR-34 is involved with the regulation of numerous cell activities that represent intervention points for cancer therapy and for therapy of other diseases and disorders (U.S. patent application Ser. No. 11/141,707 filed May 31, 2005 and Ser. No. 11/273,640 filed Nov. 14, 2005, each of which is incorporated herein by reference in its entirety). In a survey of 24 different human tissues, the inventors observed that miR-34 is preferentially or exclusively expressed in human lymph node tissues. When transformed into various cancer cell lines from humans, miR-34a inhibits the proliferation of prostate cancer cells (22Rv1), lung cancer cells (A549), basal cell carcinoma cells (TE354T), cervical cancer cells (HeLa), and leukemic T cells (Jurkat), but miR-34a had no anti-proliferative effect on normal human T cells. Upon transformation, miR-34a increased (Jurkat) or decreased (HeLa) programmed cell death (apoptosis) in cells. Uncontrolled cell proliferation is a hallmark of cancer. Apoptosis is a natural cellular process that helps control cancer by inducing death in cells with oncogenic potential. Many oncogenes function by altering induction of apoptosis. More recently, others have observed miR-34a to be over-expressed in cancerous liver cells (Meng et al., 2006).

Bioinformatics analyses suggest that any given miRNA may bind to and alter the expression of up to several hundred different genes. In addition, a single gene may be regulated by several miRNAs. Thus, each miRNA may regulate a complex interaction among genes, gene pathways, and gene networks. Mis-regulation or alteration of these regulatory pathways and networks, involving miRNAs, are likely to contribute to the development of disorders and diseases such as cancer. Although bioinformatics tools are helpful in predicting miRNA binding targets, all have limitations. Because of the imperfect complementarity with their target binding sites, it is difficult to accurately predict the mRNA targets of miRNAs with bioinformatics tools alone. Furthermore, the complicated interactive regulatory networks among miRNAs and target genes make it difficult to accurately predict which genes will actually be mis-regulated in response to a given miRNA.

Correcting gene expression errors by manipulating miRNA expression or by repairing miRNA mis-regulation represent promising methods to repair genetic disorders and cure diseases like cancer. A current, disabling limitation of this approach is that, as mentioned above, the details of the regulatory pathways and networks that are affected by any given miRNA, including miR-34, remain largely unknown. This represents a significant limitation for treatment of cancers in which miR-34 may play a role. A need exists to identify the genes, genetic pathways, and genetic networks that are regulated by or that may regulate hsa-miR-34 expression.

### SUMMARY OF THE INVENTION

The present invention provides additional compositions and methods by identifying genes that are direct targets for miR-34 regulation or that are indirect or downstream targets of regulation following the miR-34-mediated modification of another gene(s) expression. Furthermore, the invention describes gene, disease, and/or physiologic pathways and networks that are influenced by miR-34 and its family members. In certain aspects, compositions of the invention are administered to a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

In particular aspects, a subject or patient may be selected for treatment based on expression and/or aberrant expression of one or more miRNA or mRNA. In a further aspect, a subject or patient may be selected for treatment based on aberrations in one or more biologic or physiologic pathway(s), including aberrant expression of one or more gene associated with a pathway, or the aberrant expression of one or more protein encoded by one or more gene associated with a pathway. In still a further aspect, a subject or patient may be selected based on aberrations in miRNA expression, or biologic and/or physiologic pathway(s). A subject may be assessed for sensitivity, resistance, and/or efficacy of a therapy or treatment regime based on the evaluation and/or analysis of miRNA or mRNA expression or lack thereof. A subject may be evaluated for amenability to certain therapy prior to, during, or after administration of one or therapy to a subject or patient. Typically, evaluation or assessment may be done by analysis of miRNA and/or mRNA, as well as combination of other assessment methods that include but are not limited to histology, immunohistochemistry, blood work, etc.

In some embodiments, an infectious disease or condition includes a bacterial, viral, parasite, or fungal infection. Many of these genes and pathways are associated with various cancers and other diseases. Cancerous conditions include, but are not limited to astrocytoma, anaplastic large cell lymphoma, acute lymphoblastic leukemia, acute myeloid leukemia, angiosarcoma, breast carcinoma, B-cell lymphoma, bladder carcinoma, cervical carcinoma, carcinoma of the head and neck, chronic lymphocytic leukemia, chronic myeloid leukemia, colorectal carcinoma, endometrial carcinoma, glioma, glioblastoma, gastric carcinoma, gastrinoma, hepatoblastoma, hepatocellular carcinoma, Hodgkin lymphoma, Kaposi's sarcoma, leukemia, lung carcinoma, leiomyosarcoma, laryngeal squamous cell carcinoma, melanoma, mucosa-associated lymphoid tissue B-cell lymphoma, medulloblastoma, mantle cell lymphoma, meningioma, myeloid leukemia, multiple myeloma, high-risk myelodysplastic syndrome, mesothelioma, neurofibroma, non-Hodgkin lymphoma, non-small cell lung carcinoma, ovarian carcinoma, esophageal carcinoma, oropharyngeal carcinoma, osteosarcoma, pancreatic carcinoma, papillary carcinoma, prostate carcinoma, pheochromocytoma, rhabdomyosarcoma, squamous cell carcinoma of the head and neck, schwannoma, small cell lung cancer, salivary gland tumor, sporadic papillary renal carcinoma, thyroid carcinoma, testicular tumor, urothelial carcinoma wherein the modulation of one or more gene is sufficient for a therapeutic response. Typically a cancerous condition is an aberrant hyperproliferative condition associated with the uncontrolled growth or inability to undergo cell death, including apoptosis.

The present invention provides methods and compositions for identifying genes that are direct targets for miR-34 regulation or that are downstream targets of regulation following the miR-34-mediated modification of upstream gene expression. Furthermore, the invention describes gene pathways and networks that are influenced by miR-34 expression in biological samples. Many of these genes and pathways are associated with various cancers and other diseases. The altered expression or function of miR-34 in cells would lead to changes in the expression of these key genes and contribute to the development of disease or other conditions. Introducing miR-34 (for diseases where the miRNA is down-regulated) or a miR-34 inhibitor (for diseases where the miRNA is up-regulated) into disease cells or tissues or subjects would result in a therapeutic response. The identities of key genes that are regulated directly or indirectly by miR-34 and the disease with which they are associated are provided herein. In certain aspects a cell may be an endothelial, a mesothelial, an epithelial, a stromal, or a mucosal cell. In certain aspects the cell is a glial, a leukemic, a colorectal, an endometrial, a fat, a meninges, a lymphoid, a connective tissue, a retinal, a cervical, a uterine, a brain, a neuronal, a blood, a cervical, an esophageal, a lung, a cardiovascular, a liver, a breast, a bone, a thyroid, a glandular, an adrenal, a pancreatic, a stomach, a intestinal, a kidney, a bladder, a prostate, a uterus, an ovarian, a testicular, a splenic, a skin, a smooth muscle, a cardiac muscle, or a striated muscle cell. In certain aspects, the cell, tissue, or target may not be defective in miRNA expression yet may still respond therapeutically to expression or over expression of a miRNA. miR-34 could be used as a therapeutic target for any of these diseases. In certain embodiments miR-34 can be used to modulate the activity of miR-34 in a subject, organ, tissue, or cell.

A cell, tissue, or subject may be a cancer cell, a cancerous tissue, harbor cancerous tissue, or be a subject or patient diagnosed or at risk of developing a disease or condition. In certain aspects a cancer cell is a neuronal, glial, lung, liver, brain, breast, bladder, blood, leukemic, colon, colorectal, endometrial, stomach, skin, ovarian, fat, bone, cervical, esophageal, pancreatic, prostate, kidney, epithelial, intestinal, lymphoid, muscle, adrenal, salivary gland, testicular, or thyroid cell. In still a further aspect cancer includes, but is not limited to astrocytoma, anaplastic large cell lymphoma, acute lymphoblastic leukemia, acute myeloid leukemia, angiosarcoma, breast carcinoma, B-cell lymphoma, bladder carcinoma, cervical carcinoma, carcinoma of the head and neck, chronic lymphocytic leukemia, chronic myeloid leukemia, colorectal carcinoma, endometrial carcinoma, glioma, glioblastoma, gastric carcinoma, gastrinoma, hepatoblastoma, hepatocellular carcinoma, Hodgkin lymphoma, Kaposi's sarcoma, leukemia, lung carcinoma, leiomyosarcoma, laryngeal squamous cell carcinoma, melanoma, mucosa-associated lymphoid tissue B-cell lymphoma, medulloblastoma, mantle cell lymphoma, meningioma, myeloid leukemia, multiple myeloma, high-risk myelodysplastic syndrome, mesothelioma, neurofibroma, non-Hodgkin lymphoma, non-small cell lung carcinoma, ovarian carcinoma, esophageal carcinoma, oropharyngeal carcinoma, osteosarcoma, pancreatic carcinoma, papillary carcinoma, prostate carcinoma, pheochromocytoma, rhabdomyosarcoma, squamous cell carcinoma of the head and neck, schwannoma, small cell lung cancer, salivary gland tumor, sporadic papillary renal carcinoma, thyroid carcinoma, testicular tumor, urothelial carcinoma. In certain aspects the cancerous condition is lung carcinoma. In a further aspect the lung carcinoma is a non-small cell carcinoma. In yet a further aspect the non-small cell carcinoma is an adenocarcinoma, a squamous cell carcinoma, a large cell carcinoma, an adenosquamous cell carcinoma, or a bronchioalveolar carcinoma. In certain aspects the cancerous condition is prostate carcinoma. In a further aspect the prostate carcinoma can be PSA positive or negative and/or androgen dependent or independent.

Embodiments of the invention include methods of modulating gene expression, or biologic or physiologic pathways in a cell, a tissue, or a subject comprising administering to the cell, tissue, or subject an amount of an isolated nucleic acid or mimetic thereof comprising a miR-34 nucleic acid, mimetic, or inhibitor sequence in an amount sufficient to modulate the expression of a gene positively or negatively modulated by a miR-34 miRNA. A “miR-34 nucleic acid sequence” or “miR-34 inhibitor” includes the full length precursor of miR-34, or complement thereof or processed (i.e., mature) sequence of miR-34 and related sequences set forth herein, as well as 5, 6, 7, 8, 9, 10, 11, 12, 13, 14, 15, 16, 17, 18, 19, 20, 21, 22, 23, 24, 25, 26, 27, 28, 29 or more nucleotides of a precursor miRNA or its processed sequence, or complement thereof, including all ranges and integers there between. In certain embodiments, the miR-34 nucleic acid sequence or miR-34 inhibitor contains the full-length processed miRNA sequence or complement thereof and is referred to as the “miR-34 full-length processed nucleic acid sequence” or “miR-34 full-length processed inhibitor sequence.” In still further aspects, the miR-34 nucleic acid comprises at least 5, 6, 7, 8, 9, 10, 11, 12, 13, 14, 15, 16, 17, 18, 19, 20, 21, 22, 23, 24, 25, 50 nucleotide (including all ranges and integers there between) segment or complementary segment of a miR-34 that is at least 75, 80, 85, 90, 95, 98, 99 or 100% identical to SEQ ID NO:1 to SEQ ID NO:73. The general term miR-34 includes all members of the miR-34 family that share at least part of a mature miR-34 sequence. Mature miR-34 sequences include hsa-miR-34a UGGCAGUGUCUUAGCUGGUUGUU (MIMAT0000255; SEQ ID NO:1); hsa-miR-34b UAGGCAGUGUCAUUAGCUGAUUG (MIMAT0000685; SEQ ID NO:2); hsa-miR-34c AGGCAGUGUAGUUAGCUGAUUGC (MIMAT0000686; SEQ ID NO:3); cbr-miR-34 AGGCAGUGUGGUUAGCUGGUUG (MIMAT0000466; SEQ ID NO:4); mo-miR-34b UAGGCAGUGUAAUUAGCUGAUUG (MIMAT0000813; SEQ ID NO:5); dps-miR-34 UGGCAGUGUGGUUAGCUGGUUG (MIMAT0001223; SEQ ID NO:6); cel-miR-34 AGGCAGUGUGGUUAGCUGGUUG (MIMAT0000005; SEQ ID NO:7); mml-miR-34a UGGCAGUGUCUUAGCUGGUUGU (MIMAT0002499; SEQ ID NO:8); mmu-miR-34b UAGGCAGUGUAAUUAGCUGAUUG (MIMAT0000382; SEQ ID NO:9); sla-miR-34a UGGCAGUGUCUUAGCUGGUUGU (MIMAT0002500; SEQ ID NO:10); ppy-miR-34a UGGCAGUGUCUUAGCUGGUUGU (MIMAT0002497; SEQ ID NO:11); bta-miR-34c AGGCAGUGUAGUUAGCUGAUUG (MIMAT0003854; SEQ ID NO:12); dre-miR-34c AGGCAGUGCAGUUAGUUGAUUAC (MIMAT0003759; SEQ ID NO:13); mmu-miR-34a UGGCAGUGUCUUAGCUGGUUGUU (MIMAT0000542; SEQ ID NO:14); rno-miR-34a UGGCAGUGUCUUAGCUGGUUGUU (MIMAT0000815; SEQ ID NO:15); bta-miR-34b AGGCAGUGUAAUUAGCUGAUUG (MIMAT0003549; SEQ ID NO:16); dme-miR-34 UGGCAGUGUGGUUAGCUGGUUG (MIMAT0000350; SEQ ID NO:17); ggo-miR-34a UGGCAGUGUCUUAGCUGGUUGU (MIMAT0002494; SEQ ID NO:18); mdo-miR-34a UGGCAGUGUCUUAGCUGGUUGUU (MIMAT0004096; SEQ ID NO:19); gga-miR-34a UGGCAGUGUCUUAGCUGGUUGUU (MIMAT0001173; SEQ ID NO:20); age-miR-34a UGGCAGUGUCUUAGCUGGUUGU (MIMAT0002495; SEQ ID NO:21); gga-miR-34b CAGGCAGUGUAGUUAGCUGAUUG (MIMAT0001179; SEQ ID NO:22); lla-miR-34a UGGCAGUGUCUUAGCUGGUUGU (MIMAT0002501; SEQ ID NO:23); gga-miR-34c AGGCAGUGUAGUUAGCUGAUUGC (MIMAT0001180; SEQ ID NO:24); xtr-miR-34b CAGGCAGUGUAGUUAGCUGAUUG (MIMAT0003579; SEQ ID NO:25); ppa-miR-34a UGGCAGUGUCUUAGCUGGUUGU (MIMAT0002496; SEQ ID NO:26); mmu-miR-34c AGGCAGUGUAGUUAGCUGAUUGC (MIMAT0000381; SEQ ID NO:27); dre-miR-34 UGGCAGUGUCUUAGCUGGUUGU (MIMAT0001269; SEQ ID NO:28); xtr-miR-34a UGGCAGUGUCUUAGCUGGUUGUU (MIMAT0003578; SEQ ID NO:29); bmo-miR-34 UGGCAGUGUGGUUAGCUGGUUG (MIMAT0004197; SEQ ID NO:30); dre-miR-34b UAGGCAGUGUUGUUAGCUGAUUG (MIMAT0003346; SEQ ID NO:31); rno-miR-34c AGGCAGUGUAGUUAGCUGAUUGC (MIMAT0000814; SEQ ID NO:32); mne-miR-34a UGGCAGUGUCUUAGCUGGUUGU (MIMAT0002502; SEQ ID NO:33); ptr-miR-34a UGGCAGUGUCUUAGCUGGUUGU (MIMAT0002498; SEQ ID NO:34) or a complement thereof. In certain aspects, a subset of these miRNAs will be used that include some but not all of the listed miR-34 family members. In one aspect, miR-34 sequences have a consensus sequence of SEQ ID NO:72. In one embodiment only sequences comprising the consensus sequence of WGGCAGUGUV[R]UUAGGUGRUUG (wherein the bracketed nucleotide is optional) (SEQ ID NO:73) will be included with all other miRNAs excluded. The term miR-34 includes all members of the miR-34 family unless specifically identified. In certain aspects, a subset of these miRNAs will be used that include some but not all of the listed miR-34 family members. For instance, in one embodiment only sequences comprising the consensus sequence of SEQ ID NO: 73 will be included with all other miRNAs excluded.

In a further aspect, a “miR-34 nucleic acid sequence” includes all or a segment of the full length precursor of miR-34 family members. Stem-loop sequences of miR-34 family members include hsa-mir-34a GGCCAGCUGUGAGUGUUUCUUUGGCAGUGUCUUAGCUGGUUGUUGUGAGCAAUA GUAAGGAAGCAAUCAGCAAGUAUACUGCCCUAGAAGUGCUGCACGUUGUGGGGC cc (MI0000268; SEQ ID NO:35); hsa-mir-34b GUGCUCGG UUUGUAGGCAGUGUCAUUAGCUGAUUGUACUGUGGUGGUUACAAUCACUAACUC CACUGCCAUCAAAACAAGGCAC (MI0000742; SEQ ID NO:36); hsa-mir-34c AGUCUAGUUACUAGGCAGUGUAGUUAGCUGAUUGCUAAUAGUACCAAUCACUAA CCACACGGCCAGGUAAAAAGAUU (MI0000743; SEQ ID NO:37); gga-mir-34c AGCCUGGUUACCAGGCAGUGUAGUUAGCUGAUUGCCACCAGGACCAA UCACUAACCACACAGCCAGGUAAAAAG (MI0001261; SEQ ID NO:38); xtr-mir-34b-4 UUCAGGCAGUGUAGUUAGCUGAUUGUGUUAUAUCAAAUUUGCAAU CACUAGCUAAACUACCAUAAAA (MI0004818; SEQ ID NO:39); age-mir-34a GGCCAGCUGUGAGUGUUUCUUUGGCAGUGUCUUAGCUGGUUGUUGUGUGCAAUA GUGAAGGAAGCAAUCAGCAAGUAUACUGCCCUAGAAGUGCUGCACGUUGUGGGG CCC (MI0002797; SEQ ID NO:40); ptr-mir-34a GGCCAGCUGUGAGUGUUUCUUUGGCAGUGUCUUAGCUGGUUGUUGUGAGCAAUA GUAAGGAAGCAAUCAGCAAGUAUACUGCCCUAGAAGUGCUGCACGUUGUGGGGC CC (MI0002800; SEQ ID NO:41); bta-mir-34b GUGCUCGGUUUGUAGGCAGUGUAAUUAGCUGAUUGUACUCUCAUGCUUACAAUC ACUAGUUCCACUGCCAUCAAAACAAGGCAC (MI0004763; SEQ ID NO:42); mne-mir-34a GGCCAGCUGUGAGUGUUUCUUUGG CAGUGUCUUAGCUGGUUGUUGUGAGCAAUAGUAAGGAAGCAAUCAGCAAGUAUA CUGCCCUAGAAGUGCUACACAUUGUGGGGCCU (MI0002804; SEQ ID NO:43); gga-mir-34b GUGCUUGGUUUGCAGGCAGUGUAGUUAGCUG AUUGUACCCAGCGCCCCACAAUCACUAAAUUCACUGCCAUCAAAACAAGGCAC (MI0001260; SEQ ID NO:44); rno-mir-34c AGUCUAGUUACUAGG CAGUGUAGUUAGCUGAUUGCUAAUAGUACCAAUCACUAACCACACAGCCAGGUA AAAAGACU (MI0000876; SEQ ID NO:45); xtr-mir-34b-2 UUCAGGCAGUGU AGUUAGCUGAUUGUGUUAUAUCAAAUUUGCAAUCACUAGCUAAACUACCAUAAA A (MI0004817; SEQ ID NO:46); xtr-mir-34a CUGUGAGUGUU UCUUUGGCAGUGUCUUAGCUGGUUGUUGUGGCACGUUAUAGAAGUAGCAAUCAG CAAAUAUACUGCCCUAGAAGUUCUGCACAUU (MI0004816; SEQ ID NO:47); mmu-mir-34c AGUCUAGUUACUAGGCAGUGUAGUUAGCUGAUUG CUAAUAGUACCAAUCACUAACCACACAGCCAGGUAAAAAGACU (MI0000403; SEQ ID NO:48); lla-mir-34a GGCCAGCUGUGAGUGUUUCUUUGGCAGUGUCUU AGCUGGUUGUUGUGAGCAAUAGUGAAGGAAGCAAUCAGCAAGUAUACUGCCCUA GAAGUGCUGCACGUUGUGGGGCCC (MI0002803; SEQ ID NO:49); bmo-mir-34 AGAAUCAGGGUAGACCGCGUUGGCAGUGUGGUUAGCUGGUUGUG UAUGGAAAUGACAACAGCCACUAACGACACUGCUCCUGCGUGCACCCUAAAUCA (MI0004975; SEQ ID NO:50); sla-mir-34a GGCCGGCU GUGAGUGUUUCUUUGGCAGUGUCUUAGCUGGUUGUUGUGAGCAAUAGUGAAGGA AGCAAUCAGCAAGUAUACUGCCCUAGAAGUGCUGCACGUUGUGGGGCCC (MI0002802; SEQ ID NO:51); dre-mir-34c UGCUGUGUGGUCA CCAGGCAGUGCAGUUAGUUGAUUACAAUCCAUAAAGUAAUCACUAACCUCACUAC CAGGUGAAGGCUAGUA (MI0004774; SEQ ID NO:52); rno-mir-34b GUGCUCGGUUUGUAGGCAGUGUAAUUAGCUGAUUGUAGUGCGGUGCUGACAAUC ACUAACUCCACUGCCAUCAAAACAAGGCAC (MI0000875; SEQ ID NO:53); mdo-mir-34a GGCCAGCUGUGAGUGUUUCUUUGGCAGUGUCUUAGCU GGUUGUUGUGAGUAAUAGAUAAGGAAGCAAUCAGCAAGUAUACUGCCCUAGAAG UGCUGCACGUUGUUAGGCCC (MI0005280; SEQ ID NO:54); ggo-mir-34a GGCCAGCUGUGAGUGUUUCUUUGGCAGUGUCUUAGCUGGUUGUUGUGAGCAAUA GUAAGGAAGCAAUCAGCAAGUAUACUGCCCUAGAAGUGCUGCACGUUGUGGGGC cc (MI0002796; SEQ ID NO:55); mml-mir-34a GGCCAGC UGUGAGUGUUUCUUUGGCAGUGUCUUAGCUGGUUGUUGUGAGCAAUAGUAAGGA AGCAAUCAGCAAGUAUACUGCCCUAGAAGUGCUACACAUUGUGGGGCCU (MI0002801; SEQ ID NO:56); dre-mir-34b GGGGUUGGU CUGUAGGCAGUGUUGUUAGCUGAUUGUUUCAUAUGAACUAUAAUCACUAACCAU ACUGCCAACACAACAACCUACA (MI0003690; SEQ ID NO:57); dre-mir-34 CUGCUGUGAGUGGUUCUCUGGCAGUGUCUUAGCUGGUUGUUGUGUGGAGUGAGA ACGAAGCAAUCAGCAAGUAUACUGCCGCAGAAACUCGUCACCUU (MI0001365; SEQ ID NO:58); mmu-mir-34a CCAGCUGUGA GUAAUUCUUUGGCAGUGUCUUAGCUGGUUGUUGUGAGUAUUAGCUAAGGAAGCA AUCAGCAAGUAUACUGCCCUAGAAGUGCUGCACAUUGU (MI0000584; SEQ ID NO:59); ppa-mir-34a GGCCAGCUGUGAGUGUUUCUUUG GCAGUGUCUUAGCUGGUUGUUGUGAGCAAUAGUAAGGAAGCAAUCAGCAAGUAU ACUGCCCUAGAAGUGCUGCACGUUGUGGCCCCC (MI0002798; SEQ ID NO:60); rno-mir-34a CCGGCUGUGAGUAAUUCUUUGGCAGUGUCUUAGCUGGU UGUUGUGAGUAUUAGCUAAGGAAGCAAUCAGCAAGUAUACUGCCCUAGAAGUGC UGCACGUUGU (MI0000877; SEQ ID NO:61); xtr-mir-34b-1 UGUUG GGUUUUCAGGCAGUGUAGUUAGCUGAUUGUGUUAACAUAAGACUUGCAAUCACU AGCUAAACUACCAGCAAAACUAAACA (MI0004925; SEQ ID NO:62); ppy-mir-34a GGCCAGCUGUGAGUGUUUCUUUGGCAGUGUCUUAGCUGGUUG UUGUGAGCAAUAGUAAGGAAGCAAUCAGCAAGUAUACUGCCCUAGAAGUGCUGC ACGUUGUGGGGCCC (MI0002799; SEQ ID NO:63); xtr-mir-34b-3 UGUUGGGUUUUCAGGCAGUGUAGUUAGCUGAUUGUGUUAACAUAAGACUUGCAA UCACUAGCUAAACUACCAGCAAAACUAAACA (MI0004924; SEQ ID NO:64); cbr-mir-34 AAGCACUCAUGGUCGUGAGGCAGUGUGGUUAGCUGGUUG CAUACACAGGUUGACAACGGCUACCUUCACUGCCACCCCGAACAUGUAGUCCUC (MI0000494; SEQ ID NO:65); gga-mir-34a GCCAGCUGUGA GUGUUUCUUUGGCAGUGUCUUAGCUGGUUGUUGUGAGCAAUAGUUAAGGAAGCA AUCAGCAAGUAUACUGCCCUAGAAGUGCUACACAUUGUUGGGCC (MI0001251; SEQ ID NO:66); bta-mir-34c AGUCUAGUU ACUAGGCAGUGUAGUUAGCUGAUUGCUAAUAAUACCAAUCACUAACCACACGGCC AGGUAAAAAGAUU (MI0005068; SEQ ID NO:67); dps-mir-34 AAUUG GCUAUGCGCUUUGGCAGUGUGGUUAGCUGGUUGUGUAGCCAAAAUAUUGCCUUU GACCAUUCACAGCCACUAUCUUCACUGCCGCCGCGACAAGC (MI0001317; SEQ ID NO:68); dme-mir-34 AAUUGGCUAUGCGCUUUGGC AGUGUGGUUAGCUGGUUGUGUAGCCAAUUAUUGCCGUUGACAAUUCACAGCCAC UAUCUUCACUGCCGCCGCGACAAGC (MI0000371; SEQ ID NO:69); mmu-mir-34b GUGCUCGGUUUGUAGGCAGUGUAAUUAGCUGAUUGUAGUGCGG UGCUGACAAUCACUAACUCCACUGCCAUCAAAACAAGGCAC (MI0000404; SEQ ID NO:70); cel-mir-34 CGGACAAUGCUCGAGAGGCAGUGUGGUUA GCUGGUUGCAUAUUUCCUUGACAACGGCUACCUUCACUGCCACCCCGAACAUGUC GUCCAUCUUUGAA (MI0000005; SEQ ID NO:71) or a complement thereof.

In certain aspects, a nucleic acid miR-34 nucleic acid, or a segment or a mimetic thereof, will comprise 5, 6, 7, 8, 9, 10, 11, 12, 13, 14, 15, 16, 17, 18, 19, 20, 21, 22, 23, 24, 25, 26, 27, 28, 29 or more nucleotides of the precursor miRNA or its processed sequence, including all ranges and integers there between. In certain embodiments, the miR-34 nucleic acid sequence contains the full-length processed miRNA sequence and is referred to as the “miR-34 full-length processed nucleic acid sequence.” In still further aspects, a miR-34 comprises at least one 5, 6, 7, 8, 9, 10, 11, 12, 13, 14, 15, 16, 17, 18, 19, 20, 21, 22, 23, 24, 25, 50 nucleotide (including all ranges and integers there between) segment of miR-34 that is at least 75, 80, 85, 90, 95, 98, 99 or 100% identical to SEQ ID NOs provided herein.

In specific embodiments, a miR-34 or miR-34 inhibitor containing nucleic acid is hsa-miR-34 or hsa-miR-34 inhibitor, or a variation thereof. miR-34 can be hsa-miR-34a or hsa-miR-34b or hsa-miR-34c. In a further aspect, a miR-34 nucleic acid or miR-34 inhibitor can be administered with 1, 2, 3, 4, 5, 6, 7, 8, 9, 10 or more miRNAs or miRNA inhibitors. miRNAs or their complements can be administered concurrently, in sequence, or in an ordered progression. In certain aspects, a miR-34 or miR-34 inhibitor can be administered in combination with one or more of a let-7, let-7b, let-7c, let-7g, miR-15, miR-16, miR-20, miR-21, miR-26a, miR-124a, miR-126, miR-143, miR-147, miR-188, miR-200, miR-215, miR-216, miR-292-3p, and/or miR-331 nucleic acid. All or combinations of miRNAs or inhibitors thereof may be administered in a single formulation. Administration may be before, during or after a second therapy. miR-34 nucleic acids or complements thereof may also include various heterologous nucleic acid sequence, i.e., those sequences not typically found operatively coupled with miR-34 in nature, such as promoters, enhancers, and the like. The miR-34 nucleic acid is a recombinant nucleic acid, and can be a ribonucleic acid or a deoxyribonucleic acid. The recombinant nucleic acid may comprise a miR-34 or miR-34 inhibitor expression cassette, i.e., a nucleic acid segment that expresses a nucleic acid when introduce into an environment containing components for nucleic acid synthesis. In a further aspect, the expression cassette is comprised in a viral vector, or plasmid DNA vector or other therapeutic nucleic acid vector or delivery vehicle, including liposomes and the like. In certain aspects a nucleic acid is a RNA and/or a synthetic nucleic acid. In a particular aspect, the miR-34 nucleic acid is a synthetic nucleic acid. Moreover, nucleic acids of the invention may be fully or partially synthetic. In certain aspects, viral vectors can be administered at 1×10², 1×10³, 1×10⁴ 1×10⁵, 1×10⁶, 1×10⁷, 1×10⁸, 1×10⁹, 1×10¹⁰, 1×10¹¹, 1×10¹², 1×10¹³, 1×10¹⁴ pfu or viral particle (vp).

In a particular aspect, the miR-34 nucleic acid or miR-34 inhibitor is a synthetic nucleic acid. Moreover, nucleic acids of the invention may be fully or partially synthetic. In still further aspects, a DNA encoding such a nucleic acid of the invention can be administered at 0.001, 0.01, 0.1, 1, 10, 20, 30, 40, 50, 100, 200, 400, 600, 800, 1000, 2000, to 4000 μg or mg, including all values and ranges there between. In yet a further aspect, nucleic acids of the invention, including synthetic nucleic acid, can be administered at 0.001, 0.01, 0.1, 1, 10, 20, 30, 40, 50, 100, to 200 μg or mg per kilogram (kg) of body weight. Each of the amounts described herein may be administered over a period of time, including 0.5, 1, 2, 3, 4, 5, 6, 7, 8, 9, 10, minutes, hours, days, weeks, months or years, including all values and ranges there between.

In certain embodiments, administration of the composition(s) can be enteral or parenteral. In certain aspects, enteral administration is oral. In further aspects, parenteral administration is intralesional, intravascular, intracranial, intrapleural, intratumoral, intraperitoneal, intramuscular, intralymphatic, intraglandular, subcutaneous, topical, intrabronchial, intratracheal, intranasal, inhaled, or instilled. Compositions of the invention may be administered regionally or locally and not necessarily directly into a lesion.

In certain aspects, the gene or genes modulated comprises 1, 2, 3, 4, 5, 6, 7, 8, 9, 10, 11, 12, 13, 14, 15, 20, 25, 30, 35, 40, 45, 50, 100, 150, 200 or more genes or combinations of genes identified in Tables 1, 3, 4, and/or 5. In still further aspects, the gene or genes modulated may exclude 1, 2, 3, 4, 5, 6, 7, 8, 9, 10, 11, 12, 13, 14, 15, 20, 25, 30, 35, 40, 45, 50, 100, 150, 175 or more genes or combinations of genes identified in Tables 1, 3, 4, and/or 5. Modulation includes modulating transcription, mRNA levels, mRNA translation, and/or protein levels in a cell, tissue, or organ. In certain aspects the expression of a gene or level of a gene product, such as mRNA or encoded protein, is down-regulated or up-regulated. In a particular aspect the gene modulated comprises or is selected from (and may even exclude) 1, 2, 3, 4, 5, 6, 7, 8, 9, 10, 11, 12, 13, 14, 15, 16, 17, 18, 19, 20, 21, 22, 23, 24, 25, 26. 27, 28, or all of the genes identified in Tables 1, 3, 4, and/or 5, or any combinations thereof. In certain embodiments a gene modulated or selected to be modulated is from Table 1. In further embodiments a gene modulated or selected to be modulated is from Table 3. In still further embodiments a gene modulated or selected to be modulated is from Table 4. In yet further embodiments a gene modulated or selected to be modulated is from Table 5. Embodiments of the invention may also include obtaining or assessing a gene expression profile or miRNA profile of a target cell prior to selecting the mode of treatment, e.g., administration of a miR-34 nucleic acid, inhibitor of miR-34, or mimetics thereof . . . . The database content related to all nucleic acids and genes designated by an accession number or a database submission are incorporated herein by reference as of the filing date of this application. In certain aspects of the invention one or more miRNA or miRNA inhibitor may modulate a single gene. In a further aspect, one or more genes in one or more genetic, cellular, or physiologic pathways can be modulated by one or more miRNAs or complements thereof, including miR-34 nucleic acids and miR-34 inhibitors in combination with other miRNAs.

miR-34 nucleic acids may also include various heterologous nucleic acid sequence, i.e., those sequences not typically found operatively coupled with miR-34 in nature, such as promoters, enhancers, and the like. The miR-34 nucleic acid is a recombinant nucleic acid, and can be a ribonucleic acid or a deoxyribonucleic acid. The recombinant nucleic acid may comprise a miR-34 expression cassette. In a further aspect, the expression cassette is comprised in a viral, or plasmid DNA vector or other therapeutic nucleic acid vector or delivery vehicle, including liposomes and the like. In a particular aspect, the miR-34 nucleic acid is a synthetic nucleic acid. Moreover, nucleic acids of the invention may be fully or partially synthetic.

A further embodiment of the invention is directed to methods of modulating a cellular pathway comprising administering to the cell an amount of an isolated nucleic acid comprising a miR-34 nucleic acid sequence in an amount sufficient to modulate the expression, function, status, or state of a cellular pathway, in particular those pathways described in Table 2 or the pathways known to include one or more genes from Table 1, 3, 4, and/or 5. Modulation of a cellular pathway includes, but is not limited to modulating the expression of one or more gene. Modulation of a gene can include inhibiting the function of an endogenous miRNA or providing a functional miRNA to a cell, tissue, or subject. Modulation refers to the expression levels or activities of a gene or its related gene product or protein, e.g., the mRNA levels may be modulated or the translation of an mRNA may be modulated, etc. Modulation may increase or up regulate a gene or gene product or it may decrease or down regulate a gene or gene product.

Still a further embodiment includes methods of treating a patient with a pathological condition comprising one or more of step (a) administering to the patient an amount of an isolated nucleic acid comprising a miR-34 nucleic acid sequence in an amount sufficient to modulate the expression of a cellular pathway; and (b) administering a second therapy, wherein the modulation of the cellular pathway sensitizes the patient to the second therapy. A cellular pathway may include, but is not limited to one or more pathway described in Table 2 below or a pathway that is know to include one or more genes of Tables 1, 3, 4, and/or 5. A second therapy can include administration of a second miRNA or therapeutic nucleic acid, or may include various standard therapies, such as chemotherapy, radiation therapy, drug therapy, immunotherapy, and the like. Embodiments of the invention may also include the determination or assessment of a gene expression profile for the selection of an appropriate therapy.

Embodiments of the invention include methods of treating a subject with a pathological condition comprising one or more of the steps of (a) determining an expression profile of one or more genes selected from Table 1, 3, 4, and/or 5; (b) assessing the sensitivity of the subject to therapy based on the expression profile; (c) selecting a therapy based on the assessed sensitivity; and (d) treating the subject using selected therapy. Typically, the pathological condition will have as a component, indicator, or result the mis-regulation of one or more gene of Table 1, 3, 4, and/or 5.

Further embodiments include the identification and assessment of an expression profile indicative of miR-34 status in a cell or tissue comprising expression assessment of one or more gene from Table 1, 3, 4, and/or 5, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers from Tables 1, 3, 4, and/or 5); it is contemplated that the nucleic acid profile can be obtained using a set of RNAs, using for example nucleic acid amplification or hybridization techniques well know to one of ordinary skill in the art. The difference in the expression profile in the sample from the patient and a reference expression profile, such as an expression profile from a normal or non-pathologic sample, is indicative of a pathologic, disease, or cancerous condition. A nucleic acid or probe set comprising or identifying a segment of a corresponding mRNA can include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 100, 200, 500, or more nucleotides, including any integer or range derivable there between, of a gene, genetic marker, a nucleic acid, mRNA or a probe representative thereof that is listed in Tables 1, 3, 4, and/or 5 or identified by the methods described herein.

Certain embodiments of the invention are directed to compositions and methods for assessing, prognosing, or treating a pathological condition in a patient comprising measuring or determining an expression profile of one or more marker(s) in a sample from the patient, wherein a difference in the expression profile in the sample from the patient and an expression profile of a normal sample or reference expression profile is indicative of pathological condition and particularly cancer (e.g., In certain aspects of the invention, the cellular pathway, gene, or genetic marker is or is representative of one or more pathway or marker described in Table 1, 3, 4, and/or 5, including any combination thereof.

Aspects of the invention include diagnosing, assessing, or treating a pathologic condition or preventing a pathologic condition from manifesting. For example, the methods can be used to screen for a pathological condition; assess prognosis of a pathological condition; stage a pathological condition; assess response of a pathological condition to therapy; or to modulate the expression of a gene, genes, or related pathway as a first therapy or to render a subject sensitive or more responsive to a second therapy. In particular aspects, assessing the pathological condition of the patient can be assessing prognosis of the patient. Prognosis may include, but is not limited to an estimation of the time or expected time of survival, assessment of response to a therapy, and the like. In certain aspects, the altered expression of one or more gene or marker is prognostic for a patient having a pathologic condition, wherein the marker is one or more of Table 1, 3, 4, and/or 5, including any combination thereof.

TABLE 1
Genes with increased (positive values) or decreased (negative
values) expression following transfection of human cancer cells
with pre-miR hsa-miR-34a.

| | RefSeq Transcript ID | |
|---|---|---|
| Gene Symbol | (Pruitt et al., 2005) | Δ log₂ |
| 15E1.2 | NM_176818 | −0.855883 |
| AADAC | NM_001086 | 1.4245 |
| ABAT | NM_000663///NM_020686 | 2.09337 |
| ABCA1 | NM_005502 | 1.74697 |
| ABCB6///ATG9A | NM_005689///NM_024085 | −1.58186 |
| ABHD3 | NM_138340 | 0.867787 |
| ABLIM3 | NM_014945 | 1.3482 |
| ADARB1 | NM_001033049///NM_001112/// | 0.842409 |
| | NM_015833///NM_015834 | |
| ADM | NM_001124 | 1.0206 |
| ADRB2 | NM_000024 | 0.987993 |
| AER61 | NM_173654 | 1.06132 |
| AGR2 | NM_006408 | −0.735648 |
| AIP | NM_003977 | −0.81314 |
| AKAP12 | NM_005100///NM_144497 | 1.06844 |
| AKAP2/// | NM_001004065///NM_007203/// | 1.41369 |
| PALM2-AKAP2 | NM_147150 | |
| AMBP | NM_001633 | 1.8111 |
| ANG///RNASE4 | NM_001145///NM_002937/// | −1.06683 |
| | NM_194430///NM_194431 | |
| ANK3 | NM_001149///NM_020987 | −1.95944 |
| ANKRD46 | NM_198401 | 2.27544 |
| ANXA10 | NM_007193 | 1.47535 |
| ANXA6 | NM_001155///NM_004033 | 1.04941 |
| AOX1 | NM_001159 | 0.985795 |
| APBA2BP | NM_031231///NM_031232 | 1.38542 |
| APBB2 | NM_173075 | 1.01175 |
| APOH | NM_000042 | −1.01185 |
| APOL1 | NM_003661///NM_145343/// | 1.41657 |
| | NM_145344 | |
| APOL2 | NM_030882///NM_145637 | 1.32603 |
| APOL6 | NM_030641 | 1.01053 |
| APP | NM_000484///NM_201413/// | 0.81516 |
| | NM_201414 | |
| APPBP2 | NM_006380 | 1.03917 |
| AQP3 | NM_004925 | −0.829627 |
| ARAF | NM_001654 | −1.33921 |
| AREG | NM_001657 | −2.00723 |
| ARHGAP1 | NM_004308 | −1.34595 |
| ARHGDIA | NM_004309 | −1.3822 |
| ARHGDIB | NM_001175 | 0.78956 |
| ARL2BP | NM_012106 | 1.41631 |
| ARMC9 | NM_025139 | 1.27907 |
| ARTS-1 | NM_016442 | 0.777184 |
| ATF3 | NM_001030287///NM_001674/// | 0.803548 |
| | NM_004024 | |
| ATF5 | NM_012068 | −0.820316 |
| ATP1B3 | NM_001679 | −1.26175 |
| ATP6V0E | NM_003945 | 1.62158 |
| ATRX | NM_000489///NM_138270/// | 0.701236 |
| | NM_138271 | |
| ATXN1 | NM_000332 | 0.762227 |
| AURKB | NM_004217 | −1.21558 |
| AVPI1 | NM_021732 | −1.15695 |
| AXL | NM_001699///NM_021913 | −1.04756 |
| B3GNT6 | NM_006876 | 0.742494 |
| B4GALT1 | NM_001497 | −1.09541 |
| BASP1 | NM_006317 | −1.09986 |
| BCL10 | NM_003921 | 0.945297 |
| BCL2A1 | NM_004049 | 1.79572 |
| BEAN | XM_375359 | 1.43239 |
| BFSP1 | NM_001195 | 1.83387 |
| BIRC3 | NM_001165///NM_182962 | 1.38727 |
| BIRC5 | NM_001012270///NM_001012271/// | −1.24824 |
| | NM_001168 | |
| BRCA1 | NM_007294///NM_007295/// | −1.22874 |
| | NM_007296///NM_007297/// | |
| | NM_007298///NM 007299 | |
| BRCA2 | NM_000059 | −1.1312 |
| BRD4 | NM_014299///NM_058243 | −1.07112 |
| BTN3A2 | NM_007047 | 1.0274 |
| BUB1 | NM_004336 | −0.713041 |
| C10orf6 | NM_018121 | 1.01113 |
| C11orf9 | NM_013279 | −1.08113 |
| C14orf45 | NM_025057 | 2.47389 |
| C14orf87 | NM_016417 | −1.18865 |
| C16orf35 | NM_012075 | −1.19951 |
| C19orf21 | NM_173481 | −1.30656 |
| C1orf121 | NM_016076 | −1.21093 |
| C1QL1 | NM_006688 | −1.26437 |
| C1R | NM_001733 | 1.02369 |
| C20orf27 | NM_017874 | −1.14465 |
| C20orf28 | NM_015417 | 1.30003 |
| C3 | NM_000064 | 0.937791 |
| C5orf13 | NM_004772 | −1.07726 |
| C5orf15 | NM_020199 | 0.944249 |
| C8orf1 | NM_004337 | 0.861254 |
| C9orf116 | NM_144654 | 1.38283 |
| C9orf9 | NM_018956 | 1.421 |
| C9orf95 | NM_017881 | 1.55696 |
| CA11 | NM_001217 | −1.18345 |
| CA8 | NM_004056 | 1.55625 |
| CABYR | NM_012189///NM_138643/// | 1.04961 |
| | NM_138644///NM_153768/// | |
| | NM_153769///NM_153770 | |
| CACNA1G | NM_018896///NM_198376/// | −0.901954 |
| | NM_198377///NM_198378// | |
| | NM_198379///NM_198380 | |
| CALM1 | NM_006888 | 0.813961 |
| CAP1 | NM_006367 | −0.896135 |
| CAP2 | NM_006366 | 1.09193 |
| CASP2 | NM_001224///NM_032982/// | −1.28474 |
| | NM_032983 | |
| CASP7 | NM_001227///NM_033338/// | 1.03974 |
| | NM_033339///NM_033340 | |
| CCL2 | NM_002982 | 1.36514 |
| CCL20 | NM_004591 | 1.62138 |
| CCNA2 | NM_001237 | −1.41379 |
| CCND1 | NM_053056 | −0.930676 |
| CCND3 | NM_001760 | −0.771789 |
| CDC23 | NM_004661 | −1.32857 |
| CDC42BPA | NM_003607///NM_014826 | 0.74279 |
| CDCP1 | NM_022842///NM_178181 | 1.1641 |
| CDH17 | NM_004063 | −1.03903 |
| CDK4 | NM_000075 | −1.76673 |
| CDK5R1 | NM_003885 | 1.09117 |
| CDKN2C | NM_001262///NM_078626 | −0.851676 |
| CDKN3 | NM_005192 | −1.19066 |
| CDR2 | NM_001802 | 1.24562 |
| CDS1 | NM_001263 | 0.88342 |
| Cep290 | NM_025114 | 0.813496 |
| CFH | NM_000186///NM_001014975 | −1.05346 |
| CFH///CFHL1 | NM_000186///NM_001014975/// | −1.6016 |
| | NM_002113 | |
| CFLAR | NM_003879 | 1.07147 |
| CGI-48 | NM_016001 | 1.12004 |
| CHAF1A | NM_005483 | −1.42704 |
| CHES1 | NM_005197 | −2.11775 |
| CHGB | NM_001819 | −0.857594 |
| CHST11 | NM_018413 | 1.40436 |
| CLCN4 | NM_001830 | 1.14064 |
| CLDN1 | NM_021101 | 1.28975 |
| CLDN3 | NM_001306 | 0.900833 |
| CLDN4 | NM_001305 | 1.28122 |
| CLN8 | NM_018941 | 1.24729 |
| CLU | NM_001831///NM_203339 | 0.953076 |
| CMAS | NM_018686 | 1.01336 |
| CMKOR1 | NM_020311 | 2.19002 |
| COL11A1 | NM_001854///NM_080629/// | 1.3148 |
| | NM_080630 | |
| COL13A1 | NM_005203///NM_080798/// | 0.853876 |
| | NM_080799///NM_080800/// | |
| | NM_080801///NM 080802 | |
| COL4A1 | NM_001845 | 1.56564 |
| COL5A1 | NM_000093 | 1.15906 |
| COL6A1 | NM_001848 | 1.59125 |
| COL6A2 | NM_001849///NM_058174/// | 2.06239 |
| | NM_058175 | |
| COL7A1 | NM_000094 | 0.793168 |
| CPS1 | NM_001875 | −2.32498 |
| CPT2 | NM_000098 | 1.00281 |
| CRIP2 | NM_001312 | −0.922219 |
| CRISPLD2 | NM_031476 | 2.81469 |
| CSF2RA | NM_006140///NM_172245/// | 1.00137 |
| | NM_172246///NM_172247/// | |
| | NM_172248///NM_172249 | |
| CTDSPL | NM_001008392 ///NM_005808 | −1.2227 |
| CTGF | NM_001901 | 2.2556 |
| CTH | NM_001902///NM_153742 | 0.748163 |
| CTNND1 | NM_001331 | −1.28384 |
| CTSB | NM_001908///NM_147780/// | −1.17728 |
| | NM_147781///NM_147782/// | |
| | NM_147783 | |
| CTSS | NM_004079 | 1.6643 |
| CXCL1 | NM_001511 | 1.86327 |
| CXCL2 | NM_002089 | 0.973392 |
| CXCL3 | NM_002090 | 1.63863 |
| CXCL5 | NM_002994 | 1.64645 |
| CXCR4 | NM_001008540///NM_003467 | 2.06112 |
| CXX1 | NM_003928 | −1.38111 |
| CYB5-M | NM_030579 | −1.01749 |
| CYP2C9///CYP2C9 | NM_000769///NM_000771 | 1.17496 |
| CYP2C9 | NM_000771 | 1.05268 |
| CYP2R1 | NM_024514 | −1.13015 |
| CYP3A5 | NM_000777 | 1.13947 |
| CYP4F11 | NM_021187 | 0.775712 |
| CYR61 | NM_001554 | 1.08188 |
| D2LIC | NM_001012665///NM_015522/// | 1.14403 |
| | NM_016008 | |
| DCBLD2 | NM_080927 | 0.827395 |
| DCP2 | NM_152624 | 2.01114 |
| DDAH1 | NM_012137 | 1.95701 |
| DDC | NM_000790 | −0.79769 |
| DDX3Y | NM_004660 | 1.33289 |
| DDX58 | NM_014314 | 1.23454 |
| DGAT1 | NM_012079 | −1.47631 |
| DHFR | NM_000791 | −1.11281 |
| DIPA | NM_006848 | −1.01009 |
| DKFZP564B147 | — | −1.39981 |
| DKFZP564J102 | NM_001006655///NM_015398 | 1.24965 |
| DKFZp564K142 | NM_032121 | −1.75645 |
| DKK3 | NM_001018057///NM_013253/// | 1.3607 |
| | NM_015881 | |
| DNAJB4 | NM_007034 | 1.02763 |
| DOCK4 | NM_014705 | 1.59892 |
| DPYSL3 | NM_001387 | 1.11349 |
| DSU | NM_018000 | 1.07415 |
| DTL | NM_016448 | −1.32027 |
| DTYMK | NM_012145 | −1.11353 |
| DUSP10 | NM_007207///NM_144728/// | 1.01454 |
| | NM_144729 | |
| DUSP6 | NM_001946///NM_022652 | 1.14972 |
| E2F5 | NM_001951 | −1.68328 |
| E2F8 | NM_024680 | −1.2799 |
| EEF1D | NM_001960///NM_032378 | 0.808336 |
| EFHD2 | NM_024329 | −1.13016 |
| EHF | NM_012153 | 0.820509 |
| EI24 | NM_001007277///NM_004879 | −0.767372 |
| EIF2C2 | NM_012154 | 1.22563 |
| EIF3S3 | NM_003756 | −1.08841 |
| ELOVL6 | NM_024090 | 0.749146 |
| EML1 | NM_001008707///NM_004434 | 0.992653 |
| ENO2 | NM_001975 | 1.0967 |
| ENTPD7 | NM_020354 | 1.23228 |
| F3 | NM_001993 | 1.53096 |
| F8 | NM_000132///NM_019863 | −1.39114 |
| F8A1 | NM_012151 | −1.18147 |
| FA2H | NM_024306 | 0.714692 |
| FAM18B | NM_016078 | 1.0362 |
| FAM63B | NM_019092 | 1.02997 |
| FAS | NM_000043///NM_152871/// | 0.737731 |
| | NM_152872///NM_152873/// | |
| | NM_152874///NM_152875 | |
| FBN1 | NM_000138 | 1.06594 |
| FBN2 | NM_001999 | 1.11832 |
| FBXO17 | NM_024907///NM_148169 | −1.12512 |
| FBXO5 | NM_012177 | −1.05957 |
| FCHO1 | NM_015122 | −1.09992 |
| FEN1 | NM_004111 | −1.20162 |
| FGB | NM_005141 | −0.991096 |
| FGG | NM_000509///NM_021870 | −1.78384 |
| FKBP1B | NM_004116///NM_054033 | −0.996887 |
| FLJ11259 | NM_018370 | 1.30773 |
| FLJ13646 | NM_024584 | 1.0188 |
| FLJ13868 | NM_022744 | −1.04136 |
| FLJ13910 | NM_022780 | 1.17407 |
| FLJ13912 | NM_022770 | −1.55113 |
| FLJ14054 | NM_024563 | 1.12612 |
| FLJ14154 | NM_024845 | −1.12589 |
| FLJ20035 | NM_017631 | 1.07444 |
| FLJ20232 | NM_019008 | −0.851064 |
| FLJ20489 | NM_017842 | −1.26837 |
| FLJ20641 | NM_017915 | −1.02578 |
| FLOT2 | NM_004475 | −1.00905 |
| FLRT3 | NM_013281///NM_198391 | −1.49078 |
| FNBP1 | NM_015033 | 0.999242 |
| FOSL1 | NM_005438 | −1.0541 |
| FOXM1 | NM_021953///NM_202002/// | −1.34628 |
| | NM_202003 | |
| FSTL1 | NM_007085 | 1.29027 |
| FXYD2 | NM_001680///NM_021603 | −0.920405 |
| FYN | NM_002037///NM_153047/// | 1.28966 |
| | NM_153048 | |
| G0S2 | NM_015714 | 1.60366 |
| G1P2 | NM_005101 | 0.807471 |
| GABRA5 | NM_000810 | −1.43837 |
| GALNT12 | NM_024642 | 1.75421 |
| GALNT7 | NM_017423 | −1.14234 |
| GATA6 | NM_005257 | 1.09598 |
| GBP1 | NM_002053 | 1.32314 |
| GCC2 | NM_014635///NM_181453 | 1.23268 |
| GFPT1 | NM_002056 | 1.19864 |
| GFPT2 | NM_005110 | 1.45232 |
| GK | NM_000167///NM_203391 | 0.735192 |
| GLI2 | NM_005270///NM_030379/// | −1.02394 |
| | NM_030380///NM_030381 | |
| GLIPR1 | NM_006851 | 0.816274 |
| GLRB | NM_000824 | 1.12977 |
| GLS | NM_014905 | 1.38843 |
| GMNN | NM_015895 | −1.55685 |
| GNPDA1 | NM_005471 | −1.14252 |
| GORASP2 | NM_015530 | −1.22635 |
| GPNMB | NM_001005340///NM_002510 | −0.703249 |
| GPR64 | NM_005756 | −0.77618 |
| GRB14 | NM_004490 | −1.12651 |
| GREB1 | NM_014668///NM_033090/// | 1.51175 |
| | NM_148903 | |
| GREM1 | NM_013372 | −0.893265 |
| GRN | NM_001012479///NM_002087 | −1.11409 |
| GTSE1 | NM_016426 | −1.27331 |
| GTSE1/// | NM_016426///XM_498882 | −1.0392 |
| LOC440834 | | |
| GYG2 | NM_003918 | 0.926289 |
| HAS2 | NM_005328 | −1.34767 |
| HCFC1R1 | NM_001002017///NM_001002018/// | −1.0654 |
| | NM_017885 | |
| HDAC1 | NM_004964 | −1.05125 |
| HEG | XM_087386 | 1.19039 |
| HEG1 | XM_087386 | 1.06359 |
| HGD | NM_000187 | −1.27525 |
| HIC2 | NM_015094 | 0.843232 |
| HIPK3 | NM_005734 | 0.799874 |
| HIST1H2BC | NM_003526 | 1.4508 |
| HIST1H3H | NM_003536 | −1.03906 |
| HLX1 | NM_021958 | 1.53759 |
| HMGCS1 | NM_002130 | 0.733341 |
| HMGN4 | NM_006353 | −1.07679 |
| HMMR | NM_012484///NM_012485 | −1.06157 |
| HMOX1 | NM_002133 | 0.893265 |
| HOMER3 | NM_004838 | 1.01188 |
| HOXA1 | NM_005522///NM_153620 | 1.31491 |
| HS3ST1 | NM_005114 | 1.03666 |
| HSPB8 | NM_014365 | 1.31482 |
| ID1 | NM_002165///NM_181353 | −1.3088 |
| ID2 | NM_002166 | −1.50607 |
| ID2///ID2B | NM_002166 | −1.61007 |
| ID3 | NM_002167 | −1.03804 |
| IDH2 | NM_002168 | 1.16927 |
| IER3IP1 | NM_016097 | 0.98312 |
| IFI16 | NM_005531 | 0.99528 |
| IFIH1 | NM_022168 | 0.938476 |
| IFIT1 | NM_001001887///NM_001548 | 1.76266 |
| IFRD1 | NM_001007245///NM_001550 | 0.812747 |
| IFRD2 | NM_006764 | −1.20507 |
| IGFBP4 | NM_001552 | −1.01275 |
| IL11 | NM_000641 | 1.10331 |
| IL1A | NM_000575 | 1.88862 |
| IL1R1 | NM_000877 | −0.832301 |
| IL1RAP | NM_002182///NM_134470 | 1.56258 |
| IL27RA | NM_004843 | 1.01889 |
| IL32 | NM_001012631///NM_001012632/// | 2.58763 |
| | NM_001012633///NM_001012634/// | |
| | NM_001012635 | |
| IL6ST | NM_002184///NM_175767 | 1.20628 |
| IL8 | NM_000584 | 2.90711 |
| INHBB | NM_002193 | −1.01429 |
| INHBC | NM_005538 | 0.916297 |
| INSL4 | NM_002195 | −2.29905 |
| IQCG | NM_032263 | 1.29597 |
| IRF1 | NM_002198 | 1.09282 |
| IRF7 | NM_001572///NM_004029/// | 1.24714 |
| | NM_004030///NM_004031 | |
| ITGA2 | NM_002203 | 1.3846 |
| ITGAM | NM_000632 | 1.03569 |
| ITGB3 | NM_000212 | 2.03731 |
| ITGB6 | NM_000888 | 1.06132 |
| ITPR2 | NM_002223 | 1.54371 |
| JUN | NM_002228 | 1.11893 |
| KCNE4 | NM_080671 | 1.31528 |
| KCNK3 | NM_002246 | −0.767345 |
| KCNMA1 | NM_001014797///NM_002247 | 1.01352 |
| KIAA0101 | NM_001029989///NM_014736 | −1.27609 |
| KIAA0527 | XM_171054 | 1.01808 |
| KIAA0746 | NM_015187 | 1.22625 |
| KIAA0754 | — | 2.35948 |
| KIAA0882 | NM_015130 | 0.882798 |
| KIAA1164 | NM_019092 | 1.35213 |
| KIF11 | NM_004523 | −1.2027 |
| KLC2 | NM_022822 | −0.758469 |
| KLF4 | NM_004235 | −0.76891 |
| KRT15 | NM_002275 | 0.729419 |
| KRT20 | NM_019010 | 1.03241 |
| KRT7 | NM_005556 | 0.796089 |
| LAMC2 | NM_005562///NM_018891 | 1.19341 |
| LARP6 | NM_018357///NM_197958 | 0.84099 |
| LASS6 | NM_203463 | −1.05783 |
| LEPR | NM_001003679///NM_001003680/// | 1.42733 |
| | NM_002303 | |
| LEPREL1 | NM_018192 | −0.824854 |
| LGR4 | NM_018490 | −1.37431 |
| LHX2 | NM_004789 | −0.793849 |
| LITAF | NM_004862 | −1.40923 |
| LMAN1 | NM_005570 | −1.21429 |
| LMAN2L | NM_030805 | −1.16601 |
| LMO4 | NM_006769 | −1.1335 |
| LNK | NM_005475 | 1.36739 |
| LOC137886 | XM_059929 | −0.909709 |
| LOC146909 | XM_085634 | −1.13528 |
| LOC492304 | NM_001007139 | 1.00913 |
| LOC54103 | NM_017439 | 1.16544 |
| LOC93349 | NM_138402 | 1.36353 |
| LOXL2 | NM_002318 | 0.949739 |
| LPIN1 | NM_145693 | 0.823449 |
| LRP12 | NM_013437 | 0.734031 |
| LRP8 | NM_001018054///NM_004631/// | 1.22738 |
| | NM_017522///NM_033300 | |
| LRRC40 | NM_017768 | −1.24993 |
| LRRC48 | NM_031294 | 1.14188 |
| LRRC54 | NM_015516 | −1.2155 |
| LSM2 | NM_021177 | −1.23146 |
| LUM | NM_002345 | −0.973319 |
| LY6E | NM_002346 | −1.06222 |
| LYPD1 | NM_144586 | 0.70258 |
| LYST | NM_000081///NM_001005736 | 1.42511 |
| LZTFL1 | NM_020347 | 1.40668 |
| MAFF | NM_012323///NM_152878 | 2.14921 |
| MAP1B | NM_005909///NM_032010 | 1.22773 |
| MAP3K1 | XM_042066 | 1.11883 |
| MAP3K11 | NM_002419 | −1.57495 |
| MAP7 | NM_003980 | −1.28946 |
| MARCH8 | NM_001002265///NM_001002266/// | −1.25289 |
| | NM_145021 | |
| MCAM | NM_006500 | 1.0908 |
| MCL1 | NM_021960///NM_182763 | 1.03645 |
| MCM10 | NM_018518///NM_182751 | −1.04264 |
| MCM2 | NM_004526 | −1.57773 |
| MCM3 | NM_002388 | −1.51854 |
| MCM5 | NM_006739 | −1.91411 |
| MEG3 | XR_000167///XR_000277 | 1.08666 |
| MERTK | NM_006343 | 1.0367 |
| MET | NM_000245 | −1.20442 |
| MFN2 | NM_014874 | −0.815974 |
| MGAM | NM_004668 | 0.708327 |
| MGC35048 | NM_153208 | 1.00046 |
| MGC5508 | NM_024092 | −1.37543 |
| MGC5618 | — | 1.1505 |
| MICAL1 | NM_022765 | 1.12473 |
| MK167 | NM_002417 | −1.30259 |
| MKL1 | NM_020831 | −1.03444 |
| MLF1 | NM_022443 | 0.859795 |
| MMP7 | NM_002423 | 1.42996 |
| MPHOSPH6 | NM_005792 | −1.07128 |
| MTUS1 | NM_001001924///NM_001001925/// | −1.42746 |
| | NM_001001927///NM_001001931/// | |
| | NM_020749 | |
| MXD4 | NM_006454 | 1.0247 |
| MYBL2 | NM_002466 | −1.10263 |
| MYL5 | NM_002477 | 1.66702 |
| MYL9 | NM_006097///NM_181526 | 0.803112 |
| NALP1 | NM_001033053///NM_014922/// | 2.07583 |
| | NM_033004///NM_033006/// | |
| | NM_033007 | |
| NAP1L3 | NM_004538 | 1.09345 |
| NAV3 | NM_014903 | 0.770001 |
| NCF2 | NM_000433 | 2.29517 |
| NEFL | NM_006158 | 1.17139 |
| NF1 | NM_000267 | −0.778589 |
| NF2 | NM_000268///NM_016418/// | 1.00874 |
| | NM_181825///NM_181826/// | |
| | NM_181827///NM_181828 | |
| NFE2L3 | NM_004289 | 1.08319 |
| NFKB2 | NM_002502 | 1.35547 |
| NFYC | NM_014223 | −1.09134 |
| NID1 | NM_002508 | 1.17206 |
| NINJ1 | NM_004148 | −1.06946 |
| NMT2 | NM_004808 | 1.02347 |
| NMU | NM_006681 | −1.88419 |
| NNMT | NM_006169 | 0.739662 |
| NPC1 | NM_000271 | 0.893962 |
| NPR3 | NM_000908 | 1.52387 |
| NPTX1 | NM_002522 | −1.77152 |
| NR1D2 | NM_005126 | 0.808897 |
| NR4A2 | NM_006186///NM_173171/// | −1.74346 |
| | NM_173172///NM_173173 | |
| NRP2 | NM_003872///NM_018534/// | 1.23016 |
| | NM_201264///NM_201266/// | |
| | NM_201267///NM_201279 | |
| NT5E | NM_002526 | 1.91748 |
| NUCKS | NM_022731 | 1.3771 |
| NUMA1 | NM_006185 | −1.01356 |
| NUP210 | NM_024923 | −1.4912 |
| NXN | NM_022463 | 1.0689 |
| OBSL1 | XM_051017 | 0.804699 |
| OLFM1 | NM_006334///NM_014279/// | 1.31915 |
| | NM_058199 | |
| OLR1 | NM_002543 | 1.31356 |
| OPLAH | NM_017570 | 1.35807 |
| OPTN | NM_001008211///NM_001008212/// | 0.915075 |
| | NM_001008213///NM_021980 | |
| OSTM1 | NM_014028 | 1.16133 |
| OXTR | NM_000916 | 1.33936 |
| P4HA2 | NM_001017973///NM_001017974/// | 1.251 |
| | NM_004199 | |
| PALM2-AKAP2 | NM_007203///NM_147150 | 1.06286 |
| PAOX | NM_152911///NM_207125/// | 1.32238 |
| | NM_207126///NM_207127/// | |
| | NM_207128///NM_207129 | |
| PARP12 | NM_022750 | 1.27777 |
| PBX1 | NM_002585 | −1.08862 |
| PCDH9 | NM_020403///NM_203487 | −1.05152 |
| PCTK1 | NM_006201///NM_033018 | −0.814496 |
| PDCD2 | NM_002598///NM_144781 | −0.90548 |
| PDE4B | NM_002600 | −1.7473 |
| PDE4D | NM_006203 | −1.12303 |
| PDZK1IP1 | NM_005764 | 1.13804 |
| PEF1 | NM_012392 | −1.28292 |
| PEG10 | XM_496907///XM_499343 | −1.64969 |
| PELI1 | NM_020651 | 1.0763 |
| PER2 | NM_003894///NM_022817 | −1.64048 |
| Pfs2 | NM_016095 | −1.22956 |
| PGK1 | NM_000291 | 1.53422 |
| PHTF2 | NM_020432 | 1.08747 |
| PICALM | NM_001008660///NM_007166 | 1.1885 |
| PIK3CD | NM_005026 | 1.29341 |
| PLA2G4A | NM_024420 | −1.19118 |
| PLAT | NM_000930///NM_000931/// | 2.06312 |
| | NM_033011 | |
| PLAU | NM_002658 | 1.21635 |
| PLK1 | NM_005030 | −1.10785 |
| PLK2 | NM_006622 | 1.14877 |
| PMAIP1 | NM_021127 | 1.0331 |
| PMCH | NM_002674 | 0.725383 |
| PNMA2 | NM_007257 | 1.10051 |
| PODXL | NM_001018111///NM_005397 | 0.921137 |
| POLD1 | NM_002691 | −1.00577 |
| PON3 | NM_000940 | −1.26855 |
| PPIF | NM_005729 | 1.61265 |
| PPL | NM_002705 | 0.826009 |
| PPM1H | XM_350880 | 0.821443 |
| PPP1R11 | NM_021959///NM_170781 | −1.67093 |
| PRG1 | NM_002727 | 1.04852 |
| PRKAG2 | NM_016203 | 1.13711 |
| PR01843 | — | 0.847903 |
| PROSC | NM_007198 | −0.990835 |
| PRRG1 | NM_000950 | 1.04821 |
| PSF1 | NM_021067 | −1.54127 |
| PSMB8 | NM_004159///NM_148919 | 1.00254 |
| PSMB9 | NM_002800///NM_148954 | 1.29194 |
| PSME3 | NM_005789///NM_176863 | −1.18026 |
| PTD008 | NM_016145 | −1.07111 |
| PTENP1 | — | 0.949168 |
| PTGES | NM_004878///NM_198797 | 1.11408 |
| PTHLH | NM_002820///NM_198964/// | 1.17104 |
| | NM_198965///NM_198966 | |
| PTK9 | NM_002822///NM_198974 | 0.721157 |
| PTMS | NM_002824 | −1.31775 |
| PTPN13 | NM_006264///NM_080683/// | 1.36372 |
| | NM_080684///NM_080685 | |
| PTPRE | NM_006504///NM_130435 | 1.05644 |
| PTX3 | NM_002852 | 0.863389 |
| PYCARD | NM_013258///NM_145182/// | 1.62445 |
| | NM_145183 | |
| QDPR | NM_000320 | −0.887924 |
| QKI | NM_006775///NM_206853/// | 1.48545 |
| | NM_206854///NM_206855 | |
| R3HDM1 | NM_015361 | −1.54935 |
| RAB11FIP1 | NM_001002233///NM_001002814/// | 1.18165 |
| | NM_025151 | |
| RAB2 | NM_002865 | 1.62595 |
| RAB32 | NM_006834 | 0.740628 |
| RAB40B | NM_006822 | 1.14546 |
| RABL2B/// | NM_001003789///NM_007081/// | |
| RABL2A | NM_007082///NM_013412 | 1.00643 |
| RAFTLIN | NM_015150 | 2.59733 |
| RAI14 | NM_015577 | 1.02269 |
| RARRES3 | NM_004585 | 2.02476 |
| RASGRP1 | NM_005739 | 1.60245 |
| RASSF2 | NM_014737///NM_170773/// | 1.07132 |
| | NM_170774 | |
| RBL1 | NM_002895///NM_183404 | −0.72568 |
| RFC3 | NM_002915///NM_181558 | −1.20326 |
| RFC5 | NM_007370///NM_181578 | −0.923417 |
| RGS2 | NM_002923 | 0.835083 |
| RGS20 | NM_003702///NM_170587 | 0.993551 |
| RHEB | NM_005614 | 1.18155 |
| RHOB | NM_004040 | 0.954741 |
| RHOBTB1 | NM_001032380///NM_014836/// | 0.946447 |
| | NM_198225 | |
| RIG | — | 1.78907 |
| RIP | NM_001033002///NM_032308 | 1.2185 |
| RIT1 | NM_006912 | 1.32862 |
| RNASE4 | NM_002937///NM_194430/// | −1.4534 |
| | NM_194431 | |
| RP2 | NM_006915 | 2.06464 |
| RPL38 | NM_000999 | 1.08656 |
| RPS11 | NM_001015 | 0.858194 |
| RPS6KA5 | NM_004755///NM_182398 | 1.22551 |
| RRAD | NM_004165 | 0.849368 |
| RRAS | NM_006270 | −1.79851 |
| RRM2 | NM_001034 | −0.831449 |
| RSAD1 | NM_018346 | −0.772167 |
| S100P | NM_005980 | −0.746607 |
| SAC3D1 | NM_013299 | −1.247 |
| SAMD4 | NM_015589 | 1.21723 |
| SCML1 | NM_006746 | 0.853621 |
| SCYL3 | NM_020423///NM_181093 | 1.19418 |
| SDC1 | NM_001006946///NM_002997 | −0.818833 |
| SEC14L1 | NM_003003 | 1.44887 |
| SEC23B | NM_006363///NM_032985/// | 1.0317 |
| | NM_032986 | |
| SEC24A | XM_094581 | 1.18465 |
| SEMA3C | NM_006379 | 0.835585 |
| SERPINB9 | NM_004155 | 0.82615 |
| SERPINE1 | NM_000602 | 1.30668 |
| SERPINE2 | NM_006216 | 1.32701 |
| SGPP1 | NM_030791 | −1.67675 |
| SGSH | NM_000199 | 1.00616 |
| SH3GL1 | NM_003025 | −1.28343 |
| SHCBP1 | NM_024745 | −1.26362 |
| SHOX2 | NM_003030///NM_006884 | 0.907587 |
| SIRT1 | NM_012238 | −1.12384 |
| SLC11A2 | NM_000617 | 0.999393 |
| SLC1A1 | NM_004170 | 2.35948 |
| SLC29A1 | NM_004955 | −1.75863 |
| SLC35B1 | NM_005827 | −0.71379 |
| SLC4A4 | NM_003759 | −0.800469 |
| SLC6A6 | NM_003043 | 1.00156 |
| SLC7A11 | NM_014331 | 0.710721 |
| SLC7A5 | NM_003486 | −1.19768 |
| SLCO2B1 | NM_007256 | 1.19404 |
| SMAD3 | NM_005902 | 1.17331 |
| SMURF2 | NM_022739 | 1.68208 |
| SNX16 | NM_022133///NM_152836/// | 1.09618 |
| | NM_152837 | |
| SOD2 | NM_000636///NM_001024465/// | 1.45843 |
| | NM_001024466 | |
| SOX18 | NM_018419 | 1.41328 |
| SPARC | NM_003118 | 1.52227 |
| SPBC25 | NM_020675 | −1.4866 |
| SPFH1 | NM_006459 | −1.8131 |
| SPFH2 | NM_001003790///NM_001003791/// | 0.942632 |
| | NM_007175 | |
| SPHK1 | NM_021972///NM_182965 | 1.1223 |
| SPTBN1 | NM_003128///NM_178313 | 0.857646 |
| SQRDL | NM_021199 | 1.28491 |
| SRM | NM_003132 | −1.08855 |
| STC1 | NM_003155 | 1.03121 |
| STX3A | NM_004177 | 0.728912 |
| STYK1 | NM_018423 | 0.98547 |
| SULT1C1 | NM_001056///NM_176825 | 1.99731 |
| SUMO2 | NM_001005849///NM_006937 | 1.04086 |
| SVIL | NM_003174///NM_021738 | 1.26107 |
| SWAP70 | NM_015055 | 1.08597 |
| SYNCRIP | NM_006372 | −0.70921 |
| SYNE1 | NM_015293///NM_033071/// | 0.78963 |
| | NM_133650///NM_182961 | |
| SYT1 | NM_005639 | −1.51651 |
| TACSTD1 | NM_002354 | −1.62205 |
| TANK | NM_004180///NM_133484 | 1.19308 |
| TAPBPL | NM_018009 | 1.01656 |
| TBXAS1 | NM_001061///NM_030984 | 1.22107 |
| TDO2 | NM_005651 | 0.720423 |
| TFG | NM_001007565///NM_006070 | 0.737363 |
| TGFB2 | NM_003238 | 0.757903 |
| TGFBR2 | NM_001024847///NM_003242 | −0.760439 |
| THBD | NM_000361 | −1.03072 |
| TIMM13 | NM_012458 | −1.00078 |
| TJP2 | NM_004817///NM_201629 | 0.721283 |
| TK1 | NM_003258 | −2.0118 |
| TLR1 | NM_003263 | 2.35 |
| TLR3 | NM_003265 | 0.972191 |
| TM4SF20 | NM_024795 | −1.36784 |
| TM4SF4 | NM_004617 | −1.87733 |
| TM7SF1 | NM_003272 | 1.42643 |
| TMEM45A | NM_018004 | −1.31309 |
| TMEM48 | NM_018087 | −1.55691 |
| TMF1 | NM_007114 | −0.791138 |
| TMOD1 | NM_003275 | 1.92937 |
| TNC | NM_002160 | 1.22931 |
| TNFAIP3 | NM_006290 | 0.835162 |
| TNFAIP6 | NM_007115 | 3.25281 |
| TNFRSF9 | NM_001561 | 0.806509 |
| TNRC9 | XM_049037 | −0.835259 |
| TOP1 | NM_003286 | 0.756531 |
| TP53I3 | NM_004881///NM_147184 | 1.07792 |
| TPD52 | NM_001025252///NM_001025253/// | −2.00612 |
| | NM_005079 | |
| TPI1 | NM_000365 | −0.72538 |
| TPM1 | NM_000366///NM_001018004/// | 1.27399 |
| | NM_001018005///NM_001018006/// | |
| | NM 001018007// | |
| TRA1 | NM_003299 | 1.71538 |
| TRIM14 | NM_014788///NM_033219/// | −1.15248 |
| | NM_033220///NM_033221 | |
| TRIM22 | NM_006074 | 2.11688 |
| TRIM8 | NM_030912 | 1.36446 |
| TRIO | NM_007118 | 1.05084 |
| TRPA1 | NM_007332 | 1.71335 |
| TRPC1 | NM_003304 | 0.703632 |
| TSC22D3 | NM_0010158///NM_004089/// | 1.09737 |
| | NM_198057 | |
| TSN | NM_004622 | −1.13575 |
| TSPAN7 | NM_004615 | 1.43844 |
| TTC10 | NM_006531///NM_175605 | 1.19076 |
| TTMP | NM_024616 | 1.49839 |
| TTRAP | NM_016614 | 0.977696 |
| TUBB | NM_178014 | −1.04629 |
| TUBB2 | NM_001069 | 1.31933 |
| TUBB- | NM_178012 | 1.42413 |
| PARALOG | | |
| TXN | NM_003329 | 1.56098 |
| UBE2H | NM_003344///NM_182697 | 1.12195 |
| UBE2L3 | NM_003347///NM_198157 | −1.00846 |
| UBE2L6 | NM_004223///NM_198183 | 1.33829 |
| UGCG | NM_003358 | 1.01016 |
| UROS | NM_000375 | −1.09209 |
| USP46 | NM_022832 | 0.730964 |
| VDAC3 | NM_005662 | 1.19978 |
| VIL2 | NM_003379 | 0.951191 |
| VLDLR | NM_001018056///NM_003383 | 1.49472 |
| VPS4A | NM_013245 | −1.3102 |
| WDR19 | NM_025132 | 1.86855 |
| WDR47 | NM_014969 | 1.27531 |
| WDR76 | NM_024908 | −1.09373 |
| WHSC1 | NM_007331///NM_014919/// | −0.795359 |
| | NM_133330///NM_133331/// | |
| | NM_133332///NM_133333 | |
| WIPI49 | NM_017983 | 1.16833 |
| WIZ | XM_372716 | −0.911496 |
| WNT7B | NM_058238 | −0.755357 |
| XBP1 | NM_005080 | −1.02439 |
| XTP2 | NM_015172 | 1.01515 |
| YKT6 | NM_006555 | −1.12573 |
| YOD1 | NM_018566 | 1.13406 |
| YRDC | NM_024640 | 0.717093 |
| ZBTB10 | NM_023929 | 0.894651 |
| ZFHX1B | NM_014795 | 1.19961 |
| ZFYVE21 | NM_024071 | 0.815726 |
| ZMYM6 | NM_007167 | 0.920391 |
| ZNF22 | NM_006963 | −1.21289 |
| ZNF232 | NM_014519 | −1.35052 |
| ZNF238 | NM_006352///NM_205768 | 1.09124 |
| ZNF281 | NM_012482 | −0.825036 |
| ZNF331 | NM_018555 | −1.18107 |
| ZNF544 | NM_014480 | −1.54 |
| ZNF551 | NM_138347 | −1.26671 |
| ZNF573 | NM_152360 | −0.794295 |
| ZNF580 | NM_016202///NM_207115 | −1.90207 |
| ZNF652 | NM_014897 | 0.911137 |

A further embodiment of the invention is directed to methods of modulating a cellular pathway comprising administering to the cell an amount of an isolated nucleic acid comprising a miR-34 nucleic acid sequence or a miR-34 inhibitor. A cell, tissue, or subject may be a cancer cell, a cancerous tissue or harbor cancerous tissue, or a cancer patient. The database content related to all nucleic acids and genes designated by an accession number or a database submission are incorporated herein by reference as of the filing date of this application.

A further embodiment of the invention is directed to methods of modulating a cellular pathway comprising administering to the cell an amount of an isolated nucleic acid comprising a nucleic acid sequence in an amount sufficient to modulate the expression, function, status, or state of a cellular pathway, in particular those pathways described in Table 2 or the pathways known to include one or more genes from Table 1, 3, 4, and/or 5. Modulation of a cellular pathway includes, but is not limited to modulating the expression of one or more gene(s). Modulation of a gene can include inhibiting the function of an endogenous miRNA or providing a functional miRNA to a cell, tissue, or subject. Modulation refers to the expression levels or activities of a gene or its related gene product (e.g., mRNA) or protein, e.g., the mRNA levels may be modulated or the translation of an mRNA may be modulated. Modulation may increase or up regulate a gene or gene product or it may decrease or down regulate a gene or gene product (e.g., protein levels or activity).

Still a further embodiment includes methods of administering an miRNA or mimic thereof, and/or treating a subject or patient having, suspected of having, or at risk of developing a pathological condition comprising one or more of step (a) administering to a patient or subject an amount of an isolated nucleic acid comprising a miR-34 nucleic acid sequence or a miR-34 inhibitor in an amount sufficient to modulate expression of a cellular pathway; and (b) administering a second therapy, wherein the modulation of the cellular pathway sensitizes the patient or subject, or increases the efficacy of a second therapy. An increase in efficacy can include a reduction in toxicity, a reduced dosage or duration of the second therapy, or an additive or synergistic effect. A cellular pathway may include, but is not limited to one or more pathway described in Table 2 below or a pathway that is know to include one or more genes of Tables 1, 3, 4, and/or 5. The second therapy may be administered before, during, and/or after the isolated nucleic acid or miRNA or inhibitor is administered

A second therapy can include administration of a second miRNA or therapeutic nucleic acid such as a siRNA or antisense oligonucleotide, or may include various standard therapies, such as pharmaceuticals, chemotherapy, radiation therapy, drug therapy, immunotherapy, and the like. Embodiments of the invention may also include the determination or assessment of gene expression or gene expression profile for the selection of an appropriate therapy. In a particular aspect, a second therapy is a chemotherapy. A chemotherapy can include, but is not limited to paclitaxel, cisplatin, carboplatin, doxorubicin, oxaliplatin, larotaxel, taxol, lapatinib, docetaxel, methotrexate, capecitabine, vinorelbine, cyclophosphamide, gemcitabine, amrubicin, cytarabine, etoposide, camptothecin, dexamethasone, dasatinib, tipifamib, bevacizumab, sirolimus, temsirolimus, everolimus, lonafarnib, cetuximab, erlotinib, gefitinib, imatinib mesylate, rituximab, trastuzumab, nocodazole, sorafenib, sunitinib, bortezomib, alemtuzumab, gemtuzumab, tositumomab or ibritumomab.

Embodiments of the invention include methods of treating a subject with a disease or condition comprising one or more of the steps of (a) determining an expression profile of one or more genes selected from Table 1, 3, 4, and/or 5; (b) assessing the sensitivity of the subject to therapy based on the expression profile; (c) selecting a therapy based on the assessed sensitivity; and (d) treating the subject using a selected therapy. Typically, the disease or condition will have as a component, indicator, or resulting mis-regulation of one or more gene of Table 1, 3, 4, and/or 5.

In certain aspects, 2, 3, 4, 5, 6, 7, 8, 9, 10, or more miRNA may be used in sequence or in combination; for instance, any combination of miR-34 or a miR-34 inhibitor with another miRNA. Further embodiments include the identification and assessment of an expression profile indicative of miR-34 status in a cell or tissue comprising expression assessment of one or more gene from Table 1, 3, 4, and/or 5, or any combination thereof.

The term “miRNA” is used according to its ordinary and plain meaning and refers to a microRNA molecule found in eukaryotes that is involved in RNA-based gene regulation. See, e.g., Carrington and Ambros, 2003, which is hereby incorporated by reference. The term can be used to refer to the single-stranded RNA molecule processed from a precursor or in certain instances the precursor itself.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker or miRNA in the sample (e.g., a plurality of nucleic acid probes that identify one or more markers from Tables 1, 3, 4, and/or 5); it is contemplated that the nucleic acid profile can be obtained using a set of RNAs, using for example nucleic acid amplification or hybridization techniques well know to one of ordinary skill in the art. The difference in the expression profile in the sample from the patient and a reference expression profile, such as an expression profile of one or more genes or miRNAs, are indicative of which miRNAs to be administered.

In certain aspects, miR-34 or miR-34 inhibitor and let-7 can be administered to patients with breast carcinoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, non-small cell lung carcinoma, ovarian carcinoma, oesophageal carcinoma, pancreatic carcinoma, prostate carcinoma, squamous cell carcinoma of the head and neck, thyroid carcinoma.

Further aspects include administering miR-34 or miR-34 inhibitor and miR-15 to patients with breast carcinoma, B-cell lymphoma, cervical carcinoma, colorectal carcinoma, glioma, glioblastoma, gastric carcinoma, hepatocellular carcinoma, lung carcinoma, multiple myeloma, non-small cell lung carcinoma, ovarian carcinoma, oesophageal carcinoma, pancreatic carcinoma, prostate carcinoma, rhabdomyosarcoma, squamous cell carcinoma of the head and neck, thyroid carcinoma.

In still further aspects, miR-34 or miR-34 inhibitor and miR-16 are administered to patients with breast carcinoma, B-cell lymphoma, colorectal carcinoma, glioblastoma, gastric carcinoma, hepatocellular carcinoma, multiple myeloma, non-small cell lung carcinoma, ovarian carcinoma, oesophageal carcinoma, pancreatic carcinoma, prostate carcinoma, rhabdomyosarcoma, squamous cell carcinoma of the head and neck, thyroid carcinoma.

In certain aspects, miR-34 or miR-34 inhibitor and miR-20 are administered to patients with breast carcinoma, cervical carcinoma, colorectal carcinoma, glioma, glioblastoma, gastric carcinoma, hepatocellular carcinoma, leukemia, lipoma, multiple myeloma, non-small cell lung carcinoma, ovarian carcinoma, oesophageal carcinoma, osteosarcoma, pancreatic carcinoma, prostate carcinoma, squamous cell carcinoma of the head and neck, thyroid carcinoma.

Aspects of the invention include methods where miR-34 or miR-34 inhibitor and miR-21 are administered to patients with breast carcinoma, colorectal carcinoma, glioma, glioblastoma, gastric carcinoma, hepatocellular carcinoma, non-small cell lung carcinoma, ovarian carcinoma, oesophageal carcinoma, pancreatic carcinoma, prostate carcinoma, rhabdomyosarcoma, squamous cell carcinoma of the head and neck.

In still further aspects, miR-34 or miR-34 inhibitor and miR-26a are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, non-small cell lung carcinoma, ovarian carcinoma, oesophageal carcinoma, osteosarcoma, pancreatic carcinoma, prostate carcinoma, rhabdomyosarcoma, testicular tumor.

In yet further aspects, miR-34 or miR-34 inhibitor and miR-126 are administered to patients with breast carcinoma, cervical carcinoma, colorectal carcinoma, glioma, glioblastoma, gastric carcinoma, hepatocellular carcinoma, leukemia, lung carcinoma, mesothelioma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma.

In a further aspect, miR-34 or miR-34 inhibitor and miR-143 are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, non-small cell lung carcinoma, ovarian carcinoma, oesophageal carcinoma, osteosarcoma, pancreatic carcinoma, prostate carcinoma, squamous cell carcinoma of the head and neck, thyroid carcinoma, testicular tumor.

In still a further aspect, miR-34 or miR-34 inhibitor and miR-147 are administered to patients with breast carcinoma, cervical carcinoma, colorectal carcinoma, glioma, glioblastoma, gastric carcinoma, hepatocellular carcinoma, leukemia, lipoma, multiple myeloma, non-small cell lung carcinoma, ovarian carcinoma, oesophageal carcinoma, osteosarcoma, pancreatic carcinoma, prostate carcinoma, squamous cell carcinoma of the head and neck, thyroid carcinoma.

In yet another aspect, miR-34 or miR-34 inhibitor and miR-188 are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, non-small cell lung carcinoma, ovarian carcinoma, esophageal carcinoma, pancreatic carcinoma, prostate carcinoma, squamous cell carcinoma of the head and neck, thyroid carcinoma, testicular tumor.

In yet a further aspect, miR-34 or miR-34 inhibitor and miR-200 are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, mesothelioma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, testicular tumor.

In other aspects, miR-34 or miR-34 inhibitor and miR-215 are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, lipoma, multiple myeloma, mesothelioma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, testicular tumor.

In certain aspects, miR-34 or miR-34 inhibitor and miR-216 are administered to patients with breast carcinoma, cervical carcinoma, colorectal carcinoma, glioma, glioblastoma, gastric carcinoma, hepatocellular carcinoma, leukemia, lung carcinoma, non-small cell lung carcinoma, ovarian carcinoma, oesophageal carcinoma, osteosarcoma, prostate carcinoma, squamous cell carcinoma of the head and neck, testicular tumor.

In a further aspect, miR-34 or miR-34 inhibitor and miR-292-3p are administered to patients with anaplastic large cell lymphoma, breast carcinoma, B-cell lymphoma, cervical carcinoma, colorectal carcinoma, glioma, glioblastoma, gastric carcinoma, hepatocellular carcinoma, leukemia, lung carcinoma, lipoma, multiple myeloma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, testicular tumor.

In still a further aspect, miR-34 or miR-34 inhibitor and miR-331 are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, testicular tumor.

It is contemplated that when miR-34 or a miR-34 inhibitor is given in combination with one or more other miRNA molecules, the two different miRNAs or inhibitors may be given at the same time or sequentially. In some embodiments, therapy proceeds with one miRNA or inhibitor and that therapy is followed up with therapy with the other miRNA or inhibitor 1, 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, or 1, 2, 3, 4, 5, 6, 7, 8, 9, 10, 11, or 12 months or any such combination later.

Further embodiments include the identification and assessment of an expression profile indicative of miR-34 status in a cell or tissue comprising expression assessment of one or more gene from Table 1, 3, 4, and/or 5, or any combination thereof.

The term “miRNA” is used according to its ordinary and plain meaning and refers to a microRNA molecule found in eukaryotes that is involved in RNA-based gene regulation. See, e.g., Carrington and Ambros, 2003, which is hereby incorporated by reference. The term can be used to refer to the single-stranded RNA molecule processed from a precursor or in certain instances the precursor itself or a mimetic thereof.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more miRNA marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers or genes from Tables 1, 3, 4, and/or 5); it is contemplated that the nucleic acid profile can be obtained using a set of RNAs, using for example nucleic acid amplification or hybridization techniques well know to one of ordinary skill in the art. The difference in the expression profile in the sample from a patient and a reference expression profile, such as an expression profile from a normal or non-pathologic sample, or a digitized reference, is indicative of a pathologic, disease, or cancerous condition. In certain aspects the expression profile is an indicator of a propensity to or probability of (i.e., risk factor for a disease or condition) developing such a condition(s). Such a risk or propensity may indicate a treatment, increased monitoring, prophylactic measures, and the like. A nucleic acid or probe set may comprise or identify a segment of a corresponding mRNA and may include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 100, 200, 500, or more segments, including any integer or range derivable there between, of a gene or genetic marker, or a nucleic acid, mRNA or a probe representative thereof that is listed in Tables 1, 3, 4, and/or 5 or identified by the methods described herein.

Certain embodiments of the invention are directed to compositions and methods for assessing, prognosing, or treating a pathological condition in a patient comprising measuring or determining an expression profile of one or more miRNA or marker(s) in a sample from the patient, wherein a difference in the expression profile in the sample from the patient and an expression profile of a normal sample or reference expression profile is indicative of pathological condition and particularly cancer (e.g., In certain aspects of the invention, the miRNAs, cellular pathway, gene, or genetic marker is or is representative of one or more pathway or marker described in Table 1, 2, 3, 4, and/or 5, including any combination thereof.

Aspects of the invention include diagnosing, assessing, or treating a pathologic condition or preventing a pathologic condition from manifesting. For example, the methods can be used to screen for a pathological condition; assess prognosis of a pathological condition; stage a pathological condition; assess response of a pathological condition to therapy; or to modulate the expression of a gene, genes, or related pathway as a first therapy or to render a subject sensitive or more responsive to a second therapy. In particular aspects, assessing the pathological condition of the patient can be assessing prognosis of the patient. Prognosis may include, but is not limited to an estimation of the time or expected time of survival, assessment of response to a therapy, and the like. In certain aspects, the altered expression of one or more gene or marker is prognostic for a patient having a pathologic condition, wherein the marker is one or more of Table 1, 3, 4, and/or 5, including any combination thereof.

TABLE 2
Significantly affected functional cellular pathways
following hsa-miR-34a over-expression in human cancer cells.

| Number | |
|---|---|
| Of | |
| Genes | Pathway Functions |
| 35 | Cellular Growth and Proliferation, Cellular Movement, |
| | Cell Death |
| 35 | Gene Expression, Cellular Growth and Proliferation, Cell Death |
| 25 | Gene Expression, DNA Replication, Recombination, and Repair, |
| | Cell Cycle |
| 23 | DNA Replication, Recombination, and Repair, Cell Cycle, |
| | Cellular Development |
| 19 | Cardiovascular Disease, Hematological Disease, Organismal |
| | Injury and Abnormalities |
| 19 | Cancer, Cell Cycle, Hepatic System Disease |
| 19 | Immune Response, Cell Signaling, Molecular Transport |
| 18 | Cancer, Cellular Growth and Proliferation, Neurological Disease |
| 17 | Immune Response, Cellular Movement, Hematological System |
| | Development and Function |
| 17 | Lipid Metabolism, Molecular Transport, Small Molecule |
| | Biochemistry |
| 17 | Cell Cycle, Cancer, Cellular Growth and Proliferation |
| 16 | Cell-To-Cell Signaling and Interaction, Cellular Movement, |
| | Hematological System Development and Function |
| 16 | Cellular Movement, Cellular Development, Cardiovascular |
| | System Development and Function |
| 15 | Organ Development, Gene Expression, Developmental Disorder |
| 15 | Cell Death, Cancer, Cellular Growth and Proliferation |
| 15 | Carbohydrate Metabolism, Small Molecule Biochemistry, |
| | Lipid Metabolism |
| 15 | Cellular Assembly and Organization, Cell Cycle, |
| | Connective Tissue Development and Function |
| 15 | DNA Replication, Recombination, and Repair, Gene Expression, |
| | Cancer |
| 14 | Hematological System Development and Function, Immune |
| | Response, Immune and Lymphatic System Development and |
| | Function |
| 14 | Protein Synthesis, Cell Signaling, Nucleic Acid Metabolism |
| 7 | Cell Death, Neurological Disease, Cellular Development |
| 1 | Cellular Assembly and Organization, Cell Morphology, |
| | Cellular Compromise |
| 1 | Cell Cycle, Cellular Assembly and Organization, |
| | DNA Replication, Recombination, and Repair |
| 1 | Cancer, Cell Death, Reproductive System Disease |
| 1 | Amino Acid Metabolism, Molecular Transport, Small |
| | Molecule Biochemistry |
| 1 | Cell Cycle, Cancer, Cell Death |
| 1 | Cell Death |
| 1 | Cellular Compromise, Auditory and Vestibular |
| | System Development and Function, Protein Trafficking |
| 1 | Cell Morphology, Cellular Assembly and Organization, |
| | Cellular Compromise |
| 1 | Cellular Assembly and Organization, Cell Morphology, |
| | Molecular Transport |
| 1 | Cardiovascular System Development and Function, Organ |
| | Morphology, Neurological Disease |
| 1 | Cellular Assembly and Organization, Cell Morphology, |
| | Cellular Function and Maintenance |
| 1 | Cell Signaling, Molecular Transport, Neurological Disease |

TABLE 3
Predicted target genes of hsa-miR-34a.

| | Ref Seq | |
|---|---|---|
| | Transcript ID | |
| Gene Symbol | (Pruitt et al., 2005) | Description |
| A1BG | NM_130786 | alpha 1B-glycoprotein |
| AADACL1 | NM_020792 | arylacetamide deacetylase-like 1 |
| AASDHPPT | NM_015423 | aminoadipate-semialdehyde |
| ABCA1 | NM_005502 | ATP-binding cassette, sub-family A member 1 |
| ABCC1 | NM_004996 | ATP-binding cassette, sub-family C, member 1 |
| ABCC12 | NM_033226 | ATP-binding cassette protein C12 |
| ABCC13 | NM_172024 | ATP-binding cassette protein C13 isoform b |
| ABCC4 | NM_005845 | ATP-binding cassette, sub-family C, member 4 |
| ABCC5 | NM_005688 | ATP-binding cassette, sub-family C, member 5 |
| ABCD1 | NM_000033 | ATP-binding cassette, sub-family D (ALD), member |
| ABCE1 | NM_002940 | ATP-binding cassette, sub-family E, member 1 |
| ABCF2 | NM_007189 | ATP-binding cassette, sub-family F, member 2 |
| ABCF3 | NM_018358 | ATP-binding cassette, sub-family F (GCN20), |
| ABCG4 | NM_022169 | ATP-binding cassette, sub-family G, member 4 |
| ABHD12 | NM_015600 | abhydrolase domain containing 12 |
| ABHD4 | NM_022060 | abhydrolase domain containing 4 |
| ABI3 | NM_016428 | NESH protein |
| ABL1 | NM_005157 | v-abl Abelson murine leukemia viral oncogene |
| ABLIM1 | NM_001003407 | actin-binding LIM protein 1 isoform b |
| ABLIM3 | NM_014945 | actin binding LIM protein family, member 3 |
| ABR | NM_001092 | active breakpoint cluster region-related |
| ACACA | NM_198834 | acetyl-Coenzyme A carboxylase alpha isoform 1 |
| ACAD11 | NM_032169 | putative acyl-CoA dehydrogenase |
| ACAD8 | NM_014384 | acyl-Coenzyme A dehydrogenase family, member 8 |
| ACADL | NM_001608 | acyl-Coenzyme A dehydrogenase, long chain |
| ACADS | NM_000017 | acyl-Coenzyme A dehydrogenase, C-2 to C-3 short |
| ACADSB | NM_001609 | acyl-Coenzyme A dehydrogenase, short/branched |
| ACADVL | NM_000018 | acyl-Coenzyme A dehydrogenase, very long chain |
| ACBD3 | NM_022735 | acyl-Coenzyme A binding domain containing 3 |
| ACCN1 | NM_001094 | amiloride-sensitive cation channel 1, neuronal |
| ACE | NM_152831 | angiotensin I converting enzyme isoform 3 |
| ACOT11 | NM_147161 | thioesterase, adipose associated isoform BFIT2 |
| ACP5 | NM_001611 | tartrate resistant acid phosphatase 5 precursor |
| ACPP | NM_001099 | prostatic acid phosphatase precursor |
| ACPT | NM_080789 | testicular acid phosphatase isoform b precursor |
| ACSL1 | NM_001995 | acyl-CoA synthetase long-chain family member I |
| ACSL3 | NM_004457 | acyl-CoA synthetase long-chain family member 3 |
| ACSL4 | NM_004458 | acyl-CoA synthetase long-chain family member 4 |
| ACSS2 | NM_018677 | acyl-CoA synthetase short-chain family member 2 |
| ACTBL1 | NM_001004053 | protein expressed in prostate, ovary, testis, |
| ACTL6A | NM_004301 | actin-like 6A isoform 1 |
| ACTL8 | NM_030812 | actin like protein |
| ACTN2 | NM_001103 | actinin, alpha 2 |
| ACTN4 | NM_004924 | actinin, alpha 4 |
| ACTR1A | NM_005736 | ARP1 actin-related protein 1 homolog A, |
| ACTR5 | NM_024855 | ARP5 actin-related protein 5 homolog |
| ACTR8 | NM_022899 | actin-related protein 8 |
| ACVR1B | NM_004302 | activin A type IB receptor isoform a precursor |
| ADAM10 | NM_001110 | ADAM metallopeptidase domain 10 |
| ADAM11 | NM_002390 | ADAM metallopeptidase domain 11 preproprotein |
| ADAM12 | NM_003474 | ADAM metallopeptidase domain 12 isoform 1 |
| ADAM19 | NM_033274 | ADAM metallopeptidase domain 19 isoform 2 |
| ADAMTS1 | NM_006988 | ADAM metallopeptidase with thrombospondin type 1 |
| ADAMTS10 | NM_030957 | ADAM metallopeptidase with thrombospondin type 1 |
| ADAMTS4 | NM_005099 | ADAM metallopeptidase with thrombospondin type 1 |
| ADAMTSL1 | NM_139264 | ADAMTS-like 1 isoform 3 |
| ADAMTSL4 | NM_019032 | thrombospondin repeat containing 1 isoform 1 |
| ADAT1 | NM_012091 | adenosine deaminase, tRNA-specific 1 |
| ADCY1 | NM_021116 | brain adenylate cyclase 1 |
| ADCY2 | NM_020546 | adenylate cyclase 2 |
| ADCY7 | NM_001114 | adenylate cyclase 7 |
| ADD2 | NM_001617 | adducin 2 isoform a |
| ADIPOQ | NM_004797 | adiponectin precursor |
| ADIPOR2 | NM_024551 | adiponectin receptor 2 |
| ADK | NM_001123 | adenosine kinase isoform a |
| ADM2 | NM_024866 | adrenomedullin 2 precusor |
| ADNP | NM_015339 | activity-dependent neuroprotector |
| ADORA2A | NM_000675 | adenosine A2a receptor |
| ADPN | NM_025225 | adiponutrin |
| ADPRH | NM_001125 | ADP-ribosylarginine hydrolase |
| ADRA1A | NM_033302 | alpha-1A-adrenergic receptor isoform 3 |
| ADRA1D | NM_000678 | alpha-1D-adrenergic receptor |
| ADRA2A | NM_000681 | alpha-2A-adrenergic receptor |
| ADRA2B | NM_000682 | alpha-2B-adrenergic receptor |
| ADRBK2 | NM_005160 | beta adrenergic receptor kinase 2 |
| AFAP | NM_021638 | actin filament associated protein |
| AFF2 | NM_002025 | fragile X mental retardation 2 |
| AFF3 | NM_001025108 | AF4/FMR2 family, member 3 isoform 2 |
| AFF4 | NM_014423 | ALL1 fused gene from 5q31 |
| AFG3L1 | NM_001031805 | AFG3 ATPase family gene 3-like 1 isoform 2 |
| AGTR1 | NM_000685 | angiotensin II receptor, type 1 |
| AGTRAP | NM_020350 | angiotensin II receptor-associated protein |
| AHNAK | NM_001620 | AHNAK nucleoprotein isoform 1 |
| AIPL1 | NM_001033054 | aryl hydrocarbon receptor interacting |
| AJAP1 | NM_018836 | transmembrane protein SHREW1 |
| AK2 | NM_013411 | adenylate kinase 2 isoform b |
| AK3 | NM_016282 | adenylate kinase 3 |
| AKAP1 | NM_139275 | A-kinase anchor protein 1 isoform 2 precursor |
| AKAP13 | NM_006738 | A-kinase anchor protein 13 isoform 1 |
| AKAP6 | NM_004274 | A-kinase anchor protein 6 |
| AKAP7 | NM_004842 | A-kinase anchor protein 7 isoform alpha |
| AKR1CL1 | NM_001007536 | aldo-keto reductase family 1, member C-like 1 |
| ALAD | NM_000031 | delta-aminolevulinic acid dehydratase isoform b |
| ALCAM | NM_001627 | activated leukocyte cell adhesion molecule |
| ALDH1A2 | NM_003888 | aldehyde dehydrogenase 1A2 isoform 1 |
| ALDH1A3 | NM_000693 | aldehyde dehydrogenase 1A3 |
| ALDH3B2 | NM_000695 | aldehyde dehydrogenase 3B2 |
| ALDH5A1 | NM_001080 | aldehyde dehydrogenase 5A1 precursor, isoform 2 |
| ALDH6A1 | NM_005589 | aldehyde dehydrogenase 6A1 precursor |
| ALDOA | NM_000034 | aldolase A |
| ALF | NM_172196 | TFIIA-alpha/beta-like factor isoform 2 |
| ALG1 | NM_019109 | beta-1,4-mannosyltransferase |
| ALG12 | NM_024105 | asparagine-linked glycosylation 12 |
| ALOX5 | NM_000698 | arachidonate 5-lipoxygenase |
| ALS2CL | NM_147129 | ALS2 C-terminal like isoform 1 |
| ALS2CR13 | NM_173511 | amyotrophic lateral sclerosis 2 (juvenile) |
| ALS2CR15 | NM_138468 | Ica69-related protein |
| ALX3 | NM_006492 | aristaless-like homeobox 3 |
| AMACR | NM_014324 | alpha-methylacyl-CoA racemase isoform 1 |
| AMD1 | NM_001033059 | S-adenosylmethionine decarboxylase 1 isoform 2 |
| AMID | NM_032797 | apoptosis-inducing factor (AIF)-like |
| AMMECR1 | NM_001025580 | AMMECR1 protein isoform 2 |
| AMOTL2 | NM_016201 | angiomotin like 2 |
| AMPD2 | NM_004037 | adenosine monophosphate deaminase 2 (isoform L) |
| AMPD3 | NM_000480 | erythrocyte adenosine monophosphate deaminase |
| AMZ1 | NM_133463 | archaemetzincin-1 |
| ANGEL1 | NM_015305 | angel homolog 1 |
| ANGPTL7 | NM_021146 | angiopoietin-like 7 |
| ANK2 | NM_001148 | ankyrin 2 isoform 1 |
| ANK3 | NM_001149 | ankyrin 3 isoform 2 |
| ANKFY1 | NM_016376 | ankyrin repeat and FYVE domain containing 1 |
| ANKRD1 | NM_014391 | cardiac ankyrin repeat protein |
| ANKRD10 | NM_017664 | ankyrin repeat domain 10 |
| ANKRD12 | NM_015208 | ankyrin repeat domain 12 |
| ANKRD13 | NM_033121 | ankyrin repeat domain 13 |
| ANKRD17 | NM_032217 | ankyrin repeat domain protein 17 isoform a |
| ANKRD23 | NM_144994 | diabetes related ankyrin repeat protein |
| ANKRD25 | NM_015493 | ankyrin repeat domain 25 |
| ANKS1A | NM_015245 | ankyrin repeat and sterile alpha motif domain |
| ANKS1B | NM_181670 | cajalin 2 isoform b |
| ANKS6 | NM_173551 | sterile alpha motif domain containing 6 |
| ANP32A | NM_006305 | acidic (leucine-rich) nuclear phosphoprotein 32 |
| ANP32B | NM_006401 | acidic (leucine-rich) nuclear phosphoprotein 32 |
| ANTXR1 | NM_032208 | tumor endothelial marker 8 isoform 1 precursor |
| ANXA11 | NM_001157 | annexin A11 |
| ANXA5 | NM_001154 | annexin 5 |
| AP1B1 | NM_001127 | adaptor-related protein complex 1 beta 1 subunit |
| AP1G1 | NM_001030007 | adaptor-related protein complex 1, gamma 1 |
| AP1GBP1 | NM_007247 | AP1 gamma subunit binding protein 1 isoform 1 |
| AP1S2 | NM_003916 | adaptor-related protein complex 1 sigma 2 |
| AP2S1 | NM_004069 | adaptor-related protein complex 2, sigma 1 |
| AP3M1 | NM_012095 | adaptor-related protein complex 3, mu 1 subunit |
| AP3M2 | NM_006803 | adaptor-related protein complex 3, mu 2 subunit |
| AP3S2 | NM_005829 | adaptor-related protein complex 3, sigma 2 |
| AP4S1 | NM_007077 | adaptor-related protein complex 4, sigma 1 |
| APBA1 | NM_001163 | amyloid beta A4 precursor protein-binding, |
| APBB3 | NM_133175 | amyloid beta precursor protein-binding, family |
| APH1A | NM_016022 | anterior pharynx defective 1 homolog A |
| APITD1 | NM_199294 | apoptosis-inducing, TAF9-like domain 1 isoform |
| APLP2 | NM_001642 | amyloid beta (A4) precursor-like protein 2 |
| APOB | NM_000384 | apolipoprotein B precursor |
| APOLD1 | NM_030817 | apolipoprotein L domain containing 1 |
| APPBP2 | NM_006380 | amyloid beta precursor protein-binding protein |
| AQP1 | NM_198098 | aquaporin 1 |
| AQP10 | NM_080429 | aquaporin 10 |
| AQP3 | NM_004925 | aquaporin 3 |
| AQP8 | NM_001169 | aquaporin 8 |
| AREG | NM_001657 | amphiregulin preproprotein |
| ARF3 | NM_001659 | ADP-ribosylation factor 3 |
| ARFGAP3 | NM_014570 | ADP-ribosylation factor GTPase activating |
| ARFGEF2 | NM_006420 | ADP-ribosylation factor guanine |
| ARG2 | NM_001172 | arginase, type II precursor |
| ARHGAP1 | NM_004308 | Rho GTPase activating protein 1 |
| ARHGAP19 | NM_032900 | Rho GTPase activating protein 19 |
| ARHGAP26 | NM_015071 | GTPase regulator associated with the focal |
| ARHGAP29 | NM_004815 | PTPL1-associated RhoGAP 1 |
| ARHGAP30 | NM_001025598 | Rho GTPase activating protein 30 isoform 1 |
| ARHGDIB | NM_001175 | Rho GDP dissociation inhibitor (GDI) beta |
| ARHGEF10L | NM_001011722 | Rho guanine nucleotide exchange factor (GEF) |
| ARHGEF12 | NM_015313 | Rho guanine nucleotide exchange factor (GEF) 12 |
| ARHGEF2 | NM_004723 | rho/rac guanine nucleotide exchange factor 2 |
| ARHGEF3 | NM_019555 | Rho guanine nucleotide exchange factor 3 |
| ARHGEF4 | NM_032995 | Rho guanine nucleotide exchange factor 4 isoform |
| ARHGEF5 | NM_001002861 | rho guanine nucleotide exchange factor 5 isoform |
| ARHGEF6 | NM_004840 | Rac/Cdc42 guanine nucleotide exchange factor 6 |
| ARHGEF7 | NM_003899 | Rho guanine nucleotide exchange factor 7 isoform |
| ARHGEF9 | NM_015185 | Cdc42 guanine exchange factor 9 |
| ARID2 | NM_152641 | AT rich interactive domain 2 (ARID, RFX-like) |
| ARID3B | NM_006465 | AT rich interactive domain 3B (BRIGHT-like) |
| ARID4A | NM_002892 | retinoblastoma-binding protein 1 isoform I |
| ARID4B | NM_016374 | AT rich interactive domain 4B isoform 1 |
| ARID5A | NM_006673 | AT rich interactive domain 5A isoform 2 |
| ARIH2 | NM_006321 | ariadne homolog 2 |
| ARL4C | NM_005737 | ADP-ribosylation factor-like 4C |
| ARL5B | NM_178815 | ADP-ribosylation factor-like 8 |
| ARL6IP4 | NM_001002252 | SRp25 nuclear protein isoform 4 |
| ARL8A | NM_138795 | ADP-ribosylation factor-like 10B |
| ARL8B | NM_018184 | ADP-ribosylation factor-like 10C |
| ARMC5 | NM_024742 | armadillo repeat containing 5 |
| ARMC6 | NM_033415 | armadillo repeat containing 6 |
| ARMC7 | NM_024585 | armadillo repeat containing 7 |
| ARMC8 | NM_015396 | armadillo repeat containing 8 isoform 2 |
| ARMCX4 | NM_152583 | hypothetical protein LOC158947 |
| ARPC5 | NM_005717 | actin related protein 2/3 complex subunit 5 |
| ARPP-19 | NM_006628 | cyclic AMP phosphoprotein, 19 kD |
| ARPP-21 | NM_001025068 | cyclic AMP-regulated phosphoprotein, 21 kD |
| ARRDC3 | NM_020801 | arrestin domain containing 3 |
| ARSB | NM_000046 | arylsulfatase B isoform 1 precursor |
| ARSJ | NM_024590 | arylsulfatase J |
| ARTS-1 | NM_016442 | type 1 tumor necrosis factor receptor shedding |
| ARVP6125 | NM_001030078 | hypothetical protein LOC442092 |
| ARX | NM_139058 | aristaless related homeobox |
| AS3MT | NM_020682 | arsenic (+3 oxidation state) methyltransferase |
| ASB1 | NM_016114 | ankyrin repeat and SOCS box-containing protein |
| ASB13 | NM_024701 | ankyrin repeat and SOCS box-containing protein |
| ASB5 | NM_080874 | ankyrin repeat and SOCS box-containing protein |
| ASB6 | NM_017873 | ankyrin repeat and SOCS box-containing 6 isoform |
| ASCIZ | NM_015251 | ATM/ATR-Substrate Chk2-Interacting Zn2+-finger |
| ASCL1 | NM_004316 | achaete-scute complex homolog-like 1 |
| ASH2L | NM_004674 | ash2 (absent, small, or homeotic)-like |
| ASTN | NM_004319 | astrotactin isoform 1 |
| ASXL1 | NM_015338 | additional sex combs like 1 |
| ASXL2 | NM_018263 | additional sex combs like 2 |
| ATG4B | NM_013325 | APG4 autophagy 4 homolog B isoform a |
| ATG5 | NM_004849 | APG5 autophagy 5-like |
| ATG9A | NM_024085 | APG9 autophagy 9-like 1 |
| ATM | NM_000051 | ataxia telangiectasia mutated protein isoform 1 |
| ATP13A1 | NM_020410 | ATPase type 13A1 |
| ATP1A2 | NM_000702 | Na+/K+-ATPase alpha 2 subunit proprotein |
| ATP1B3 | NM_001679 | Na+/K+-ATPase beta 3 subunit |
| ATP2A3 | NM_005173 | sarco/endoplasmic reticulum Ca2+-ATPase isoform |
| ATP2C1 | NM_001001485 | calcium-transporting ATPase 2C1 isoform 1c |
| ATP4A | NM_000704 | ATPase, H+/K+ exchanging, alpha polypeptide |
| ATP5D | NM_001001975 | ATP synthase, H+ transporting, mitochondrial F1 |
| ATP5S | NM_001003805 | ATP synthase, H+ transporting, mitochondrial F0 |
| ATP6V0A2 | NM_012463 | ATPase, H+ transporting, lysosomal V0 subunit a |
| ATP6V0D1 | NM_004691 | ATPase, H+ transporting, lysosomal, V0 subunit |
| ATP6V1C1 | NM_001007254 | ATPase, H+ transporting, lysosomal 42kDa, V1 |
| ATP6V1E1 | NM_001696 | vacuolar H+ ATPase E1 isoform a |
| ATP7B | NM_000053 | ATPase, Cu++ transporting, beta polypeptide |
| ATP8B4 | NM_024837 | ATPase class I type 8B member 4 |
| ATP9A | NM_006045 | ATPase, Class II, type 9A |
| ATPBD4 | NM_080650 | ATP binding domain 4 |
| ATPIF1 | NM_178191 | ATPase inhibitory factor 1 isoform 3 precursor |
| ATXN1 | NM_000332 | ataxin 1 |
| ATXN2L | NM_007245 | ataxin 2 related protein isoform A |
| ATXN7L2 | NM_153340 | ataxin 7-like 2 |
| AVPR1B | NM_000707 | arginine vasopressin receptor 1B |
| AXIN2 | NM_004655 | axin 2 |
| AXL | NM_001699 | AXL receptor tyrosine kinase isoform 2 |
| AYTL2 | NM_024830 | hypothetical protein FLJ12443 |
| B3GALNT1 | NM_003781 | UDP-Gal:betaGlcNAc beta |
| B3GALT5 | NM_006057 | UDP-Gal:betaGlcNAc beta |
| B3GAT1 | NM_018644 | beta-1,3-glucuronyltransferase 1 |
| B3GAT3 | NM_012200 | beta-1,3-glucuronyltransferase 3 |
| B3GNT3 | NM_014256 | UDP-GlcNAc:betaGal |
| B4GALT1 | NM_001497 | UDP-Gal:betaGlcNAc beta 1,4- |
| B4GALT2 | NM_001005417 | UDP-Gal:betaGlcNAc beta 1,4- |
| BAALC | NM_001024372 | brain and acute leukemia, cytoplasmic isoform 2 |
| BAAT | NM_001701 | bile acid Coenzyme A: amino acid |
| BACE1 | NM_012104 | beta-site APP-cleaving enzyme 1 isoform A |
| BACH2 | NM_021813 | BTB and CNC homology 1, basic leucine zipper |
| BAD | NM_004322 | BCL2-antagonist of cell death protein |
| BAI2 | NM_001703 | brain-specific angiogenesis inhibitor 2 |
| BAK1 | NM_001188 | BCL2-antagonist/killer 1 |
| BAT1 | NM_004640 | HLA-B associated transcript 1 |
| BATF2 | NM_138456 | basic leucine zipper transcription factor, |
| BAX | NM_004324 | BCL2-associated X protein isoform beta |
| BAZ2A | NM_013449 | bromodomain adjacent to zinc finger domain, 2A |
| BBS1 | NM_024649 | Bardet-Biedl syndrome 1 |
| BBS10 | NM_024685 | hypothetical protein LOC79738 |
| BCAN | NM_021948 | brevican isoform 1 |
| BCAP29 | NM_001008407 | B-cell receptor-associated protein BAP29 isoform |
| BCAS3 | NM_017679 | breast carcinoma amplified sequence 3 |
| BCCIP | NM_078469 | BRCA2 and CDKN1A-interacting protein isoform C |
| BCKDK | NM_005881 | branched chain ketoacid dehydrogenase kinase |
| BCL10 | NM_003921 | B-cell CLL/lymphoma 10 |
| BCL11B | NM0_22898 | B-cell CLL/lymphoma 11B isoform 2 |
| BCL2 | NM_000633 | B-cell lymphoma protein 2 alpha isoform |
| BCL6 | NM_001706 | B-cell lymphoma 6 protein |
| BCL7A | NM_001024808 | B-cell CLL/lymphoma 7A isoform b |
| BCL9L | NM_182557 | B-cell CLL/lymphoma 9-like |
| BCORL1 | NM_021946 | BCL6 co-repressor-like 1 |
| BDKRB2 | NM_000623 | bradykinin receptor B2 |
| BET1L | NM_016526 | blocked early in transport 1 homolog (S. |
| BFAR | NM_016561 | apoptosis regulator |
| BHLHB5 | NM_152414 | basic helix-loop-helix domain containing, class |
| BICD1 | NM_001003398 | bicaudal D homolog 1 isoform 2 |
| BIK | NM_001197 | BCL2-interacting killer |
| BIRC1 | NM_004536 | baculoviral IAP repeat-containing 1 |
| BIRC5 | NM_001012270 | baculoviral IAP repeat-containing protein 5 |
| BM88 | NM_016564 | BM88 antigen |
| BMF | NM_001003940 | Bcl2 modifying factor isoform bmf-1 |
| BMP1 | NM_006129 | bone morphogenetic protein 1 isoform 3, |
| BMP6 | NM_001718 | bone morphogenetic protein 6 precursor |
| BMP7 | NM_001719 | bone morphogenetic protein 7 precursor |
| BMP8B | NM_001720 | bone morphogenetic protein 8B preproprotein |
| BMPR2 | NM_001204 | bone morphogenetic protein receptor type II |
| BNC2 | NM_017637 | basonuclin 2 |
| BOLA2 | NM_001031833 | BolA-like protein 2 isoform b |
| BRCA1 | NM_007306 | breast cancer 1, early onset isoform |
| BRD4 | NM_014299 | bromodomain-containing protein 4 isoform short |
| BRE | NM_004899 | brain and reproductive organ-expressed (TNFRSF1A |
| BRPF1 | NM_001003694 | bromodomain and PHD finger-containing protein 1 |
| BRPF3 | NM_015695 | bromodomain and PHD finger containing, 3 |
| BRRN1 | NM_015341 | barren |
| BRUNOL6 | NM_052840 | bruno-like 6, RNA binding protein |
| BRWD1 | NM_033656 | bromodomain and WD repeat domain containing 1 |
| BSDC1 | NM_018045 | BSD domain containing 1 |
| BSN | NM_003458 | bassoon protein |
| BSPRY | NM_017688 | B-box and SPRY domain containing |
| BTBD11 | NM_001017523 | BTB (POZ) domain containing 11 isoform 2 |
| BTBD12 | NM_032444 | BTB (POZ) domain containing 12 |
| BTBD2 | NM_017797 | BTB (POZ) domain containing 2 |
| BTBD3 | NM_014962 | BTB/POZ domain containing protein 3 isoform a |
| BTBD4 | NM_025224 | BTB (POZ) domain containing 4 |
| BTBD7 | NM_001002860 | BTB (POZ) domain containing 7 isoform I |
| BTG2 | NM_006763 | B-cell translocation gene 2 |
| BTG4 | NM_017589 | B-cell translocation gene 4 |
| BTN1A1 | NM_001732 | butyrophilin, subfamily 1, member Al |
| BTN3A2 | NM_007047 | butyrophilin, subfamily 3, member A2 precursor |
| BTNL9 | NM_152547 | butyrophilin-like 9 |
| BTRC | NM_003939 | beta-transducin repeat containing protein |
| C10orf10 | NM_007021 | fasting induced gene |
| C10orf13 | NM_152429 | hypothetical protein LOG143282 |
| C10orf22 | NM_032804 | hypothetical protein LOC84890 |
| C10orf26 | NM_017787 | hypothetical protein LOC54838 |
| C10orf28 | NM_014472 | growth inhibition and differentiation related |
| C10orf32 | NM_144591 | hypothetical protein MGC27171 |
| C10orf38 | NM_001010924 | hypothetical protein LOC221061 |
| C10orf4 | NM_145246 | FRA10AC1 protein isoform FRA10AC1-1 |
| C10orf42 | NM_138357 | hypothetical protein LOC90550 |
| C10orf49 | NM_145314 | hypothetical protein LOC221044 |
| C10orf53 | NM_182554 | hypothetical protein LOC282966 |
| C10orf54 | NM_022153 | hypothetical protein LOC64115 |
| C10orf55 | NM_001001791 | hypothetical protein LOC414236 |
| C10orf56 | NM_153367 | hypothetical protein LOC219654 |
| C10orf57 | NM_025125 | hypothetical protein LOC80195 |
| C10orf58 | NM_032333 | hypothetical protein LOC84293 |
| C10orf63 | NM_145010 | enkurin |
| C10orf65 | NM_138413 | hypothetical protein LOC112817 |
| C10orf72 | NM_001031746 | hypothetical protein LOC196740 isoform 1 |
| C10orf76 | NM_024541 | hypothetical protein LOC79591 |
| C10orf77 | NM_024789 | hypothetical protein LOC79847 |
| C10orf83 | NM_178832 | hypothetical protein LOC118812 |
| C10orf89 | NM_153336 | hypothetical protein LOC118672 |
| C10orf91 | NM_173541 | hypothetical protein LOC170393 |
| C10orf95 | NM_024886 | hypothetical protein LOC79946 |
| C11orf1 | NM_022761 | hypothetical protein LOC64776 |
| C11orf11 | NM_006133 | neural stem cell-derived dendrite regulator |
| C11orf17 | NM_020642 | chromosome 11 open reading frame 17 |
| C11orf30 | NM_020193 | EMSY protein |
| C11orf38 | NM_212555 | hypothetical protein LOC399967 |
| C11orf44 | NM_173580 | hypothetical protein LOC283171 |
| C11orf45 | NM_145013 | hypothetical protein LOC219833 |
| C11orf49 | NM_001003676 | hypothetical protein LOC79096 isoform 1 |
| C11orf57 | NM_018195 | hypothetical protein LOC55216 |
| C11orf68 | NM_031450 | basophilic leukemia expressed protein BLES03 |
| C11orf9 | NM_013279 | hypothetical protein LOC745 |
| C12orf29 | NM_001009894 | hypothetical protein LOC91298 |
| C12orf31 | NM_032338 | hypothetical protein LOC84298 |
| C12orf32 | NM_031465 | hypothetical protein LOC83695 |
| C12orf43 | NM_022895 | hypothetical protein LOC64897 |
| C12orf54 | NM_152319 | hypothetical protein LOC121273 |
| C12orf57 | NM_138425 | C10 protein |
| C12orf59 | NM_153022 | hypothetical protein LOC120939 |
| C12orf61 | NM_175895 | hypothetical protein LOC283416 |
| C13orf1 | NM_020456 | hypothetical protein LOC57213 |
| C13orf23 | NM_025138 | hypothetical protein LOC80209 |
| C14orf121 | NM_138360 | hypothetical protein LOC90668 |
| C14orf132 | NM_020215 | hypothetical protein LOC56967 |
| C14orf140 | NM_024643 | hypothetical protein LOC79696 |
| C14orf151 | NM_032714 | hypothetical protein LOC84800 |
| C14orf153 | NM_032374 | hypothetical protein LOC84334 |
| C14orf173 | NM_001031714 | hypothetical protein LOC64423 isoform 1 |
| C14orf28 | NM_001017923 | hypothetical protein LOC122525 |
| C14orf32 | NM_144578 | MAPK-interacting and spindle-stabilizing |
| C14orf4 | NM_024496 | chromosome 14 open reading frame 4 |
| C14orf43 | NM_194278 | hypothetical protein LOC91748 |
| C14orf58 | NM_017791 | hypothetical protein LOC55640 |
| C14orf68 | NM_207117 | chromosome 14 open reading frame 68 |
| C14orf79 | NM_174891 | hypothetical protein LOC122616 |
| C14orf92 | NM_014828 | epidermal Langerhans cell protein LCP1 |
| C15orf20 | NM_025049 | DNA helicase homolog PIF1 |
| C15orf37 | NM_175898 | hypothetical protein LOC283687 |
| C15orf38 | NM_182616 | hypothetical protein LOC348110 |
| C16orf25 | NM_173476 | hypothetical protein LOC124093 isoform 2 |
| C16orf3 | NM_001214 | hypothetical protein LOC750 |
| C16orf34 | NM_144570 | chromosome 16 open reading frame 34 |
| C16orf5 | NM_013399 | cell death inducing protein |
| C16orf50 | NM_032269 | chromosome 16 open reading frame 50 |
| C16orf54 | NM_175900 | hypothetical protein LOC283897 |
| C16orf57 | NM_024598 | hypothetical protein LOC79650 |
| C16orf58 | NM_022744 | hypothetical protein LOC64755 |
| C16orf7 | NM_004913 | chromosome 16 open reading frame 7 |
| C17orf27 | NM_020914 | chromosome 17 open reading frame 27 |
| C17orf28 | NM_030630 | hypothetical protein LOC283987 |
| C17orf32 | NM_152464 | hypothetical protein LOC147007 |
| C17orf53 | NM_024032 | hypothetical protein LOC78995 |
| C17orf55 | NM_178519 | hypothetical protein LOC284185 |
| C17orf65 | NM_178542 | hypothetical protein LOC339201 |
| C17orf74 | NM_175734 | hypothetical protein LOC201243 |
| C18orf1 | NM_001003674 | hypothetical protein LOC753 isoform gamma 1 |
| C18orf19 | NM_152352 | hypothetical protein LOC125228 |
| C18orf25 | NM_001008239 | chromosome 18 open reading frame 25 isoform b |
| C18orf4 | NM_032160 | hypothetical protein LOC92126 |
| C18orf43 | NM_006553 | chromosome 18 open reading frame 43 |
| C18orf54 | NM_173529 | hypothetical protein LOC162681 |
| C19orf21 | NM_173481 | hypothetical protein LOC126353 |
| C19orf25 | NM_152482 | hypothetical protein LOC148223 |
| C19orf28 | NM_174983 | hypothetical protein LOC126321 |
| C19orf31 | NM_001014373 | hypothetical protein LOC404664 |
| C19orf37 | NM_182498 | hypothetical protein LOC126299 |
| C19orf4 | NM_012109 | brain-specific membrane-anchored protein |
| C19orf6 | NM_001033026 | membralin isoform 1 |
| C1orf106 | NM_018265 | hypothetical protein LOC55765 |
| C1orf107 | NM_014388 | hypothetical protein LOC27042 |
| C1orf109 | NM_017850 | hypothetical protein LOC54955 |
| C1orf115 | NM_024709 | hypothetical protein LOC79762 |
| C1orf116 | NM_023938 | specifically androgen-regulated protein |
| C1orf119 | NM_020141 | hypothetical protein LOC56900 |
| C1orf126 | NM_182534 | hypothetical protein LOC200197 |
| C1orf128 | NM_020362 | thioredoxin family Trp26 |
| C1orf144 | NM_015609 | putative MAPK activating protein PM20, PM21 |
| C1orf145 | NM_001025495 | hypothetical protein LOC574407 |
| C1orf147 | NM_001025592 | hypothetical protein LOC574431 |
| C1orf151 | NM_001032363 | chromosome 1 open reading frame 151 protein |
| C1orf159 | NM_017891 | hypothetical protein LOC54991 |
| C1orf162 | NM_174896 | hypothetical protein LOC128346 |
| C1orf163 | NM_023077 | hypothetical protein LOC65260 |
| C1orf183 | NM_019099 | hypothetical protein LOC55924 isoform 1 |
| C1orf19 | NM_052965 | hypothetical protein LOC116461 |
| C1orf21 | NM_030806 | chromosome 1 open reading frame 21 |
| C1orf24 | NM_052966 | niban protein isoform 2 |
| C1orf26 | NM_017673 | hypothetical protein LOC54823 |
| C1orf38 | NM_004848 | basement membrane-induced gene isoform 1 |
| C1orf49 | NM_032126 | hypothetical protein LOC84066 |
| C1orf62 | NM_152763 | hypothetical protein LOC254268 |
| C1orf69 | NM_001010867 | hypothetical protein LOC200205 |
| C1orf71 | NM_152609 | hypothetical protein LOC163882 |
| C1orf74 | NM_152485 | hypothetical protein LOC148304 |
| C1orf82 | NM_024813 | hypothetical protein LOC79871 |
| C1orf84 | NM_182518 | RP11-506B15.1 protein isoform 3 |
| C1orf9 | NM_014283 | chromosome 1 open reading frame 9 protein |
| C1orf91 | NM_019118 | hypothetical protein LOC56063 |
| C1orf93 | NM_152371 | hypothetical protein LOC127281 |
| C1orf95 | NM_001003665 | hypothetical protein LOC375057 |
| C1orf96 | NM_145257 | hypothetical protein LOC126731 |
| C1QG | NM_172369 | complement component 1, subcomponent, gamma |
| C1QDC1 | NM_001002259 | C1q domain containing 1 isoform 1 |
| C1QL1 | NM_006688 | complement component 1, q subcomponent-like 1 |
| C1QTNF1 | NM_030968 | C1q and tumor necrosis factor related protein 1 |
| C1QTNF7 | NM_031911 | C1q and tumor necrosis factor related protein 7 |
| C1QTNF8 | NM_207419 | hypothetical protein LOC390664 |
| C2 | NM_000063 | complement component 2 precursor |
| C20orf100 | NM_032883 | chromosome 20 open reading frame 100 |
| C20orf102 | NM_080607 | hypothetical protein LOC128434 |
| C20orf11 | NM_017896 | chromosome 20 open reading frame 11 |
| C20orf112 | NM_080616 | hypothetical protein LOC140688 |
| C20orf117 | NM_080627 | hypothetical protein LOC140710 isoform 1 |
| C20orf118 | NM_080628 | hypothetical protein LOC140711 |
| C20orf134 | NM_001024675 | hypothetical protein LOC170487 |
| C20orf173 | NM_080828 | hypothetical protein LOC140873 |
| C20orf20 | NM_018270 | MRG-binding protein |
| C20orf39 | NM_024893 | hypothetical protein LOC79953 |
| C20orf42 | NM_017671 | chromosome 20 open reading frame 42 |
| C20orf43 | NM_016407 | hypothetical protein LOC51507 |
| C20orf77 | NM_021215 | hypothetical protein LOC58490 |
| C20orf98 | NM_024958 | hypothetical protein LOC80023 |
| C21orf124 | NM_032920 | hypothetical protein LOC85006 |
| C21orf128 | NM_152507 | hypothetical protein LOC150147 |
| C21orf129 | NM_152506 | hypothetical protein LOC150135 |
| C21orf25 | NM_199050 | hypothetical protein LOC25966 |
| C21orf58 | NM_199071 | hypothetical protein LOC54058 isoform 2 |
| C21orf6 | NM_016940 | hypothetical protein LOC10069 |
| C21orf69 | NM_058189 | chromosome 21 open reading frame 69 |
| C21orf7 | NM_020152 | chromosome 21 open reading frame 7 |
| C21orf70 | NM_058190 | hypothetical protein LOC85395 |
| C21orf93 | NM_145179 | hypothetical protein LOC246704 |
| C22orf15 | NM_182520 | hypothetical protein LOC150248 |
| C22orf23 | NM_032561 | hypothetical protein LOC84645 |
| C22orf25 | NM_152906 | hypothetical protein LOC128989 |
| C22orf5 | NM_012264 | chromosome 22 open reading frame 5 |
| C22orf9 | NM_001009880 | hypothetical protein LOC23313 isoform b |
| C2orf15 | NM_144706 | hypothetical protein LOC150590 |
| C2orf16 | NM_032266 | hypothetical protein LOC84226 |
| C2orf18 | NM_017877 | hypothetical protein LOC54978 |
| C3orf17 | NM_001025072 | hypothetical protein LOC25871 isoform b |
| C3orf18 | NM_016210 | hypothetical protein LOC51161 |
| C3orf45 | NM_153215 | hypothetical protein LOC132228 |
| C3orf58 | NM_173552 | hypothetical protein LOC205428 |
| C3orf62 | NM_198562 | hypothetical protein LOC375341 |
| C3orf63 | NM_015224 | retinoblastoma-associated protein 140 |
| C4orf12 | NM_205857 | FBI4 protein |
| C4orf13 | NM_001029998 | hypothetical protein LOC84068 isoform b |
| C5orf16 | NM_173828 | hypothetical protein LOC285613 |
| C5orf23 | NM_024563 | hypothetical protein LOC79614 |
| C4orf24 | NM_152409 | hypothetical protein LOC134553 |
| C6orf106 | NM_022758 | chromosome 6 open reading frame 106 isoform b |
| C6orf117 | NM_138409 | hypothetical protein LOC112609 |
| C6orf120 | NM_001029863 | hypothetical protein LOC387263 |
| C6orf122 | NM_207502 | chromosome 6 open reading frame 122 |
| C6orf134 | NM_024909 | hypothetical protein LOC79969 isoform 2 |
| C6orf145 | NM_183373 | hypothetical protein LOC221749 |
| C6orf149 | NM_020408 | hypothetical protein LOC527128 |
| C6orf151 | NM_152551 | U11/U12 snRNP 48K |
| C6orf153 | NM_033112 | hypothetical protein LOC88745 |
| C6orf199 | NM_145025 | hypothetical protein LOC221264 |
| C6orf35 | NM_018452 | hypothetical protein LOC55836 |
| C6orf47 | NM_021184 | G4 protein |
| C6orf49 | NM_013397 | over-expressed breast tumor protein |
| C6orf71 | NM_203395 | chromosome 6 open reading frame 71 |
| C6orf89 | NM_152734 | hypothetical protein LOC221477 |
| C7orf27 | NM_152743 | hypothetical protein LOC221927 |
| C7orf34 | NM_178829 | hypothetical protein LOC135927 |
| C8orf1 | NM_004337 | hypothetical protein LOC734 |
| C8orf13 | NM_053279 | hypothetical protein LOC83648 |
| C8orf30A | NM_016458 | brain protein 16 |
| C8orf33 | NM_023080 | hypothetical protein LOC65265 |
| C8orf37 | NM_177965 | hypothetical protein LOC157657 |
| C8orf44 | NM_019607 | hypothetical protein LOC56260 |
| C8orf46 | NM_152765 | hypothetical protein LOC254778 |
| C8orf49 | NM_001031839 | hypothetical protein LOC606553 |
| C8orf51 | NM_024035 | hypothetical protein LOC78998 |
| C8orf55 | NM_016647 | mesenchymal stem cell protein DSCD75 |
| C8orf58 | NM_001013842 | hypothetical protein LOC541565 |
| C8orf78 | NM_182525 | hypothetical protein LOC157376 |
| C9orf106 | NM_001012715 | hypothetical protein LOC414318 |
| C9orf10OS | NM_198841 | hypothetical protein LOC158293 |
| C9orf111 | NM_152286 | chromosome 9 open reading frame 111 |
| C9orf114 | NM_016390 | hypothetical protein LOC51490 |
| C9orf125 | NM_032342 | hypothetical protein LOC84302 |
| C9orf140 | NM_178448 | hypothetical protein LOC89958 |
| C9orf152 | NM_001012993 | hypothetical protein LOC401546 |
| C9orf23 | NM_148178 | hypothetical protein LOC138716 |
| C9orf25 | NM_147202 | hypothetical protein LOC203259 |
| C9orf28 | NM_001011703 | hypothetical protein LOC89853 isoform 2 |
| C9orf42 | NM_138333 | hypothetical protein LOC116224 |
| C9orf45 | NM_030814 | hypothetical protein L0C81571 |
| C9orf47 | NM_001001938 | hypothetical protein L0C286223 |
| C9orf58 | NM_001002260 | chromosome 9 open reading frame 58 isoform 2 |
| C9orf7 | NM_017586 | hypothetical protein LOC11094 |
| C9orf75 | NM_173691 | hypothetical protein LOC286262 |
| C9orf86 | NM_024718 | hypothetical protein LOC55684 |
| C9orf97 | NM_139246 | hypothetical protein LOC158427 |
| CA10 | NM_020178 | carbonic anhydrase X |
| CA12 | NM_001218 | carbonic anhydrase XII isoform 1 precursor |
| CA7 | NM_001014435 | carbonic anhydrase VII isoform 2 |
| CA9 | NM_001216 | carbonic anhydrase IX precursor |
| CABLES2 | NM_031215 | Cdk5 and Ab1 enzyme substrate 2 |
| CABP1 | NM_001033677 | calcium binding protein 1 isoform 3 |
| CACHD1 | NM_020925 | cache domain containing 1 |
| CACNA1E | NM_000721 | calcium channel, voltage-dependent, alpha 1E |
| CACNA1I | NM_001003406 | voltage-dependent T-type calcium channel |
| CACNA2D2 | NM_001005505 | calcium channel, voltage-dependent, alpha |
| CACNA2D4 | NM_001005737 | voltage-gated calcium channel alpha(2)delta-4 |
| CACNB1 | NM_000723 | calcium channel, voltage-dependent, beta 1 |
| CACNB3 | NM_000725 | calcium channel, voltage-dependent, beta 3 |
| CACNG4 | NM_014405 | voltage-dependent calcium channel gamma-4 |
| CADPS | NM_003716 | Ca2+-dependent secretion activator isoform 1 |
| CALB1 | NM_004929 | calbindin 1 |
| CALCA | NM_001033953 | calcitonin isoform CGRP preproprotein |
| CALCB | NM_000728 | calcitonin-related polypeptide, beta |
| CALCOCO2 | NM_005831 | calcium binding and coiled-coil domain 2 |
| CALCR | NM_001742 | calcitonin receptor |
| CALM3 | NM_005184 | calmodulin 3 |
| CALML3 | NM_005185 | calmodulin-like 3 |
| CALML5 | NM_017422 | calmodulin-like skin protein |
| CALN1 | NM_001017440 | calneuron 1 |
| CAMK2B | NM_001220 | calcium/calmodulin-dependent protein kinase IIB |
| CAMKK1 | NM_032294 | calcium/calmodulin-dependent protein kinase 1 |
| CAMKK2 | NM_172214 | calcium/calmodulin-dependent protein kinase |
| CAMLG | NM_001745 | calcium modulating ligand |
| CAMSAP1 | NM_015447 | calmodulin regulated spectrin-associated protein |
| CAMTA1 | NM_015215 | calmodulin-binding transcription activator 1 |
| CAMTA2 | NM_015099 | calmodulin binding transcription activator 2 |
| CAP1 | NM_006367 | adenylyl cyclase-associated protein |
| CAPN3 | NM_212467 | calpain 3 isoform h |
| CAPN5 | NM_004055 | calpain 5 |
| CAPN6 | NM_014289 | calpain 6 |
| CAPN9 | NM_016452 | calpain 9 isoform 2 |
| CAPNS1 | NM_001003962 | calpain, small subunit 1 |
| CARD4 | NM_006092 | caspase recruitment domain family, member 4 |
| CARD9 | NM_052813 | caspase recruitment domain protein 9 |
| CARKL | NM_013276 | carbohydrate kinase-like |
| CARM1 | NM_199141 | coactivator-associated arginine |
| CASKIN1 | NM_020764 | CASK interacting protein 1 |
| CASKIN2 | NM_020753 | cask-interacting protein 2 |
| CASP2 | NM_032982 | caspase 2 isoform 1 preproprotein |
| CASP4 | NM_033307 | caspase 4 isoform delta |
| CASP6 | NM_001226 | caspase 6 isoform alpha preproprotein |
| CASP7 | NM_001227 | caspase 7 isoform alpha precursor |
| CASR | NM_000388 | calcium-sensing receptor |
| CAST1 | NM_015576 | cytomatrix protein p110 |
| CASZ1 | NM_017766 | castor homolog 1, zinc finger |
| CAV1 | NM_001753 | caveolin 1 |
| CAV2 | NM_001233 | caveolin 2 isoform a and b |
| CAV3 | NM_001234 | caveolin 3 |
| CBFA2T2 | NM_001032999 | core-binding factor, runt domain, alpha subunit |
| CBFA2T3 | NM_005187 | myeloid translocation gene-related protein 2 |
| CBFB | NM_001755 | core-binding factor, beta subunit isoform 2 |
| CBLC | NM_012116 | Cas-Br-M (murine) ecotropic retroviral |
| CBLN1 | NM_004352 | cerebellin 1 precursor |
| CBLN4 | NM_080617 | cerebellin 4 precursor |
| CBS | NM_000071 | cystathionine-beta-synthase |
| CBX2 | NM_005189 | chromobox homolog 2 isoform 1 |
| CBX3 | NM_007276 | chromobox homolog 3 |
| CBX6 | NM_014292 | chromobox homolog 6 |
| CCBL1 | NM_004059 | cytoplasmic cysteine conjugate-beta lyase |
| CCDC28B | NM_024296 | coiled-coil domain containing 28B |
| CCDC3 | NM_031455 | coiled-coil domain containing 3 |
| CCDC33 | NM_182791 | hypothetical protein LOC80125 |
| CCDC43 | NM_144609 | hypothetical protein LOC124808 |
| CCDC48 | NM_024768 | hypothetical protein LOC79825 |
| CCDC49 | NM_017748 | hypothetical protein LOC54883 |
| CCDC50 | NM_174908 | Ymer protein short isoform |
| CCDC52 | NM_144718 | coiled-coil domain containing 52 |
| CCDC6 | NM_005436 | coiled-coil domain containing 6 |
| CCDC68 | NM_025214 | CTCL tumor antigen se57-1 |
| CCDC69 | NM_015621 | hypothetical protein LOC26112 |
| CCDC86 | NM_024098 | coiled-coil domain containing 86 |
| CCDC97 | NM_052848 | hypothetical protein LOC90324 |
| CCL22 | NM_002990 | small inducible cytokine A22 precursor |
| CCND1 | NM_053056 | cyclin D1 |
| CCND2 | NM_001759 | cyclin D2 |
| CCND3 | NM_001760 | cyclin D3 |
| CCNE2 | NM_057735 | cyclin E2 isoform 2 |
| CCNF | NM_001761 | cyclin F |
| CCNG1 | NM_004060 | cyclin G1 |
| CCNJ | NM_019084 | cyclin J |
| CCR1 | NM_001295 | chemokine (C-C motif) receptor 1 |
| CCRL1 | NM_016557 | chemokine (C-C motif) receptor-like 1 |
| CD109 | NM_133493 | CD109 |
| CD14 | NM_000591 | CD14 antigen precursor |
| CD151 | NM_004357 | CD151 antigen |
| CD160 | NM_007053 | CD160 antigen |
| CD164L2 | NM_207397 | CD164 sialomucin-like 2 |
| CD180 | NM_005582 | CD180 antigen |
| CD200 | NM_001004196 | CD200 antigen isoform b |
| CD247 | NM_000734 | T-cell receptor zeta chain isoform 2 precursor |
| CD276 | NM_001024736 | CD276 antigen isoform a |
| CD28 | NM_006139 | CD28 antigen |
| CD3E | NM_000733 | CD3E antigen, epsilon polypeptide (TiT3 |
| CD40LG | NM_000074 | CD40 ligand |
| CD44 | NM_000610 | CD44 antigen isoform 1 precursor |
| CD46 | NM_002389 | CD46 antigen, complement regulatory protein |
| CD47 | NM_001025079 | CD47 molecule isoform 3 precursor |
| CD59 | NM_000611 | CD59 antigen p18-20 |
| CD84 | NM_003874 | CD84 antigen (leukocyte antigen) |
| CD86 | NM_006889 | CD86 antigen isoform 2 precursor |
| CD8A | NM_001768 | CD8 antigen alpha polypeptide isoform 1 |
| CD97 | NM_001025160 | CD97 antigen isoform 3 precursor |
| CD99L2 | NM_031462 | CD99 antigen-like 2 isoform E3′-E4′-E3-E4 |
| CDA | NM_001785 | cytidine deaminase |
| CDADC1 | NM_030911 | cytidine and dCMP deaminase domain containing 1 |
| CDAN1 | NM_138477 | codanin 1 |
| CDC23 | NM_004661 | cell division cycle protein 23 |
| CDC25A | NM_001789 | cell division cycle 25A isoform a |
| CDC2L6 | NM_015076 | cyclin-dependent kinase (CDC2-like) 11 |
| CDC37 | NM_007065 | CDC37 homolog |
| CDC40 | NM_015891 | cell division cycle 40 homolog |
| CDC42BPB | NM_006035 | CDC42-bindin protein kinase beta |
| CDC42EP1 | NM_007061 | CDC42 effector protein 1 isoform b |
| CDC42EP4 | NM_012121 | Cdc42 effector protein 4 |
| CDC42SE1 | NM_020239 | CDC42 small effector 1 |
| CDCA5 | NM_080668 | cell division cycle associated 5 |
| CDCA8 | NM_018101 | cell division cycle associated 8 |
| CDGAP | NM_020754 | Cdc42 GTPase-activating protein |
| CDH13 | NM_001257 | cadherin 13 preproprotein |
| CDH16 | NM_004062 | cadherin 16 precursor |
| CDH17 | NM_004063 | cadherin 17 precursor |
| CDH6 | NM_004932 | cadherin 6, type 2 preproprotein |
| CDH9 | NM_016279 | cadherin 9, type 2 preproprotein |
| CDK10 | NM_052988 | cyclin-dependent kinase 10 isoform 3 |
| CDK2AP1 | NM_004642 | CDK2-associated protein 1 |
| CDK5R2 | NM_003936 | cyclin-dependent kinase 5, regulatory subunit 2 |
| CDK6 | NM_001259 | cyclin-dependent kinase 6 |
| CDKN1B | NM_004064 | cyclin-dependent kinase inhibitor 1B |
| CDON | NM_016952 | surface glycoprotein, Ig superfamily member |
| CDRT4 | NM_173622 | hypothetical protein LOC284040 |
| CEACAM1 | NM_001024912 | carcinoembryonic antigen-related cell adhesion |
| CEACAM21 | NM_033543 | carcinoembryonic antigen-related cell adhesion |
| CEACAM7 | NM_006890 | carcinoembryonic antigen-related cell adhesion |
| CEACAM8 | NM_001816 | carcinoembryonic antigen-related cell adhesion |
| CEECAM1 | NM_016174 | cerebral endothelial cell adhesion molecule 1 |
| CELSR1 | NM_014246 | cadherin EGF LAG seven-pass G-type receptor 1 |
| CELSR2 | NM_001408 | cadherin EGF LAG seven-pass G-type receptor 2 |
| CELSR3 | NM_001407 | cadherin EGF LAG seven-pass G-type receptor 3 |
| CENPB | NM_001810 | centromere protein B |
| CENTG1 | NM_014770 | centaurin, gamma 1 |
| CEP192 | NM_018069 | hypothetical protein LOC55125 isoform 2 |
| CEP250 | NM_007186 | centrosomal protein 2 isoform 1 |
| CEP55 | NM_018131 | centrosomal protein 55 kDa |
| CEP72 | NM_018140 | centrosomal protein 72 kDa |
| CERK | NM_022766 | ceramide kinase isoform a |
| CFD | NM_001928 | complement factor D preproprotein |
| CFTR | NM_000492 | cystic fibrosis transmembrane conductance |
| CGA | NM_000735 | glycoprotein hormones, alpha polypeptide |
| CGGBP1 | NM_001008390 | CGG triplet repeat binding protein 1 |
| CGNL1 | NM_032866 | cingulin-like 1 |
| CHCHD5 | NM_032309 | coiled-coil-helix-coiled-coil-helix domain |
| CHCHD7 | NM_001011667 | coiled-coil-helix-coiled-coil-helix domain |
| CHD1 | NM_001270 | chromodomain helicase DNA binding protein 1 |
| CHD2 | NM_001271 | chromodomain helicase DNA binding protein 2 |
| CHD3 | NM_001005271 | chromodomain helicase DNA binding protein 3 |
| CHD5 | NM_015557 | chromodomain helicase DNA binding protein 5 |
| CHERP | NM_006387 | calcium homeostasis endoplasmic reticulum |
| CHES1 | NM_005197 | checkpoint suppressor 1 |
| CHKB | NM_152253 | choline/ethanolamine kinase isoform b |
| CHMP4A | NM_014169 | chromatin modifying protein 4A |
| CHMP7 | NM_152272 | CHMP family, member 7 |
| CHR415SYT | NM_001014372 | chr415 synaptotagmin |
| CHRAC1 | NM_017444 | chromatin accessibility complex 1 |
| CHRD | NM_177978 | chordin isoform b |
| CHRFAM7A | NM_139320 | CHRNA7-FAM7A fusion isoform 1 |
| CHRNA7 | NM_000746 | cholinergic receptor, nicotinic, alpha 7 |
| CHRNE | NM_000080 | nicotinic acetylcholine receptor epsilon |
| CHST1 | NM_003654 | carbohydrate (keratan sulfate Gal-6) |
| CHST10 | NM_004854 | HNK-1 sulfotransferase |
| CHST12 | NM_018641 | carbohydrate (chondroitin 4) sulfotransferase |
| CHST13 | NM_152889 | carbohydrate (chondroitin 4) sulfotransferase |
| CHST3 | NM_004273 | carbohydrate (chondroitin 6) sulfotransferase 3 |
| CIB2 | NM_006383 | DNA-dependent protein kinase catalytic |
| CIRBP | NM_001280 | cold inducible RNA binding protein |
| CITED2 | NM_006079 | Cbp/p300-interacting transactivator, with |
| CITED4 | NM_133467 | Cbp/p300-interacting transactivator, with |
| CKAP1 | NM_001281 | cytoskeleton associated protein 1 |
| CKAP4 | NM_006825 | cytoskeleton-associated protein 4 |
| CLASP1 | NM_015282 | CLIP-associating protein 1 |
| CLDN1 | NM_021101 | claudin 1 |
| CLDN12 | NM_012129 | claudin 12 |
| CLDN15 | NM_014343 | claudin 15 isoform 1 |
| CLDN18 | NM_001002026 | claudin 18 isoform 2 |
| CLDN19 | NM_148960 | claudin 19 |
| CLDN2 | NM_020384 | claudin 2 |
| CLDN6 | NM_021195 | claudin 6 |
| CLDN9 | NM_020982 | claudin 9 |
| CLDND1 | NM_019895 | claudin domain containing 1 protein isoform a |
| CLEC2A | NM_207375 | C-type lectin domain family 2, member A |
| CLIC5 | NM_016929 | chloride intracellular channel 5 |
| CLIC6 | NM_053277 | chloride intracellular channel 6 |
| CLIPR-59 | NM_015526 | CLIP-170-related protein |
| CLLU1 | NM_001025233 | hypothetical protein LOC574028 |
| CLN6 | NM_017882 | CLN6 protein |
| CLOCK | NM_004898 | clock |
| CLPB | NM_030813 | suppressor of potassium transport defect 3 |
| CLSTN2 | NM_022131 | calsyntenin 2 |
| CMIP | NM_030629 | c-Maf-inducing protein Tc-mip isoform |
| CMTM4 | NM_181521 | chemokine-like factor superfamily 4 isoform 2 |
| CMYA1 | NM_194293 | cardiomyopathy associated 1 |
| CNFN | NM_032488 | cornifelin |
| CNGA2 | NM_005140 | cyclic nucleotide gated channel alpha 2 |
| CNGA3 | NM_001298 | cyclic nucleotide gated channel alpha 3 |
| CNGB1 | NM_001297 | cyclic nucleotide gated channel beta 1 |
| CNKSR3 | NM_173515 | CNKSR family member 3 |
| CNNM3 | NM_017623 | cyclin M3 isoform 1 |
| CNNM4 | NM_020184 | cyclin M4 |
| CNOT4 | NM_001008225 | CCR4-NOT transcription complex, subunit 4 |
| CNOT6 | NM_015455 | CCR4-NOT transcription complex, subunit 6 |
| CNOT7 | NM_054026 | CCR4-NOT transcription complex, subunit 7 |
| CNP | NM_033133 | 2′,3′-cyclic nucleotide 3′ phosphodiesterase |
| CNTF | NM_000614 | ciliary neurotrophic factor |
| CNTN2 | NM_005076 | contactin 2 precursor |
| CNTN3 | NM_020872 | contactin 3 |
| CNTN4 | NM_175607 | contactin 4 isoform a precursor |
| CNTNAP1 | NM_003632 | contactin associated protein 1 |
| CNTNAP2 | NM_014141 | cell recognition molecule Caspr2 precursor |
| CNTNAP4 | NM_033401 | cell recognition protein CASPR4 isoform 1 |
| CNTNAP5 | NM_130773 | contactin associated protein-like 5 isoform 1 |
| COBRA1 | NM_015456 | cofactor of BRCA1 |
| COG3 | NM_031431 | component of golgi transport complex 3 |
| COG6 | NM_020751 | component of oligomeric golgi complex 6 |
| COL12A1 | NM_004370 | collagen, type XII, alpha 1 long isoform |
| COL18A1 | NM_030582 | alpha 1 type XVIII collagen isoform 1 precursor |
| COL1A1 | NM_000088 | alpha 1 type I collagen preproprotein |
| COL20A1 | NM_020882 | collagen-like protein |
| COL22A1 | NM_152888 | collagen, type XXII, alpha 1 |
| COL23A1 | NM_173465 | collagen, type XXIII, alpha 1 |
| COL25A1 | NM_032518 | collagen, type XXV, alpha 1 isoform 2 |
| COL2A1 | NM_001844 | alpha 1 type II collagen isoform 1 |
| COL4A2 | NM_001846 | alpha 2 type IV collagen preproprotein |
| COL4A4 | NM_000092 | alpha 4 type IV collagen precursor |
| COL5A1 | NM_000093 | alpha 1 type V collagen preproprotein |
| COL6A2 | NM_058175 | alpha 2 type VI collagen isoform 2C2a precursor |
| COMMD3 | NM_012071 | COMM domain containing 3 |
| COMMD4 | NM_017828 | COMM domain containing 4 |
| COMMD5 | NM_014066 | hypertension-related calcium-regulated gene |
| COMMD9 | NM_014186 | COMM domain containing 9 |
| COPS7B | NM_022730 | COP9 constitutive photomorphogenic homolog |
| COPZ1 | NM_016057 | coatomer protein complex, subunit zeta 1 |
| COQ9 | NM_020312 | hypothetical protein LOC57017 |
| CORIN | NM_006587 | corin |
| CORO1B | NM_001018070 | coronin, actin binding protein, 1B |
| CORO1C | NM_014325 | coronin, actin binding protein, 1C |
| CORO2B | NM_006091 | coronin, actin binding protein, 2B |
| CORO6 | NM_032854 | coronin 6 |
| COVA1 | NM_006375 | cytosolic ovarian carcinoma antigen 1 isoform a |
| COX10 | NM_001303 | heme A:farnesyltransferase |
| COX7A2 | NM_001865 | cytochrome c oxidase subunit Vila polypeptide 2 |
| CPA4 | NM_016352 | carboxypeptidase A4 preproprotein |
| CPA6 | NM_020361 | carboxypeptidase B precursor |
| CPD | NM_001304 | carboxypeptidase D precursor |
| CPEB2 | NM_182485 | cytoplasmic polyadenylation element binding |
| CPEB3 | NM_014912 | cytoplasmic polyadenylation element binding |
| CPLX2 | NM_001008220 | complexin 2 |
| CPM | NM_001005502 | carboxypeptidase M precursor |
| CPNE5 | NM_020939 | copine V |
| CPSF4 | NM_006693 | cleavage and polyadenylation specific factor 4, |
| CPSF6 | NM_007007 | cleavage and polyadenylation specific factor 6, |
| CR2 | NM_001006658 | complement component (3d/Epstein Barr virus) |
| CRABP2 | NM_001878 | cellular retinoic acid binding protein 2 |
| CRAMP1L | NM_020825 | Crm, cramped-like |
| CRB1 | NM_201253 | crumbs homolog 1 precursor |
| CRB2 | NM_173689 | crumbs homolog 2 |
| CRB3 | NM_139161 | crumbs 3 isoform a precursor |
| CREB3L1 | NM_052854 | cAMP responsive element binding protein 3-like |
| CREB3L2 | NM_194071 | cAMP responsive element binding protein 3-like |
| CREB3L3 | NM_032607 | cAMP responsive element binding protein 3-like |
| CREB5 | NM_001011666 | cAMP responsive element binding protein 5 |
| CREG1 | NM_003851 | cellular repressor of E1A-stimulated genes |
| CREG2 | NM_153836 | cellular repressor of E1A-stimulated genes 2 |
| CRHR1 | NM_004382 | corticotropin releasing hormone receptor 1 |
| CRI1 | NM_014335 | CREBBP/EP300 inhibitor 1 |
| CRIP2 | NM_001312 | cysteine-rich protein 2 |
| CRISPLD2 | NM_031476 | cysteine-rich secretory protein LCCL domain |
| CRK | NM_005206 | v-crk sarcoma virus CT10 oncogene homolog |
| CRMP1 | NM_001014809 | collapsin response mediator protein 1 isoform 1 |
| CRNKL1 | NM_016652 | crooked neck-like 1 protein |
| CRP | NM_000567 | C-reactive protein, pentraxin-related |
| CRSP7 | NM_004831 | cofactor required for Sp1 transcriptional |
| CRSP8 | NM_004269 | cofactor required for Sp1 transcriptional |
| CRTAP | NM_006371 | cartilage associated protein precursor |
| CRTC1 | NM_015321 | mucoepidermoid carcinoma translocated 1 isoform |
| CRTC3 | NM_022769 | transducer of regulated CREB protein 3 |
| CRY2 | NM_021117 | cryptochrome 2 (photolyase-like) |
| CRYZL1 | NM_145858 | crystallin, zeta-like 1 |
| CSDC2 | NM_014460 | RNA-binding protein pippin |
| CSF1R | NM_005211 | colony stimulating factor 1 receptor precursor |
| CSMD1 | NM_033225 | CUB and Sushi multiple domains 1 |
| CSNK1A1 | NM_001025105 | casein kinase 1, alpha 1 isoform 1 |
| CSNK1G1 | NM_001011664 | casein kinase 1, gamma 1 isoform L |
| CSNK1G3 | NM_001031812 | casein kinase 1, gamma 3 isoform 2 |
| CSRP1 | NM_004078 | cysteine and glycine-rich protein 1 |
| CST9 | NM_001008693 | cystatin 9 |
| CTCF | NM_006565 | CCCTC-binding factor |
| CTCFL | NM_080618 | CCCTC-binding factor-like protein |
| CTDSP1 | NM_021198 | CTD (carboxy-terminal domain, RNA polymerase II, |
| CTDSP2 | NM_005730 | nuclear LIM interactor-interacting factor 2 |
| CTDSPL | NM_001008392 | small CTD phosphatase 3 isoform 1 |
| CTF1 | NM_001330 | cardiotrophin 1 |
| CTNNBIP1 | NM_001012329 | catenin, beta interacting protein 1 |
| CTNND1 | NM_001331 | catenin (cadherin-associated protein), delta 1 |
| CTNND2 | NM_001332 | catenin (cadherin-associated protein), delta 2 |
| CTPS | NM_001905 | CTP synthase |
| CTPS2 | NM_019857 | cytidine triphosphate synthase II |
| CTSB | NM_001908 | cathepsin B preproprotein |
| CTSC | NM_148170 | cathepsin C isoform b precursor |
| CTSW | NM_001335 | cathepsin W preproprotein |
| CTTNBP2NL | NM_018704 | hypothetical protein LOC55917 |
| CUEDC1 | NM_017949 | CUE domain-containing 1 |
| CUGBP2 | NM_001025076 | CUG triplet repeat, RNA binding protein 2 |
| CUL5 | NM_003478 | Vasopressin-activated calcium-mobilizing |
| CUTL2 | NM_015267 | cut-like 2 |
| CX3CR1 | NM_001337 | chemokine (C-X3-C motif) receptor 1 |
| CXCL1 | NM_001511 | chemokine (C-X-C motif) ligand 1 |
| CXCL10 | NM_001565 | small inducible cytokine B10 precursor |
| CXCL11 | NM_005409 | small inducible cytokine B11 precursor |
| CXCL12 | NM_000609 | chemokine (C-X-C motif) ligand 12 (stromal |
| CXCL14 | NM_004887 | small inducible cytokine B14 precursor |
| CXCL16 | NM_022059 | chemokine (C-X-C motif) ligand 16 |
| CXCL2 | NM_002089 | chemokine (C-X-C motif) ligand 2 |
| CXCL5 | NM_002994 | chemokine (C-X-C motif) ligand 5 precursor |
| CXCR3 | NM_001504 | chemokine (C-X-C motif) receptor 3 |
| CXorf12 | NM_003492 | chromosome X open reading frame 12 |
| CXorf15 | NM_018360 | gamma-taxilin |
| CXorf9 | NM_018990 | SH3 protein expressed in lymphocytes |
| CYB561D2 | NM_007022 | cytochrome b-561 domain containing 2 |
| CYB5B | NM_030579 | cytochrome b5 outer mitochondrial membrane |
| CYB5R2 | NM_001001336 | cytochrome b5 reductase b5R.2 isoform 2 |
| CYBASC3 | NM_153611 | cytochrome b, ascorbate dependent 3 |
| CYBRD1 | NM_024843 | cytochrome b reductase 1 |
| CYCS | NM_018947 | cytochrome c |
| CYP11B1 | NM_000497 | cytochrome P450, family 11, subfamily B, |
| CYP19A1 | NM_000103 | cytochrome P450, family 19 |
| CYP20A1 | NM_020674 | cytochrome P450, family 20, subfamily A, |
| CYP27B1 | NM_000785 | cytochrome P450, family 27, subfamily B, |
| CYP4F3 | NM_000896 | cytochrome P450, family 4, subfamily F, |
| CYP4F8 | NM_007253 | cytochrome P450, family 4, subfamily F, |
| CYR61 | NM_001554 | cysteine-rich, angiogenic inducer, 61 |
| CYYR1 | NM_052954 | cysteine and tyrosine-rich 1 protein precursor |
| D15Wsu75e | NM_015704 | hypothetical protein LOC27351 |
| D2HGDH | NM_152783 | D-2-hydroxyglutarate dehydrogenase |
| D4ST1 | NM_130468 | dermatan 4 sulfotransferase 1 |
| DAAM1 | NM_014992 | dishevelled-associated activator of |
| DAAM2 | NM_015345 | dishevelled associated activator of |
| DAB2IP | NM_032552 | DAB2 interacting protein isoform 1 |
| DAG1 | NM_004393 | dystroglycan 1 precursor |
| DAK | NM_015533 | dihydroxyacetone kinase 2 |
| DAO | NM_001917 | D-amino-acid oxidase |
| DAPK2 | NM_014326 | death-associated protein kinase 2 |
| DARC | NM_002036 | Duffy blood group |
| DBC1 | NM_014618 | deleted in bladder cancer 1 |
| DBF4B | NM_145663 | DBF4 homolog B isoform 1 |
| DBNDD1 | NM_024043 | dysbindin (dystrobrevin binding protein 1) |
| DBNDD2 | NM_033542 | SCF apoptosis response protein 1 isoform 2 |
| DBNL | NM_001014436 | drebrin-like isoform b |
| DCBLD1 | NM_173674 | discoidin, CUB and LCCL domain containing 1 |
| DCLRE1B | NM_022836 | DNA cross-link repair 1B (PSO2 homolog, S. |
| DCST2 | NM_144622 | hypothetical protein LOC127579 |
| DCTN5 | NM_032486 | dynactin 4 |
| DCUN1D3 | NM_173475 | hypothetical protein LOC123879 |
| DCX | NM_000555 | doublecortin isoform a |
| DDB1 | NM_001923 | damage-specific DNA binding protein 1 |
| DDEF1 | NM_018482 | development and differentiation enhancing factor |
| DDEF2 | NM_003887 | development-and differentiation-enhancing |
| DDN | NM_015086 | dendrin |
| DDX10 | NM_004398 | DEAD (Asp-Glu-Ala-Asp) box polypeptide 10 |
| DDX11 | NM_004399 | DEAD/H (Asp-Glu-Ala-Asp/His) box polypeptide 11 |
| DDX17 | NM_006386 | DEAD box polypeptide 17 isoform p82 |
| DDX19A | NM_018332 | DDX19-like protein |
| DDX19B | NM_001014449 | DEAD (Asp-Glu-Ala-As) box polypeptide 19 isoform |
| DDX19-DDX19L | NM_001015047 | DDX19-DDX19L protein |
| DDX21 | NM_004728 | DEAD (Asp-Glu-Ala-Asp) box polypeptide 21 |
| DDX26B | NM_182540 | hypothetical protein LOC203522 |
| DDX41 | NM_016222 | DEAD-box protein abstrakt |
| DDX58 | NM_014314 | DEAD/H (Asp-Glu-Ala-Asp/His) box polypeptide |
| DDX59 | NM_031306 | DEAD (Asp-Glu-Ala-Asp) box polypeptide 59 |
| DEADC1 | NM_182503 | deaminase domain containing 1 |
| DEDD2 | NM_133328 | death effector domain-containing DNA binding |
| DENND1A | NM_020946 | hypothetical protein LOC57706 isoform 1 |
| DENND2D | NM_024901 | DENN/MADD domain containing 2D |
| DEPDC5 | NM_014662 | DEP domain containing 5 isoform 1 |
| DEPDC6 | NM_022783 | DEP domain containing 6 |
| DERL3 | NM_001002862 | derlin-3 protein isoform b |
| DFFA | NM_004401 | DNA fragmentation factor, 45 kDa, alpha |
| DFNB31 | NM_015404 | CASK-interacting protein C1P98 |
| DGAT1 | NM_012079 | diacylglycerol O-acyltransferase 1 |
| DGAT2 | NM_032564 | diacylglycerol O-acyltransferase homolog 2 |
| DGAT2L6 | NM_198512 | diacylglycerol O-acyltransferase 2 like 6 |
| DGCR13 | NM_001024733 | DiGeorge syndrome gene H |
| DGCR2 | NM_005137 | integral membrane protein DGCR2 |
| DGCR6 | NM_005675 | DiGeorge syndrome critical region protein 6 |
| DGCR6L | NM_033257 | DiGeorge syndrome critical region gene 6 like |
| DGKB | NM_145695 | diacylglycerol kinase, beta isoform 2 |
| DGKI | NM_004717 | diacylglycerol kinase, iota |
| DGKZ | NM_003646 | diacylglycerol kinase, zeta 104 kDa isoform 2 |
| DHCR24 | NM_014762 | 24-dehydrocholesterol reductase precursor |
| DHCR7 | NM_001360 | 7-dehydrocholesterol reductase |
| DHTKDI | NM_018706 | dehydrogenase E1 and transketolase domain |
| DHX34 | NM_194428 | DEAH (Asp-Glu-Ala-His) box polypeptide 34 |
| DHX40 | NM_024612 | DEAH (Asp-Glu-Ala-His) box polypeptide 40 |
| DIABLO | NM_019887 | diablo isoform 1 precursor |
| DICER1 | NM_030621 | dicer 1 |
| DIDO1 | NM_022105 | death inducer-obliterator 1 isoform a |
| DIP | NM_015124 | death-inducing-protein |
| DIP2C | NM_014974 | hypothetical protein LOC22982 |
| DIRAS1 | NM_145173 | small GTP-binding tumor suppressor 1 |
| DISC1 | NM_001012957 | disrupted in schizophrenia 1 isoform Lv |
| DISP2 | NM_033510 | dispatched B |
| DIXDC1 | NM_033425 | DIX domain containing 1 isoform b |
| DKFZp434I1020 | NM_194295 | hypothetical protein LOC196968 |
| DKFZp451A211 | NM_001003399 | hypothetical protein LOC400169 |
| DKFZp564K142 | NM_032121 | implantation-associated protein |
| DKFZp686O24166 | NM_001009913 | hypothetical protein LOC374383 |
| DKFZp761B107 | NM_173463 | hypothetical protein LOC91050 |
| DKFZP761H1710 | NM_031297 | hypothetical protein LOC83459 |
| DKFZp779B1540 | NM_001010903 | hypothetical protein LOC389384 |
| DKK1 | NM_012242 | dickkopf homolog 1 precursor |
| DLAT | NM_001931 | dihydrolipoamide S-acetyltransferase (E2 |
| DLEC1 | NM_007335 | deleted in lung and esophageal cancer 1 isoform |
| DLG5 | NM_004747 | discs large homolog 5 |
| DLGAP2 | NM_004745 | discs large-associated protein 2 |
| DLL1 | NM_005618 | delta-like 1 |
| DLL4 | NM_019074 | delta-like 4 protein precursor |
| DLX1 | NM_178120 | distal-less homeobox 1 isoform 1 |
| DLX3 | NM_005220 | distal-less homeobox 3 |
| DMRTC1 | NM_033053 | DMRT-like family C1 |
| DMWD | NM_004943 | dystrophia myotonica-containing WD repeat motif |
| DNAH10 | NM_207437 | dynein, axonemal, heavy polypeptide 10 |
| DNAJB1 | NM_006145 | DnaJ (Hsp40) homolog, subfamily B, member 1 |
| DNAJB12 | NM_001002762 | DnaJ (Hsp40) homolog, subfamily B, member 12 |
| DNAJB2 | NM_006736 | DnaJ (Hsp40) homolog, subfamily B, member 2 |
| DNAJC10 | NM_018981 | DnaJ (Hsp40) homolog, subfamily C, member 10 |
| DNAJC11 | NM_018198 | DnaJ (Hsp40) homolog, subfamily C, member 11 |
| DNAJC14 | NM_032364 | dopamine receptor interacting protein |
| DNAJC18 | NM_152686 | DnaJ (Hsp40) homolog, subfamily C, member 18 |
| DNAL4 | NM_005740 | dynein light chain 4, axonemal |
| DNALI1 | NM_003462 | axonemal dynein light chain |
| DNASE1L2 | NM_001374 | deoxyribonuclease I-like 2 |
| DNMIL | NM_005690 | dynamin 1-like protein isoform 3 |
| DNM3 | NM_015569 | dynamin 3 |
| DNMT3A | NM_175630 | DNA cytosine methyltransferase 3 alpha isoform |
| DOCK3 | NM_004947 | dedicator of cytokinesis 3 |
| DOCK8 | NM_203447 | dedicator of cytokinesis 8 |
| DOCK9 | NM_015296 | dedicator of cytokinesis 9 |
| DOK4 | NM_018110 | downstream of tyrosine kinase 4 |
| DOK5 | NM_018431 | DOK5 protein isoform a |
| DOLPP1 | NM_020438 | dolichyl pyrophosphate phosphatase 1 |
| DPF2 | NM_006268 | D4, zinc and double PHD fingers family 2 |
| DPF3 | NM_012074 | D4, zinc and double PHD fingers, family 3 |
| DPH1 | NM_001383 | diptheria toxin resistance protein required for |
| DPP3 | NM_005700 | dipeptidyl peptidase III |
| DPP4 | NM_001935 | dipeptidylpeptidase IV |
| DPY19L3 | NM_207325 | dpy-19-like 3 |
| DPYD | NM_000110 | dihydropyrimidine dehydrogenase |
| DPYSL3 | NM_001387 | dihydropyrimidinase-like 3 |
| DPYSL4 | NM_006426 | dihydropyrimidinase-like 4 |
| DR1 | NM_001938 | down-regulator of transcription 1 |
| DRD2 | NM_000795 | dopamine receptor D2 isoform long |
| DSC3 | NM_001941 | desmocollin 3 isoform Dsc3a preproprotein |
| DSCR1 | NM_004414 | calcipressin 1 isoform a |
| DSCR3 | NM_006052 | Down syndrome critical region protein 3 |
| DTNA | NM_001390 | dystrobrevin alpha isoform 1 |
| DTX3L | NM_138287 | deltex 3-like |
| DULLARD | NM_015343 | dullard homolog |
| DUOX1 | NM_017434 | dual oxidase 1 precursor |
| DUOX2 | NM_014080 | dual oxidase 2 precursor |
| DUSP13 | NM_001007271 | muscle-restricted dual specificity phosphatase |
| DUSP22 | NM_020185 | dual specificity phosphatase 22 |
| DUSP3 | NM_004090 | dual specificity phosphatase 3 |
| DUSP5 | NM_004419 | dual specificity phosphatase 5 |
| DYNC1LI1 | NM_016141 | dynein light chain-A |
| DYRK2 | NM_003583 | dual-specificity tyrosine-(Y)-phosphorylation |
| E2F2 | NM_004091 | E2F transcription factor 2 |
| E2F3 | NM_001949 | E2F transcription factor 3 |
| E2F5 | NM_001951 | E2F transcription factor 5 |
| EAF1 | NM_033083 | ELL associated factor 1 |
| EARS2 | NM_133451 | hypothetical protein LOC124454 |
| ECEL1 | NM_004826 | endothelin converting enzyme-like 1 |
| ECHDC3 | NM_024693 | enoyl Coenzyme A hydratase domain containing 3 |
| ECOP | NM_030796 | EGFR-coamplified and overexpressed protein |
| EDAR | NM_022336 | ectodysplasin A receptor |
| EDARADD | NM_080738 | EDAR-associated death domain isoform B |
| EDEM3 | NM_025191 | ER degradation enhancer, mannosidase alpha-like |
| EDG3 | NM_005226 | endothelial differentiation, sphingolipid |
| EDG4 | NM_004720 | endothelial differentiation, lysophosphatidic |
| EDN2 | NM_001956 | endothelin 2 |
| EDNRA | NM_001957 | endothelin receptor type A |
| EDNRB | NM_000115 | endothelin receptor type B isoform 1 |
| EEF2K | NM_013302 | elongation factor-2 kinase |
| EEFSEC | NM_021937 | elongation factor for selenoprotein translation |
| EFCAB1 | NM_024593 | EF-hand calcium binding domain 1 |
| EFHD1 | NM_025202 | EF hand domain family, member D1 |
| EFHD2 | NM_024329 | EF hand domain family, member D2 |
| EFNA3 | NM_004952 | ephrin A3 |
| EFNB1 | NM_004429 | ephrin-B1 precursor |
| EFNB3 | NM_001406 | ephrin-B3 precursor |
| EGLN3 | NM_022073 | egl nine homolog 3 |
| EGR2 | NM_000399 | early growth response 2 protein |
| EHD2 | NM_014601 | EH-domain containing 2 |
| EHD4 | NM_139265 | EH-domain containing 4 |
| EI24 | NM_001007277 | etoposide induced 2.4 isoform 2 |
| EIF2AK1 | NM_014413 | heme-regulated initiation factor 2-alpha kinase |
| EIF2B5 | NM_003907 | eukaryotic translation initiation factor 2B, |
| EIF2C1 | NM_012199 | eukaryotic translation initiation factor 2C, 1 |
| EIF2C4 | NM_017629 | eukaryotic translation initiation factor 2C. 4 |
| EIF2S2 | NM_003908 | eukaryotic translation initiation factor 2 beta |
| EIF4EBP2 | NM_004096 | eukaryotic translation initiation factor 4E |
| EIF4G1 | NM_004953 | eukaryotic translation initiation factor 4 |
| ELF2 | NM_006874 | E74-like factor 2 (ets domain transcription |
| ELF5 | NM_001422 | E74-like factor 5 ESE-2b |
| ELL | NM_006532 | elongation factor RNA polymerase II |
| Ells1 | NM_152793 | hypothetical protein LOC222166 |
| ELMO1 | NM_014800 | engulfment and cell motility 1 isoform 1 |
| ELMOD1 | NM_018712 | ELMO domain containing 1 |
| ELP3 | NM_018091 | elongation protein 3 homolog |
| ELSPBP1 | NM_022142 | epididymal sperm binding protein 1 |
| EMD | NM_000117 | emerin |
| EME1 | NM_152463 | essential meiotic endonuclease 1 homolog 1 |
| EML5 | NM_183387 | echinoderm microtubule associated protein like |
| EMP1 | NM_001423 | epithelial membrane protein 1 |
| EMR2 | NM_013447 | egf-like module-containing, mucin-like, hormone |
| EMR3 | NM_152939 | egf-like module-containing mucin-like receptor 3 |
| EN2 | NM_001427 | engrailed homolog 2 |
| ENAM | NM_031889 | enamelin |
| ENPP1 | NM_006208 | ectonucleotide pyrophosphatase/phosphodiesterase |
| ENSA | NM_207043 | endosulfine alpha isoform 2 |
| ENTPD3 | NM_001248 | ectonucleoside triphosphate diphosphohydrolase |
| EPB41 | NM_004437 | erythrocyte membrane protein band 4.1 |
| EPHA4 | NM_004438 | ephrin receptor EphA4 |
| EPHB2 | NM_004442 | ephrin receptor EphB2 isoform 2 precursor |
| EPN2 | NM_014964 | epsin 2 isoform b |
| EPN3 | NM_017957 | epsin 3 |
| EPS15L1 | NM_021235 | epidermal growth factor receptor pathway |
| EPSTI1 | NM_033255 | epithelial stromal interaction 1 isoform 2 |
| ERBB2 | NM_001005862 | erbB-2 isoform b |
| ERGIC1 | NM_001031711 | endoplasmic reticulum-golgi intermediate |
| ERMAP | NM_001017922 | erythroblast membrane-associated protein |
| ESPN | NM_031475 | espin |
| ESRRA | NM_004451 | estrogen-related receptor alpha |
| ESRRG | NM_001438 | estrogen-related receptor gamma isoform 1 |
| ETS1 | NM_005238 | v-ets erythroblastosis virus E26 oncogene |
| ETV6 | NM_001987 | ets variant gene 6 |
| EVA1 | NM_144765 | epithelial V-like antigen 1 precursor |
| EVC | NM_153717 | Ellis van Creveld syndrome protein |
| EVI5L | NM_145245 | hypothetical protein LOC115704 |
| EXOSC6 | NM_058219 | homolog of yeast mRNA transport regulator 3 |
| F11R | NM_016946 | F11 receptor isoform a precursor |
| F2RL2 | NM_004101 | coagulation factor II (thrombin) receptor-like 2 |
| F8 | NM_000132 | coagulation factor VIII isoform a precursor |
| FABP3 | NM_004102 | fatty acid binding protein 3 |
| FAIM2 | NM_012306 | Fas apoptotic inhibitory molecule 2 |
| FAM100B | NM_182565 | hypothetical protein LOC283991 |
| FAM101A | NM_181709 | hypothetical protein LOC144347 |
| FAM101B | NM_182705 | hypothetical protein LOC359845 |
| FAM102A | NM_203305 | early estrogen-induced gene 1 protein isoform b |
| FAM104A | NM_032837 | hypothetical protein LOC84923 |
| FAM105B | NM_138348 | hypothetical protein LOC90268 |
| FAM107A | NM_007177 | downregulated in renal cell carcinoma |
| FAM109A | NM_144671 | hypothetical protein LOC144717 |
| FAM109B | NM_001002034 | hypothetical protein LOC150368 |
| FAM111B | NM_198947 | hypothetical protein LOC374393 |
| FAM112A | NM_001008901 | hypothetical protein LOC149699 isoform 2 |
| FAM11A | NM_032508 | family with sequence similarity 11, member A |
| FAM26C | NM_001001412 | hypothetical protein LOC255022 |
| FAM36A | NM_198076 | family with sequence similarity 36, member A |
| FAM38A | NM_014745 | family with sequence similarity 38, member A |
| FAM3A | NM_021806 | family 3, member A protein |
| FAM3C | NM_014888 | family with sequence similarity 3, member C |
| FAM3D | NM_138805 | family with sequence similarity 3, member D |
| FAM49B | NM_016623 | hypothetical protein LOC51571 |
| FAM51A1 | NM_017856 | family with sequence similarity 51, member Al |
| FAM53A | NM_001013622 | dorsal neural-tube nuclear protein |
| FAM53B | NM_014661 | hypothetical protein LOC9679 |
| FAM53C | NM_016605 | family 53, member C protein |
| FAM55C | NM_145037 | hypothetical protein LOC91775 |
| FAM60A | NM_021238 | family with sequence similarity 60, member A |
| FAM62C | NM_031913 | family with sequence similarity 62 (C2 domain |
| FAM64A | NM_019013 | hypothetical protein LOC54478 |
| FAM70A | NM_017938 | hypothetical protein LOC55026 |
| FAM71A | NM_153606 | hypothetical protein LOC149647 |
| FAM73B | NM_032809 | hypothetical protein LOC84895 |
| FAM76A | NM_152660 | family with sequence similarity 76, member A |
| FAM77C | NM_024522 | hypothetical protein LOC79570 |
| FAM78A | NM_033387 | hypothetical protein LOC286336 |
| FAM81A | NM_152450 | hypothetical protein LOC145773 |
| FAM83A | NM_032899 | hypothetical protein LOC84985 isoform a |
| FAM84A | NM_145175 | NSE1 |
| FAM86A | NM_201400 | hypothetical protein LOC196483 isoform 1 |
| FAM86B1 | NM_032916 | hypothetical protein LOC85002 |
| FAM86C | NM_018172 | hypothetical protein LOC55199 isoform 1 |
| FAM89B | NM_152832 | Mouse Mammary Turmor Virus Receptor homolog 1 |
| FAM8A1 | NM_016255 | Autosomal Highly Conserved Protein |
| FAM92B | NM_198491 | hypothetical protein LOC339145 |
| FAM9C | NM_174901 | family with sequence similarity 9, member C |
| FANCA | NM_000135 | Fanconi anemia, complementation group A isoform |
| FANCC | NM_000136 | Fanconi anemia, complementation group C |
| FANCE | NM_021922 | Fanconi anemia, complementation group E |
| FANCM | NM_020937 | Fanconi anemia, complementation group M |
| FARP2 | NM_014808 | FERM, RhoGEF and pleckstrin domain protein 2 |
| FARSLB | NM_005687 | phenylalanine-tRNA synthetase-like, beta |
| FAS | NM_000043 | tumor necrosis factor receptor superfamily, |
| FASN | NM_004104 | fatty acid synthase |
| FAT2 | NM_001447 | FAT tumor suppressor 2 precursor |
| FATE1 | NM_033085 | fetal and adult testis expressed transcript |
| FBLN1 | NM_006485 | fibulin 1 isoform B precursor |
| FBXL17 | NM_022824 | F-box and leucine-rich repeat protein 17 |
| FBXL19 | NM_019085 | F-box and leucine-rich repeat protein 19 |
| FBXL8 | NM_018378 | F-box and leucine-rich repeat protein 8 |
| FBXO16 | NM_172366 | F-box only protein 16 |
| FBXO17 | NM_024907 | F-box protein FBG4 isoform 2 |
| FBXO25 | NM_012173 | F-box only protein 25 isoform 3 |
| FBXO30 | NM_032145 | F-box only protein 30 |
| FBXO34 | NM_017943 | F-box only protein 34 |
| FBXO39 | NM_153230 | F-box protein 39 |
| FBXO40 | NM_016298 | F-box protein 40 |
| FBXO44 | NM_001014765 | F-box protein 44 isoform 1 |
| FBXW4 | NM_022039 | F-box and WD-40 domain protein 4 |
| FBXW9 | NM_032301 | F-box and WD-40 domain protein 9 |
| FCER1G | NM_004106 | Fc fragment of IgE, high affinity 1, receptor |
| FCGR2B | NM_001002273 | Fc fragment of IgG, low affinity IIb, receptor |
| FCHO2 | NM_138782 | FCH domain only 2 |
| FCHSD2 | NM_014824 | FCH and double SH3 domains 2 |
| FCMD | NM_006731 | fukutin |
| FCRL5 | NM_031281 | Fc receptor-like 5 |
| FDFT1 | NM_004462 | farnesyl-diphosphate farnesyltransferase 1 |
| FEM1A | NM_018708 | fem-1 homolog a (C. elegans) |
| FEM1C | NM_020177 | feminization 1 homolog a |
| FES | NM_002005 | V-FES feline sarcoma viral/V-FPS fujinami avian |
| FETUB | NM_014375 | fetuin B |
| FGD2 | NM_173558 | FYVE, RhoGEF and PH domain containing 2 |
| FGD3 | NM_033086 | FYVE, RhoGEF and PH domain containing 3 |
| FGD6 | NM_018351 | FYVE, RhoGEF and PH domain containing 6 |
| FGF13 | NM_004114 | fibroblast growth factor 13 isoform 1A |
| FGF2 | NM_002006 | fibroblast growth factor 2 |
| FGF23 | NM_020638 | fibroblast growth factor 23 precursor |
| FGF7 | NM_002009 | fibroblast growth factor 7 precursor |
| FGFR1 | NM_000604 | fibroblast growth factor receptor 1 isoform 1 |
| FGFR2 | NM_000141 | fibroblast growth factor receptor 2 isoform 1 |
| FGFRL1 | NM_001004356 | fibroblast growth factor receptor-like 1 |
| FIGN | NM_018086 | fidgetin |
| FKBP1A | NM_054014 | FK506-binding protein 1A |
| FKBP1B | NM_004116 | FK506-binding protein 1B isoform a |
| FKBP8 | NM_012181 | FK506-binding protein 8 |
| FKBP9 | NM_007270 | FK506 binding protein 9 |
| FKBP9L | NM_182827 | FK506 binding protein 9-like |
| FKSG24 | NM_032683 | hypothetical protein LOC84769 |
| FLJ10081 | NM_017991 | hypothetical protein LOC55683 |
| FLJ10159 | NM_018013 | hypothetical protein LOC55084 |
| FLJ10241 | NM_018035 | hypothetical protein LOC55101 |
| FLJ10324 | NM_018059 | hypothetical protein LOC55698 |
| FLJ10404 | NM_019057 | hypothetical protein LOC54540 |
| FLJ10769 | NM_018210 | hypothetical protein LOC55739 |
| FLJ10803 | NM_018224 | hypothetical protein LOC55744 |
| FLJ10815 | NM_018231 | amino acid transporter |
| FLJ10945 | NM_018280 | hypothetical protein LOC55267 |
| FLJ11292 | NM_018382 | hypothetical protein LOC55338 |
| FLJ11506 | NM_024666 | hypothetical protein LOC79719 |
| FLJ12331 | NM_024986 | hypothetical protein LOC80052 |
| FLJ12505 | NM_024749 | hypothetical protein LOC79805 |
| FLJ12529 | NM_024811 | pre-mRNA cleavage factor I, 59 kDa subunit |
| FLJ12700 | NM_024910 | hypothetical protein LOC79970 |
| FLJ12949 | NM_023008 | hypothetical protein LOC65095 isoform 1 |
| FLJ13197 | NM_024614 | hypothetical protein LOC79667 |
| FLJ14001 | NM_024677 | hypothetical protein LOC79730 |
| FLJ14154 | NM_024845 | hypothetical protein LOC79903 |
| FLJ14768 | NM_032836 | hypothetical protein FLJ14768 |
| FLJ14816 | NM_032845 | hypothetical protein LOC84931 |
| FLJ14834 | NM_032849 | hypothetical protein LOC84935 |
| FLJ16165 | NM_001004318 | hypothetical protein LOC390928 |
| FLJ16171 | NM_001004348 | hypothetical protein LOC441116 |
| FLJ16323 | NM_001004352 | hypothetical protein LOC441390 |
| FLJ20152 | NM_019000 | hypothetical protein LOC54463 isoform 2 |
| FLJ20232 | NM_019008 | hypothetical protein LOC54471 |
| FLJ20297 | NM_017751 | hypothetical protein LOC55627 isoform 1 |
| FLJ20489 | NM_017842 | hypothetical protein LOC55652 |
| FLJ20699 | NM_017931 | hypothetical protein LOC55020 |
| FLJ20701 | NM_017933 | hypothetical protein LOC55022 |
| FLJ20758 | NM_017952 | hypothetical protein LOC55037 |
| FLJ20850 | NM_017967 | hypothetical protein LOC55049 |
| FLJ20859 | NM_001029992 | FLJ20859 protein isoform 3 |
| FLJ21742 | NM_032207 | hypothetical protein LOC84167 |
| FLJ21820 | NM_021925 | hypothetical protein LOC60526 |
| FLJ21945 | NM_025203 | hypothetical protein LOC80304 |
| FLJ22795 | NM_025084 | hypothetical protein LOC80154 |
| FLJ23322 | NM_024955 | hypothetical protein LOC80020 |
| FLJ23447 | NM_024825 | hypothetical protein LOC79883 |
| FLJ25102 | NM_182626 | hypothetical protein LOC348738 |
| FLJ25222 | NM_199163 | hypothetical protein LOC374666 |
| FLJ25371 | NM_152543 | hypothetical protein LOC152940 |
| FLJ25996 | NM_001001699 | hypothetical protein LOC401109 |
| FLJ26850 | NM_001001687 | hypothetical protein LOC400710 |
| FLJ30058 | NM_144967 | hypothetical protein LOC158763 |
| FLJ30707 | NM_145019 | hypothetical protein LOC220108 |
| FLJ30834 | NM_152399 | hypothetical protein LOC132332 |
| FLJ31132 | NM_001004355 | hypothetical protein LOC441522 |
| FLJ31568 | NM_152509 | hypothetical protein LOC150244 |
| FLJ31951 | NM_144726 | hypothetical protein LOC153830 |
| FLJ32011 | NM_182516 | hypothetical protein LOC148930 |
| FLJ32206 | NM_152497 | hypothetical protein LOC149421 |
| FLJ33534 | NM_182586 | hypothetical protein LOC285150 |
| FLJ33641 | NM_152687 | hypothetical protein LOC202309 |
| FLJ33708 | NM_173675 | hypothetical protein LOC285780 |
| FLJ33814 | NM_173510 | hypothetical protein LOC150275 |
| FLJ34870 | NM_207481 | hypothetical protein LOC401013 |
| FLJ34931 | NM_001029883 | hypothetical protein LOC388939 |
| FLJ35424 | NM_173661 | hypothetical protein LOC285492 |
| FLJ35429 | NM_001003807 | hypothetical protein LOC285830 |
| FLJ35695 | NM_207444 | hypothetical protein LOC400359 |
| FLJ35725 | NM_152544 | hypothetical protein LOC152992 isoform 2 |
| FLJ36070 | NM_182574 | hypothetical protein LOC284358 |
| FLJ36268 | NM_207511 | hypothetical protein LOC401563 |
| FLJ37464 | NM_173815 | hypothetical protein LOC283848 |
| FLJ37478 | NM_178557 | hypothetical protein LOC339983 |
| FLJ37543 | NM_173667 | hypothetical protein LOC285668 |
| FLJ38723 | NM_173805 | hypothetical protein FLJ38723 |
| FLJ38973 | NM_153689 | hypothetical protein LOC205327 |
| FLJ39155 | NM_182798 | hypothetical protein LOC133584 isoform 2 |
| FLJ39378 | NM_178314 | hypothetical protein LOC353116 |
| FLJ39531 | NM_207445 | hypothetical protein LOC400360 |
| FLJ39599 | NM_173803 | Mpv17-like protein type 2 |
| FLJ39827 | NM_152424 | hypothetical protein LOC139285 |
| FLJ40172 | NM_173649 | hypothetical protein LOC285051 |
| FLJ40852 | NM_173677 | hypothetical protein LOC285962 |
| FLJ41131 | NM_198476 | hypothetical protein LOC284325 |
| FLJ41423 | NM_001001679 | hypothetical protein LOC399886 |
| FLJ41603 | NM_001001669 | hypothetical protein LOC389337 |
| FLJ41733 | NM_207473 | hypothetical protein LOC400870 |
| FLJ41993 | NM_001001694 | hypothetical protein LOC400935 |
| FLJ42280 | NM_207503 | hypothetical protein LOC401388 |
| FLJ42291 | NM_207367 | hypothetical protein LOC346547 |
| FLJ42393 | NM_207488 | hypothetical protein LOC401105 |
| FLJ42957 | NM_207436 | hypothetical protein LOC400077 |
| FLJ43339 | NM_207380 | hypothetical protein LOC388115 |
| FLJ43752 | NM_207497 | hypothetical protein LOC401253 |
| FLJ43806 | NM_201628 | hypothetical protein LOC399563 |
| FLJ43870 | NM_001001686 | hypothetical protein LOC400686 |
| FLJ44006 | NM_001001696 | hypothetical protein LOC400997 |
| FLJ44076 | NM_207486 | hypothetical protein LOC401080 |
| FLJ44385 | NM_207478 | hypothetical protein LOC400934 |
| FLJ44635 | NM_207422 | hypothetical protein LOC392490 |
| FLJ44790 | NM_001001691 | hypothetical protein LOC400850 |
| FLJ44815 | NM_207454 | hypothetical protein LOC400591 |
| FLJ44955 | NM_207500 | hypothetical protein LOC401278 |
| FLJ45121 | NM_207451 | hypothetical protein LOC400556 |
| FLJ45224 | NM_207510 | hypothetical protein LOC401562 |
| FLJ45244 | NM_207443 | hypothetical protein LOC400242 |
| FLJ45337 | NM_207465 | hypothetical protein LOC400754 |
| FLJ45422 | NM_001004349 | hypothetical protein LOC441140 |
| FLJ45455 | NM_207386 | hypothetical protein LOC388336 |
| FLJ45537 | NM_001001709 | hypothetical protein LOC401535 |
| FLJ45684 | NM_207462 | hypothetical protein LOC400666 |
| FLJ45831 | NM_001001684 | hypothetical protein LOC400576 |
| FLJ45850 | NM_207395 | hypothetical protein LOC388569 |
| FLJ46026 | NM_207458 | hypothetical protein LOC400627 |
| FLJ46154 | NM_198462 | FLJ46154 protein |
| FLJ46230 | NM_207463 | hypothetical protein LOC400679 |
| FLJ46266 | NM_207430 | hypothetical protein LOC399949 |
| FLJ46300 | NM_001001677 | hypothetical protein LOC399827 |
| FLJ46836 | NM_207509 | hypothetical protein LOC401554 |
| FLJ90680 | NM_207475 | hypothetical protein LOC400926 |
| FLOT2 | NM_004475 | flotillin 2 |
| FLRT3 | NM_013281 | fibronectin leucine rich transmembrane protein 3 |
| FLYWCH1 | NM_032296 | FLYWCH-type zinc finger 1 isoform a |
| FMNL2 | NM_052905 | formin-like 2 |
| FMNL3 | NM_175736 | formin-like 3 isoform 1 |
| FMO2 | NM_001460 | flavin containing monooxygenase 2 |
| FMO5 | NM_001461 | flavin containing monooxygenase 5 |
| FNBP1L | NM_001024948 | formin binding protein 1-like isoform 1 |
| FNDC3B | NM_022763 | fibronectin type III domain containing 3B |
| FNDC5 | NM_153756 | fibronectin type III domain containing 5 |
| FNDC8 | NM_017559 | hypothetical protein LOC54752 |
| FOS | NM_005252 | v-fos FBJ murine osteosarcoma viral oncogene |
| FOSB | NM_006732 | FBJ murine osteosarcoma viral oncogene homolog |
| FOSL1 | NM_005438 | FOS-like antigen 1 |
| FOSL2 | NM_005253 | FOS-like antigen 2 |
| FOXE1 | NM_004473 | forkhead box E1 |
| FOXG1B | NM_005249 | forkhead box G1B |
| FOXI1 | NM_012188 | forkhead box I1 isoform a |
| FOXJ1 | NM_001454 | forkhead box J1 |
| FOXJ2 | NM_018416 | forkhead box J2 |
| FOXK2 | NM_004514 | forkhead box K2 isoform 1 |
| FOXL2 | NM_023067 | forkhead box L2 |
| FOXM1 | NM_021953 | forkhead box M1 isoform 2 |
| FOXP1 | NM_032682 | forkhead box P1 isoform 1 |
| FOXQ1 | NM_033260 | forkhead box Q1 |
| FOXR2 | NM_198451 | forkhead box R2 |
| FOXRED1 | NM_017547 | FAD-dependent oxidoreductase domain containing |
| FRAG1 | NM_014489 | FGF receptor activating protein 1 |
| FREM1 | NM_144966 | FRAS1 related extracellular matrix 1 |
| FRK | NM_002031 | fyn-related kinase |
| FRMD1 | NM_024919 | FERM domain containing 1 |
| FRMD4A | NM_018027 | FERM domain containing 4A |
| FSD1L | NM_207647 | fibronectin type III and SPRY domain containing |
| FSTL1 | NM_007085 | follistatin-like 1 precursor |
| FSTL3 | NM_005860 | follistatin-like 3 glycoprotein precursor |
| FSTL4 | NM_015082 | follistatin-like 4 |
| FSTL5 | NM_020116 | follistatin-like 5 |
| FTS | NM_001012398 | fused toes homolog |
| FUK | NM_145059 | fucokinase |
| FUNDC1 | NM_173794 | FUN14 domain containing 1 |
| FURIN | NM_002569 | furin preproprotein |
| FUT1 | NM_000148 | fucosyltransferase 1 |
| FUT10 | NM_032664 | fucosyltransferase 10 |
| FUT5 | NM_002034 | fucosyltransferase 5 |
| FUT8 | NM_004480 | fucosyltransferase 8 isoform b |
| FXC1 | NM_012192 | fracture callus 1 homolog |
| FXN | NM_000144 | frataxin isoform 1 preproprotein |
| FXYD2 | NM_001680 | FXYD domain-containing ion transport regulator 2 |
| FYCO1 | NM_024513 | FYVE and coiled-coil domain containing 1 |
| FZD1 | NM_003505 | frizzled 1 |
| FZD4 | NM_012193 | frizzled 4 |
| FZR1 | NM_016263 | Fzrl protein |
| GABARAPL2 | NM_007285 | GABA(A) receptor-associated protein-like 2 |
| GABBR2 | NM_005458 | G protein-coupled receptor 51 |
| GABRA1 | NM_000806 | gamma-aminobutyric acid (GABA) A receptor, alpha |
| GABRE | NM_004961 | gamma-aminobutyric acid (GABA) A receptor, |
| GABRG1 | NM_173536 | gamma-aminobutyric acid A receptor, gamma 1 |
| GADD45G | NM_006705 | growth arrest and DNA-damage-inducible, gamma |
| GALM | NM_138801 | galactose mutarotase (aldose 1-epimerase) |
| GALNT2 | NM_004481 | polypeptide N-acetylgalactosaminyltransferase 2 |
| GALNT7 | NM_017423 | polypeptide N-acetylgalactosaminyltransferase 7 |
| GALNTL1 | NM_020692 | UDP-N-acetyl-alpha-D-galactosamine: polypeptide |
| GARNL4 | NM_015085 | GTPase activating Rap/RanGAP domain-like 4 |
| GAS1 | NM_002048 | growth arrest-specific 1 |
| GAS8 | NM_001481 | growth arrest-specific 8 |
| GATA3 | NM_001002295 | GATA binding protein 3 isoform 1 |
| GATA5 | NM_080473 | GATA binding protein 5 |
| GATAD2A | NM_017660 | GATA zinc finger domain containing 2A |
| GATAD2B | NM_020699 | GATA zinc finger domain containing 2B |
| GATS | NM_178831 | opposite strand transcription unit to STAG3 |
| GBA3 | NM_020973 | cytosolic beta-glucosidase |
| GBF1 | NM_004193 | golgi-specific brefeldin A resistance factor 1 |
| GBL | NM_022372 | G protein beta subunit-like |
| GBP2 | NM_004120 | guanylate binding protein 2, |
| GBP4 | NM_052941 | guanylate binding protein 4 |
| GCAT | NM_014291 | glycine C-acetyltransferase precursor |
| GCH1 | NM_000161 | GTP cyclohydrolase 1 isoform 1 |
| GCLM | NM_002061 | glutamate-cysteine ligase regulatory protein |
| GCM1 | NM_003643 | glial cells missing homolog a |
| GCNT1 | NM_001490 | beta-1,3-galactosyl-O-glycosyl-glycoprotein |
| GCNT2 | NM_001491 | glucosaminyl (N-acetyl) transferase 2, |
| Gcom1 | NM_001018097 | GRINL1A combined protein isoform 8 |
| GDA | NM_004293 | guanine deaminase |
| GDAP1L1 | NM_024034 | ganglioside-induced differentiation-associated |
| GDF2 | NM_016204 | growth differentiation factor 2 |
| GDF5 | NM_000557 | growth differentiation factor 5 preproprotein |
| GDF8 | NM_005259 | growth differentiation factor 8 |
| Gene_symbol | has-miR-34a target | Gene_name |
| GENX-3414 | NM_003943 | genethonin 1 |
| GFAP | NM_002055 | glial fibrillary acidic protein |
| GFER | NM_005262 | erv1-like growth factor |
| GFRA3 | NM_001496 | GDNF family receptor alpha 3 preproprotein |
| GIMAP6 | NM_001007224 | GTPase, IMAP family member 6 isoform 3 |
| GINS3 | NM_022770 | hypothetical protein LOC64785 |
| GIPC1 | NM_005716 | regulator of G-protein signalling 19 interacting |
| GIPC2 | NM_017655 | PDZ domain protein GIPC2 |
| GJA5 | NM_005266 | gap junction protein, alpha 5 |
| GJC1 | NM_152219 | gap junction protein, chi 1, 31.9 kDa (connexin |
| GLCE | NM_015554 | D-glucuronyl C5-epimerase |
| GL14 | NM_138465 | GLI-Kruppel family member GLI4 |
| GLIS2 | NM_032575 | GLIS family zinc finger 2 |
| GLP1R | NM_002062 | glucagon-like peptide 1 receptor |
| GLRA3 | NM_006529 | glycine receptor, alpha 3 |
| GLRX | NM_002064 | glutaredoxin (thioltransferase) |
| GLRX5 | NM_016417 | glutaredoxin 5 |
| GLS | NM_014905 | glutaminase C |
| GLT25D1 | NM_024656 | glycosyltransferase 25 domain containing 1 |
| GLT25D2 | NM_015101 | glycosyltransferase 25 domain containing 2 |
| GLT8D1 | NM_001010983 | glycosyltransferase 8 domain containing 1 |
| GLTP | NM_016433 | glycolipid transfer protein |
| GM2A | NM_000405 | GM2 ganglioside activator precursor |
| GM632 | NM_020713 | hypothetical protein LOC57473 |
| GMFB | NM_004124 | glia maturation factor, beta |
| GMIP | NM_016573 | GEM interacting protein |
| GMNN | NM_015895 | geminin |
| GNA12 | NM_007353 | guanine nucleotide binding protein (G protein) |
| GNAI2 | NM_002070 | guanine nucleotide binding protein (G protein), |
| GNAL | NM_002071 | guanine nucleotide binding protein (G protein), |
| GNAS | NM_016592 | guanine nucleotide binding protein, alpha |
| GNAZ | NM_002073 | guanine nucleotide binding protein, alpha z |
| GNB3 | NM_002075 | guanine nucleotide-binding protein, beta-3 |
| GNG10 | NM_001017998 | guanine nucleotide binding protein (G protein), |
| GNG12 | NM_018841 | G-protein gamma-12 subunit |
| GNG2 | NM_053064 | guanine nucleotide binding protein (G protein), |
| GNG7 | NM_052847 | guanine nucleotide binding protein (G protein), |
| GNPDA1 | NM_005471 | glucosamine-6-phosphate deaminase 1 |
| GNPNAT1 | NM_198066 | glucosamine-phosphate N-acetyltransferase 1 |
| GNPTAB | NM_024312 | N-acetylglucosamine-1-phosphate transferase |
| GNRHR | NM_000406 | gonadotropin-releasing hormone receptor isoform |
| GNS | NM_002076 | glucosamine (N-acetyl)-6-sulfatase precursor |
| GOLGA4 | NM_002078 | golgi autoantigen, golgin subfamily a, 4 |
| GOLGB1 | NM_004487 | golgi autoantigen, golgin subfamily b, |
| GOLPH3 | NM_022130 | golgi phosphoprotein 3 |
| GOLPH3L | NM_018178 | GPP34-related protein |
| GOLT1B | NM_016072 | golgi transport 1 homolog B |
| GORASP2 | NM_015530 | golgi reassembly stacking protein 2 |
| GOSR1 | NM_001007024 | golgi SNAP receptor complex member 1 isoform 3 |
| GOSR2 | NM_001012511 | golgi SNAP receptor complex member 2 isoform C |
| GP5 | NM_004488 | glycoprotein V (platelet) |
| GPATC3 | NM_022078 | G patch domain containing 3 |
| GPC4 | NM_001448 | glypican 4 |
| GPHB5 | NM_145171 | glycoprotein beta 5 |
| GPR124 | NM_032777 | G protein-coupled receptor 124 |
| GPR135 | NM_022571 | G protein-coupled receptor 135 |
| GPR143 | NM_000273 | G protein-coupled receptor 143 |
| GPR17 | NM_005291 | G protein-coupled receptor 17 |
| GPR26 | NM_153442 | G protein-coupled receptor 26 |
| GPR3 | NM_005281 | G protein-coupled receptor 3 |
| GPR37L1 | NM_004767 | G-protein coupled receptor 37 like 1 |
| GPR4 | NM_005282 | G protein-coupled receptor 4 |
| GPR44 | NM_004778 | G protein-coupled receptor 44 |
| GPR55 | NM_005683 | G protein-coupled receptor 55 |
| GPR56 | NM_005682 | G protein-coupled receptor 56 isoform a |
| GPR6 | NM_005284 | G protein-coupled receptor 6 |
| GPR64 | NM_005756 | G protein-coupled receptor 64 |
| GPR83 | NM_016540 | G protein-coupled receptor 83 |
| GPR84 | NM_020370 | inflammation-related G protein-coupled receptor |
| GPR85 | NM_018970 | G protein-coupled receptor 85 |
| GPR97 | NM_170776 | G protein-coupled receptor 97 |
| GPRC5B | NM_016235 | G protein-coupled receptor, family C, group 5, |
| GPS2 | NM_004489 | G protein pathway suppressor 2 |
| GPX3 | NM_002084 | plasma glutathione peroxidase 3 precursor |
| GRAMD2 | NM_001012642 | hypothetical protein LOC196996 |
| GRAP | NM_006613 | GRB2-related adaptor protein |
| GRB10 | NM_001001549 | growth factor receptor-bound protein 10 isoform |
| GREM2 | NM_022469 | gremlin 2 precursor |
| GRHL1 | NM_014552 | leader-binding protein 32 isoform 1 |
| GRHL2 | NM_024915 | transcription factor CP2-like 3 |
| GRHL3 | NM_021180 | sister-of-mammalian grainyhead protein isoform |
| GRID1 | NM_017551 | glutamate receptor, ionotropic, delta 1 |
| GRIN1 | NM_000832 | NMDA receptor 1 isoform NR1-1 precursor |
| GRIN3A | NM_133445 | glutamate receptor, ionotropic, |
| GRK6 | NM_001004106 | G protein-coupled receptor kinase 6 isoform A |
| GRM1 | NM_000838 | glutamate receptor, metabotropic 1 |
| GRM2 | NM_000839 | glutamate receptor, metabotropic 2 precursor |
| GRM7 | NM_000844 | glutamate receptor, metabotropic 7 isoform a |
| GRSF1 | NM_002092 | G-rich RNA sequence bmdin factor 1 |
| GSDML | NM_018530 | hypothetical protein LOC55876 |
| GSG1 | NM_031289 | germ cell associated 1 isoform 1 |
| GSPT2 | NM_018094 | peptide chain release factor 3 |
| GSTM3 | NM_000849 | glutathione S-transferase M3 |
| GSTM5 | NM_000851 | glutathione S-transferase M5 |
| GTF2F1 | NM_002096 | general transcription factor hF, polypeptide 1, |
| GTF3C4 | NM_012204 | general transcription factor IIIC, polypeptide |
| GTSE1 | NM_016426 | G-2 and S-phase expressed 1 |
| GUCA2A | NM_033553 | guanylate cyclase activator 2A |
| GYG1 | NM_004130 | glycogenin |
| GYG2 | NM_003918 | glycogenin 2 |
| GYPE | NM_198682 | glycophorin E precursor |
| H2AFV | NM_138635 | H2A histone family, member V isoform 2 |
| H2AFX | NM_002105 | H2A histone family, member X |
| H6PD | NM_004285 | hexose-6-phosphate dehydrogenase precursor |
| HAAO | NM_012205 | 3-hydroxyanthranilate 3,4-dioxygenase |
| HABP2 | NM_004132 | hyaluronan binding protein 2 |
| HACE1 | NM_020771 | HECT domain and ankyrin repeat containing, E3 |
| HADH2 | NM_004493 | hydroxyacyl-Coenzyme A dehydrogenase, type II |
| HAP1 | NM_003949 | huntingtin-associated protein 1 isoform 1 |
| HAPLN3 | NM_178232 | hyaluronan and proteoglycan link protein 3 |
| HAPLN4 | NM_023002 | brain link protein 2 |
| HARS2 | NM_080820 | histidyl-tRNA synthetase 2 |
| HAS3 | NM_005329 | hyaluronan synthase 3 isoform a |
| HAVCR2 | NM_032782 | T cell immunoglobulin mucin 3 |
| HBG1 | NM_000559 | A-gamma globin |
| HBG2 | NM_000184 | G-gamma globin |
| HBS1L | NM_006620 | HBS1-like |
| HCFC1 | NM_005334 | host cell factor Cl (VP16-accessory protein) |
| HCG9 | NM_005844 | hypothetical protein LOC10255 |
| HCN3 | NM_020897 | hyperpolarization activated cyclic |
| HD | NM_002111 | huntingtin |
| HDAC1 | NM_004964 | histone deacetylase 1 |
| HDAC4 | NM_006037 | histone deacetylase 4 |
| HDAC7A | NM_015401 | histone deacetylase 7A isoform a |
| HDGFL1 | NM_138574 | hepatoma derived growth factor-like 1 |
| HDLBP | NM_005336 | high density lipoprotein binding protein |
| HEBP1 | NM_015987 | heme binding protein 1 |
| HECA | NM_016217 | headcase |
| HECW1 | NM_015052 | NEDD4-like ubiguitin-protein ligase 1 |
| HECW2 | NM_020760 | HECT, C2 and WW domain containing E3 ubiquitin |
| HEMK1 | NM_016173 | HemK methyltransferase family member 1 |
| HERC6 | NM_001013000 | hect domain and RLD 6 isoform c |
| HES2 | NM_019089 | hairy and enhancer of split homolog 2 |
| HES3 | NM_001024598 | hairy and enhancer of split 3 |
| HES6 | NM_018645 | hairy and enhancer of split 6 |
| HEY1 | NM_012258 | hairy/enhancer-of-split related with YRPW motif |
| HEYL | NM_014571 | hairy/enhancer-of-split related with YRPW |
| HGF | NM_001010934 | hepatocyte growth factor isoform 5 precursor |
| HGS | NM_004712 | hepatocyte growth factor-regulated tyrosine |
| HIATL1 | NM_032558 | hypothetical protein LOC84641 |
| HIC2 | NM_015094 | hypermethylated in cancer 2 |
| HIF1AN | NM_017902 | hypoxia-inducible factor 1, alpha subunit |
| HIF3A | NM_152794 | hypoxia-inducible factor-3 alpha isoform a |
| HIP1 | NM_005338 | huntingtin interacting protein 1 |
| HIP1R | NM_003959 | huntingtin interacting protein-1-related |
| HIP2 | NM_005339 | huntingtin interacting protein 2 |
| HIPK1 | NM_181358 | homeodomain-interacting protein kinase 1 isoform |
| HK1 | NM_000188 | hexokinase 1 isoform HKI |
| HKR2 | NM_181846 | GLI-Kruppel family member HKR2 |
| HLA-DQA1 | NM_002122 | major histocompatibility complex, class II, DQ |
| HLA-DQA2 | NM_020056 | major histocompatibility complex, class II, DQ |
| HLX1 | NM_021958 | H2.0-like homeo box 1 |
| HM13 | NM_178580 | minor histocompatibility antigen 13 isoform 2 |
| HMBOX1 | NM_024567 | hypothetical protein LOC79618 |
| HMBS | NM_000190 | hydroxymethylbilane synthase isoform 1 |
| HMG20A | NM_018200 | high-mobility group 20A |
| HMGA1 | NM_002131 | high mobility group AT-hook 1 isoform b |
| HMGB1 | NM_002128 | high-mobility group box 1 |
| HMGCS1 | NM_002130 | 3-hydroxy-3-methylglutaryl-Coenzyme A synthase 1 |
| HMGN4 | NM_006353 | high mobility group nucleosomal binding domain |
| HMMR | NM_012484 | hyaluronan-mediated motility receptor isoform a |
| HMP19 | NM_015980 | HMP19 protein |
| HMX1 | NM_018942 | homeo box (H6 family) 1 |
| HN1 | NM_001002032 | hematological and neurological expressed 1 |
| HNF4A | NM_000457 | hepatocyte nuclear factor 4 alpha isoform b |
| HNF4G | NM_004133 | hepatocyte nuclear factor 4, gamma |
| HNRPUL1 | NM_007040 | E1B-55 kDa-associated protein 5 isoform a |
| HOMER2 | NM_004839 | homer 2 isoform 1 |
| HOXA13 | NM_000522 | homeobox A13 |
| HOXA3 | NM_030661 | homeobox A3 isoform a |
| HOXB4 | NM_024015 | homeobox B4 |
| HOXB8 | NM_024016 | homeobox B8 |
| HOXC13 | NM_017410 | homeobox C13 |
| HOXC8 | NM_022658 | homeobox C8 |
| HOXD9 | NM_014213 | homeobox D9 |
| HPCAL1 | NM_002149 | hippocalcin-like 1 |
| HPCAL4 | NM_016257 | hippocalcin-like protein 4 |
| HPS1 | NM_182637 | Hermansky-Pudlak syndrome 1 protein isoform b |
| HPSE | NM_006665 | heparanase |
| HR | NM_005144 | hairless protein isoform a |
| HRH3 | NM_007232 | histamine receptor H3 |
| HRH4 | NM_021624 | histamine H4 receptor |
| HS1BP3 | NM_022460 | HS1-binding protein 3 |
| HS2ST1 | NM_012262 | heparan sulfate 2-O-sulfotransferase 1 |
| HS6ST1 | NM_004807 | heparan sulfate 6-O-sulfotransferase |
| HSBP1 | NM_001537 | heat shock factor binding protein 1 |
| HSD11B2 | NM_000196 | hydroxysteroid (11-beta) dehydrogenase 2 |
| HSPA12B | NM_052970 | heat shock 70 kD protein 12B |
| HSPA1A | NM_005345 | heat shock 70 kDa protein 1A |
| HSPA1B | NM_005346 | heat shock 70 kDa protein 1B |
| HSPA5 | NM_005347 | heat shock 70 kDa protein 5 (glucose-regulated |
| HSPB6 | NM_144617 | heat shock protein, alpha-crystallin-related, |
| HSPBP1 | NM_012267 | hsp70-interacting protein |
| HSPC117 | NM_014306 | hypothetical protein LOC51493 |
| HSPG2 | NM_005529 | heparan sulfate proteoglycan 2 |
| HTATIP | NM_006388 | HIV-1 Tat interactive protein, 60 kDa isoform 2 |
| HTLF | NM_002158 | T-cell leukemia virus enhancer factor |
| HTR2A | NM_000621 | 5-hydroxytryptamine (serotonin) receptor 2A |
| HTR2C | NM_000868 | 5-hydroxytryptamine (serotonin) receptor 2C |
| HTR4 | NM_199453 | serotonin 5-HT4 receptor isoform g |
| HTRA1 | NM_002775 | HtrA serine peptidase 1 |
| HUS1 | NM_004507 | HUS1 checkpoint protein |
| HYAL3 | NM_003549 | hyaluronoglucosaminidase 3 |
| IBRDC2 | NM_182757 | IBR domain containing 2 |
| ICA1 | NM_004968 | islet cell autoantigen 1 |
| ICMT | NM_012405 | isoprenylcysteine carboxyl methyltransferase |
| ICOS | NM_012092 | inducible T-cell co-stimulator precursor |
| ICOSLG | NM_015259 | inducible T-cell co-stimulator ligand |
| IDH1 | NM_005896 | isocitrate dehydrogenase 1 (NADP+), soluble |
| IDH3A | NM_005530 | isocitrate dehydrogenase 3 (NAD+) alpha |
| IER5 | NM_016545 | immediate early response 5 |
| IFI35 | NM_005533 | interferon-induced protein 35 |
| IFIT1L | NM_001010987 | interferon-induced protein with |
| IFNAR1 | NM_000629 | interferon-alpha receptor 1 precursor |
| IFNG | NM_000619 | interferon, gamma |
| IGF1 | NM_000618 | insulin-like growth factor 1 (somatomedin C) |
| IGF1R | NM_000875 | insulin-like growth factor 1 receptor precursor |
| IGF2AS | NM_016412 | insulin-like growth factor 2 antisense |
| IGF2BP1 | NM_006546 | insulin-like growth factor 2 mRNA binding |
| IGF2BP2 | NM_001007225 | insulin-like growth factor 2 mRNA binding |
| IGF2BP3 | NM_006547 | insulin-like growth factor 2 mRNA binding |
| IGFBP1 | NM_000596 | insulin-like growth factor binding protein 1 |
| IGFBP3 | NM_000598 | insulin-like growth factor binding protein 3 |
| IGFBP5 | NM_000599 | insulin-like growth factor binding protein 5 |
| IGFL1 | NM_198541 | insulin growth factor-like family member 1 |
| IGSF1 | NM_205833 | immunoglobulin superfamily, member 1 isoform 2 |
| IGSF3 | NM_001007237 | immunoglobulin superfamily, member 3 isoform 2 |
| IGSF4B | NM_021189 | immunoglobulin superfamily, member 4B |
| IGSF4C | NM_145296 | immunoglobulin superfamily, member 4C |
| IGSF4D | NM_153184 | immunoglobulin superfamily, member 4D |
| IGSF9 | NM_020789 | immunoglobulin superfamily, member 9 |
| IIP45 | NM_001025374 | invasion inhibitory protein 45 isoform 2 |
| IKBKAP | NM_003640 | inhibitor of kappa light polypeptide gene |
| IKBKB | NM_001556 | inhibitor of kappa light polypeptide gene |
| IKBKE | NM_014002 | IKK-related kinase epsilon |
| IKBKG | NM_003639 | inhibitor of kappa light polypeptide gene |
| IL10RB | NM_000628 | interleukin 10 receptor, beta precursor |
| IL11RA | NM_147162 | interleukin 11 receptor, alpha isoform 2 |
| IL15RA | NM_002189 | interleukin 15 receptor, alpha isoform 1 |
| IL16 | NM_172217 | interleukin 16 isoform 2 |
| IL17RC | NM_032732 | interleukin 17 receptor C isoform 3 precursor |
| IL17RD | NM_017563 | interleukin 17 receptor D |
| IL18BP | NM_173042 | interleukin 18 binding protein precursor |
| IL1B | NM_000576 | interleukin 1, beta proprotein |
| IL1R1 | NM_000877 | interleukin 1 receptor, type 1 precursor |
| IL1RL1 | NM_003856 | interleukin 1 receptor-like 1 isoform 2 |
| IL1RN | NM_000577 | interleukin 1 receptor antagonist isoform 3 |
| IL22RA1 | NM_021258 | interleukin 22 receptor, alpha 1 |
| IL22RA2 | NM_052962 | interleukin 22-binding protein isoform 1 |
| IL28RA | NM_170743 | interleukin 28 receptor, alpha isoform 1 |
| IL2RB | NM_000878 | interleukin 2 receptor beta precursor |
| IL4R | NM_000418 | interleukin 4 receptor alpha chain isoform a |
| IL6R | NM_000565 | interleukin 6 receptor isoform 1 precursor |
| IL8RA | NM_000634 | interleukin 8 receptor alpha |
| IL9R | NM_176786 | interleukin 9 receptor isoform 2 |
| ILDR1 | NM_175924 | immunoglobulin-like domain containing receptor |
| ILF3 | NM_012218 | interleukin enhancer binding factor 3 isoform a |
| ILKAP | NM_176799 | integrin-linked kinase-associated protein |
| IMMP2L | NM_032549 | IMP2 inner mitochondrial membrane protease-like |
| IMPDH1 | NM_000883 | inosine monophosphate dehydrogenase 1 isoform a |
| INA | NM_032727 | internexin neuronal intermediate filament |
| INCENP | NM_020238 | inner centromere protein antigens 135/155 kDa |
| ING1 | NM_005537 | inhibitor of growth family, member 1 isoform D |
| ING3 | NM_198267 | inhibitor of growth family, member 3 isoform 3 |
| ING5 | NM_032329 | inhibitor of growth family, member 5 |
| INHBB | NM_002193 | inhibin beta B subunit precursor |
| INMT | NM_006774 | indolethylamine N-methyltransferase |
| INOC1 | NM_017553 | INO80 complex homolog 1 |
| INPP5B | NM_005540 | inositol polyphosphate-5-phosphatase, 75 kDa |
| INPP5D | NM_001017915 | SH2 containing inositol phosphatase isoform a |
| INSIG1 | NM_005542 | insulin induced gene 1 isoform 1 |
| INSL5 | NM_005478 | insulin-like 5 precursor |
| INSM1 | NM_002196 | insulinoma-associated 1 |
| INSM2 | NM_032594 | insulinoma-associated protein IA-6 |
| INTS2 | NM_020748 | integrator complex subunit 2 |
| INTS3 | NM_023015 | hypothetical protein LOC65123 |
| IPLA2(GAMMA) | NM_015723 | intracellular membrane-associated |
| IPO11 | NM_016338 | Ran binding protein 11 |
| IPPK | NM_022755 | inositol 1,3,4,5,6-pentakisphosphate 2-kinase |
| IQCE | NM_152558 | IQ motif containing E |
| IQGAP1 | NM_003870 | IQ motif containing GTPase activating protein 1 |
| IQGAP3 | NM_178229 | IQ motif containing GTPase activating protein 3 |
| IQSEC1 | NM_014869 | IQ motif and Sec7 domain 1 |
| IQSEC2 | NM_015075 | IQ motif and Sec7 domain 2 |
| IRAK2 | NM_001570 | interleukin-1 receptor-associated kinase 2 |
| IRAK4 | NM_016123 | interleukin-1 receptor-associated kinase 4 |
| IRF1 | NM_002198 | interferon regulatory factor 1 |
| IRF2BP1 | NM_015649 | interferon regulatory factor 2 binding protein |
| IRF4 | NM_002460 | interferon regulatory factor 4 |
| IRF6 | NM_006147 | interferon regulatory factor 6 |
| ISG20L1 | NM_022767 | interferon stimulated exonuclease gene |
| ISG20L2 | NM_030980 | interferon stimulated exonuclease gene |
| ITCH | NM_031483 | itchy homolog E3 ubiquitin protein ligase |
| ITFG3 | NM_032039 | integrin alpha FG-GAP repeat containing 3 |
| ITGA10 | NM_003637 | integrin, alpha 10 precursor |
| ITGA11 | NM_001004439 | integrin, alpha 11 precursor |
| ITGAL | NM_002209 | integrin alpha L precursor |
| ITGAM | NM_000632 | integrin alpha M precursor |
| ITGB8 | NM_002214 | integrin, beta 8 precursor |
| ITIH5 | NM_030569 | inter-alpha trypsin inhibitor heavy chain |
| ITPK1 | NM_014216 | inositol 1,3,4-triphosphate 5/6 kinase |
| ITPKB | NM_002221 | 1D-myo-inositol-trisphosphate 3-kinase B |
| ITPR2 | NM_002223 | inositol 1,4,5-triphosphate receptor, type 2 |
| ITPR3 | NM_002224 | inositol 1,4,5-triphosphate receptor, type 3 |
| ITSN1 | NM_001001132 | intersectin 1 isoform ITSN-s |
| IXL | NM_017592 | intersex-like |
| JAG1 | NM_000214 | jagged 1 precursor |
| JAK2 | NM_004972 | Janus kinase 2 |
| JAKMIP1 | NM_144720 | multiple coiled-coil GABABR1-binding protein |
| JAM3 | NM_032801 | junctional adhesion molecule 3 precursor |
| JARID2 | NM_004973 | jumonji, AT rich interactive domain 2 protein |
| JAZF1 | NM_175061 | juxtaposed with another zinc finger gene 1 |
| JMJD1C | NM_004241 | jumonji domain containing 1C |
| JMJD2C | NM_015061 | jumonji domain containing 2C |
| JMJD2D | NM_018039 | jumonji domain containing 2D |
| JMJD4 | NM_023007 | jumonji domain containing 4 |
| JMJD5 | NM_024773 | hypothetical protein LOC79831 |
| JOSD2 | NM_138334 | Josephin domain containing 2 |
| JPH1 | NM_020647 | junctophilin 1 |
| JPH3 | NM_020655 | junctophilin 3 |
| JPH4 | NM_032452 | junctophilin 4 |
| JRK | NM_003724 | jerky homolog |
| JUP | NM_002230 | junction plakoglobin |
| K6HF | NM_004693 | cytokeratin type II |
| K6IRS4 | NM_175053 | keratin 6 irs4 |
| KA36 | NM_182497 | type I hair keratin KA36 |
| KAZALD1 | NM_030929 | Kazal-type serine protease inhibitor domain 1 |
| KBTBD11 | NM_014867 | kelch repeat and BTB (POZ) domain containing 11 |
| KBTBD6 | NM_152903 | kelch repeat and BTB (POZ) domain-containing 6 |
| KCNA6 | NM_002235 | potassium voltage-gated channel, shaker-related |
| KCNAB2 | NM_003636 | potassium voltage-gated channel, shaker-related |
| KCNC3 | NM_004977 | Shaw-related voltage-gated potassium channel |
| KCNC4 | NM_004978 | Shaw-related voltage-gated potassium channel |
| KCNE1 | NM_000219 | potassium voltage-gated channel, Isk-related |
| KCNE1L | NM_012282 | potassium voltage-gated channel, Isk-related |
| KCNE3 | NM_005472 | potassium voltage-gated channel, Isk-related |
| KCNG1 | NM_172318 | potassium voltage-gated channel, subfamily G, |
| KCNH2 | NM_000238 | voltage-gated potassium channel, subfamily H, |
| KCNH5 | NM_172375 | potassium voltage-gated channel, subfamily H, |
| KCNH7 | NM_033272 | potassium voltage-gated channel, subfamily H, |
| KCNIP1 | NM_014592 | Kv channel interacting protein 1 isoform 2 |
| KCNJ10 | NM_002241 | potassium inwardly-rectifying channel, subfamily |
| KCNJ11 | NM_000525 | potassium inwardly-rectifying channel J11 |
| KCNJ14 | NM_013348 | potassium inwardly-rectifying channel J14 |
| KCNJ2 | NM_000891 | potassium inwardly-rectifying channel J2 |
| KCNJ4 | NM_004981 | potassium inwardly-rectifying channel J4 |
| KCNJ8 | NM_004982 | potassium inwardly-rectifying channel J8 |
| KCNK2 | NM_001017424 | potassium channel, subfamily K, member 2 isoform |
| KCNK3 | NM_002246 | potassium channel, subfamily K, member 3 |
| KCNK5 | NM_003740 | potassium channel, subfamily K, member 5 |
| KCNK6 | NM_004823 | potassium channel, subfamily K, member 6 |
| KCNK9 | NM_016601 | potassium channel, subfamily K, member 9 |
| KCNMA1 | NM_001014797 | large conductance calcium-activated potassium |
| KCNN1 | NM_002248 | potassium intermediate/small conductance |
| KCNQ1 | NM_000218 | potassium voltage-gated channel, KQT-like |
| KCNQ2 | NM_004518 | potassium voltage-gated channel KQT-like protein |
| KCNQ4 | NM_004700 | potassium voltage-gated channel KQT-like protein |
| KCNS2 | NM_020697 | potassium voltage-gated channel, |
| KCTD10 | NM_031954 | potassium channel tetramerisation domain |
| KCTD16 | NM_020768 | potassium channel tetramerisation domain |
| KCTD17 | NM_024681 | potassium channel tetramerisation domain |
| KCTD2 | NM_015353 | potassium channel tetramerisation domain |
| KCTD5 | NM_018992 | potassium channel tetramerisation domain |
| KCTD7 | NM_153033 | potassium channel tetramerisation domain |
| KDELR3 | NM_006855 | KDEL receptor 3 isoform a |
| KHK | NM_000221 | ketohexokinase isoform a |
| KIAA0040 | NM_014656 | hypothetical protein LOC9674 |
| KIAA0082 | NM_015050 | hypothetical protein LOC23070 |
| KIAA0090 | NM_015047 | hypothetical protein LOC23065 |
| KIAA0125 | NM_014792 | hypothetical protein LOC9834 |
| KIAA0152 | NM_014730 | hypothetical protein LOC9761 |
| KIAA0157 | NM_032182 | hypothetical protein LOC23172 |
| KIAA0179 | NM_015056 | hypothetical protein LOC23076 |
| KIAA0182 | NM_014615 | hypothetical protein LOC23199 |
| KIAA0251 | NM_015027 | hypothetical protein LOC23042 |
| KIAA0265 | NM_014997 | hypothetical protein LOC23008 |
| KIAA0286 | NM_015257 | hypothetical protein LOC23306 |
| KIAA0319 | NM_014809 | KIAA0319 |
| KIAA0319L | NM_024874 | polycystic kidney disease 1-like isoform a |
| KIAA0329 | NM_014844 | hypothetical protein LOC9895 |
| KIAA0355 | NM_014686 | hypothetical protein LOC9710 |
| KIAA0376 | NM_015330 | cytospin A |
| KIAA0404 | NM_015104 | hypothetical protein LOC23130 |
| KIAA0406 | NM_014657 | hypothetical protein LOC9675 |
| KIAA0427 | NM_014772 | hypothetical protein LOC9811 |
| KIAA0446 | NM_014655 | hypothetical protein LOC9673 |
| KIAA0495 | NM_207306 | KIAA0495 |
| KIAA0513 | NM_014732 | hypothetical protein LOC9764 |
| KIAA0523 | NM_015253 | hypothetical protein LOC23302 |
| KIAA0556 | NM_015202 | hypothetical protein LOC23247 |
| KIAA0652 | NM_014741 | hypothetical protein LOC9776 |
| KIAA0672 | NM_014859 | hypothetical protein LOC9912 |
| KIAA0683 | NM_016111 | hypothetical protein LOC9894 |
| KIAA0753 | NM_014804 | hypothetical protein LOC9851 |
| KIAA0773 | NM_001031690 | hypothetical protein LOC9715 |
| KIAA0789 | NM_014653 | hypothetical protein LOC9671 |
| KIAA0802 | NM_015210 | hypothetical protein LOC23255 |
| KIAA0828 | NM_015328 | KIAA0828 protein |
| KIAA0892 | NM_015329 | hypothetical protein LOC23383 |
| KIAA0971 | NM_014929 | hypothetical protein LOC22868 |
| KIAA1005 | NM_015272 | hypothetical protein LOC23322 |
| KIAA1008 | NM_014953 | KIAA1008 |
| KIAA1009 | NM_014895 | hypothetical protein LOC22832 |
| KIAA1018 | NM_014967 | hypothetical protein LOC22909 |
| KIAA1024 | NM_015206 | hypothetical protein LOC23251 |
| KIAA1160 | NM_020701 | hypothetical protein LOC57461 |
| KIAA1166 | NM_018684 | hepatocellular carcinoma-associated antigen 127 |
| KIAA1212 | NM_018084 | Hook-related protein 1 |
| KIAA1217 | NM_019590 | hypothetical protein LOC56243 |
| KIAA1267 | NM_015443 | hypothetical protein LOC284058 |
| KIAA1274 | NM_014431 | KIAA1274 |
| KIAA1303 | NM_020761 | raptor |
| KIAA1324 | NM_020775 | hypothetical protein LOC57535 |
| KIAA1333 | NM_017769 | hypothetical protein LOC55632 |
| KIAA1522 | NM_020888 | hypothetical protein LOC57648 |
| KIAA1609 | NM_020947 | hypothetical protein LOC57707 |
| KIAA1729 | NM_053042 | hypothetical protein LOC85460 |
| KIAA1787 | NM_001005408 | hypothetical protein LOC84461 isoform 2 |
| KIAA1815 | NM_024896 | hypothetical protein LOC79956 |
| KIAA1853 | NM_194286 | KIAA1853 protein |
| KIAA1875 | NM_032529 | KIAA1875 protein |
| KIAA1904 | NM_052906 | hypothetical protein LOC114794 |
| KIAA1909 | NM_052909 | hypothetical protein LOC153478 |
| KIAA1919 | NM_153369 | KIAA1919 protein |
| KIAA1920 | NM_052919 | hypothetical protein LOC114817 |
| KIAA1924 | NM_145294 | hypothetical protein LOC197335 |
| KIAA1958 | NM_133465 | hypothetical protein LOC158405 |
| KIAA1967 | NM_021174 | p30 DBC protein |
| KIAA2022 | NM_001008537 | hypothetical protein LOC340533 |
| KIF11 | NM_004523 | kinesin family member 11 |
| KIF13A | NM_022113 | kinesin family member 13A |
| KIF17 | NM_020816 | kinesin family member 17 |
| KIF1A | NM_004321 | axonal transport of synaptic vesicles |
| KIF1B | NM_015074 | kinesin family member lB isoform b |
| KIR2DL1 | NM_014218 | killer cell immunoglobulin-like receptor, two |
| KIR2DL2 | NM_014219 | killer cell immunoglobulin-like receptor, two |
| KIR2DL3 | NM_014511 | killer cell immunoglobulin-like receptor, two |
| KIR2DL4 | NM_002255 | killer cell immunoglobulin-like receptor, two |
| KIR2DL5A | NM_020535 | killer cell immunoglobulin-like receptor, two |
| KIR2DL5B | NM_001018081 | killer cell immunoglobulin-like receptor, two |
| KIR2DS2 | NM_012312 | killer cell immunoglobulin-like receptor, two |
| KIR2DS4 | NM_012314 | killer cell immunoglobulin-like receptor, two |
| KIR2DS5 | NM_014513 | killer cell immunoglobulin-like receptor, two |
| KIR3DL1 | NM_013289 | killer cell immunoglobulin-like receptor, three |
| KIR3DL2 | NM_006737 | killer cell immunoglobulin-like receptor, three |
| KIR3DL3 | NM_153443 | killer cell immunoglobulin-like receptor, three |
| KIT | NM_000222 | v-kit Hardy-Zuckerman 4 feline sarcoma viral |
| KITLG | NM_000899 | KIT ligand isoform b precursor |
| KL | NM_153683 | klotho isoform b |
| KLC2 | NM_022822 | likely ortholog of kinesin light chain 2 |
| KLF11 | NM_003597 | Kruppel-like factor 11 |
| KLF12 | NM_007249 | Kruppel-like factor 12 isoform a |
| KLF13 | NM_015995 | Kruppel-like factor 13 |
| KLF17 | NM_173484 | zinc finger protein 393 |
| KLF4 | NM_004235 | Kruppel-like factor 4 (Rubin and Gutmann, 2005) |
| KLF5 | NM_001730 | Kruppel-like factor 5 |
| KLF6 | NM_001008490 | Kruppel-like factor 6 |
| KLHDC3 | NM_057161 | testis intracellular mediator protein |
| KLHDC6 | NM_207335 | hypothetical protein LOC166348 |
| KLHDC7A | NM_152375 | hypothetical protein LOC127707 |
| KLHDC8B | NM_173546 | hypothetical protein LOC200942 |
| KLHL12 | NM_021633 | kelch-like 12 |
| KLHL14 | NM_020805 | kelch-like 14 |
| KLHL17 | NM_198317 | kelch-like 17 |
| KLHL18 | NM_025010 | kelch-like 18 |
| KLHL21 | NM_014851 | kelch-like 21 |
| KLHL25 | NM_022480 | BTB/POZ KELCH domain protein |
| KLHL3 | NM_017415 | kelch-like 3 (Drosophila) |
| KLK13 | NM_015596 | kallikrein 13 precursor |
| KLRD1 | NM_002262 | killer cell lectin-like receptor subfamily D, |
| KLRK1 | NM_007360 | NKG2-D type II integral membrane protein |
| KNDC1 | NM_152643 | kinase non-catalytic C-lobe domain (Lopez-Beltran et |
| | | al., 2006) |
| KREMEN2 | NM_024507 | kringle-containing transmembrane protein 2 |
| KRIT1 | NM_001013406 | krev interaction trapped 1 isoform 2 |
| KRT20 | NM_019010 | keratin 20 |
| KRT5 | NM_000424 | keratin 5 |
| KRTAP3-1 | NM_031958 | keratin associated protein 3.1 |
| KRTAP4-14 | NM_033059 | keratin associated protein 4-14 |
| KRTAP4-7 | NM_033061 | keratin associated protein 4-7 |
| KRTAP5-10 | NM_001012710 | keratin associated protein 5-10 |
| KRTAP5-2 | NM_001004325 | keratin associated protein 5-2 |
| KRTHB1 | NM_002281 | keratin, hair, basic, 1 |
| KRTHB2 | NM_033033 | keratin, hair, basic, 2 |
| KRTHB3 | NM_002282 | keratin, hair, basic, 3 |
| KSR1 | NM_014238 | kinase suppressor of ras |
| KTN1 | NM_182926 | kinectin 1 |
| L1CAM | NM_000425 | L1 cell adhesion molecule isoform 1 precursor |
| LAD1 | NM_005558 | ladinin 1 |
| LAMB2 | NM_002292 | laminin, beta 2 precursor |
| LAMC1 | NM_002293 | laminin, gamma 1 precursor |
| LANCL1 | NM_006055 | lanthionine synthetase C-like protein 1 |
| LARP5 | NM_015155 | La ribonucleoprotein domain family, member 5 |
| LASP1 | NM_006148 | LIM and SH3 protein 1 |
| LASS1 | NM_021267 | longevity assurance gene 1 isoform 1 |
| LASS2 | NM_013384 | LAG1 longevity assurance homolog 2 isoform 2 |
| LASS3 | NM_178842 | hypothetical protein LOC204219 |
| LASS5 | NM_147190 | LAG1 longevity assurance homolog 5 |
| LASS6 | NM_203463 | longevity assurance homolog 6 |
| LCE1C | NM_178351 | late cornified envelope 1C |
| LCMT2 | NM_014793 | leucine carboxyl methyltransferase 2 |
| LCP1 | NM_002298 | L-plastin |
| LDB3 | NM_007078 | LIM domain binding 3 |
| LDHA | NM_005566 | lactate dehydrogenase A |
| LDHD | NM_153486 | D-lactate dehydrogenase isoform 1 precursor |
| LDLR | NM_000527 | low density lipoprotein receptor precursor |
| LDLRAD2 | NM_001013693 | hypothetical protein LOC401944 |
| LDOC1L | NM_032287 | hypothetical protein LOC84247 |
| LEF1 | NM_016269 | lymphoid enhancer binding factor-1 |
| LELP1 | NM_001010857 | late cornified envelope-like proline-rich 1 |
| LENEP | NM_018655 | lens epithelial protein |
| LEPREL1 | NM_018192 | leprecan-like 1 |
| LEREPO4 | NM_018471 | erythropoietin 4 immediate early response |
| LETMD1 | NM_001024668 | LETM1 domain containing 1 isoform 2 |
| LGI1 | NM_005097 | leucine-rich, glioma inactivated 1 precursor |
| LGI2 | NM_018176 | leucine-rich repeat LGI family, member 2 |
| LGI3 | NM_139278 | leucine-rich repeat LGI family, member 3 |
| LGR4 | NM_018490 | leucine-rich repeat-containing G protein-coupled |
| LHCGR | NM_000233 | luteinizing hormone/choriogonadotropin receptor |
| LHFPL2 | NM_005779 | lipoma HMGIC fusion partner-like 2 |
| LHPP | NM_022126 | phospholysine phosphohistidine inorganic |
| LHX2 | NM_004789 | LIM homeobox protein 2 |
| LHX3 | NM_014564 | LIM homeobox protein 3 isoform b |
| LIF | NM_002309 | leukemia inhibitory factor (cholinergic |
| LILRB4 | NM_006847 | leukocyte immunoglobulin-like receptor, |
| LIMA1 | NM_016357 | epithelial protein lost in neoplasm beta |
| LIMD1 | NM_014240 | LIM domains containing 1 |
| LIMD2 | NM_030576 | LIM domain containing 2 |
| LIMK1 | NM_016735 | LIM domain kinase 1 isoform dLIMK |
| LIN28 | NM_024674 | lin-28 homolog |
| LIN28B | NM_001004317 | lin-28 homolog B |
| LINS1 | NM_181740 | lines homolog 1 isoform 3 |
| LITAF | NM_004862 | LPS-induced TNF-alpha factor |
| LIX1 | NM_153234 | limb expression 1 |
| LLGL1 | NM_004140 | lethal giant larvae homolog 1 |
| LMAN2L | NM_030805 | lectin, mannose-binding 2-like |
| LMBR1L | NM_018113 | lipocalin-interacting membrane receptor |
| LMNA | NM_170707 | lamin A/C isoform 1 precursor |
| LMNB2 | NM_032737 | lamin B2 |
| LMOD1 | NM_012134 | leiomodin 1 (smooth muscle) |
| LNK | NM_005475 | lymphocyte adaptor protein |
| LNX1 | NM_032622 | multi-PDZ-domain-containing protein |
| LNX2 | NM_153371 | PDZ domain containing ring finger 1 |
| LOC113386 | NM_138781 | hypothetical protein LOC113386 |
| LOC115648 | NM_145326 | hypothetical protein LOC115648 |
| LOC128439 | NM_139016 | hypothetical protein LOC128439 |
| LOC128977 | NM_173793 | hypothetical protein LOC128977 |
| LOC129138 | NM_138797 | hypothetical protein LOC129138 |
| LOC130576 | NM_177964 | hypothetical protein LOC130576 |
| LOC134145 | NM_199133 | hypothetical protein LOC134145 |
| LOC134147 | NM_138809 | hypothetical protein LOC134147 |
| LOC147650 | NM_207324 | hypothetical protein LOC147650 |
| LOC147808 | NM_203374 | hypothetical protein LOC147808 |
| LOC149620 | NM_001013621 | hypothetical protein LOC149620 |
| LOC150223 | NM_001017964 | hypothetical protein LOC150223 isoform a |
| LOC150383 | NM_001008917 | hypothetical protein LOC150383 isoform 2 |
| LOC152485 | NM_178835 | hypothetical protein LOC152485 |
| LOC153222 | NM_153607 | hypothetical protein LOC153222 |
| LOC153364 | NM_203406 | similar to metallo-beta-lactamase superfamily |
| LOC158318 | NM_001024608 | hypothetical protein LOC158318 |
| LOC159090 | NM_145284 | hypothetical protein LOC159090 |
| LOC162427 | NM_178126 | hypothetical protein LOC162427 |
| LOC165186 | NM_199280 | hypothetical protein LOC165186 |
| LOC168850 | NM_176814 | hypothetical protein LOC168850 |
| LOC200261 | NM_182535 | hypothetical protein LOC200261 |
| LOC200312 | NM_001017981 | similar to RIKEN cDNA 0610009J22 |
| LOC201181 | NM_001013624 | hypothetical protein LOC201181 |
| LOC201895 | NM_174921 | hypothetical protein LOC201895 |
| LOC221442 | NM_001010871 | hypothetical protein LOC221442 |
| LOC221955 | NM_139179 | hypothetical protein LOC221955 |
| LOC222171 | NM_175887 | hypothetical protein LOC222171 |
| LOC255374 | NM_203397 | hypothetical protein LOC255374 |
| LOC283174 | NM_001001873 | hypothetical protein LOC283174 |
| LOC283219 | NM_001029859 | hypothetical protein LOC283219 |
| LOC283487 | NM_178514 | hypothetical protein LOC283487 |
| LOC283551 | NM_001012706 | hypothetical protein LOC283551 |
| LOC284296 | NM_175908 | hypothetical protein LOC284296 |
| LOC284739 | NM_207349 | hypothetical protein LOC284739 |
| LOC285382 | NM_001025266 | hypothetical protein LOC285382 |
| LOC285636 | NM_175921 | hypothetical protein LOC285636 |
| LOC285989 | NM_001013258 | hypothetical protein LOC285989 isoform 2 |
| LOC338328 | NM_178172 | high density lipoprotein-binding protein |
| LOC339123 | NM_001005920 | hypothetical LOC339123 |
| LOC339524 | NM_207357 | hypothetical protein LOC339524 |
| LOC340061 | NM_198282 | hypothetical protein LOC340061 |
| LOC340156 | NM_001012418 | hypothetical protein LOC340156 |
| LOC340527 | NM_001013627 | hypothetical protein LOC340527 |
| LOC345222 | NM_001012982 | hypothetical protein LOC345222 |
| LOC348262 | NM_207368 | hypothetical protein LOC348262 |
| LOC349136 | NM_198285 | hypothetical protein LOC349136 |
| LOC387646 | NM_001006604 | hypothetical protein LOC387646 |
| LOC387856 | NM_001013635 | hypothetical protein LOC387856 |
| LOC388022 | NM_001013637 | hypothetical protein LOC388022 |
| LOC388610 | NM_001013642 | hypothetical protein LOC388610 |
| LOC388886 | NM_207644 | hypothetical protein LOC388886 |
| LOC388910 | NM_001012986 | hypothetical protein LOC388910 |
| LOC389151 | NM_001013650 | hypothetical protein LOC389151 |
| LOC389432 | NM_001030060 | hypothetical protein LOC389432 |
| LOC389634 | NM_001012988 | hypothetical protein LOC389634 |
| LOC389833 | NM_001033515 | hypothetical protein LOC389833 |
| LOC389936 | NM_001013656 | hypothetical protein LOC389936 |
| LOC390980 | NM_001023563 | similar to Zinc finger protein 264 |
| LOC400145 | NM_001013669 | hypothetical protein LOC400145 |
| LOC400258 | NM_001008404 | hypothetical protein LOC400258 |
| LOC400464 | NM_001013670 | hypothetical protein LOC400464 |
| LOC400509 | NM_001012391 | hypothetical protein LOC400509 |
| LOC400696 | NM_207646 | hypothetical protein LOC400696 |
| LOC400891 | NM_001013675 | hypothetical protein LOC400891 |
| LOC400965 | NM_001013677 | hypothetical protein LOC400965 |
| LOC400968 | NM_001013678 | hypothetical protein LOC400968 |
| LOC401252 | NM_001013681 | hypothetical protein LOC401252 |
| LOC401280 | NM_001013682 | hypothetical protein LOC401280 |
| LOC401286 | NM_001023565 | hypothetical protein LOC401286 |
| LOC401296 | NM_001024677 | hypothetical protein LOC401296 |
| LOC401357 | NM_001013685 | hypothetical protein LOC401357 |
| LOC401431 | NM_001008745 | hypothetical protein LOC401431 |
| LOC401589 | NM_001013687 | hypothetical protein LOC401589 |
| LOC401620 | NM_001013688 | hypothetical protein LOC401620 |
| LOC401622 | NM_001013689 | hypothetical protein LOC401622 |
| LOC401623 | NM_001018158 | hypothetical protein LOC401623 |
| LOC401720 | NM_001013690 | hypothetical protein LOC401720 |
| LOC439985 | NM_001013696 | hypothetical protein LOC439985 |
| LOC440295 | NM_198181 | hypothetical protein LOC440295 |
| LOC440313 | NM_001013704 | hypothetical protein LOC440313 |
| LOC440742 | NM_001013710 | hypothetical protein LOC440742 |
| LOC440836 | NM_001014440 | similar to MGC52679 protein |
| LOC440944 | NM_001013713 | hypothetical protein LOC440944 |
| LOC441046 | NM_001011539 | hypothetical protein LOC441046 |
| LOC441120 | NM_001013718 | hypothetical protein LOC441120 |
| LOC441179 | NM_001013721 | hypothetical protein LOC441179 |
| LOC504188 | NM_001013404 | hypothetical protein LOC504188 |
| LOC51149 | NM_001018061 | hypothetical protein LOC51149 isoform 4 |
| LOC51333 | NM_016643 | mesenchymal stem cell protein DSC43 |
| LOC552891 | NM_004125 | hypothetical protein LOC552891 |
| LOC55565 | NM_017530 | hypothetical protein LOC55565 |
| LOC619208 | NM_001033564 | hypothetical protein LOC619208 |
| LOC63920 | NM_022090 | transposon-derived Buster3 transposase-like |
| LOC89944 | NM_138342 | hypothetical protein LOC89944 |
| LOC90321 | NM_001010851 | hypothetical protein LOC90321 |
| LOC93349 | NM_138402 | hypothetical protein LOC93349 |
| LOH11CR2A | NM_014622 | BCSC-1 isoform 1 |
| LONRF3 | NM_001031855 | LON peptidase N-terminal domain and ring finger |
| LOXL3 | NM_032603 | lysyl oxidase-like 3 precursor |
| LPAL2 | NM_145727 | lipoprotein, Lp(a)-like 2 precursor |
| LPGAT1 | NM_014873 | lysophosphatidylglycerol acyltransferase 1 |
| LPHN1 | NM_001008701 | latrophilin 1 isoform 1 precursor |
| LPIN1 | NM_145693 | lipin 1 |
| LPIN2 | NM_014646 | lipin 2 |
| LPIN3 | NM_022896 | lipin 3 |
| LPO | NM_006151 | lactoperoxidase |
| LPP | NM_005578 | LIM domain containing preferred translocation |
| LPPR2 | NM_022737 | lipid phosphate phosphatase-related protein type |
| LRAT | NM_004744 | lecithin retinol acyltransferase |
| LRCH1 | NM_015116 | leucine-rich repeats and calponin homology (CH) |
| LRCH2 | NM_020871 | leucine-rich repeats and calponin homology (CH) |
| LRCH4 | NM_002319 | leucine-rich repeats and calponin homology (CH) |
| LRIG1 | NM_015541 | leucine-rich repeats and immunoglobulin-like |
| LRP1 | NM_002332 | low density lipoprotein-related protein 1 |
| LRP2BP | NM_018409 | LRP2 binding protein |
| LRP4 | NM_002334 | low density lipoprotein receptor-related protein |
| LRRC1 | NM_018214 | leucine rich repeat containing 1 |
| LRRC14 | NM_014665 | leucine rich repeat containing 14 |
| LRRC18 | NM_001006939 | leucine rich repeat containing 18 |
| LRRC2 | NM_024512 | leucine rich repeat containing 2 |
| LRRC40 | NM_017768 | leucine rich repeat containing 40 |
| LRRC44 | NM_145258 | leucine rich repeat containing 44 |
| LRRC55 | NM_001005210 | hypothetical protein LOC219527 |
| LRRC56 | NM_198075 | hypothetical protein LOC115399 |
| LRRFIP1 | NM_004735 | leucine rich repeat (in FLII) interacting |
| LRRTM2 | NM_015564 | leucine rich repeat transmembrane neuronal 2 |
| LRSAM1 | NM_001005373 | leucine rich repeat and sterile alpha motif |
| LSS | NM_002340 | lanosterol synthase |
| LTBP2 | NM_000428 | latent transforming growth factor beta binding |
| LTBR | NM_002342 | lymphotoxin beta receptor |
| LTF | NM_002343 | lactotransferrin |
| LUZP1 | NM_033631 | leucine zipper protein 1 |
| LUZP4 | NM_016383 | leucine zipper protein 4 |
| LY6G5B | NM_021221 | lymphocyte antigen 6 complex G5B |
| LY6G5C | NM_001002848 | lymphocyte antigen 6 complex G5C isoform C |
| LY6K | NM_017527 | lymphocyte antigen 6 complex, locus K |
| LY75 | NM_002349 | lymphocyte antigen 75 |
| LY9 | NM_001033667 | lymphocyte antigen 9 isoform b |
| LYCAT | NM_001002257 | lysocardiolipin acyltransferase isoform 2 |
| LYPD3 | NM_014400 | GPI-anchored metastasis-associated protein |
| LYPLA1 | NM_006330 | lysophospholipase I |
| LYPLA3 | NM_012320 | lysophospholipase 3 (lysosomal phospholipase |
| LYPLAL1 | NM_138794 | lysophospholipase-like 1 |
| LYSMD4 | NM_152449 | hypothetical protein LOC145748 |
| LYST | NM_000081 | lysosomal trafficking regulator isoform 1 |
| LYZ | NM_000239 | lysozyme precursor |
| LZTS1 | NM_021020 | leucine zipper, putative tumor suppressor 1 |
| LZTS2 | NM_032429 | leucine zipper, putative tumor suppressor 2 |
| M6PR | NM_002355 | cation-dependent mannose-6-phosphate receptor |
| MADD | NM_003682 | MAP-kinase activating death domain-containing |
| MAF1 | NM_032272 | MAF1 protein |
| MAFF | NM_012323 | transcription factor MAFF |
| MAFK | NM_002360 | v-maf musculoaponeurotic fibrosarcoma oncogene |
| MAGEA12 | NM_005367 | melanoma antigen family A, 12 |
| MAGEA2 | NM_005361 | melanoma antigen family A, 2 |
| MAGEA2B | NM_153488 | melanoma antigen family A, 2B |
| MAGEA3 | NM_005362 | melanoma antigen family A, 3 |
| MAGEA6 | NM_005363 | melanoma antigen family A, 6 |
| MAGEB2 | NM_002364 | melanoma antigen family B, 2 |
| MAGEC3 | NM_177456 | melanoma antigen family C, 3 isoform 2 |
| MAGI1 | NM_001033057 | membrane associated guanylate kinase, WW and PDZ |
| MAK3 | NM_025146 | Mak3 homolog |
| MAL | NM_002371 | T-lymphocyte maturation-associated protein |
| MAML3 | NM_018717 | mastermind-like 3 |
| MAN2A2 | NM_006122 | mannosidase, alpha, class 2A, member 2 |
| MAOA | NM_000240 | monoamine oxidase A |
| MAP1A | NM_002373 | microtubule-associated protein 1A |
| MAP2 | NM_002374 | microtubule-associated protein 2 isoform 1 |
| MAP2K1 | NM_002755 | mitogen-activated protein kinase kinase 1 |
| MAP2K3 | NM_002756 | mitogen-activated protein kinase kinase 3 |
| MAP3K14 | NM_003954 | mitogen-activated protein kinase kinase kinase |
| MAP3K15 | NM_001001671 | mitogen-activated protein kinase kinase kinase |
| MAP3K3 | NM_002401 | mitogen-activated protein kinase kinase kinase 3 |
| MAP3K7 | NM_003188 | mitogen-activated protein kinase kinase kinase 7 |
| MAP3K7IP1 | NM_006116 | mitogen-activated protein kinase kinase kinase 7 |
| MAP3K7IP2 | NM_015093 | mitogen-activated protein kinase kinase kinase 7 |
| MAP3K9 | NM_033141 | mitogen-activated protein kinase kinase kinase |
| MAP4 | NM_002375 | microtubule-associated protein 4 isoform 1 |
| MAP4K4 | NM_004834 | mitogen-activated protein kinase kinase kinase |
| MAP6 | NM_207577 | microtubule-associated protein 6 isoform 2 |
| MAP6D1 | NM_024871 | MAP6 domain containing 1 |
| MAP7 | NM_003980 | microtubule-associated protein 7 |
| MAPK1 | NM_002745 | mitogen-activated protein kinase 1 |
| MAPK10 | NM_002753 | mitogen-activated protein kinase 10 isoform 1 |
| MAPK13 | NM_002754 | mitogen-activated protein kinase 13 |
| MAPK15 | NM_139021 | mitogen-activated protein kinase 15 |
| MAPKAPK3 | NM_004635 | mitogen-activated protein kinase-activated |
| MAPRE2 | NM_014268 | microtubule-associated protein, RP/EB family, |
| MAPT | NM_005910 | microtubule-associated protein tau isoform 2 |
| MARCH4 | NM_020814 | membrane-associated ring finger (C3HC4) 4 |
| MARCH5 | NM_017824 | ring finger protein 153 |
| MARCH8 | NM_001002265 | cellular modulator of immune recognition |
| MARCH9 | NM_138396 | membrane-associated RING-CH protein IX |
| MARCKSL1 | NM_023009 | MARCKS-like 1 |
| MARK4 | NM_031417 | MAP/microtubule affinity-regulating kinase 4 |
| MARVELD1 | NM_031484 | MARVEL domain containing I |
| MARVELD3 | NM_052858 | MARVEL domain containing 3 isoform 2 |
| MASP1 | NM_001031849 | mannan-binding lectin serine protease 1 isoform |
| MASP2 | NM_006610 | mannan-binding lectin serine protease 2 isoform |
| MAT2A | NM_005911 | methionine adenosyltransferase II, alpha |
| MAWBP | NM_001033083 | MAWD binding protein isoform b |
| MAX | NM_002382 | MAX protein isoform a |
| MBD1 | NM_002384 | methyl-CpG binding domain protein 1 isoform 4 |
| MBD3 | NM_003926 | methyl-CpG binding domain protein 3 |
| MBD6 | NM_052897 | methyl-CpG binding domain protein 6 |
| MBP | NM_001025081 | myelin basic protein isoform 1 |
| MC2R | NM_000529 | melanocortin 2 receptor |
| MCART6 | NM_001012755 | hypothetical protein L0C401612 |
| MCFD2 | NM_139279 | multiple coagulation factor deficiency 2 |
| MCL1 | NM_021960 | myeloid cell leukemia sequence 1 isoform 1 |
| MCOLN1 | NM_020533 | mucolipin 1 |
| MDGA1 | NM_153487 | MAM domain containing |
| MECP2 | NM_004992 | methyl CpG binding protein 2 |
| MECR | NM_001024732 | nuclear receptor-binding factor 1 isoform b |
| MED19 | NM_153450 | mediator of RNA polymerase II transcription, |
| MED4 | NM_014166 | mediator of RNA polymerase II transcription, |
| MED8 | NM_001001651 | mediator of RNA polymerase II transcription |
| MEGF10 | NM_032446 | MEGF10 protein |
| MEOX1 | NM_004527 | mesenchyme homeobox 1 isoform 1 |
| MESP1 | NM_018670 | mesoderm posterior 1 |
| MEST | NM_002402 | mesoderm specific transcript isoform a |
| MET | NM_000245 | met proto-oncogene precursor |
| METAP1 | NM_015143 | methionyl aminopeptidase 1 |
| METT10D | NM_024086 | hypothetical protein LOC79066 |
| METTL1 | NM_005371 | methyltransferase-like protein 1 isoform a |
| METTL4 | NM_022840 | methyltransferase like 4 |
| MFAP2 | NM_002403 | microfibrillar-associated protein 2 precursor |
| MFAP4 | NM_002404 | microfibrillar-associated protein 4 |
| MFN2 | NM_014874 | mitofusin 2 |
| MFRP | NM_031433 | membrane frizzled-related protein |
| MGAT1 | NM_002406 | mannosyl (alpha-1,3-)-glycoprotein |
| MGAT3 | NM_002409 | mannosyl (beta-1,4-)-glycoprotein |
| MGAT4B | NM_014275 | mannosyl (alpha-1,3-)-glycoprotein |
| MGAT5B | NM_144677 | beta(1,6)-N-acetylglucosaminyltransferase V |
| MGC11102 | NM_032325 | hypothetical protein LOC84285 |
| MGC12981 | NM_032357 | hypothetical protein LOC84317 |
| MGC13024 | NM_152288 | hypothetical protein LOC93129 |
| MGC13114 | NM_032366 | hypothetical protein LOC84326 isoform a |
| MGC13138 | NM_033410 | hypothetical protein LOC92595 |
| MGC16169 | NM_033115 | hypothetical protein LOC93627 |
| MGC16291 | NM_032770 | hypothetical protein LOC84856 |
| MGC17330 | NM_052880 | HGFL protein |
| MGC21644 | NM_138492 | hypothetical protein LOC153768 isoform c |
| MGC21675 | NM_052861 | hypothetical protein LOC92070 |
| MGC23280 | NM_144683 | hypothetical protein LOC147015 |
| MGC24039 | NM_144973 | hypothetical protein LOC160518 |
| MGC26694 | NM_178526 | hypothetical protein LOC284439 |
| MGC2752 | NM_023939 | hypothetical protein LOC65996 |
| MGC3123 | NM_024107 | hypothetical protein LOC79089 isoform 1 |
| MGC33556 | NM_001004307 | hypothetical protein LOC339541 |
| MGC34774 | NM_203308 | hypothetical protein LOC399670 |
| MGC35440 | NM_153220 | hypothetical protein LOC147990 |
| MGC39518 | NM_173822 | hypothetical protein LOC285172 |
| MGC42367 | NM_207362 | hypothetical protein LOC343990 |
| MGC4268 | NM_031445 | hypothetical protein LOC83607 |
| MGC43122 | NM_173513 | hypothetical protein LOC151477 |
| MGC44328 | NM_001004344 | hypothetical protein LOC440757 |
| MGC45491 | NM_153246 | hypothetical protein LOC221416 |
| MGC50722 | NM_203348 | hypothetical protein LOC399693 |
| MGC52057 | NM_194317 | hypothetical protein LOC130574 |
| MGC5242 | NM_024033 | hypothetical protein LOC78996 |
| MGC70857 | NM_001001795 | hypothetical protein LOC414919 |
| MGC70870 | NM_203481 | hypothetical LOC403340 |
| MGLL | NM_001003794 | monoglyceride lipase isoform 2 |
| MIB1 | NM_020774 | mindbomb homolog 1 |
| MICAL-L1 | NM_033386 | molecule interacting with Rab13 |
| MIER2 | NM_017550 | hypothetical protein LOC54531 |
| MIER3 | NM_152622 | hypothetical protein LOC166968 |
| MIR16 | NM_016641 | membrane interacting protein of RGS16 |
| MITF | NM_000248 | microphthalmia-associated transcription factor |
| MK167 | NM_002417 | antigen identified by monoclonal antibody Ki-67 |
| MKL2 | NM_014048 | megakaryoblastic leukemia 2 protein |
| MKLN1 | NM_013255 | muskelin 1, intracellular mediator containing |
| MKNK2 | NM_199054 | MAP kinase-interacting serine/threonine kinase 2 |
| MKX | NM_173576 | hypothetical protein LOC283078 |
| MLANA | NM_005511 | melan-A |
| MLC1 | NM_015166 | megalencephalic leukoencephalopathy with |
| MLL | NM_005933 | myeloid/lymphoid or mixed-lineage leukemia |
| MLLT3 | NM_004529 | myeloid/lymphoid or mixed-lineage leukemia |
| MMAB | NM_052845 | cob(I)alamin adenosyltransferase |
| MMP11 | NM_005940 | matrix metalloproteinase 11 preproprotein |
| MMP14 | NM_004995 | matrix metalloproteinase 14 preproprotein |
| MMP15 | NM_002428 | matrix metalloproteinase 15 preproprotein |
| MMP19 | NM_001032360 | matrix metalloproteinase 19 isoform 2 precursor |
| MMP2 | NM_004530 | matrix metalloproteinase 2 preproprotein |
| MMP25 | NM_022468 | matrix metalloproteinase 25 preproprotem |
| MMS19L | NM_022362 | MMSI9-like (MET18 homolog, S. cerevisiae) |
| MNT | NM_020310 | MAX binding protein |
| MOAP1 | NM_022151 | modulator of apoptosis 1 |
| MOBKL1A | NM_173468 | MOB1, Mps One Binder kinase activator-like 1A |
| MOBKL2A | NM_130807 | MOB-LAK |
| MOBKL2C | NM_145279 | MOB1, Mps One Binder kinase activator-like 2C |
| MOV10 | NM_020963 | Mov10, Moloney leukemia virus 10, homolog |
| MOV10L1 | NM_018995 | MOV10-like 1 |
| MPHOSPH6 | NM_005792 | M-phase phosphoprotein 6 |
| MPI | NM_002435 | mannose-6-phosphate isomerase |
| MPL | NM_005373 | myeloproliferative leukemia virus oncogene |
| MPO | NM_000250 | myeloperoxidase |
| MPP2 | NM_005374 | palmitoylated membrane protein 2 |
| MPP5 | NM_022474 | membrane protein, palmitoylated 5 |
| MPPED1 | NM_001585 | hypothetical protein LOC758 |
| MPPED2 | NM_001584 | hypothetical protein LOC744 |
| MRAS | NM_012219 | muscle RAS oncogene homolog |
| MRGPRX2 | NM_054030 | MAS-related GPR, member X2 |
| M-RIP | NM_015134 | myosin phosphatase-Rho interacting protein |
| MRPL10 | NM_145255 | mitochondrial ribosomal protein L10 isoform a |
| MRPL30 | NM_145212 | mitochondrial ribosomal protein L30 |
| MRPL33 | NM_004891 | mitochondrial ribosomal protein L33 isoform a |
| MRPL47 | NM_020409 | mitochondrial ribosomal protein L47 isoform a |
| MRPL52 | NM_178336 | mitochondrial ribosomal protein L52 isoform a |
| MRPS12 | NM_021107 | mitochondrial ribosomal protein S12 precursor |
| MRPS25 | NM_022497 | mitochondrial ribosomal protein S25 |
| MRPS27 | NM_015084 | mitochondrial ribosomal protein S27 |
| MRPS7 | NM_015971 | mitochondrial ribosomal protein S7 |
| MRVI1 | NM_006069 | JAW1-related protein isoform a |
| MS4A10 | NM_206893 | membrane-spanning 4-domains, subfamily A, member |
| MS4A2 | NM_000139 | membrane-spanning 4-domains, subfamily A, member |
| MSI1 | NM_002442 | musashi 1 |
| MSL2L1 | NM_018133 | ring finger protein 184 |
| MSL3L1 | NM_078628 | male-specific lethal 3-like 1 isoform d |
| MSR1 | NM_138715 | macrophage scavenger receptor 1 isoform type 1 |
| MST150 | NM_032947 | putative small membrane protein NID67 |
| MSX2 | NM_002449 | msh homeobox 2 |
| MT1E | NM_175617 | metallothionein 1E |
| MTA2 | NM_004739 | metastasis-associated protein 2 |
| MTAP | NM_002451 | 5-methylthioadenosine phosphorylase |
| MTERFD2 | NM_182501 | MTERF domain containing 2 |
| MTFR1 | NM_014637 | chondrocyte protein with a poly-proline region |
| MTHFR | NM_005957 | 5,10-methylenetetrahydrofolate reductase |
| MTM1 | NM_000252 | myotubularin |
| MTMR12 | NM_019061 | myotubularin related protein 12 |
| MTMR2 | NM_016156 | myotubularin-related protein 2 isoform 1 |
| MTMR3 | NM_021090 | myotubularin-related protein 3 isoform c |
| MTMR4 | NM_004687 | myotubularin related protein 4 |
| MTMR9 | NM_015458 | myotubularin-related protein 9 |
| MTUS1 | NM_001001924 | mitochondrial tumor suppressor 1 isoform 1 |
| MUCDHL | NM_031265 | mu-protocadherin isoform 4 |
| MUM1 | NM_032853 | melanoma ubiquitous mutated protein |
| MXD4 | NM_006454 | MAD4 |
| MYADM | NM_001020818 | myeloid-associated differentiation marker |
| MYADML | NM_207329 | myeloid-associated differentiation marker-like |
| MYB | NM_005375 | v-myb myeloblastosis viral oncogene homolog |
| MYC | NM_002467 | myc proto-oncogene protein |
| MYCBP2 | NM_015057 | MYC binding protein 2 |
| MYCN | NM_005378 | v-myc myelocytomatosis viral related oncogene, |
| MYH6 | NM_002471 | myosin heavy chain 6 |
| MYH9 | NM_002473 | myosin, heavy polypeptide 9, non-muscle |
| MYL4 | NM_001002841 | atrial/embryonic alkali myosin light chain |
| MYL9 | NM_006097 | myosin regulatory light polypeptide 9 isoform a |
| MYLK | NM_005965 | myosin light chain kinase isoform 6 |
| MYLK2 | NM_033118 | skeletal myosin light chain kinase |
| MYO10 | NM_012334 | myosin X |
| MYO15A | NM_016239 | myosin XV |
| MYO18A | NM_078471 | myosin 18A isoform a |
| MYO1C | NM_033375 | myosin IC |
| MYO1D | NM_015194 | myosin ID |
| MYO1F | NM_012335 | myosin IF |
| MYOZ3 | NM_133371 | myozenin 3 |
| MYRIP | NM_015460 | myosin VIIA and Rab interacting protein |
| MYT1 | NM_004535 | myelin transcription factor 1 |
| N4BP1 | NM_153029 | Nedd4 binding protein 1 |
| NAGPA | NM_016256 | N-acetylglucosamine-1-phosphodiester |
| NALP2 | NM_017852 | NACHT, leucine rich repeat and PYD containing 2 |
| NAP1L2 | NM_021963 | nucleosome assembly protein 1-like 2 |
| NAP1L5 | NM_153757 | nucleosome assembly protein 1-like 5 |
| NAPE-PLD | NM_198990 | N-acyl-phosphatidylethanolamine-hydrolyzing |
| NAT10 | NM_024662 | N-acetyltransferase-like protein |
| NAT11 | NM_024771 | hypothetical protein LOC79829 |
| NAV1 | NM_020443 | neuron navigator 1 |
| NAV2 | NM_145117 | neuron navigator 2 isoform 2 |
| NAV3 | NM_014903 | neuron navigator 3 |
| NBL1 | NM_005380 | neuroblastoma, suppression of tumorigenicity 1 |
| NBN | NM_001024688 | nibrin isoform 2 |
| NBPF11 | NM_183372 | hypothetical protein LOC200030 |
| NBPF4 | NM_152488 | hypothetical protein LOC148545 |
| NBR1 | NM_005899 | neighbor of BRCA1 gene 1 |
| NCBP2 | NM_007362 | nuclear cap binding protein subunit 2, 20 kDa |
| NCDN | NM_001014839 | neurochondrin isoform 1 |
| NCK2 | NM_001004720 | NCK adaptor protein 2 isoform A |
| NCLN | NM_020170 | nicalin |
| NCOA1 | NM_003743 | nuclear receptor coactivator 1 isoform 1 |
| NCOA4 | NM_005437 | nuclear receptor coactivator 4 |
| NCOA6IP | NM_024831 | PRIP-interacting protein PIPMT |
| NCOR2 | NM_006312 | nuclear receptor co-repressor 2 |
| NCR3 | NM_147130 | natural cytotoxicity triggering receptor 3 |
| NDOR1 | NM_014434 | NADPH dependent diflavin oxidoreductase 1 |
| NDRG1 | NM_006096 | N-myc downstream regulated gene 1 |
| NDRG4 | NM_020465 | NDRG family member 4 |
| NDST1 | NM_001543 | N-deacetylase/N-sulfotransferase (heparan |
| NDUFA4L2 | NM_020142 | NADH:ubiguinone oxidoreductase MLRQ subunit |
| NDUFC2 | NM_004549 | NADH dehydrogenase (ubiquinone) 1, subcomplex |
| NDUFS6 | NM_004553 | NADH dehydrogenase (ubiquinone) Fe—S protein 6, |
| NEDD4 | NM_006154 | neural precursor cell expressed, developmentally |
| NEDD4L | NM_015277 | ubiquitin-protein ligase NEDD4-like |
| NEDD8 | NM_006156 | neural precursor cell expressed, developmentally |
| NEDD9 | NM_182966 | neural precursor cell expressed, developmentally |
| NEGR1 | NM_173808 | neuronal growth regulator 1 |
| NEK11 | NM_024800 | NIMA (never in mitosis gene a)-related kinase |
| NEK9 | NM_033116 | NIMA related kinase 9 |
| NETO1 | NM_138966 | neuropilin-and tolloid-like protein 1 isoform 3 |
| NETO2 | NM_018092 | neuropilin-and tolloid-like protein 2 |
| NEU1 | NM_000434 | neuraminidase precursor |
| NEURL | NM_004210 | neuralized-like |
| NF2 | NM_000268 | neurofibromin 2 isoform 1 |
| NFAM1 | NM_145912 | NFAT activation molecule 1 precursor |
| NFASC | NM_015090 | neurofascin precursor |
| NFAT5 | NM_006599 | nuclear factor of activated T-cells 5 isoform c |
| NFATC4 | NM_004554 | cytoplasmic nuclear factor of activated T-cells |
| NFE2L1 | NM_003204 | nuclear factor (erythroid-derived 2)-like 1 |
| NFIX | NM_002501 | nuclear factor I/X (CCAAT-binding transcription |
| NFKBIA | NM_020529 | nuclear factor of kappa light polypeptide gene |
| NFKBIE | NM_004556 | nuclear factor of kappa light polypeptide gene |
| NFX1 | NM_147134 | nuclear transcription factor, X-box binding 1 |
| NFYA | NM_002505 | nuclear transcription factor Y, alpha isoform 1 |
| NFYC | NM_014223 | nuclear transcription factor Y, gamma |
| NGB | NM_021257 | neuroglobin |
| NGFR | NM_002507 | nerve growth factor receptor precursor |
| NHLRC1 | NM_198586 | malin |
| NHS | NM_198270 | Nance-Horan syndrome protein |
| NIN | NM_020921 | ninein isoform 2 |
| NINJ1 | NM_004148 | ninjurin 1 |
| NINJ2 | NM_016533 | ninjurin 2 |
| NIPSNAP1 | NM_003634 | nipsnap homolog 1 |
| NIPSNAP3B | NM_018376 | nipsnap homolog 3B |
| NKD2 | NM_033120 | naked cuticle homolog 2 |
| NKTR | NM_001012651 | natural killer-tumor recognition sequence |
| NKX3-1 | NM_006167 | NK3 transcription factor related, locus 1 |
| NLE1 | NM_001014445 | Notchless gene homolog isoform b |
| NMNAT3 | NM_178177 | nicotinamide nucleotide adenylyltransferase 3 |
| NMT1 | NM_021079 | N-myristoyltransferase 1 |
| NMT2 | NM_004808 | glycylpeptide N-tetradecanoyltransferase 2 |
| NMUR1 | NM_006056 | neuromedin U receptor 1 |
| NNAT | NM_005386 | neuronatin isoform alpha |
| NOL1 | NM_001033714 | nucleolar protein 1, 120 kDa |
| NOL10 | NM_024894 | nucleolar protein 10 |
| NOL6 | NM_022917 | nucleolar RNA-associated protein alpha isoform |
| NONO | NM_007363 | non-POU domain containing, octamer-binding |
| NOS1AP | NM_014697 | nitric oxide synthase 1 (neuronal) adaptor |
| NOS2A | NM_000625 | nitric oxide synthase 2A isoform 1 |
| NOS3 | NM_000603 | nitric oxide synthase 3 (endothelial cell) |
| NOTCH1 | NM_017617 | notchl preproprotein |
| NOTCH2 | NM_024408 | notch 2 preproprotein |
| NOTCH3 | NM_000435 | Notch homolog 3 |
| NOTCH4 | NM_004557 | notch4 preproprotein |
| NOTUM | NM_178493 | hypothetical protein LOC147111 |
| N-PAC | NM_032569 | cytokine-like nuclear factor n-pac |
| NPAS4 | NM_178864 | HLH-PAS transcription factor NXF |
| NPC1L1 | NM_013389 | NPCl-like 1 |
| NPLOC4 | NM_017921 | nuclear protein localization 4 |
| NPNT | NM_001033047 | nephronectin |
| NPTX1 | NM_002522 | neuronal pentraxin I precursor |
| NPTX2 | NM_002523 | neuronal pentraxin II |
| NQO1 | NM_000903 | NAD(P)H menadione oxidoreductase 1, |
| NR1I2 | NM_003889 | pregnane X receptor isoform 1 |
| NR2E3 | NM_016346 | photoreceptor-specific nuclear receptor isoform |
| NR4A1 | NM_173158 | nuclear receptor subfamily 4, group A, member 1 |
| NR4A2 | NM_006186 | nuclear receptor subfamily 4, group A, member 2 |
| NR5A2 | NM_003822 | nuclear receptor subfamily 5, group A, member 2 |
| NRBP2 | NM_178564 | nuclear receptor binding protein 2 |
| NRG1 | NM_013958 | neuregulin 1 isoform HRG-beta3 |
| NRIP2 | NM_031474 | nuclear receptor interacting protein 2 |
| NRIP3 | NM_020645 | nuclear receptor interacting protein 3 |
| NRK | NM_198465 | Nik related kinase |
| NRN1 | NM_016588 | neuritin precursor |
| NRP2 | NM_003872 | neuropilin 2 isoform 2 precursor |
| NRXN2 | NM_015080 | neurexin 2 isoform alpha-1 precursor |
| NT5C2 | NM_012229 | 5′-nucleotidase, cytosolic II |
| NT5DC3 | NM_001031701 | hypothetical protein LOC51559 isoform 1 |
| NTNG2 | NM_032536 | netrin G2 |
| NTRK2 | NM_001018064 | neurotrophic tyrosine kinase, receptor, type 2 |
| NTSR1 | NM_002531 | neurotensin receptor 1 |
| NUDCD3 | NM_015332 | NudC domain containing 3 |
| NUDT13 | NM_015901 | nudix-type motif 13 |
| NUDT16L1 | NM_032349 | syndesmos |
| NUDT4 | NM_019094 | nudix-type motif 4 isoform alpha |
| NUDT8 | NM_181843 | nudix-type motif 8 |
| NUFIP1 | NM_012345 | nuclear fragile X mental retardation protein |
| NUFIP2 | NM_020772 | 82-kD FMRP Interacting Protein |
| NUMB | NM_001005743 | numb homolog isoform 1 |
| NUMBL | NM_004756 | numb homolog (Drosophila)-like |
| NUP188 | NM_015354 | nucleoporin 188 kDa |
| NUP210 | NM_024923 | nucleoporin 210 |
| NUP43 | NM_198887 | nucleoporin 43 kDa |
| NUPL1 | NM_001008564 | nucleoporin like 1 isoform b |
| NYD-SP18 | NM_032599 | testes development-related NYD-SP18 |
| NYD-SP21 | NM_032597 | testes development-related NYD-SP21 |
| NYREN18 | NM_016118 | NEDD8 ultimate buster-1 |
| OAS3 | NM_006187 | 2′-5′oligoadenylate synthetase 3 |
| OATL1 | NM_001006113 | ornithine aminotransferase-like 1 isoform 1 |
| OAZ2 | NM_002537 | ornithine decarboxylase antizyme 2 |
| OCRL | NM_000276 | phosphatidylinositol polyphosphate 5-phosphatase |
| ODF3L1 | NM_175881 | outer dense fiber of sperm tails 3-like 1 |
| ODZ1 | NM_014253 | odz, odd Oz/ten-m homolog 1 |
| OGDH | NM_002541 | oxoglutarate (alpha-ketoglutarate) dehydrogenase |
| OGFOD1 | NM_018233 | hypothetical protein LOC55239 |
| OGT | NM_003605 | O-linked GlcNAc transferase isoform 3 |
| OLFM4 | NM_006418 | olfactomedin 4 precursor |
| OLFML1 | NM_198474 | olfactomedin-like 1 |
| OLIG2 | NM_005806 | oligodendrocyte lineage transcription factor 2 |
| OLIG3 | NM_175747 | oligodendrocyte transcription factor 3 |
| OPCML | NM_001012393 | opioid binding protein/cell adhesion |
| OPHN1 | NM_002547 | oligophrenin 1 |
| OPN4 | NM_001030015 | opsin 4 isoform 2 |
| OPN5 | NM_001030051 | opsin 5 isoform 2 |
| OPRL1 | NM_000913 | opiate receptor-like 1 |
| OPRM1 | NM_001008505 | opioid receptor, mu 1 isoform MOR-1X |
| OPRS1 | NM_005866 | opioid receptor, sigma 1 isoform 1 |
| OR2H1 | NM_030883 | olfactory receptor, family 2, subfamily H, |
| OR51E2 | NM_030774 | olfactory receptor, family 51, subfamily E, |
| ORAOV1 | NM_153451 | oral cancer overexpressed 1 |
| ORMDL3 | NM_139280 | ORM1-like 3 |
| OSBPL7 | NM_017731 | oxysterol-binding protein-like protein 7 |
| OSCAR | NM_130771 | osteoclast-associated receptor isoform 3 |
| OSM | NM_020530 | oncostatin M precursor |
| OTOF | NM_004802 | otoferlin isoform b |
| OTUB2 | NM_023112 | OTU domain, ubiguitin aldehyde binding 2 |
| OTX1 | NM_014562 | orthodenticle 1 |
| OVCA2 | NM_080822 | candidate tumor suppressor in ovarian cancer 2 |
| OVOL1 | NM_004561 | OVO-like 1 binding protein |
| OVOL2 | NM_021220 | zinc finger protein 339 |
| OXSR1 | NM_005109 | oxidative-stress responsive 1 |
| P15RS | NM_018170 | hypothetical protein FLJ10656 |
| P18SRP | NM_173829 | P18SRP protein |
| P2RX4 | NM_175567 | purinergic receptor P2X4 isoform b |
| P2RX7 | NM_177427 | purinergic receptor P2X7 isoform b |
| P2RXL1 | NM_005446 | purinergic receptor P2X-like 1, orphan receptor |
| P2RY14 | NM_014879 | purinergic receptor P2Y, G-protein coupled, 14 |
| P2RY2 | NM_002564 | urinergic receptor P2Y2 |
| P2RY8 | NM_178129 | G-protein coupled purinergic receptor P2Y8 |
| P4HA3 | NM_182904 | prolyl 4-hydroxylase, alpha III subunit |
| PACS1 | NM_018026 | phosphofurin acidic cluster sorting protein 1 |
| PACSIN1 | NM_020804 | protein kinase C and casein kinase substrate in |
| PAG1 | NM_018440 | phosphoprotein associated with glycosphingolipid |
| PAICS | NM_006452 | phosphoribosylaminoimidazole carboxylase |
| PAK1 | NM_002576 | p21-activated kinase 1 |
| PAK4 | NM_001014831 | p21-activated kinase 4 isoform 1 |
| PALLD | NM_016081 | palladin |
| PALM2-AKAP2 | NM_007203 | PALM2-AKAP2 rotein isoform 1 |
| PAN3 | NM_175854 | PABPI-dependent poly A-specific ribonuclease |
| PAPOLB | NM_020144 | poly(A) polymerase beta (testis specific) |
| PAPOLG | NM_022894 | poly(A) polymerase gamma |
| PAPPA | NM_002581 | pregnancy-associated plasma protein A |
| PAPPA2 | NM_020318 | pappalysin 2 isoform 1 |
| PAPSS2 | NM_001015880 | 3′-phosphoadenosine 5′-phosphosulfate synthase 2 |
| PAQR5 | NM_017705 | membrane progestin receptor gamma |
| PAQR7 | NM_178422 | progestin and adipoQ receptor family member VII |
| PAQR8 | NM_133367 | progestin and adipoQ receptor family member |
| PARD6B | NM_032521 | PAR-6 beta |
| PARN | NM_002582 | poly(A)-specific ribonuclease (deadenylation |
| PARP10 | NM_032789 | poly (ADP-ribose) polymerase family, member 10 |
| PARP11 | NM_020367 | poly (ADP-ribose) polymerase family, member 11 |
| PARP14 | NM_017554 | poly (ADP-ribose) polymerase family, member 14 |
| PARS2 | NM_152268 | prolyl-tRNA synthetase |
| PARVA | NM_018222 | parvin, alpha |
| PARVG | NM_022141 | parvin, gamma |
| PAX2 | NM_000278 | paired box protein 2 isoform b |
| PAX8 | NM_013952 | paired box gene 8 isoform PAX8C |
| PBEF1 | NM_005746 | pre-B-cell colony enhancing factor 1 isoform a |
| PCBP4 | NM_020418 | poly(rC) binding protein 4 isoform a |
| PCDH11X | NM_032968 | protocadherin 11 X-linked isoform c |
| PCDH11Y | NM_032973 | protocadherin 11 Y-linked isoform c |
| PCDH17 | NM_014459 | protocadherin 17 |
| PCDH21 | NM_033100 | protocadherin 21 precursor |
| PCDHGA1 | NM_018912 | protocadherin gamma subfamily A, 1 isoform 1 |
| PCDHGA10 | NM_018913 | protocadherin gamma subfamily A, 10 isoform 1 |
| PCDHGA11 | NM_018914 | protocadherin gamma subfamily A, 11 isoform 1 |
| PCDHGA12 | NM_003735 | protocadherin gamma subfamily A, 12 isoform 1 |
| PCDHGA2 | NM_018915 | protocadherin gamma subfamily A, 2 isoform 1 |
| PCDHGA3 | NM_018916 | protocadherin gamma subfamily A, 3 isoform 1 |
| PCDHGA4 | NM_018917 | protocadherin gamma subfamily A, 4 isoform 1 |
| PCDHGA5 | NM_018918 | protocadherin gamma subfamily A, 5 isoform 1 |
| PCDHGA6 | NM_018919 | protocadherin gamma subfamily A, 6 isoform 1 |
| PCDHGA7 | NM_018920 | protocadherin gamma subfamily A, 7 isoform 1 |
| PCDHGA8 | NM_032088 | protocadherin gamma subfamily A, 8 isoform 1 |
| PCDHGA9 | NM_018921 | protocadherin gamma subfamily A, 9 isoform 1 |
| PCDHGB1 | NM_018922 | protocadherin gamma subfamily B, 1 isoform 1 |
| PCDHGB2 | NM_018923 | protocadherin gamma subfamily B, 2 isoform 1 |
| PCDHGB3 | NM_018924 | protocadherin gamma subfamily B, 3 isoform 1 |
| PCDHGB4 | NM_003736 | protocadherin gamma subfamily B, 4 isoform 1 |
| PCDHGB5 | NM_018925 | protocadherin gamma subfamily B, 5 isoform 1 |
| PCDHGB6 | NM_018926 | protocadherin gamma subfamily B, 6 isoform 1 |
| PCDHGB7 | NM_018927 | protocadherin gamma subfamily B, 7 isoform 1 |
| PCDHGC3 | NM_002588 | protocadherin gamma subfamily C, 3 isoform 1 |
| PCDHGC4 | NM_018928 | protocadherin gamma subfamily C, 4 isoform 1 |
| PCDHGC5 | NM_018929 | protocadherin gamma subfamily C, 5 isoform 1 |
| PCGF3 | NM_006315 | ring finger protein 3 |
| PCK2 | NM_001018073 | mitochondrial phosphoenolpyruvate carboxykinase |
| PCMTD1 | NM_052937 | hypothetical protein LOC115294 |
| PCNXL2 | NM_014801 | pecanex-like 2 |
| PCSK1N | NM_013271 | proprotein convertase subtilisin/kexin type 1 |
| PCSK7 | NM_004716 | proprotein convertase subtilisin/kexin type 7 |
| PCTK3 | NM_002596 | PCTAIRE protein kinase 3 isoform b |
| PCYOX1 | NM_016297 | prenylcysteine oxidase 1 |
| PCYT2 | NM_002861 | phosphate cytidylyltransferase 2, ethanolamine |
| PDCD10 | NM_007217 | programmed cell death 10 |
| PDCD2 | NM_144781 | programmed cell death 2 isoform 2 |
| PDCD4 | NM_014456 | programmed cell death 4 isoform 1 |
| PDE1B | NM_000924 | phosphodiesterase 1B, calmodulin-dependent |
| PDE4A | NM_006202 | phosphodiesterase 4A, cAMP-specific |
| PDE5A | NM_001083 | phosphodiesterase 5A isoform 1 |
| PDE7A | NM_002603 | phosphodiesterase 7A isoform a |
| PDE7B | NM_018945 | phosphodiesterase 7B |
| PDGFB | NM_002608 | platelet-derived growth factor beta isoform 1, |
| PDGFRA | NM_006206 | platelet-derived growth factor receptor alpha |
| PDGFRB | NM_002609 | platelet-derived growth factor receptor beta |
| PDK2 | NM_002611 | pyruvate dehydrogenase kinase, isoenzyme 2 |
| PDLIM2 | NM_021630 | PDZ and LIM domain 2 isoform 2 |
| PDLIM7 | NM_213636 | PDZ and LIM domain 7 isoform 4 |
| PDPN | NM_001006624 | lung type-I cell membrane-associated |
| PDPR | NM_017990 | pyruvate dehydrogenase phosphatase regulatory |
| PDRG1 | NM_030815 | p53 and DNA damage-regulated protein |
| PDXK | NM_003681 | pyridoxal kinase |
| PDZD11 | NM_016484 | PDZ domain containing 11 |
| PDZRN3 | NM_015009 | PDZ domain containing RING finger 3 |
| PEA15 | NM_003768 | phosphoprotein enriched in astrocytes 15 |
| PER1 | NM_002616 | period 1 |
| PER2 | NM_022817 | period 2 isoform 1 |
| PER3 | NM_016831 | period 3 |
| PERLD1 | NM_033419 | CAB2 protein |
| PES1 | NM_014303 | pescadillo homolog 1, containing BRCT domain |
| PEX11B | NM_003846 | peroxisomal biogenesis factor 11B |
| PEX11G | NM_080662 | peroxisomal biogenesis factor 11 gamma |
| PEX14 | NM_004565 | peroxisomal biogenesis factor 14 |
| PEX5L | NM_016559 | PXR2b protein |
| PEX7 | NM_000288 | peroxisomal biogenesis factor 7 |
| PFKFB1 | NM_002625 | 6-phosphofructo-2-kinase/fructose-2, |
| PFKFB3 | NM_004566 | 6-phosphofructo-2-kinase/fructose-2, |
| PFTK1 | NM_012395 | PFTAIRE protein kinase 1 |
| PGAM1 | NM_002629 | phosphoglycerate mutase 1 (brain) |
| PGAM4 | NM_001029891 | phosphoglycerate mutase family 3 |
| PGAP1 | NM_024989 | GPI deacylase |
| PGD | NM_002631 | phosphogluconate dehydrogenase |
| PGDS | NM_014485 | prostaglandin-D synthase |
| PGEA1 | NM_001002880 | PKD2 interactor, golgi and endoplasmic reticulum |
| PGF | NM_002632 | placental growth factor, vascular endothelial |
| PGLYRP2 | NM_052890 | peptidoglycan recognition protein L precursor |
| PGLYRP3 | NM_052891 | peptidoglycan recognition protein-I-alpha |
| PGM1 | NM_002633 | phosphoglucomutase 1 |
| PGM2L1 | NM_173582 | phosphoglucomutase 2-like 1 |
| PGM5 | NM_021965 | phosphoglucomutase 5 |
| PGRMC2 | NM_006320 | progesterone membrane binding protein |
| PHACTR4 | NM_023923 | phosphatase and actin regulator 4 |
| PHB | NM_002634 | prohibitin |
| PHF13 | NM_153812 | PHD finger protein 13 |
| PHF15 | NM_015288 | PHD finger protein 15 |
| PHF19 | NM_015651 | PHD finger protein 19 isoform a |
| PHF6 | NM_001015877 | PHD finger protein 6 isoform 1 |
| PHF8 | NM_015107 | PHD finger protein 8 |
| PHGDHL1 | NM_177967 | hypothetical protein LOC337867 |
| PHKB | NM_000293 | phosphorylase kinase, beta isoform a |
| PHYHIP | NM_014759 | phytanoyl-CoA hydroxylase interacting protein |
| PI16 | NM_153370 | protease inhibitor 16 precursor |
| PICK1 | NM_012407 | protein interacting with C kinase 1 |
| PIGQ | NM_004204 | phosphatidylinositol glycan, class Q isoform 2 |
| PIGZ | NM_025163 | SMP3 mannosyltransferase |
| PIK3CD | NM_005026 | phosphoinositide-3-kinase, catalytic, delta |
| PIK3R2 | NM_005027 | phosphoinositide-3-kinase, regulatory subunit 2 |
| PIK3R3 | NM_003629 | phosphoinositide-3-kinase, regulatory subunit 3 |
| PIK4CB | NM_002651 | phosphatidylinositol 4-kinase, catalytic, beta |
| PIP3-E | NM_015553 | phosphoinositide-binding protein PIP3-E |
| PIP5K1A | NM_003557 | phosphatidylinositol-4-phosphate 5-kinase, type |
| PIP5K1C | NM_012398 | phosphatidylinositol-4-phosphate 5-kinase, type |
| PIP5K2B | NM_003559 | phosphatidylinositol-4-phosphate 5-kinase type |
| PIP5K3 | NM_001002881 | phosphatidylinositol-3- |
| PIWIL2 | NM_018068 | piwi-like 2 |
| PJA2 | NM_014819 | praja 2, R1NG-H2 motif containing |
| PKHD1 | NM_138694 | polyductin isoform 1 |
| PKIA | NM_006823 | cAMP-dependent protein kinase inhibitor alpha |
| PKNOX1 | NM_004571 | PBX/knotted 1 homeobox 1 isoform 1 |
| PKNOX2 | NM_022062 | PBX/knotted 1 homeobox 2 |
| PKP1 | NM_000299 | plakophilin 1 isoform 1b |
| PKP2 | NM_001005242 | plakophilin 2 isoform 2a |
| PKP4 | NM_001005476 | plakophilin 4 isoform b |
| PLA2G2D | NM_012400 | phospholipase A2, group IID |
| PLA2G2F | NM_022819 | phospholipase A2, group IIF |
| PLA2G4D | NM_178034 | phospholipase A2, group IVD |
| PLA2G6 | NM_001004426 | phospholipase A2, group VI isoform b |
| PLAC4 | NM_182832 | placenta-specific 4 |
| PLAG1 | NM_002655 | pleiomorphic adenoma gene 1 |
| PLAGL1 | NM_002656 | pleiomorphic adenoma gene-like 1 isoform 1 |
| PLAGL2 | NM_002657 | pleiomorphic adenoma gene-like 2 |
| PLB1 | NM_153021 | phospholipase B1 |
| PLCB1 | NM_015192 | phosphoinositide-specific phospholipase C beta 1 |
| PLCD3 | NM_133373 | phospholipase C delta 3 |
| PLCE1 | NM_016341 | pancreas-enriched phospholipase C |
| PLCG1 | NM_002660 | phospholipase C gamma 1 isoform a |
| PLCXD3 | NM_001005473 | phosphatidylinositol-specific phospholipase C, X |
| PLDN | NM_012388 | pallidin |
| PLEK | NM_002664 | pleckstrin |
| PLEKHA1 | NM_001001974 | pleckstrin homology domain containing, family A |
| PLEKHA5 | NM_019012 | pleckstrin homology domain containing, family A |
| PLEKHA6 | NM_014935 | phosphoinositol 3-phosphate-binding protein-3 |
| PLEKHF1 | NM_024310 | apoptosis-inducing protein D |
| PLEKHG5 | NM_020631 | putative NFkB activating protein isoform a |
| PLEKHG6 | NM_018173 | pleckstrin homology domain containing, family G |
| PLEKHH2 | NM_172069 | pleckstrin homology domain containing, family H |
| PLEKHJ1 | NM_018049 | pleckstrin homology domain containing, family J |
| PLEKHK1 | NM_145307 | pleckstrin homology domain containing, family K |
| PLEKHM1 | NM_014798 | pleckstrin homology domain containing, family M |
| PLEKHQ1 | NM_025201 | PH domain-containing protein |
| PLIN | NM_002666 | perilipin |
| PLN | NM_002667 | phospholamban |
| PLOD1 | NM_000302 | lysyl hydroxylase precursor |
| PLS1 | NM_002670 | plastin 1 |
| PLXDC1 | NM_020405 | plexin domain containing 1 precursor |
| PLXNA1 | NM_032242 | plexin A1 |
| PLXNA3 | NM_017514 | plexin A3 |
| PMCHL1 | NM_031887 | pro-melanin-concentrating hormone-like 1 |
| PMF1 | NM_007221 | polyamine-modulated factor 1 |
| PML | NM_033238 | promyelocytic leukemia protein isoform 1 |
| PNMA3 | NM_013364 | paraneoplastic cancer-testis-brain antigen |
| PNOC | NM_006228 | prepronociceptin |
| PNRC2 | NM_017761 | proline-rich nuclear receptor coactivator 2 |
| PODXL | NM_001018111 | podocalyxin-like precursor isoform 1 |
| POFUT1 | NM_015352 | protein O-fucosyltransferase 1 isoform 1 |
| POFUT2 | NM_015227 | protein O-fucosyltransferase 2 isoform A |
| POGZ | NM_015100 | pogo transposable element with ZNF domain |
| POLD3 | NM_006591 | polymerase (DNA directed), delta 3 |
| POLG | NM_002693 | polymerase (DNA directed), gamma |
| POLL | NM_013274 | polymerase (DNA directed), lambda |
| POLQ | NM_199420 | DNA polymerase theta |
| POLR2G | NM_002696 | DNA directed RNA polymerase II polypeptide G |
| POLR2J2 | NM_032958 | DNA directed RNA polymerase II polypeptide |
| POLR3H | NM_001018050 | polymerase (RNA) III (DNA directed) polypeptide |
| POLS | NM_006999 | DNA polymerase sigma |
| POMT1 | NM_007171 | protein-O-mannosyltransferase 1 |
| POMT2 | NM_013382 | putative protein O-mannosyltransferase |
| POMZP3 | NM_012230 | POMZP3 fusion protein isoform 1 |
| PON2 | NM_000305 | paraoxonase 2 isoform 1 |
| POTE14 | NM_001005356 | protein expressed in prostate, ovary, testis, |
| POU2F1 | NM_002697 | POU domain, class 2, transcription factor 1 |
| POU4F1 | NM_006237 | POU domain, class 4, transcription factor 1 |
| POU6F1 | NM_002702 | POU domain, class 6, transcription factor 1 |
| PPAP2B | NM_003713 | phosphatidic acid phosphatase type 2B |
| PPAPDC3 | NM_032728 | phosphatidic acid phosphatase type 2 domain |
| PPARA | NM_001001928 | peroxisome proliferative activated receptor, |
| PPARD | NM_006238 | peroxisome proliferative activated receptor, |
| PPARG | NM_005037 | peroxisome proliferative activated receptor |
| PPEF2 | NM_152933 | serine/threonine protein phosphatase with |
| PPFIA1 | NM_003626 | PTPRF interacting protein alpha 1 isoform b |
| PPFIA4 | NM_015053 | protein tyrosine phosphatase, receptor type, f |
| PPGB | NM_000308 | protective protein for beta-galactosidase |
| PPIE | NM_006112 | peptidylprolyl isomerase E isoform 1 |
| PPIL2 | NM_148175 | peptidylprolyl isomerase-like 2 isoform a |
| PPL | NM_002705 | periplakin |
| PPM1A | NM_021003 | protein phosphatase 1A isoform 1 |
| PPM1F | NM_014634 | protein phosphatase 1F |
| PPM1L | NM_139245 | protein phosphatase 1 (formerly 2C)-like |
| PPM1M | NM_144641 | protein phosphatase 1M (PP2C domain containing) |
| PPP1CC | NM_002710 | protein phosphatase 1, catalytic subunit, gamma |
| PPP1R10 | NM_002714 | protein phosphatase 1, regulatory subunit 10 |
| PPP1R11 | NM_021959 | protein phosphatase 1, regulatory (inhibitor) |
| PPP1R12B | NM_002481 | protein phosphatase 1, regulatory (inhibitor) |
| PPP1R13B | NM_015316 | protein phosphatase 1, regulatory (inhibitor) |
| PPP1R14D | NM_017726 | protein phosphatase 1, regulatory subunit 14D |
| PPP1R15B | NM_032833 | protein phosphatase 1, regulatory subunit 15B |
| PPPIR16B | NM_015568 | protein phosphatase 1 regulatory inhibitor |
| PPP1R8 | NM_002713 | protein phosphatase 1 regulatory inhibitor |
| PPP2R1B | NM_002716 | beta isoform of regulatory subunit A, protein |
| PPP2R2C | NM_020416 | gamma isoform of regulatory subunit B55, protein |
| PPP2R3A | NM_002718 | protein phosphatase 2, regulatory subunit B″, |
| PPP2R4 | NM_021131 | protein phosphatase 2A, regulatory subunit B′ |
| PPP2R5A | NM_006243 | protein phosphatase 2, regulatory subunit B |
| PPP2R5C | NM_002719 | gamma isoform of regulatory subunit B56, protein |
| PPP2R5D | NM_006245 | delta isoform of regulatory subunit B56, protein |
| PPP3R1 | NM_000945 | protein phosphatase 3, regulatory subunit B, |
| PPP3R2 | NM_147180 | protein phosphatase 3 regulatory subunit B, beta |
| PPP4R1L | NM_018498 | hypothetical protein LOC55370 |
| PPTC7 | NM_139283 | T-cell activation protein phosphatase 2C |
| PQLC1 | NM_025078 | PQ loop repeat containing 1 |
| PRAP1 | NM_145202 | proline-rich acidic protein 1 |
| PRC1 | NM_003981 | protein regulator of cytokinesis 1 isoform 1 |
| PRDM13 | NM_021620 | PR domain containing 13 |
| PRDM16 | NM_022114 | PR domain containing 16 isoform 1 |
| PREB | NM_013388 | prolactin regulatory element binding protein |
| PREI3 | NM_015387 | preimplantation protein 3 isoform 1 |
| PRELP | NM_002725 | proline arginine-rich end leucine-rich repeat |
| PREP | NM_002726 | prolyl endopeptidase |
| PREPL | NM_006036 | prolyl endopeptidase-like |
| PRICKLE2 | NM_198859 | prickle-like 2 |
| PRIMA1 | NM_178013 | proline rich membrane anchor 1 |
| PRKACB | NM_002731 | cAMP-dependent protein kinase catalytic subunit |
| PRKAG1 | NM_002733 | AMP-activated protein kinase, noncatalytic |
| PRKAG3 | NM_017431 | AMP-activated protein kinase, non-catalytic |
| PRKAR1B | NM_002735 | protein kinase, cAMP-dependent, regulatory, type |
| PRKCA | NM_002737 | protein kinase C, alpha |
| PRKCB1 | NM_002738 | protein kinase C, beta isoform 2 |
| PRKCE | NM_005400 | protein kinase C, epsilon |
| PRKCH | NM_006255 | protein kinase C, eta |
| PRKCQ | NM_006257 | protein kinase C, theta |
| PRKD1 | NM_002742 | protein kinase D1 |
| PRKD3 | NM_005813 | protein kinase D3 |
| PRKG2 | NM_006259 | protein kinase, cGMP-dependent, type II |
| PRKRIP1 | NM_024653 | PRKR interacting protein 1 (IL11 inducible) |
| PRKRIR | NM_004705 | protein-kinase, interferon-inducible double |
| PRKX | NM_005044 | protein kinase, X-linked |
| PRKY | NM_002760 | protein kinase, Y-linked |
| PRLR | NM_000949 | prolactin receptor |
| PRMT2 | NM_001535 | HMT1 hnRNP methyltransferase-like 1 |
| PRMT3 | NM_005788 | HMT1 hnRNP methyltransferase-like 3 |
| PRMT5 | NM_006109 | protein arginine methyltransferase 5 isoform a |
| PRND | NM_012409 | prion-like protein doppel preproprotein |
| PROM2 | NM_144707 | prominin 2 |
| ProSAPiP1 | NM_014731 | ProSAPiP1 protein |
| PROSC | NM_007198 | proline synthetase co-transcribed homolog |
| PROZ | NM_003891 | protein Z, vitamin K-dependent plasma |
| PRPF31 | NM_015629 | pre-mRNA processing factor 31 homolog |
| PRPF38B | NM_018061 | PRP38 pre-mRNA processing factor 38 (yeast) |
| PRPS1 | NM_002764 | phosphoribosyl pyrophosphate synthetase 1 |
| PRR11 | NM_018304 | hypothetical protein LOC55771 |
| PRSS21 | NM_006799 | testisin isoform 1 |
| PRSS7 | NM_002772 | enterokinase precursor |
| PRSS8 | NM_002773 | prostasin preproprotein |
| PRX | NM_020956 | periaxin isoform 1 |
| PRY | NM_004676 | PTPN13-like, Y-linked |
| PRY2 | NM_001002758 | PTPN13-like, Y-linked 2 |
| PSCD3 | NM_004227 | pleckstrin homology, Sec7 and coiled/coil |
| PSCD4 | NM_013385 | pleckstrin homology, Sec7 and coiled/coil |
| PSD2 | NM_032289 | pleckstrin and Sec7 domain containing 2 |
| PSD3 | NM_015310 | ADP-ribosylation factor guanine nucleotide |
| PSD4 | NM_012455 | pleckstrin and Sec7 domain containing 4 |
| PSKH1 | NM_006742 | protein serine kinase H1 |
| PSMD5 | NM_005047 | proteasome 26S non-ATPase subunit 5 |
| PSMD9 | NM_002813 | proteasome 26S non-ATPase subunit 9 |
| PSME1 | NM_006263 | proteasome activator subunit 1 isoform 1 |
| PSME3 | NM_005789 | proteasome activator subunit 3 isoform 1 |
| PSRC2 | NM_144982 | hypothetical protein LOC196441 |
| PTGER4 | NM_000958 | prostaglandin E receptor 4, subtype EP4 |
| PTGES2 | NM_025072 | prostaglandin E synthase 2 isoform 1 |
| PTGFR | NM_000959 | prostaglandin F receptor isoform a precursor |
| PTGFRN | NM_020440 | prostaglandin F2 receptor negative regulator |
| PTGIR | NM_000960 | prostaglandin I2 (prostacyclin) receptor (IP) |
| PTGIS | NM_000961 | prostaglandin I2 (prostacyclin) synthase |
| PTGS1 | NM_000962 | prostaglandin-endoperoxide synthase 1 isoform 1 |
| PTK6 | NM_005975 | PTK6 protein tyrosine kinase 6 |
| PTK7 | NM_152883 | PTK7 protein tyrosine kinase 7 isoform e |
| PTOV1 | NM_017432 | prostate tumor overexpressed gene 1 |
| PTPDC1 | NM_152422 | protein tyrosine phosphatase domain containing 1 |
| PTPLAD2 | NM_001010915 | hypothetical protein LOC401494 |
| PTPLB | NM_198402 | protein tyrosine phosphatase-like (proline |
| PTPN2 | NM_080422 | protein tyrosine phosphatase, non-receptor type |
| PTPN5 | NM_032781 | protein tyrosine phosphatase, non-receptor type |
| PTPRE | NM_006504 | protein tyrosine phosphatase, receptor type, E |
| PTPRG | NM_002841 | protein tyrosine phosphatase, receptor type, G |
| PTPRM | NM_002845 | protein tyrosine phosphatase, receptor type, M |
| PTPRN | NM_002846 | protein tyrosine phosphatase, receptor type, N |
| PTPRR | NM_002849 | protein tyrosine phosphatase, receptor type, R |
| PTPRT | NM_007050 | protein tyrosine phosphatase, receptor type, T |
| PTPRU | NM_005704 | protein tyrosine phosphatase, receptor type, U |
| PTPRZ1 | NM_002851 | protein tyrosine phosphatase, receptor-type, |
| PTRF | NM_012232 | polymerase I and transcript release factor |
| PTRH1 | NM_001002913 | hypothetical protein LOC138428 |
| PUM1 | NM_001020658 | pumilio 1 isoform 1 |
| PURB | NM_033224 | purine-rich element binding protein B |
| PURG | NM_001015508 | purine-rich element binding protein G isoform B |
| PUS7L | NM_031292 | hypothetical protein LOC83448 |
| PVR | NM_006505 | poliovirus receptor |
| PVRL2 | NM_002856 | poliovirus receptor-related 2 (herpesvirus entry |
| PXMP4 | NM_007238 | peroxisomal membrane protein 4 isoform a |
| PXN | NM_002859 | paxillin |
| PYDC1 | NM_152901 | pyrin domain containing 1 |
| PYGO2 | NM_138300 | pygopus homolog 2 |
| RAB10 | NM_016131 | ras-related GTP-binding protein RAB10 |
| RAB11FIP2 | NM_014904 | RAB11 family interacting protein 2 (class I) |
| RAB11FIP4 | NM_032932 | RAB11 family interacting protein 4 (class II) |
| RAB14 | NM_016322 | GTPase Rab14 |
| RAB17 | NM_022449 | RAB17, member RAS oncogene family |
| RAB1B | NM_030981 | RAB1B, member RAS oncogene family |
| RAB21 | NM_014999 | RAB21, member RAS oncogene family |
| RAB26 | NM_014353 | RAB26, member RAS oncogene family |
| RAB30 | NM_014488 | RAB30, member RAS oncogene family |
| RAB31 | NM_006868 | RAB31, member RAS oncogene family |
| RAB34 | NM_031934 | RAB39 |
| RAB35 | NM_006861 | RAB35, member RAS oncogene family |
| RAB36 | NM_004914 | RAB36, member RAS oncogene family |
| RAB39B | NM_171998 | RAB39B, member RAS oncogene family |
| RAB3B | NM_002867 | RAB3B, member RAS oncogene family |
| RAB3C | NM_138453 | RAB3C, member RAS oncogene family |
| RAB3IL1 | NM_013401 | RAB3A interacting protein (rabin3)-like 1 |
| RAB43 | NM_198490 | RAB43 protein |
| RAB4A | NM_004578 | RAB4A, member RAS oncogene family |
| RAB5B | NM_002868 | RAB5B, member RAS oncogene family |
| RAB6B | NM_016577 | RAB6B, member RAS oncogene family |
| RAB6IP2 | NM_015064 | RAB6-interacting protein 2 isoform alpha |
| RAB7B | NM_177403 | RAB7B, member RAS oncogene family |
| RAB8B | NM_016530 | RAB8B, member RAS oncogene family |
| RABIF | NM_002871 | RAB-interacting factor |
| RABL3 | NM_173825 | RAB, member of RAS oncogene family-like 3 |
| RABL5 | NM_022777 | RAB, member RAS oncogene family-like 5 |
| RAC2 | NM_002872 | ras-related C3 botulinum toxin substrate 2 |
| RAD23B | NM_002874 | UV excision repair protein RAD23 homolog B |
| RAD50 | NM_005732 | RAD50 homolog isoform 1 |
| RAD51 | NM_002875 | RAD51 homolog protein isoform 1 |
| RAD51L3 | NM_002878 | RAD51-like 3 isoform 1 |
| RAD9A | NM_004584 | RAD9 homolog |
| RAD9B | NM_152442 | RAD9 homolog B |
| RAE1 | NM_001015885 | RAE1 (RNA export 1, S. pombe) homolog |
| RAF1 | NM_002880 | v-raf-1 murine leukemia viral oncogene homolog |
| RAI14 | NM_015577 | retinoic acid induced 14 |
| RAI16 | NM_022749 | retinoic acid induced 16 |
| RAI17 | NM_020338 | retinoic acid induced 17 |
| RALB | NM_002881 | v-ral simian leukemia viral oncogene homolog B |
| RALGDS | NM_006266 | ral guanine nucleotide dissociation stimulator |
| RALGPS1 | NM_014636 | Ral GEF with PH domain and SH3 binding motif 1 |
| RALGPS2 | NM_152663 | Ral GEF with PH domain and SH3 binding motif 2 |
| RALY | NM_007367 | RNA binding protein (autoantigenic, |
| RANBP10 | NM_020850 | RAN binding protein 10 |
| RANBP17 | NM_022897 | RAN binding protein 17 |
| RANBP3 | NM_003624 | RAN binding protein 3 isoform RANBP3-a |
| RANGAP1 | NM_002883 | Ran GTPase activating protein 1 |
| RAP1GAP | NM_002885 | RAP1, GTPase activating protein 1 |
| RAP2B | NM_002886 | RAP2B, member of RAS oncogene family |
| RAPGEF6 | NM_016340 | PDZ domain-containing guanine nucleotide |
| RAPH1 | NM_213589 | Ras association and pleckstrin homology domains |
| RARA | NM_000964 | retinoic acid receptor, alpha isoform a |
| RARB | NM_000965 | retinoic acid receptor, beta isoform 1 |
| RARG | NM_000966 | retinoic acid receptor, gamma |
| RASA3 | NM_007368 | RAS p21 protein activator 3 |
| RASA4 | NM_006989 | RAS p21 protein activator 4 |
| RASD2 | NM_014310 | RASD family, member 2 |
| RASGEF1A | NM_145313 | RasGEF domain family, member 1A |
| RASGEF1C | NM_001031799 | RasGEF domain family, member 1C isoform 2 |
| RASGRP3 | NM_170672 | RAS guanyl releasing protein 3 (calcium and |
| RASGRP4 | NM_170604 | RAS guanyl releasing protein 4 isoform 1 |
| RASL10B | NM_033315 | RAS-like, family 10, member B |
| RASL12 | NM_016563 | RAS-like, family 12 protein |
| RASSF2 | NM_014737 | Ras association domain family 2 |
| RASSF5 | NM_031437 | Ras association (RalGDS/AF-6) domain family 5 |
| RASSF6 | NM_177532 | Ras association (RalGDS/AF-6) domain family 6 |
| RASSF7 | NM_003475 | Ras association (RalGDS/AF-6) domain family 7 |
| RBAK | NM_021163 | RB-associated KRAB repressor |
| RBBP5 | NM_005057 | retinoblastoma binding protein 5 |
| RBJ | NM_016544 | Ras-associated protein Rap1 |
| RBM12 | NM_006047 | RNA binding motif protein 12 |
| RBM13 | NM_032509 | RNA binding motif protein 13 |
| RBM15B | NM_013286 | RNA binding motif protein 15B |
| RBM17 | NM_032905 | RNA binding motif protein 17 |
| RBM19 | NM_016196 | RNA binding motif protein 19 |
| RBM23 | NM_018107 | hypothetical protein LOC55147 |
| RBM24 | NM_153020 | hypothetical protein LOC221662 |
| RBM28 | NM_018077 | RNA binding motif protein 28 |
| RBM33 | NM_001008408 | hypothetical protein LOC155435 |
| RBP2 | NM_004164 | retinol binding protein 2, cellular |
| RBP5 | NM_031491 | retinol binding protein 5, cellular |
| RBPMS2 | NM_194272 | RNA binding protein with multiple splicing 2 |
| RCC2 | NM_018715 | RCC1-like |
| RDH11 | NM_016026 | androgen-regulated short-chain |
| RDH12 | NM_152443 | retinol dehydrogenase 12 (all-trans and 9-cis) |
| RDH13 | NM_138412 | retinol dehydrogenase 13 (all-trans and 9-cis) |
| RDH5 | NM_002905 | retinol dehydrogenase 5 (11-cis and 9-cis) |
| RECK | NM_021111 | RECK protein precursor |
| REEP5 | NM_005669 | receptor accessory protein 5 |
| RELN | NM_005045 | reelin isoform a |
| REM1 | NM_014012 | RAS-like GTP-binding protein REM |
| REPIN1 | NM_013400 | replication initiator 1 isoform 1 |
| REXO1L1 | NM_172239 | exonuclease GOR |
| REXO4 | NM_020385 | XPMC2 prevents mitotic catastrophe 2 homolog |
| RFP2 | NM_001007278 | ret finger protein 2 isoform 2 |
| RFX1 | NM_002918 | regulatory factor X1 |
| RGAG4 | NM_001024455 | retrotransposon gag domain containing 4 |
| RGL1 | NM_015149 | ral guanine nucleotide dissociation |
| RGMB | NM_001012761 | RGM domain family, member B isoform 1 precursor |
| RGS11 | NM_003834 | regulator of G-protein signalling 11 isoform 2 |
| RGS17 | NM_012419 | regulator of G-protein signalling 17 |
| RGS3 | NM_021106 | regulator of G-protein signalling 3 isoform 2 |
| RGS4 | NM_005613 | regulator of G-protein signaling 4 |
| RGS5 | NM_003617 | regulator of G-protein signalling 5 |
| RGS6 | NM_004296 | regulator of G-protein signalling 6 |
| RGS9BP | NM_207391 | RGS9 anchor protein |
| RHBDL3 | NM_138328 | rhomboid, veinlet-like 3 |
| RHBG | NM_020407 | Rhesus blood group, B glycoprotein |
| RHEB | NM_005614 | Ras homolog enriched in brain |
| RHEBL1 | NM_144593 | Ras homolog enriched in brain like 1 |
| RHO | NM_000539 | rhodopsin |
| RHOBTB3 | NM_014899 | rho-related BTB domain containing 3 |
| RHOJ | NM_020663 | TC10-like Rho GTPase |
| RHOT1 | NM_001033567 | ras homolog gene family, member T1 isoform 4 |
| RHOT2 | NM_138769 | ras homolog gene family, member T2 |
| RHOU | NM_021205 | ras homolog gene family, member U |
| RHOV | NM_133639 | ras homolog gene family, member V |
| RIC3 | NM_024557 | resistance to inhibitors of cholinesterase 3 |
| RIC8B | NM_018157 | resistance to inhibitors of cholinesterase 8 |
| RICS | NM_014715 | Rho GTPase-activating protein |
| RILP | NM_031430 | Rab interacting lysosomal protein |
| RIMBP2 | NM_015347 | RIM-binding protein 2 |
| RIMS3 | NM_014747 | regulating synaptic membrane exocytosis 3 |
| RIMS4 | NM_182970 | regulating synaptic membrane exocytosis 4 |
| RIN1 | NM_004292 | ras inhibitor RIN1 |
| RIP | NM_001033002 | RPA interacting protein isoform 1 |
| RIPK4 | NM_015375 | receptor interacting protein kinase 5 isoform 1 |
| RKHD2 | NM_016626 | ring finger and KH domain containing 2 |
| RNASE11 | NM_145250 | ribonuclease, RNase A family, 11 (non-active) |
| RNASEL | NM_021133 | ribonuclease L |
| RNF128 | NM_024539 | ring finger protein 128 isoform 2 |
| RNF144 | NM_014746 | ring finger protein 144 |
| RNF165 | NM_152470 | ring finger protein 165 |
| RNF182 | NM_152737 | ring finger protein 182 |
| RNF185 | NM_152267 | ring finger protein 185 |
| RNF19 | NM_015435 | ring finger protein 19 |
| RNF24 | NM_007219 | ring finger protein 24 |
| RNF31 | NM_017999 | ring finger protein 31 |
| RNF34 | NM_025126 | ring finger protein 34 isoform 2 |
| RNF38 | NM_022781 | ring finger protein 38 isoform 1 |
| RNF4 | NM_002938 | ring finger protein 4 |
| RNF40 | NM_014771 | ring finger protein 40 |
| RNF41 | NM_005785 | ring finger protein 41 isoform 1 |
| RNF44 | NM_014901 | ring finger protein 44 |
| RNF8 | NM_003958 | ring finger protein 8 isoform 1 |
| RNPC1 | NM_017495 | RNA-binding region containing protein 1 isoform |
| ROD1 | NM_005156 | ROD1 regulator of differentiation 1 |
| ROGDI | NM_024589 | leucine zipper domain protein |
| RP11-19J3.3 | NM_001012267 | hypothetical protein LOC401541 |
| RP13-15M17.2 | NM_001010866 | hypothetical protein LOC199953 |
| RP1-32F7.2 | NM_173698 | hypothetical protein LOC286499 |
| RP13-360B22.2 | NM_032227 | hypothetical protein LOC84187 |
| RPH3A | NM_014954 | rabphilin 3A homolog |
| RPH3AL | NM_006987 | rabphilin 3A-like (without C2 domains) |
| RPIA | NM_144563 | ribose 5-phosphate isomerase A (ribose |
| RPL13A | NM_012423 | ribosomal protein L13a |
| RPL28 | NM_000991 | ribosomal protein L28 |
| RPL32 | NM_000994 | ribosomal protein L32 |
| RPL7L1 | NM_198486 | ribosomal protein L7-like 1 |
| RPS23 | NM_001025 | ribosomal protein S23 |
| RPS29 | NM_001030001 | ribosomal protein S29 isoform 2 |
| RPS6KA4 | NM_001006944 | ribosomal protein S6 kinase, 90 kDa, polypeptide |
| RPS6KB1 | NM_003161 | ribosomal protein S6 kinase, 70 kDa, polypeptide |
| RPS6KL1 | NM_031464 | ribosomal protein S6 kinase-like 1 |
| RPUSD1 | NM_058192 | RNA pseudouridylate synthase domain containing |
| RRAD | NM_004165 | Ras-related associated with diabetes |
| RRAS | NM_006270 | related RAS viral (r-ras) oncogene homolog |
| RRAS2 | NM_012250 | related RAS viral (r-ras) oncogene homolog 2 |
| RSAD2 | NM_080657 | radical S-adenosyl methionine domain containing |
| RSPO2 | NM_178565 | R-spondin family, member 2 |
| RSPO4 | NM_001029871 | R-spondin family, member 4 isoform 1 precursor |
| RTF1 | NM_015138 | Paf1/RNA polymerase II complex component |
| RTN4 | NM_007008 | reticulon 4 isoform C |
| RTN4RL1 | NM_178568 | reticulon 4 receptor-like 1 |
| RUNX2 | NM_001015051 | runt-related transcription factor 2 isoform b |
| RUNX3 | NM_001031680 | runt-related transcription factor 3 isoform 1 |
| RUTBC1 | NM_014853 | RUN and TBC1 domain containing 1 |
| RWDD1 | NM_001007464 | RWD domain containing 1 isoform b |
| RXRA | NM_002957 | retinoid X receptor, alpha |
| S100A7L1 | NM_176823 | S100 calcium binding protein A7-like 1 |
| S100PBP | NM_022753 | S100P binding protein Riken isoform a |
| SACM1L | NM_014016 | suppressor of actin 1 |
| SAMD3 | NM_152552 | sterile alpha motif domain containing 3 isoform |
| SAMD4B | NM_018028 | sterile alpha motif domain containing 4B |
| SAP30BP | NM_013260 | transcriptional regulator protein |
| SAPS2 | NM_014678 | hypothetical protein LOC9701 |
| SAR1A | NM_020150 | SAR1a gene homolog 1 |
| SARM1 | NM_015077 | sterile alpha and TIR motif containing 1 |
| SART1 | NM_005146 | squamous cell carcinoma antigen recognized by T |
| SATB1 | NM_002971 | special AT-rich sequence binding protein 1 |
| SATB2 | NM_015265 | SATB family member 2 |
| SAV1 | NM_021818 | WW45 protein |
| SBK1 | NM_001024401 | SH3-binding domain kinase 1 |
| SCAMP5 | NM_138967 | secretory carrier membrane protein S |
| SCARA3 | NM_016240 | scavenger receptor class A, member 3 isoform 1 |
| SCARA5 | NM_173833 | hypothetical protein LOC286133 |
| SCARF1 | NM_145349 | scavenger receptor class F, member 1 isoform 2 |
| SCARF2 | NM_153334 | scavenger receptor class F, member 2 isoform 1 |
| SCCPDH | NM_016002 | saccharopine dehydrogenase (putative) |
| SCD | NM_005063 | stearoyl-CoA desaturase |
| SCMH1 | NM_001031694 | sex comb on midleg homolog 1 isoform 1 |
| SCML2 | NM_006089 | sex comb on midleg-like 2 |
| SCN1B | NM_001037 | sodium channel, voltage-gated, type I, beta |
| SCN2B | NM_004588 | sodium channel, voltage-gated, type II, beta |
| SCN3A | NM_006922 | sodium channel, voltage-gated, type III, alpha |
| SCN3B | NM_018400 | voltage-gated sodium channel beta-3 subunit |
| SCN4A | NM_000334 | voltage-gated sodium channel type 4 alpha |
| SCN4B | NM_174934 | sodium channel, voltage-gated, type IV, beta |
| SCN5A | NM_000335 | voltage-gated sodium channel type V alpha |
| SCNN1A | NM_001038 | sodium channel, nonvoltage-gated 1 alpha |
| SCNN1D | NM_002978 | sodium channel, nonvoltage-gated 1, delta |
| SCNN1G | NM_001039 | sodium channel, nonvoltage-gated 1, gamma |
| SCP2 | NM_001007098 | sterol carrier protein 2 isoform 2 |
| SCRIB | NM_015356 | scribble isoform b |
| SCUBE3 | NM_152753 | signal peptide, CUB domain, EGF-like 3 |
| SDAD1 | NM_018115 | SDA1 domain containing 1 |
| SDC1 | NM_001006946 | syndecan 1 precursor |
| SDHC | NM_003001 | succinate dehydrogenase complex, subunit C |
| SDK2 | NM_019064 | sidekick 2 |
| SEC13L1 | NM_030673 | SEC13-like 1 isoform a |
| SEC31L2 | NM_015490 | S. cerevisiae SEC3 1-like 2 isoform a |
| SEC61A1 | NM_013336 | Sec6l alpha 1 subunit |
| SEH1L | NM_031216 | sec13-like protein isoform 2 |
| SELPLG | NM_003006 | selectin P ligand |
| SEMA3E | NM_012431 | semaphorin 3E |
| SEMA3G | NM_020163 | semaphorin sem2 |
| SEMA4B | NM_020210 | semaphorin 4B precursor |
| SEMA4C | NM_017789 | semaphorin 4C |
| SEMA4D | NM_006378 | semaphorin 4D |
| SEMA4F | NM_004263 | semaphorin W |
| SEMA4G | NM_017893 | semaphorin 4G |
| SEMA5A | NM_003966 | semaphorin 5A |
| SEMA6A | NM_020796 | sema domain, transmembrane domain (TM), and |
| SEMA6C | NM_030913 | semaphorin Y |
| SEMA6D | NM_020858 | semaphorin 6D isoform 1 precursor |
| SENP2 | NM_021627 | SUMO1/sentrin/SMT3 specific protease 2 |
| SENP3 | NM_015670 | SUMO1/sentrin/SMT3 specific protease 3 |
| SENP6 | NM_015571 | SUMO1/sentrin specific protease 6 |
| SEPN1 | NM_020451 | selenoprotein N, 1 isoform 1 precursor |
| SEPT1 | NM_052838 | septin 1 |
| SEPT11 | NM_018243 | septin 11 |
| SEPT3 | NM_019106 | septin 3 isoform B |
| SEPT4 | NM_004574 | septin 4 isoform 1 |
| SEPT6 | NM_145800 | septin 6 isoform A |
| SEPT9 | NM_006640 | septin 9 |
| SERINC1 | NM_020755 | tumor differentially expressed 2 |
| SERPINB2 | NM_002575 | serine (or cysteine) proteinase inhibitor, clade |
| SERPINB5 | NM_002639 | serine (or cysteine) proteinase inhibitor, clade |
| SERPINB8 | NM_002640 | serine (or cysteine) proteinase inhibitor, clade |
| SERPINE1 | NM_000602 | plasminogen activator inhibitor-1 |
| SERPINF2 | NM_000934 | alpha-2-plasmin inhibitor |
| SESN2 | NM_031459 | sestrin 2 |
| SETD4 | NM_001007258 | hypothetical protein LOC54093 isoform b |
| SF3A2 | NM_007165 | splicing factor 3a, subunit 2 |
| SF3B3 | NM_012426 | splicing factor 3b, subunit 3 |
| SFRS8 | NM_152235 | splicing factor, arginine/serine-rich 8 isoform |
| SFT2D3 | NM_032740 | SFT2 domain containing 3 |
| SFTPA2 | NM_006926 | surfactant, pulmonary-associated protein A2 |
| SFXN2 | NM_178858 | sideroflexin 2 |
| SFXN3 | NM_030971 | sideroflexin 3 |
| SFXN5 | NM_144579 | sideroflexin 5 |
| SGPP1 | NM_030791 | sphingosine-1-phosphatase |
| SGSH | NM_000199 | N-sulfoglucosamine sulfohydrolase (sulfamidase) |
| SGTA | NM_003021 | small glutamine-rich tetratricopeptide |
| SH3BGRL2 | NM_031469 | SH3 domain binding glutamic acid-rich protein |
| SH3BP2 | NM_003023 | SH3-domain binding protein 2 |
| SH3BP4 | NM_014521 | SH3-domain binding protein 4 |
| SH3GL1 | NM_003025 | SH3-domain GRB2-like 1 |
| SH3PX3 | NM_153271 | SH3 and PX domain containing 3 |
| SH3PXD2A | NM_014631 | SH3 multiple domains 1 |
| SH3PXD2B | NM_001017995 | SH3 and PX domains 2B |
| SH3TC2 | NM_024577 | SH3 domain and tetratricopeptide repeats 2 |
| SHANK2 | NM_012309 | SH3 and multiple ankyrin repeat domains 2 |
| SHC4 | NM_203349 | rai-like protein |
| SHE | NM_001010846 | Src homology 2 domain containing E |
| SHKBP1 | NM_138392 | SH3KBP1 binding protein 1 |
| SHMT1 | NM_004169 | serine hydroxymethyltransferase 1 (soluble) |
| SHOC2 | NM_007373 | soc-2 suppressor of clear homolog |
| SHOX | NM_006883 | short stature homeobox isoform b |
| SIAE | NM_170601 | cytosolic sialic acid 9-O-acetylesterase |
| SIDT1 | NM_017699 | SID1 transmembrane family, member 1 |
| SIGLEC11 | NM_052884 | sialic acid binding Ig-like lectin 11 |
| SIM2 | NM_009586 | single-minded homolog 2 short isoform |
| SIPA1 | NM_006747 | signal-induced proliferation-associated protein |
| SIRPA | NM_080792 | signal-regulatory protein alpha precursor |
| SIRPB1 | NM_006065 | signal-regulatory protein beta 1 precursor |
| SIRT1 | NM_012238 | sirtuin 1 |
| SIRT5 | NM_031244 | sirtuin 5 isoform 2 |
| SIRT6 | NM_016539 | sirtuin 6 |
| SIT1 | NM_014450 | SHP2-interacting transmembrane adaptor protein |
| SIX5 | NM_175875 | sine oculis homeobox homolog 5 |
| SKI | NM_003036 | v-ski sarcoma viral oncogene homolog |
| SKIP | NM_016532 | skeletal muscle and kidney enriched inositol |
| SLAMF6 | NM_052931 | activating NK receptor precursor |
| SLC10A2 | NM_000452 | solute carrier family 10 (sodium/bile acid |
| SLC12A2 | NM_001046 | solute carrier family 12 |
| SLC12A7 | NM_006598 | solute carrier family 12 (potassium/chloride |
| SLC15A2 | NM_021082 | solute carrier family 15 (H+/peptide |
| SLC16A1 | NM_003051 | solute carrier family 16, member 1 |
| SLC16A14 | NM_152527 | solute carrier family 16 (monocarboxylic acid |
| SLC16A2 | NM_006517 | solute carrier family 16, member 2 |
| SLC16A8 | NM_013356 | solute carrier family 16, member 8 |
| SLC17A4 | NM_005495 | solute carrier family 17 (sodium phosphate), |
| SLC18A1 | NM_003053 | solute carrier family 18 (vesicular monoamine), |
| SLC19A2 | NM_006996 | solute carrier family 19, member 2 |
| SLC1A4 | NM_003038 | solute carrier family 1, member 4 |
| SLC22A12 | NM_144585 | urate anion exchanger 1 isoform a |
| SLC22A15 | NM_018420 | solute carrier family 22 (organic cation |
| SLC22A3 | NM_021977 | solute carrier family 22 member 3 |
| SLC22A7 | NM_006672 | solute carrier family 22 member 7 isoform a |
| SLC22A9 | NM_080866 | solute carrier family 22 (organic anion/cation |
| SLC24A6 | NM_024959 | solute carrier family 24 member 6 |
| SLC25A13 | NM_014251 | solute carrier family 25, member 13 (citrin) |
| SLC25A17 | NM_006358 | solute carrier family 25 (mitochondrial carrier; |
| SLC25A22 | NM_024698 | mitochondrial glutamate carrier 1 |
| SLC25A23 | NM_024103 | solute carrier family 25 (mitochondrial carrier; |
| SLC25A34 | NM_207348 | solute carrier family 25, member 34 |
| SLC26A1 | NM_022042 | solute carrier family 26, member 1 isoform a |
| SLC26A10 | NM_133489 | solute carrier family 26, member 10 isoform 2 |
| SLC26A2 | NM_000112 | solute carrier family 26 member 2 |
| SLC26A7 | NM_052832 | solute carrier family 26, member 7 isoform a |
| SLC26A9 | NM_052934 | solute carrier family 26, member 9 isoform a |
| SLC27A4 | NM_005094 | solute carrier family 27 (fatty acid |
| SLC29A1 | NM_004955 | solute carrier family 29 (nucleoside |
| SLC29A3 | NM_018344 | solute carrier family 29 (nucleoside |
| SLC29A4 | NM_153247 | solute carrier family 29 (nucleoside |
| SLC2A12 | NM_145176 | solute carrier family 2 (facilitated glucose |
| SLC2A13 | NM_052885 | solute carrier family 2 (facilitated glucose |
| SLC2A4RG | NM_020062 | SLC2A4 regulator |
| SLC2A8 | NM_014580 | solute carrier family 2, (facilitated glucose |
| SLC30A10 | NM_001004433 | solute carrier family 30 (zinc transporter), |
| SLC30A2 | NM_001004434 | solute carrier family 30, member 2 isoform 1 |
| SLC30A3 | NM_003459 | solute carrier family 30 (zinc transporter), |
| SLC30A4 | NM_013309 | solute carrier family 30 (zinc transporter), |
| SLC30A7 | NM_133496 | zinc transporter like 2 |
| SLC30A9 | NM_006345 | solute carrier family 30 (zinc transporter), |
| SLC31A2 | NM_001860 | solute carrier family 31 (copper transporters), |
| SLC35D2 | NM_007001 | solute carrier family 35, member D2 |
| SLC36A1 | NM_078483 | solute carrier family 36 member 1 |
| SLC37A2 | NM_198277 | solute carrier family 37 (glycerol-3-phosphate |
| SLC37A3 | NM_207113 | solute carrier family 37 (glycerol-3-phosphate |
| SLC38A1 | NM_030674 | amino acid transporter system A1 |
| SLC38A4 | NM_018018 | solute carrier family 38, member 4 |
| SLC39A10 | NM_020342 | solute carrier family 39 (zinc transporter), |
| SLC39A13 | NM_152264 | solute carrier family 39 (zinc transporter), |
| SLC39A14 | NM_015359 | solute carrier family 39 (zinc transporter), |
| SLC39A3 | NM_213568 | solute carrier family 39 (zinc transporter), |
| SLC43A2 | NM_152346 | solute carrier family 43, member 2 |
| SLC44A2 | NM_020428 | CTL2 protein |
| SLC45A3 | NM_033102 | prostein |
| SLC4A2 | NM_003040 | solute carrier family 4, anion exchanger, member |
| SLC4A7 | NM_003615 | solute carrier family 4, sodium bicarbonate |
| SLC5A10 | NM_152351 | solute carrier family 5 (sodium/glucose |
| SLC5A12 | NM_178498 | solute carrier family 5 (sodium/glucose |
| SLC5A8 | NM_145913 | solute carrier family 5 (iodide transporter), |
| SLC6A1 | NM_003042 | solute carrier family 6 (neurotransmitter |
| SLC6A12 | NM_003044 | solute carrier family 6 (neurotransmitter |
| SLC6A14 | NM_007231 | solute carrier family 6 (amino acid |
| SLC6A17 | NM_001010898 | solute carrier family 6, member 17 |
| SLC6A3 | NM_001044 | solute carrier family 6 (neurotransmitter |
| SLC6A6 | NM_003043 | solute carrier family 6 (neurotransmitter |
| SLC6A9 | NM_001024845 | solute carrier family 6 member 9 isoform 3 |
| SLC7A1 | NM_003045 | solute carrier family 7 (cationic amino acid |
| SLC7A10 | NM_019849 | solute carrier family 7, member 10 |
| SLC7A2 | NM_001008539 | solute carrier family 7, member 2 isoform 1 |
| SLC7A6 | NM_003983 | solute carrier family 7 (cationic amino acid |
| SLC7A6OS | NM_032178 | solute carrier family 7, member 6 opposite |
| SLC7A8 | NM_012244 | solute carrier family 7 (cationic amino acid |
| SLC8A3 | NM_033262 | solute carrier family 8 member 3 isoform A |
| SLC9A3R1 | NM_004252 | solute carrier family 9 (sodium/hydrogen |
| SLC9A8 | NM_015266 | Na+/H+ exchanger isoform 8 |
| SLCO1C1 | NM_017435 | solute carrier organic anion transporter family, |
| SLCO2A1 | NM_005630 | solute carrier organic anion transporter family, |
| SLCO3A1 | NM_013272 | solute carrier organic anion transporter family, |
| SLFN5 | NM_144975 | schlafen family member 5 |
| SMAD3 | NM_005902 | MAD, mothers against decapentaplegic homolog 3 |
| SMAD5 | NM_001001419 | SMAD, mothers against DPP homolog 5 |
| SMAD7 | NM_005904 | MAD, mothers against decapentaplegic homolog 7 |
| SMAP1 | NM_021940 | stromal membrane-associated protein |
| SMARCAD1 | NM_020159 | SWI/SNF-related, matrix-associated |
| SMARCC1 | NM_003074 | SWI/SNF-related matrix-associated |
| SMC1L1 | NM_006306 | SMC1 structural maintenance of chromosomes |
| SMCY | NM_004653 | Smcy homolog, Y-linked |
| SMO | NM_005631 | smoothened |
| SMOC1 | NM_022137 | secreted modular calcium-binding protein 1 |
| SMOX | NM_175839 | polyamine oxidase isoform 1 |
| SMPD1 | NM_000543 | sphingomyelin phosphodiesterase 1, acid |
| SMPD3 | NM_018667 | sphingomyelin phosphodiesterase 3, neutral |
| SMTN | NM_134270 | smoothelin isoform a |
| SMTNL2 | NM_198501 | hypothetical protein LOC342527 |
| SMURF1 | NM_020429 | Smad ubiguitination regulatory factor 1 isoform |
| SMYD1 | NM_198274 | SET and MYND domain containing 1 |
| SNAI1 | NM_005985 | snail 1 homolog |
| SNAI3 | NM_178310 | snail homolog 3 |
| SNAP25 | NM_003081 | synaptosomal-associated protein 25 isoform |
| SNAP29 | NM_004782 | synaptosomal-associated protein 29 |
| SNAPC1 | NM_003082 | small nuclear RNA activating complex, |
| SNAPC2 | NM_003083 | small nuclear RNA activating complex, |
| SNCA | NM_000345 | alpha-synuclein isoform NACP140 |
| SNCG | NM_003087 | synuclein, gamma (breast cancer-specific protein |
| SND1 | NM_014390 | staphylococcal nuclease domain containing 1 |
| SNF1LK2 | NM_015191 | SNF1-like kinase 2 |
| SNPH | NM_014723 | syntaphilin |
| SNTA1 | NM_003098 | acidic alpha 1 syntrophin |
| SNURF | NM_005678 | SNRPN upstream reading frame protein |
| SNX1 | NM_003099 | sorting nexin 1 isoform a |
| SNX10 | NM_013322 | sorting nexin 10 |
| SNX13 | NM_015132 | sorting nexin 13 |
| SNX15 | NM_013306 | sorting nexin 15 isoform A |
| SNX19 | NM_014758 | sorting nexin 19 |
| SNX4 | NM_003794 | sorting nexin 4 |
| SNX9 | NM_016224 | sorting nexin 9 |
| SOCS3 | NM_003955 | suppressor of cytokine signaling 3 |
| SOCS4 | NM_080867 | suppressor of cytokine signaling 4 |
| SOD3 | NM_003102 | superoxide dismutase 3, extracellular |
| SORBS1 | NM_015385 | sorbin and SH3 domain containing 1 isoform 2 |
| SORCS2 | NM_020777 | VPS10 domain receptor protein SORCS 2 |
| SOST | NM_025237 | sclerostin precursor |
| SOX10 | NM_006941 | SRY (sex determining region Y)-box 10 |
| SOX4 | NM_003107 | SRY (sex determining region Y)-box 4 |
| SOX6 | NM_017508 | SRY (sex determining region Y)-box 6 isoform 1 |
| SOX9 | NM_000346 | transcription factor SOX9 |
| SP1 | NM_138473 | Sp1 transcription factor |
| SP2 | NM_003110 | Sp2 transcription factor |
| SP7 | NM_152860 | osterix |
| SPAG11 | NM_058200 | sperm associated antigen 11 isoform G precursor |
| SPARC | NM_003118 | secreted protein, acidic, cysteine-rich |
| SPATA12 | NM_181727 | spermatogenesis associated 12 |
| SPATA2 | NM_006038 | spermatogenesis associated 2 |
| SPATA20 | NM_022827 | sperm protein SSP411 |
| SPATA3 | NM_139073 | testis and spermatogenesis cell apoptosis |
| SPBC24 | NM_182513 | spindle pole body component 24 homolog |
| SPCS2 | NM_014752 | signal peptidase complex subunit 2 homolog |
| SPDEF | NM_012391 | SAM pointed domain containing ets transcription |
| SPEN | NM_015001 | spen homolog, transcriptional regulator |
| SPFH1 | NM_006459 | SPFH domain family, member 1 |
| SPG21 | NM_016630 | acid cluster protein 33 |
| SPG7 | NM_003119 | paraplegin isoform 1 |
| SPI1 | NM_003120 | spleen focus forming virus (SFFV) proviral |
| SPINK2 | NM_021114 | serine protease inhibitor, Kazal type 2 |
| SPINK5 | NM_006846 | serine peptidase inhibitor, Kazal type 5 |
| SPINLW1 | NM_020398 | serine peptidase inhibitor-like, with Kunitz and |
| SPN | NM_001030288 | sialophorin |
| SPOCK1 | NM_004598 | sparc/osteonectin, cwcv and kazal-like domains |
| SPOCK2 | NM_014767 | sparc/osteonectin, cwcv and kazal-like domains |
| SPON1 | NM_006108 | spondin 1, extracellular matrix protein |
| SPOP | NM_001007226 | speckle-type POZ protein |
| SPP1 | NM_000582 | secreted phosphoprotein 1 isoform b |
| SPPL3 | NM_139015 | SPPL3 protein |
| SPRN | NM_001012508 | shadow of prion protein |
| SPRR2A | NM_005988 | small proline-rich protein 2A |
| SPRR2B | NM_001017418 | small proline-rich protein 2B |
| SPRR2D | NM_006945 | small proline-rich protein 2D |
| SPRR2F | NM_001014450 | small proline-rich protein 2F |
| SPRY1 | NM_005841 | sprouty homolog 1, antagonist of FGF signaling |
| SPRY3 | NM_005840 | sprouty homolog 3 |
| SPRYD4 | NM_207344 | hypothetical protein LOC283377 |
| SPSB3 | NM_080861 | SPRY domain-containing SOCS box protein SSB-3 |
| SPSB4 | NM_080862 | SPRY domain-containing SOCS box protein SSB-4 |
| SPTB | NM_001024858 | spectrin beta isoform a |
| SPTBN2 | NM_006946 | spectrin, beta, non-erythrocytic 2 |
| SPTLC2 | NM_004863 | serine palmitoyltransferase, long chain base |
| SPTY2D1 | NM_194285 | hypothetical protein LOC144108 |
| SRC | NM_005417 | proto-oncogene tyrosine-protein kinase SRC |
| SRF | NM_003131 | serum response factor (c-fos serum response |
| SRGAP1 | NM_020762 | SLIT-ROBO Rho GTPase-activating protein 1 |
| SRGAP2 | NM_015326 | SLIT-ROBO Rho GTPase activating protein 2 |
| SRGAP3 | NM_001033116 | SLIT-ROBO Rho GTPase activating protein 3 |
| SRPR | NM_003139 | signal recognition particle receptor (′docking |
| SRR | NM_021947 | serine racemase |
| SRXN1 | NM_080725 | sulfiredoxin 1 homolog |
| SSR2 | NM_003145 | signal sequence receptor, beta precursor |
| SSR3 | NM_007107 | signal sequence receptor gamma subunit |
| SSTR2 | NM_001050 | somatostatin receptor 2 |
| SSX2IP | NM_014021 | synovial sarcoma, X breakpoint 2 interacting |
| SSX5 | NM_021015 | synovial sarcoma, X breakpoint 5 isoform a |
| SSX6 | NM_173357 | synovial sarcoma, X breakpoint 6 |
| ST18 | NM_014682 | suppression of tumorigenicity 18 |
| ST3GAL3 | NM_006279 | sialyltransferase 6 isoform j |
| ST6GAL1 | NM_003032 | sialyltransferase 1 isoform a |
| ST6GALNAC4 | NM_175039 | sialyltransferase 7D isoform a |
| ST6GALNAC6 | NM_013443 | ST6 |
| ST8SIA2 | NM_006011 | ST8 alpha-N-acetyl-neuraminide |
| STAB2 | NM_017564 | stabilin 2 precursor |
| STAC | NM_003149 | SH3 and cysteine rich domain |
| STAC2 | NM_198993 | SH3 and cysteine rich domain 2 |
| STAG2 | NM_006603 | stromal antigen 2 |
| STARD3 | NM_006804 | steroidogenic acute regulatory protein related |
| STARD8 | NM_014725 | START domain containing 8 |
| STAT1 | NM_139266 | signal transducer and activator of transcription |
| STAT3 | NM_003150 | signal transducer and activator of transcription |
| STC1 | NM_003155 | stanniocalcin 1 precursor |
| STC2 | NM_003714 | stanniocalcin 2 precursor |
| STCH | NM_006948 | stress 70 protein chaperone, |
| STEAP2 | NM_152999 | six transmembrane epithelial antigen of the |
| STIL | NM_003035 | SCL/TAL1 interrupting locus |
| STIM1 | NM_003156 | stromal interaction molecule 1 precursor |
| STK10 | NM_005990 | serine/threonine kinase 10 |
| STK25 | NM_006374 | serine/threonine kinase 25 |
| STK35 | NM_080836 | serine/threonine kinase 35 |
| STK39 | NM_013233 | serine threonine kinase 39 (STE20/SPS1 homolog, |
| STK4 | NM_006282 | serine/threonine kinase 4 |
| STMN3 | NM_015894 | SCG10-like-protein |
| STOM | NM_004099 | stomatin isoform a |
| STON1 | NM_006873 | stonin 1 |
| STRAP | NM_007178 | serine/threonine kinase receptor associated |
| STRBP | NM_018387 | spermatid perinuclear RNA-binding protein |
| STRN3 | NM_014574 | nuclear autoantigen |
| STS | NM_000351 | steryl-sulfatase precursor |
| STS-1 | NM_032873 | Cbl-interacting protein Sts-1 |
| STX17 | NM_017919 | syntaxin 17 |
| STX1A | NM_004603 | syntaxin 1A (brain) |
| STX5 | NM_003164 | syntaxin 5 |
| STXBP1 | NM_001032221 | syntaxin binding protein 1 isoform b |
| SUFU | NM_016169 | suppressor of fused |
| SUHW2 | NM_080764 | suppressor of hairy wing homolog 2 |
| SULF1 | NM_015170 | sulfatase 1 |
| SULF2 | NM_018837 | sulfatase 2 isoform a precursor |
| SULT4A1 | NM_014351 | sulfotransferase family 4A, member 1 |
| SUOX | NM_000456 | sulfite oxidase |
| SUPT16H | NM_007192 | chromatin-specific transcription elongation |
| SUPT6H | NM_003170 | suppressor of Ty 6 homolog |
| SURF1 | NM_003172 | surfeit 1 |
| SURF4 | NM_033161 | surfeit 4 |
| SURF6 | NM_006753 | surfeit 6 |
| SUV39H1 | NM_003173 | suppressor of variegation 3-9 homolog 1 |
| SUV420H1 | NM_016028 | suppressor of variegation 4-20 homolog 1 isoform |
| SUV420H2 | NM_032701 | suppressor of variegation 4-20 homolog 2 |
| SV2A | NM_014849 | synaptic vesicle glycoprotein 2 |
| SVIL | NM_003174 | supervillin isoform 1 |
| SVOP | NM_018711 | SV2 related protein |
| SWAP70 | NM_015055 | SWAP-70 protein |
| SYN2 | NM_003178 | synapsin II isoform IIb |
| SYNC1 | NM_030786 | syncoilin, intermediate filament 1 |
| SYNGR1 | NM_004711 | synaptogyrin 1 isoform 1a |
| SYNGR2 | NM_004710 | synaptogyrin 2 |
| SYNGR3 | NM_004209 | synaptogyrin 3 |
| SYNJ1 | NM_003895 | synaptojanin 1 isoform a |
| SYNPR | NM_144642 | synaptoporin |
| SYT1 | NM_005639 | synaptotagmin I |
| SYT11 | NM_152280 | synaptotagmin 12 |
| SYT13 | NM_020826 | synaptotagmin XIII |
| SYT15 | NM_031912 | synaptotagmin XV isoform a |
| SYT4 | NM_020783 | synaptotagmin IV |
| SYT6 | NM_205848 | synaptotagmin VI |
| SYT9 | NM_175733 | synaptotagmin IX |
| SYVN1 | NM_032431 | synoviolin 1 isoform a |
| TACC1 | NM_006283 | transforming, acidic coiled-coil containing |
| TACR1 | NM_001058 | tachykinin receptor 1 isoform long |
| TACSTD2 | NM_002353 | tumor-associated calcium signal transducer 2 |
| TAF1A | NM_005681 | TBP-associated factor 1A isoform 1 |
| TAF2 | NM_003184 | TBP-associated factor 2 |
| TAF5 | NM_006951 | TBP-associated factor 5 |
| TAF5L | NM_001025247 | PCAF associated factor 65 beta isoform b |
| TAGAP | NM_054114 | T-cell activation Rho GTPase-activating protein |
| TAGLN | NM_001001522 | transgelin |
| TAIP-2 | NM_024969 | TGF-beta induced apoptosis protein 2 |
| TAL1 | NM_003189 | T-cell acute lymphocytic leukemia 1 |
| TAOK1 | NM_020791 | TAO kinase 1 |
| TAPBP | NM_003190 | tapasin isoform 1 precursor |
| TARBP2 | NM_004178 | TAR RNA binding protein 2 isoform b |
| TARP | NM_001003799 | TCR gamma alternate reading frame protein |
| TAS1R1 | NM_177539 | sweet taste receptor T1r isoform a |
| TATDN2 | NM_014760 | TatD DNase domain containing 2 |
| TAX1BP3 | NM_014604 | Tax1 (human T-cell leukemia virus type I) |
| TBC1D13 | NM_018201 | TBC1 domain family, member 13 |
| TBC1D2 | NM_018421 | TBC1 domain family, member 2 |
| TBC1D22B | NM_017772 | TBC1 domain family, member 22B |
| TBC1D2B | NM_015079 | TBC1 domain family, member 2B |
| TBC1D5 | NM_014744 | TBC1 domain family, member 5 |
| TBCD | NM_001033052 | beta-tubulin cofactor D isoform 2 |
| TBL1XR1 | NM_024665 | nuclear receptor co-repressor/HDAC3 complex |
| TBRG1 | NM_032811 | transforming growth factor beta regulator 1 |
| TBX4 | NM_018488 | T-box 4 |
| TCF1 | NM_000545 | transcription factor 1, hepatic |
| TCF12 | NM_003205 | transcription factor 12 isoform b |
| TCF2 | NM_006481 | transcription factor 2 isoform b |
| TCF3 | NM_003200 | transcription factor 3 |
| TCF7 | NM_003202 | transcription factor 7 (T-cell specific, |
| TCOF1 | NM_001008657 | Treacher Collins-Franceschetti syndrome 1 |
| TCTA | NM_022171 | T-cell leukemia translocation altered gene |
| TCTEX1D1 | NM_152665 | hypothetical protein LOC200132 |
| TEF | NM_003216 | thyrotrophic embryonic factor |
| TENC1 | NM_015319 | tensin like C1 domain containing phosphatase |
| TERT | NM_198253 | telomerase reverse transcriptase isoform 3 |
| TESK1 | NM_006285 | testis-specific protein kinase 1 |
| TETRAN | NM_001120 | tetracycline transporter-like protein |
| TEX13B | NM_031273 | testis expressed sequence 13B |
| TEX261 | NM_144582 | testis expressed sequence 261 |
| TEX264 | NM_015926 | testis expressed sequence 264 |
| TFAP2A | NM_001032280 | transcription factor AP-2 alpha isoform b |
| TFCP2L1 | NM_014553 | LBP-9 |
| TFDP2 | NM_006286 | transcription factor Dp-2 (E2F dimerization |
| TFE3 | NM_006521 | transcription factor binding to IGHM enhancer 3 |
| TFEB | NM_007162 | transcription factor EB |
| TFRC | NM_003234 | transferrin receptor |
| TGFA | NM_003236 | transforming growth factor, alpha |
| TGFB3 | NM_003239 | transforming growth factor, beta 3 |
| TGFBI | NM_000358 | transforming growth factor, beta-induced, 68 kDa |
| TGFBR1 | NM_004612 | transforming growth factor, beta receptor I |
| TGFBR2 | NM_001024847 | TGF-beta type II receptor isoform A precursor |
| TGFBR3 | NM_003243 | transforming growth factor, beta receptor III |
| TGIF2 | NM_021809 | TGFB-induced factor 2 |
| TGM2 | NM_004613 | transglutaminase 2 isoform a |
| TGOLN2 | NM_006464 | trans-golgi network protein 2 |
| TH | NM_000360 | tyrosine hydroxylase isoform b |
| TH1L | NM_198976 | TH1-like protein |
| THADA | NM_198554 | thyroid adenoma associated isoform 2 |
| THBD | NM_000361 | thrombomodulin precursor |
| THBS1 | NM_003246 | thrombospondin 1 precursor |
| THEM4 | NM_176853 | thioesterase superfamily member 4 isoform b |
| THEM5 | NM_182578 | thioesterase superfamily member 5 |
| THOP1 | NM_003249 | thimet oligopeptidase 1 |
| THPO | NM_000460 | thrombopoietin isoform 1 precursor |
| THRA | NM_199334 | thyroid hormone receptor, alpha isoform 1 |
| THRAP1 | NM_005121 | thyroid hormone receptor associated protein 1 |
| THSD4 | NM_024817 | hypothetical protein LOC79875 |
| TICAM2 | NM_021649 | toll-like receptor adaptor molecule 2 |
| TIGD6 | NM_030953 | hypothetical protein LOC81789 |
| TIMM10 | NM_012456 | translocase of inner mitochondrial membrane 10 |
| TIMM13 | NM_012458 | translocase of inner mitochondrial membrane 13 |
| TIMM17B | NM_005834 | translocase of inner mitochondrial membrane 17 |
| TIMM22 | NM_013337 | translocase of inner mitochondrial membrane 22 |
| TIMM8A | NM_004085 | translocase of inner mitochondrial membrane 8 |
| TINP1 | NM_014886 | TGF beta-inducible nuclear protein 1 |
| TK1 | NM_003258 | thymidine kinase 1, soluble |
| TLK1 | NM_012290 | tousled-like kinase 1 |
| TLR4 | NM_138554 | toll-like receptor 4 precursor |
| TLX1 | NM_005521 | T-cell leukemia, homeobox 1 |
| TLX2 | NM_016170 | T-cell leukemia, homeobox 2 |
| TM4SF1 | NM_014220 | transmembrane 4 superfamily member 1 |
| TM4SF4 | NM_004617 | transmembrane 4 superfamily member 4 |
| TM9SF2 | NM_004800 | transmembrane 9 superfamily member 2 |
| TM9SF3 | NM_020123 | endomembrane protein emp70 precursor isolog |
| TM9SF4 | NM_014742 | transmembrane 9 superfamily protein member 4 |
| TMBIM1 | NM_022152 | transmembrane BAX inhibitor motif containin 1 |
| TMC2 | NM_080751 | transmembrane cochlear-expressed protein 2 |
| TMC5 | NM_024780 | transmembrane channel-like 5 |
| TMCC1 | NM_001017395 | transmembrane and coiled-coil domains 1 isoform |
| TMCC3 | NM_020698 | transmembrane and coiled-coil domains 3 |
| TMED10 | NM_006827 | transmembrane trafficking protein |
| TMEFF1 | NM_003692 | transmembrane protein with EGF-like and two |
| TMEM104 | NM_017728 | hypothetical protein LOC54868 |
| TMEM109 | NM_024092 | transmembrane protein 109 |
| TMEM129 | NM_138385 | hypothetical protein LOC92305 |
| TMEM130 | NM_152913 | hypothetical protein LOC222865 |
| TMEM133 | NM_032021 | hypothetical protein LOC83935 |
| TMEM141 | NM_032928 | transmembrane protein 141 |
| TMEM143 | NM_018273 | hypothetical protein LOC55260 |
| TMEM144 | NM_018342 | hypothetical protein LOC55314 |
| TMEM16K | NM_018075 | hypothetical protein LOC55129 |
| TMEM22 | NM_025246 | transmembrane protein 22 |
| TMEM25 | NM_032780 | transmembrane protein 25 |
| TMEM28 | NM_015686 | transmembrane protein 28 |
| TMEM29 | NM_014138 | hypothetical protein LOC29057 |
| TMEM33 | NM_018126 | transmembrane protein 33 |
| TMEM35 | NM_021637 | transmembrane protein 35 |
| TMEM39A | NM_018266 | transmembrane protein 39A |
| TMEM43 | NM_024334 | transmembrane protein 43 |
| TMEM48 | NM_018087 | transmembrane protein 48 |
| TMEM55A | NM_018710 | transmembrane protein 55A |
| TMEM57 | NM_018202 | transmembrane protein 57 |
| TMEM63A | NM_014698 | transmembrane protein 63A |
| TMEM63C | NM_020431 | transmembrane protein 63C |
| TMEM79 | NM_032323 | hypothetical protein LOC84283 |
| TMEM80 | NM_174940 | hypothetical protein LOC283232 |
| TMEM86A | NM_153347 | hypothetical protein LOC144110 |
| TMEM87A | NM_015497 | hypothetical protein LOC25963 |
| TMEPAI | NM_020182 | transmembrane prostate androgen-induced protein |
| TMLHE | NM_018196 | trimethyllysine hydroxylase, epsilon |
| TMOD2 | NM_014548 | tropomodulin 2 (neuronal) |
| TMPRSS13 | NM_032046 | transmembrane protease, serine 13 |
| TMPRSS4 | NM_019894 | transmembrane protease, serine 4 isoform 1 |
| TMPRSS5 | NM_030770 | transmembrane protease, serine 5 |
| TMPRSS6 | NM_153609 | transmembrane protease, serine 6 |
| TMSB10 | NM_021103 | thymosin, beta 10 |
| TMTC2 | NM_152588 | hypothetical protein LOC160335 |
| TNFAIP1 | NM_021137 | tumor necrosis factor, alpha-induced protein 1 |
| TNFAIP8L2 | NM_024575 | tumor necrosis factor, alpha-induced protein |
| TNFRSF10D | NM_003840 | tumor necrosis factor receptor superfamily, |
| TNFRSF11B | NM_002546 | osteoprotegerin precursor |
| TNFRSF14 | NM_003820 | tumor necrosis factor receptor superfamily, |
| TNFRSF19 | NM_148957 | tumor necrosis factor receptor superfamily, |
| TNFRSF19L | NM_032871 | tumor necrosis factor receptor superfamily, |
| TNFRSF8 | NM_001243 | tumor necrosis factor receptor superfamily, |
| TNFRSF9 | NM_001561 | tumor necrosis factor receptor superfamily, |
| TNIP2 | NM_024309 | A20-binding inhibitor of NF-kappaB activation 2 |
| TNK1 | NM_003985 | tyrosine kinase, non-receptor, 1 |
| TNNI1 | NM_003281 | troponin I, skeletal, slow |
| TNNI3 | NM_000363 | troponin I, cardiac |
| TNP1 | NM_003284 | transition protein 1 (during histone to |
| TNPO2 | NM_013433 | transportin 2 (importin 3, karyopherin beta 2b) |
| TNRC4 | NM_007185 | trinucleotide repeat containing 4 |
| TNRC6B | NM_001024843 | trinucleotide repeat containing 6B isoform 2 |
| TNS3 | NM_022748 | tensin-like SH2 domain containing 1 |
| TNS4 | NM_032865 | C-terminal tensin-like |
| TNT | NM_182831 | hypothetical protein LOC162083 |
| TNXB | NM_019105 | tenascin XB isoform 1 |
| TOB2 | NM_016272 | transducer of ERBB2, 2 |
| TOLLIP | NM_019009 | toll interacting protein |
| TOM1 | NM_005488 | target of myb1 |
| TOM1L2 | NM_001033551 | target of myb1-like 2 isoform 1 |
| TOMM22 | NM_020243 | mitochondrial import receptor Tom22 |
| TOMM40L | NM_032174 | translocase of outer mitochondrial membrane 40 |
| TOP2B | NM_001068 | DNA topoisomerase II, beta isozyme |
| TOPORS | NM_005802 | topoisomerase I binding, arginine/serine-rich |
| TOR1A | NM_000113 | torsin A |
| TOR1B | NM_014506 | torsin family 1, member B (torsin B) |
| TOR3A | NM_022371 | torsin family 3, member A |
| TOX | NM_014729 | thymus high mobility group box protein TOX |
| TP53I11 | NM_006034 | p53-induced protein |
| TP53INP1 | NM_033285 | tumor protein p53 inducible nuclear protein 1 |
| TP53INP2 | NM_021202 | tumor protein p53 inducible nuclear protein 2 |
| TP53TG3 | NM_016212 | hypothetical protein LOC24150 |
| TP73L | NM_003722 | tumor protein p73-like |
| TPD52 | NM_001025252 | tumor protein D52 isoform 1 |
| TPD52L3 | NM_001001875 | protein kinase NYD-SP25 isoform 3 |
| TPI1 | NM_000365 | triosephosphate isomerase 1 |
| TPP1 | NM_000391 | tripeptidyl-peptidase I precursor |
| TPPP | NM_007030 | brain-specific protein p25 alpha |
| TPSAB1 | NM_003294 | tryptase alpha/beta 1 precursor |
| TPSB2 | NM_024164 | tryptase beta 2 precursor |
| TRAF1 | NM_005658 | TNF receptor-associated factor 1 |
| TRAF3IP3 | NM_025228 | TRAF3-interacting JNK-activating modulator |
| TRAF7 | NM_206835 | ring finger and WD repeat domain 1 isoform 2 |
| TRAFD1 | NM_006700 | FLN29 gene product |
| TRAPPC6A | NM_024108 | trafficking protein particle complex 6A |
| TREML4 | NM_198153 | triggering receptor expressed on myeloid |
| TRERF1 | NM_018415 | transcriptional regulating factor 1 isoform 3 |
| TREX1 | NM_032166 | three prime repair exonuclease 1 isoform c |
| TRIAD3 | NM_207111 | TRIAD3 protein isoform a |
| TRIB2 | NM_021643 | tribbles homolog 2 |
| TRIM10 | NM_052828 | tripartite motif-containing 10 isoform 2 |
| TRIM14 | NM_014788 | tripartite motif protein TRIM14 isoform alpha |
| TRIM2 | NM_015271 | tripartite motif-containing 2 |
| TRIM21 | NM_003141 | 52 kD Ro/SSA autoantigen |
| TRIM22 | NM_006074 | tripartite motif-containing 22 |
| TRIM25 | NM_005082 | tripartite motif-containing 25 |
| TRIM29 | NM_012101 | tripartite motif protein TRIM29 isoform alpha |
| TRIM32 | NM_012210 | TAT-interactive protein, 72-KD |
| TRIM33 | NM_015906 | tripartite motif-containing 33 protein isoform |
| TRIM35 | NM_015066 | tripartite motif-containing 35 isoform 1 |
| TRIM37 | NM_015294 | tripartite motif-containing 37 protein |
| TRIM41 | NM_033549 | tripartite motif-containing 41 isform 1 |
| TRIM54 | NM_032546 | ring finger protein 30 isoform 1 |
| TRIM62 | NM_018207 | tripartite motif-containing 62 |
| TRIM67 | NM_001004342 | hypothetical protein LOC440730 |
| TRIM68 | NM_018073 | ring finger protein 137 |
| TRIM9 | NM_052978 | tripartite motif protein 9 isoform 2 |
| TRIO | NM_007118 | triple functional domain (PTPRF interacting) |
| TRMT5 | NM_020810 | tRNA-(N1G37) methyltransferase |
| TRPC3 | NM_003305 | transient receptor potential cation channel, |
| TRPC5 | NM_012471 | transient receptor potential cation channel, |
| TRPV4 | NM_021625 | transient receptor potential cation channel, |
| TSC1 | NM_000368 | tuberous sclerosis 1 protein isoform 1 |
| TSHR | NM_000369 | thyroid stimulating hormone receptor isoform 1 |
| TSHZ2 | NM_173485 | zinc finger protein 218 |
| TSN | NM_004622 | translin |
| TSPAN14 | NM_030927 | tetraspanin 14 |
| TSPAN17 | NM_001006616 | transmembrane 4 superfamily member 17 isoform c |
| TSPAN18 | NM_130783 | tetraspanin 18 isoform 2 |
| TSPAN32 | NM_139024 | tumor-suppressing subtransferable candidate 6 |
| TSPAN33 | NM_178562 | penumbra |
| TSPYL1 | NM_003309 | TSPY-like 1 |
| TSPYL4 | NM_021648 | TSPY-like 4 |
| TSPYL5 | NM_033512 | TSPY-like 5 |
| TSPYL6 | NM_001003937 | TSPY-like 6 |
| TSR1 | NM_018128 | hypothetical protein LOC55720 |
| TSSC4 | NM_005706 | tumor suppressing subtransferable candidate 4 |
| TTBK1 | NM_032538 | tau tubulin kinase 1 |
| TTC1 | NM_003314 | tetratricopeptide repeat domain 1 |
| TTC19 | NM_017775 | tetratricopeptide repeat domain 19 |
| TTL | NM_153712 | tubulin tyrosine ligase |
| TTLL12 | NM_015140 | hypothetical protein LOC23170 |
| TTLL3 | NM_015644 | tubulin tyrosine ligase-like family, member 3 |
| TTMB | NM_001003682 | hypothetical protein LOC399474 |
| TTYH2 | NM_032646 | tweety 2 isoform 1 |
| TTYH3 | NM_02S250 | tweety 3 |
| TUBA2 | NM_006001 | tubulin, alpha 2 isoform 1 |
| TUBA4 | NM_025019 | tubulin, alpha 4 |
| TUBB | NM_178014 | tubulin, beta polypeptide |
| TUBB1 | NM_030773 | beta tubulin 1, class VI |
| TUFT1 | NM_020127 | tuftelin 1 |
| TULP1 | NM_003322 | tubby like protein 1 |
| TULP3 | NM_003324 | tubby like protein 3 |
| TULP4 | NM_001007466 | tubby like protein 4 isoform 2 |
| TUSC5 | NM_172367 | LOST1 |
| TXN2 | NM_012473 | thioredoxin 2 precursor |
| TXNDC13 | NM_021156 | thioredoxin domain containing 13 |
| TXNDC4 | NM_015051 | thioredoxin domain containing 4 (endoplasmic |
| TXNDC9 | NM_005783 | ATP binding protein associated with cell |
| TXNIP | NM_006472 | thioredoxin interacting protein |
| UACA | NM_001008224 | uveal autoantigen with coiled-coil domains and |
| UBAP2 | NM_018449 | ubiquitin associated protein 2 |
| UBASH3A | NM_001001895 | ubiquitin associated and SH3 domain containing, |
| UBE1 | NM_003334 | ubiquitin-activating enzyme E1 |
| UBE1DC1 | NM_024818 | ubiquitin-activating enzyme E1-domain containing |
| UBE2G1 | NM_003342 | ubiquitin-conjugating enzyme E2G 1 isoform 1 |
| UBE2J2 | NM_058167 | ubiquitin conjugating enzyme E2, J2 isoform 2 |
| UBE2L3 | NM_003347 | ubiquitin-conjugating enzyme E2L 3 isoform 1 |
| UBE2NL | NM_001012989 | hypothetical protein LOC389898 |
| UBE2O | NM_022066 | ubiquitin-conjugating enzyme E2O |
| UBE2Q1 | NM_017582 | ubiquitin-conjugating enzyme E2Q |
| UBE2R2 | NM_017811 | ubiquitin-conjugating enzyme UBC3B |
| UBE2W | NM_001001481 | hypothetical protein LOC55284 isoform 1 |
| UBE3B | NM_183414 | ubiquitin protein ligase E3B isoform b |
| UBL4A | NM_014235 | ubiquitin-like 4 |
| UBL7 | NM_032907 | ubiquitin-like 7 (bone marrow stromal |
| UBN1 | NM_016936 | ubinuclein 1 |
| UBOX5 | NM_014948 | U-box domain containing 5 isoform a |
| UBP1 | NM_014517 | upstream binding protein 1 (LBP-1a) |
| UBQLN4 | NM_020131 | ataxin-1 ubiquitin-like interacting protein |
| UBTF | NM_014233 | upstream binding transcription factor, RNA |
| UBXD3 | NM_152376 | UBX domain containing 3 |
| UBXD8 | NM_014613 | UBX domain containing 8 |
| UCN2 | NM_033199 | urocortin 2 preproprotein |
| UCP3 | NM_003356 | uncoupling protein 3 isoform UCP3L |
| UFD1L | NM_005659 | ubiquitin fusion degradation 1-like isoform A |
| UGT3A1 | NM_152404 | UDP glycosyltransferase 3 family, polypeptide |
| UHRF2 | NM_152896 | Np95-like ring finger protein isoform b |
| ULBP2 | NM_025217 | UL16 binding protein 2 |
| ULK1 | NM_003565 | unc-51-like kinase 1 |
| UNC119 | NM_005148 | unc119 (C. elegans) homolog isoform a |
| UNC13B | NM_006377 | UNC13 (C. elegans)-like |
| UNC45A | NM_018671 | smooth muscle cell associated protein-1 isoform |
| UNC45B | NM_001033576 | cardiomyopathy associated 4 isoform 2 |
| UNC5A | NM_133369 | netrin receptor Unc5h1 |
| UNC5C | NM_003728 | unc5C |
| UNC5D | NM_080872 | netrin receptor Unc5h4 |
| UNC84A | NM_025154 | unc-84 homolog A |
| UNG | NM_003362 | uracil-DNA glycosylase isoform UNG1 precursor |
| UQCR | NM_006830 | ubiquinol-cytochrome c reductase, 6.4 kDa |
| UROC1 | NM_144639 | urocanase domain containing 1 |
| UROS | NM_000375 | uroporphyrinogen III synthase |
| URP2 | NM_031471 | UNC-112 related protein 2 short form |
| USF1 | NM_007122 | upstream stimulatory factor 1 isoform 1 |
| USP15 | NM_006313 | ubiguitin specific protease 15 |
| USP25 | NM_013396 | ubiguitin specific protease 25 |
| USP3 | NM_006537 | ubiquitin specific protease 3 |
| USP47 | NM_017944 | ubiquitin specific protease 47 |
| UST | NM_005715 | uronyl-2-sulfotransferase |
| UTS2D | NM_198152 | urotensin 2 domain containing |
| UTY | NM_007125 | tetratricopeptide repeat protein isoform 3 |
| VAMP1 | NM_014231 | vesicle-associated membrane protein 1 isoform 1 |
| VAMP2 | NM_014232 | vesicle-associated membrane protein 2 |
| VAMP3 | NM_004781 | vesicle-associated membrane protein 3 |
| VANGL2 | NM_020335 | yang-like 2 (van gogh, Drosophila) |
| VAPA | NM_003574 | vesicle-associated membrane protein-associated |
| VARSL | NM_020442 | valyl-tRNA synthetase 2-like |
| VASH1 | NM_014909 | vasohibin 1 |
| VAT1 | NM_006373 | vesicle amine transport protein 1 |
| VAV2 | NM_003371 | vav 2 oncogene |
| VAX1 | NM_199131 | ventral anterior homeobox 1 |
| VCAM1 | NM_001078 | vascular cell adhesion molecule 1 isoform a |
| VCL | NM_003373 | vinculin isoform VCL |
| VCP | NM_007126 | valosin-containing protein |
| VCPIP1 | NM_025054 | valosin containing protein (p97)/p47 complex |
| VDR | NM_000376 | vitamin D (1,25-dihydroxyvitamin D3) receptor |
| VEGF | NM_001025366 | vascular endothelial growth factor isoform a |
| VEZT | NM_017599 | transmembrane protein vezatin |
| VGLL3 | NM_016206 | colon carcinoma related protein |
| VISA | NM_020746 | virus-induced signaling adapter |
| VMD2 | NM_004183 | bestrophin |
| VMD2L1 | NM_017682 | vitelliform macular dystrophy 2-like 1 |
| VMD2L2 | NM_153274 | vitelliform macular dystrophy 2-like 2 |
| VMP | NM_080723 | vesicular membrane protein p24 |
| VPS13A | NM_001018037 | vacuolar protein sorting 13A isoform C |
| VPS13B | NM_017890 | vacuolar protein sorting 13B isoform 5 |
| VPS13C | NM_017684 | vacuolar protein sorting 13C protein isoform 1A |
| VPS13D | NM_015378 | vacuolar protein sorting 13D isoform 1 |
| VPS24 | NM_001005753 | vacuolar rotein sortin 24 isoform 2 |
| VPS36 | NM_016075 | vacuolar protein sorting 36 |
| VPS37A | NM_152415 | hepatocellular carcinoma related protein 1 |
| VPS37B | NM_024667 | vacuolar protein sorting 37B |
| VPS39 | NM_015289 | vacuolar protein sorting 39 |
| VPS41 | NM_014396 | vacuolar protein sorting 41 (yeast homolog) |
| VPS4A | NM_013245 | vacuolar protein sorting factor 4A |
| VPS52 | NM_022553 | suppressor of actin mutations 2-like |
| VSIG1 | NM_182607 | V-set and immunoglobulin domain containing 1 |
| VTCN1 | NM_024626 | V-set domain containing T cell activation |
| VWA1 | NM_022834 | von Willebrand factor A domain-related protein |
| VWCE | NM_152718 | hypothetical protein LOC220001 |
| WASF1 | NM_001024934 | Wiskott-Aldrich syndrome protein family member |
| WASF2 | NM_006990 | WAS protein family, member 2 |
| WASL | NM_003941 | Wiskott-Aldrich syndrome gene-like protein |
| WBSCR16 | NM_030798 | Williams-Beuren syndrome chromosome region 16 |
| WBSCR17 | NM_022479 | UDP-GaINAc:polypeptide |
| WBSCR18 | NM_032317 | Williams Beuren syndrome chromosome region 18 |
| WDFY3 | NM_014991 | WD repeat and FYVE domain containing 3 isoform |
| WDR22 | NM_003861 | Breakpoint cluster region protein, uterine |
| WDR23 | NM_025230 | WD repeat domain 23 isoform 1 |
| WDR3 | NM_006784 | WD repeat-containing protein 3 |
| WDR33 | NM_001006623 | WD repeat domain 33 isoform 3 |
| WDR35 | NM_001006657 | WD repeat domain 35 isoform 1 |
| WDR37 | NM_014023 | WD repeat domain 37 |
| WDR39 | NM_004804 | WD repeat domain 39 |
| WDR41 | NM_018268 | WD repeat domain 41 |
| WDR5B | NM_019069 | WD repeat domain 5B |
| WDR68 | NM_001003725 | WD-repeat protein |
| WDR77 | NM_024102 | methylosome protein 50 |
| WFDC5 | NM_145652 | WAP four-disulfide core domain 5 precursor |
| WFIKKN2 | NM_175575 | WFIKKN2 protein |
| WHSC1 | NM_007331 | Wolf-Hirschhorn syndrome candidate 1 protein |
| WHSC1L1 | NM_023034 | WHSC1L1 protein isoform long |
| WIPI2 | NM_001033518 | hypothetical protein LOC26100 isoform c |
| WIRE | NM_133264 | WIRE protein |
| WISP2 | NM_003881 | WNT1 inducible signaling pathway protein 2 |
| WIT1 | NM_015855 | Wilms tumor upstream neighbor 1 |
| WNT1 | NM_005430 | wingless-type MMTV integration site family, |
| WNT2 | NM_003391 | wingless-type MMTV integration site family |
| WNT2B | NM_004185 | wingless-type MMTV integration site family, |
| WNT5B | NM_030775 | wingless-type MMTV integration site family, |
| WNT9B | NM_003396 | wingless-type MMTV integration site family, |
| WTAP | NM_004906 | Wilms′ tumour 1-associating protein isoform 1 |
| WWC3 | NM_015691 | hypothetical protein LOC55841 |
| XBP1 | NM_005080 | X-box binding protein 1 |
| XKR5 | NM_207411 | XK-related protein 5a |
| XKR9 | NM_001011720 | XK-related protein 9 |
| XLKD1 | NM_006691 | extracellular link domain containing 1 |
| XPC | NM_004628 | xeroderma pigmentosum, complementation group C |
| XPO5 | NM_020750 | exportin 5 |
| XPO6 | NM_015171 | exportin 6 |
| XPR1 | NM_004736 | xenotropic and polytropic retrovirus receptor |
| XRCC2 | NM_005431 | X-ray repair cross complementing protein 2 |
| XRCC3 | NM_005432 | X-ray repair cross complementing protein 3 |
| XRN1 | NM_019001 | 5′-3′ exoribonuclease 1 |
| XYLB | NM_005108 | xylulokinase homolog |
| XYLT1 | NM_022166 | xylosyltransferase I |
| YEATS2 | NM_018023 | YEATS domain containing 2 |
| YIPF2 | NM_024029 | Yipl domain family, member 2 |
| YIPF5 | NM_030799 | smooth muscle cell associated protein 5 |
| YKT6 | NM_006555 | YKT6 v-SNARE protein |
| YPEL1 | NM_013313 | yippee-like 1 |
| YPEL2 | NM_001005404 | yippee-like 2 |
| YPEL4 | NM_145008 | yippee-like 4 |
| YTHDC1 | NM_001031732 | splicing factor YT521-B isoform 1 |
| YTHDF1 | NM_017798 | YTH domain family, member 1 |
| YWHAG | NM_012479 | tyrosine 3-monooxygenase/tryptophan |
| YWHAZ | NM_003406 | tyrosine 3/tryptophan 5 -monooxygenase |
| YY1 | NM_003403 | YY1 transcription factor |
| ZBED1 | NM_004729 | Ac-like transposable element |
| ZBTB39 | NM_014830 | zinc finger and BTB domain containing 39 |
| ZBTB4 | NM_020899 | zinc finger and BTB domain containing 4 |
| ZBTB40 | NM_014870 | zinc finger and BTB domain containing 40 |
| ZBTB43 | NM_014007 | zinc finger protein 297B |
| ZBTB5 | NM_014872 | zinc finger and BTB domain containing 5 |
| ZBTB9 | NM_152735 | zinc finger and BTB domain containing 9 |
| ZC3H11A | NM_014827 | hypothetical protein LOC9877 |
| ZC3H12B | NM_001010888 | hypothetical protein LOC340554 |
| ZC3H3 | NM_015117 | zinc finger CCCH-type domain containing 3 |
| ZC3H7A | NM_014153 | zinc finger CCCH-type domain containing 7 |
| ZC3H7B | NM_017590 | zinc finger CCCH-type containing 7B |
| ZC3HAV1 | NM_020119 | zinc finger antiviral protein isoform 1 |
| ZCCHC17 | NM_016505 | putative S1 RNA binding domain protein |
| ZCSL3 | NM_181706 | zinc finger, CSL domain containing 3 |
| ZDHHC16 | NM_032327 | Abl-philin 2 isoform 1 |
| ZDHHC17 | NM_015336 | huntingtin interacting protein 14 |
| ZDHHC18 | NM_032283 | zinc finger, DHHC domain containing 18 |
| ZDHHC22 | NM_174976 | zinc finger, DHHC domain containing 22 |
| ZDHHC23 | NM_173570 | zinc finger, DHHC domain containing 23 |
| ZDHHC3 | NM_016598 | DHHC1 protein |
| ZDHHC8 | NM_013373 | zinc finger, DHHC domain containing 8 |
| ZFHX2 | NM_033400 | zinc finger homeobox 2 |
| ZFHX4 | NM_024721 | zinc finger homeodomain 4 |
| ZFP2 | NM_030613 | zinc finger protein 2 homolog |
| ZFP36 | NM_003407 | zinc finger protein 36, C3H type, homolog |
| ZFP41 | NM_173832 | zinc finger protein 41 homolog |
| ZFP90 | NM_133458 | zinc finger protein 90 homolog |
| ZFP91 | NM_170768 | zinc finger protein 91 isoform 2 |
| ZFPL1 | NM_006782 | zinc finger protein-like 1 |
| ZGPAT | NM_181484 | zinc finger, CCCH-type with G patch domain |
| ZHX2 | NM_014943 | zinc fingers and homeoboxes 2 |
| ZHX3 | NM_015035 | zinc fingers and homeoboxes 3 |
| ZMPSTE24 | NM_005857 | zinc metalloproteinase STE24 homolog |
| ZMYM3 | NM_005096 | zinc finger protein 261 |
| ZMYM4 | NM_00509S | zinc finger protein 262 |
| ZMYND11 | NM_006624 | zinc finger, MYND domain containing 11 isoform |
| ZNF137 | NM_003438 | zinc finger protein 137 (clone pHZ-30) |
| ZNF148 | NM_021964 | zinc finger protein 148 (pHZ-52) |
| ZNF16 | NM_001029976 | zinc finger protein 16 isoform 2 |
| ZNF179 | NM_007148 | zinc finger protein 179 |
| ZNF180 | NM_013256 | zinc finger protein 180 (HHZ168) |
| ZNF182 | NM_001007088 | zinc finger protein 21 isoform 2 |
| ZNF184 | NM_007149 | zinc finger protein 184 (Kruppel-like) |
| ZNF189 | NM_003452 | zinc finger protein 189 isoform 1 |
| ZNF193 | NM_006299 | zinc finger protein 193 |
| ZNF2 | NM_001017396 | zinc finger protein 2 isoform b |
| ZNF207 | NM_003457 | zinc finger protein 207 isoform a |
| ZNF213 | NM_004220 | zinc finger protein 213 |
| ZNF235 | NM_004234 | zinc finger protein 93 homolog |
| ZNF238 | NM_006352 | zinc finger protein 238 isoform 2 |
| ZNF248 | NM_021045 | zinc finger protein 248 |
| ZNF264 | NM_003417 | zinc finger protein 264 |
| ZNF274 | NM_016324 | zinc finger protein 274 isoform b |
| ZNF276 | NM_152287 | zinc finger protein 276 homolog |
| ZNF281 | NM_012482 | zinc finger protein 281 |
| ZNF282 | NM_003575 | zinc finger protein 282 |
| ZNF285 | NM_152354 | zinc finger protein 285 |
| ZNF3 | NM_017715 | zinc finger protein 3 isoform 1 |
| ZNF302 | NM_001012320 | zinc finger protein 302 |
| ZNF304 | NM_020657 | zinc finger protein 304 |
| ZNF312 | NM_018008 | zinc finger protein 312 |
| ZNF317 | NM_020933 | zinc finger protein 317 |
| ZNF324 | NM_014347 | zinc finger protein 324 |
| ZNF329 | NM_024620 | zinc finger protein 329 |
| ZNF33B | NM_006955 | zinc finger protein 33B |
| ZNF346 | NM_012279 | zinc finger protein 346 |
| ZNF358 | NM_018083 | zinc finger protein 358 |
| ZNF365 | NM_199451 | zinc finger protein 365 isoform C |
| ZNF367 | NM_153695 | zinc finger protein 367 |
| ZNF37A | NM_001007094 | zinc finger protein 37a |
| ZNF395 | NM_018660 | zinc finger protein 395 |
| ZNF397 | NM_032347 | zinc finger protein 397 |
| ZNF398 | NM_020781 | zinc finger 398 isoform b |
| ZNF406 | NM_001029939 | zinc finger protein 406 isoform TR-ZFAT |
| ZNF418 | NM_133460 | zinc finger protein 418 |
| ZNF436 | NM_030634 | zinc finger protein 436 |
| ZNF445 | NM_181489 | zinc finger protein 445 |
| ZNF446 | NM_017908 | zinc finger protein 446 |
| ZNF449 | NM_152695 | zinc finger protein 449 |
| ZNF45 | NM_003425 | zinc finger protein 45 |
| ZNF471 | NM_020813 | zinc finger protein 471 |
| ZNF480 | NM_144684 | zinc finger protein 480 |
| ZNF493 | NM_175910 | zinc finger protein 493 |
| ZNF497 | NM_198458 | zinc finger protein 497 |
| ZNF501 | NM_145044 | zinc finger protein 501 |
| ZNF502 | NM_033210 | zinc finger protein 502 |
| ZNF510 | NM_014930 | zinc finger protein 510 |
| ZNF512 | NM_032434 | zinc finger protein 512 |
| ZNF513 | NM_144631 | zinc finger protein 513 |
| ZNF526 | NM_133444 | zinc finger protein 526 |
| ZNF530 | NM_020880 | zinc finger protein 530 |
| ZNF532 | NM_018181 | zinc finger protein 532 |
| ZNF540 | NM_152606 | zinc finger protein 540 |
| ZNF551 | NM_138347 | zinc finger protein 551 |
| ZNF553 | NM_152652 | zinc finger protein 553 |
| ZNF561 | NM_152289 | zinc finger protein 561 |
| ZNF562 | NM_017656 | zinc finger protein 562 |
| ZNF579 | NM_152600 | zinc finger protein 579 |
| ZNF580 | NM_016202 | zinc finger protein 580 |
| ZNF587 | NM_032828 | zinc finger protein 587 |
| ZNF600 | NM_198457 | zinc finger protein 600 |
| ZNF605 | NM_183238 | zinc finger protein 605 |
| ZNF614 | NM_025040 | zinc finger protein 614 |
| ZNF621 | NM_198484 | zinc finger protein 621 |
| ZNF623 | NM_014789 | zinc finger protein 623 |
| ZNF628 | NM_033113 | zinc finger protein 628 |
| ZNF641 | NM_152320 | zinc finger protein 641 |
| ZNF644 | NM_016620 | zinc finger protein 644 isoform 2 |
| ZNF651 | NM_145166 | zinc finger protein 651 |
| ZNF652 | NM_014897 | zinc finger protein 652 |
| ZNF662 | NM_207404 | zinc finger protein 662 |
| ZNF671 | NM_024833 | zinc finger protein 671 |
| ZNF672 | NM_024836 | zinc finger protein 672 |
| 7NF689 | NM_138447 | zinc finger protein HIT-39 |
| ZNF694 | NM_001012981 | zinc finger protein 694 |
| ZNF70 | NM_021916 | zinc finger protein 70 |
| ZNF706 | NM_016096 | HSPC038 protein |
| ZNF707 | NM_173831 | zinc finger protein 707 |
| ZNF710 | NM_198526 | zinc finger protein 710 |
| ZNF76 | NM_003427 | zinc finger protein 76 (expressed in testis) |
| ZNFN1A1 | NM_006060 | zinc finger protein, subfamily 1A, 1 (Ikaros) |
| ZNFN1A4 | NM_022465 | zinc finger protein, subfamily 1A, 4 |
| ZNFX1 | NM_021035 | zinc finger, NFX1-type containing 1 |
| ZNHIT1 | NM_006349 | zinc finger, HIT domain containing 1 |
| ZSCAN2 | NM_181877 | zinc finger protein 29 isoform 1 |
| ZSWIM4 | NM_023072 | zinc finger, SWIM domain containing 4 |
| ZXDB | NM_007157 | zinc finger, X-linked, duplicated B |
| ZXDC | NM_025112 | ZXD family zinc finger C |
| ZYG11B | NM_024646 | hypothetical protein LOC79699 |
| ZYG11BL | NM_006336 | zyg-11 homolog B (C. elegans)-like |
| ZZEF1 | NM_015113 | zinc finger, ZZ type with EF hand domain 1 |
| ZZZ3 | NM_015534 | zinc finger, ZZ domain containing 3 |

TABLE 4
Predicted hsa-miR-34a targets that exhibited altered mRNA expression levels in human
cancer cells after transfection with pre-miR hsa-miR-34a.

| | RefSeq | |
|---|---|---|
| | Transcript ID | |
| Gene Symbol | (Pruitt et al., 2005) | Description |
| ABCA1 | NM_005502 | ATP-binding cassette, sub-family A member 1 |
| ABLIM3 | NM_014945 | actin binding LIM protein family, member 3 |
| ANK3 | NM_001149 | ankyrin 3 isoform 2 |
| APPBP2 | NM_006380 | amyloid beta precursor protein-binding protein |
| AQP3 | NM_004925 | aquaporin 3 |
| AREG | NM_001657 | amphiregulin preproprotein |
| ARHGAP1 | NM_004308 | Rho GTPase activating protein 1 |
| ARHGDIB | NM_001175 | Rho GDP dissociation inhibitor (GDI) beta |
| ARTS-1 | NM_016442 | type 1 tumor necrosis factor receptor shedding |
| ATP1B3 | NM_001679 | Na+/K+ -ATPase beta 3 subunit |
| ATXN1 | NM_000332 | ataxin 1 |
| AXL | NM_001699 | AXL receptor tyrosine kinase isoform 2 |
| B4GALT1 | NM_001497 | UDP-Gal:betaGlcNAc beta 1,4- |
| BCL10 | NM_003921 | B-cell CLL/lymphoma 10 |
| BIRC5 | NM_001012270 | baculoviral IAP repeat-containing protein 5 |
| BRCA1 | NM_007306 | breast cancer 1, early onset isoform |
| BRD4 | NM_014299 | bromodomain-containing protein 4 isoform short |
| BTN3A2 | NM_007047 | butyrophilin, subfamily 3, member A2 precursor |
| C11orf9 | NM_013279 | hypothetical protein LOC745 |
| C19orf21 | NM_173481 | hypothetical protein LOC126353 |
| C1QL1 | NM_006688 | complement component 1, q subcomponent-like 1 |
| C8orf1 | NM_004337 | hypothetical protein LOC734 |
| CAP1 | NM_006367 | adenylyl cyclase-associated protein |
| CASP2 | NM_032982 | caspase 2 isoform 1 preproprotein |
| CASP7 | NM_001227 | caspase 7 isoform alpha precursor |
| CCND1 | NM_053056 | cyclin D1 |
| CCND3 | NM_001760 | cyclin D3 |
| CDC23 | NM_004661 | cell division cycle protein 23 |
| CDH17 | NM_004063 | cadherin 17 precursor |
| CHES1 | NM_005197 | checkpoint suppressor 1 |
| CLDN1 | NM_021101 | claudin 1 |
| COL5A1 | NM_000093 | alpha 1 type V collagen preproprotein |
| COL6A2 | NM_058175 | alpha 2 type VI collagen isoform 2C2a precursor |
| CRIP2 | NM_001312 | cysteine-rich protein 2 |
| CRISPLD2 | NM_031476 | cysteine-rich secretory protein LCCL domain |
| CTDSPL | NM_001008392 | small CTD phosphatase 3 isoform 1 |
| CTNND1 | NM_001331 | catenin (cadherin-associated protein), delta 1 |
| CTSB | NM_001908 | cathepsin B preproprotein |
| CXCL1 | NM_001511 | chemokine (C-X-C motif) ligand 1 |
| CXCL2 | NM_002089 | chemokine (C-X-C motif) ligand 2 |
| CXCL5 | NM_002994 | chemokine (C-X-C motif) ligand 5 precursor |
| CYR61 | NM_001554 | cysteine-rich, angiogenic inducer, 61 |
| DDX58 | NM_014314 | DEAD/H (Asp-Glu-Ala-Asp/His) box polypeptide |
| DGAT1 | NM_012079 | diacylglycerol O-acyltransferase 1 |
| DKFZp564K142 | NM_032121 | implantation-associated protein |
| DPYSL3 | NM_001387 | dihydropyrimidinase-like 3 |
| E2F5 | NM_001951 | E2F transcription factor 5 |
| EFHD2 | NM_024329 | EF hand domain family, member D2 |
| EI24 | NM_001007277 | etoposide induced 2.4 isoform 2 |
| F8 | NM_000132 | coagulation factor VIII isoform a precursor |
| FAS | NM_000043 | tumor necrosis factor receptor superfamily, |
| FBXO17 | NM_024907 | F-box protein FBG4 isoform 2 |
| FKBP1B | NM_004116 | FK506-binding protein 1B isoform a |
| FLJ14154 | NM_024845 | hypothetical protein LOC79903 |
| FLJ20232 | NM_019008 | hypothetical protein LOC54471 |
| FLJ20489 | NM_017842 | hypothetical protein LOC55652 |
| FLOT2 | NM_004475 | flotillin 2 |
| FLRT3 | NM_013281 | fibronectin leucine rich transmembrane protein 3 |
| FOSL1 | NM_005438 | FOS-like antigen 1 |
| FOXM1 | NM_021953 | forkhead box M1 isoform 2 |
| FSTL1 | NM_007085 | follistatin-like 1 precursor |
| FXYD2 | NM_001680 | FXYD domain-containing ion transport regulator 2 |
| GALNT7 | NM_017423 | polypeptide N-acetylgalactosaminyltransferase 7 |
| GLS | NM_014905 | glutaminase C |
| GMNN | NM_015895 | geminin |
| GNPDA1 | NM_005471 | glucosamine-6-phosphate deaminase 1 |
| GORASP2 | NM_015530 | golgi reassembly stacking protein 2 |
| GPR64 | NM_005756 | G protein-coupled receptor 64 |
| GTSE1 | NM_016426 | G-2 and S-phase expressed 1 |
| GYG2 | NM_003918 | glycogenin 2 |
| HDAC1 | NM_004964 | histone deacetylase 1 |
| HIC2 | NM_015094 | hypermethylated in cancer 2 |
| HLX1 | NM_021958 | H2.0-like homeo box 1 |
| HMGCS1 | NM_002130 | 3-hydroxy-3-methylglutaryl-Coenzyme A synthase 1 |
| HMGN4 | NM_006353 | high mobility group nucleosomal binding domain |
| HMMR | NM_012484 | hyaluronan-mediated motility receptor isoform a |
| IL1RL1 | NM_003856 | interleukin 1 receptor-like 1 isoform 2 |
| INHBB | NM_002193 | inhibin beta B subunit precursor |
| IRF1 | NM_002198 | interferon regulatory factor 1 |
| ITGAM | NM_000632 | integrin alpha M precursor |
| ITPR2 | NM_002223 | inositol 1,4,5-triphosphate receptor, type 2 |
| KCNK3 | NM_002246 | potassium channel, subfamily K, member 3 |
| KCNMA1 | NM_001014797 | large conductance calcium-activated potassium |
| KIF11 | NM_004523 | kinesin family member 11 |
| KLC2 | NM_022822 | likely ortholog of kinesin light chain 2 |
| KLF4 | NM_004235 | Kruppel-like factor 4 |
| KRT20 | NM_019010 | keratin 20 |
| LEPREL1 | NM_018192 | leprecan-like 1 |
| LGR4 | NM_018490 | leucine-rich repeat-containing G protein-coupled |
| LHX2 | NM_004789 | LIM homeobox protein 2 |
| LITAF | NM_004862 | LPS-induced TNF-alpha factor |
| LMAN2L | NM_030805 | lectin, mannose-binding 2-like |
| LNK | NM_005475 | lymphocyte adaptor protein |
| LOC93349 | NM_138402 | hypothetical protein LOC93349 |
| LPIN1 | NM_145693 | lipin 1 |
| LRRC40 | NM_017768 | leucine rich repeat containing 40 |
| LYST | NM_000081 | lysosomal trafficking regulator isoform 1 |
| MAFF | NM_012323 | transcription factor MAFF |
| MAP7 | NM_003980 | microtubule-associated protein 7 |
| MARCH8 | NM_001002265 | cellular modulator of immune recognition |
| MCL1 | NM_021960 | myeloid cell leukemia sequence 1 isoform 1 |
| MET | NM_000245 | met proto-oncogene precursor |
| MFN2 | NM_014874 | mitofusin 2 |
| MK167 | NM_002417 | antigen identified by monoclonal antibody Ki-67 |
| MPHOSPH6 | NM_005792 | M-phase phosphoprotein 6 |
| MTUS1 | NM_001001924 | mitochondrial tumor suppressor 1 isoform 1 |
| MXD4 | NM_006454 | MAD4 |
| MYL9 | NM_006097 | myosin regulatory light polypeptide 9 isoform a |
| NAV3 | NM_014903 | neuron navigator 3 |
| NF2 | NM_000268 | neurofibromin 2 isoform 1 |
| NFYC | NM_014223 | nuclear transcription factor Y, gamma |
| NINJ1 | NM_004148 | ninjurin 1 |
| NMT2 | NM_004808 | glycylpeptide N-tetradecanoyltransferase 2 |
| NPTX1 | NM_002522 | neuronal pentraxin I precursor |
| NR4A2 | NM_006186 | nuclear receptor subfamily 4, group A, member 2 |
| NRP2 | NM_003872 | neuropilin 2 isoform 2 precursor |
| NUP210 | NM_024923 | nucleoporin 210 |
| PALM2-AKAP2 | NM_007203 | PALM2-AKAP2 protein isoform 1 |
| PDCD2 | NM_144781 | programmed cell death 2 isoform 2 |
| PER2 | NM_022817 | period 2 isoform 1 |
| PIK3CD | NM_005026 | phosphoinositide-3-kinase, catalytic, delta |
| PODXL | NM_001018111 | podocalyxin-like precursor isoform 1 |
| PPL | NM_002705 | Periplakin |
| PPP1R11 | NM_021959 | protein phosphatase 1, regulatory (inhibitor) |
| PROSC | NM_007198 | proline synthetase co-transcribed homolog |
| PSME3 | NM_005789 | proteasome activator subunit 3 isoform 1 |
| PTPRE | NM_006504 | protein tyrosine phosphatase, receptor type, E |
| RAI14 | NM_015577 | retinoic acid induced 14 |
| RASSF2 | NM_014737 | Ras association domain family 2 |
| RHEB | NM_005614 | Ras homolog enriched in brain |
| RIP | NM_001033002 | RPA interacting protein isoform 1 |
| RRAD | NM_004165 | Ras-related associated with diabetes |
| RRAS | NM_006270 | related RAS viral (r-ras) oncogene homolog |
| SERPINE1 | NM_000602 | plasminogen activator inhibitor-1 |
| SGPP1 | NM_030791 | sphingosine-1-phosphatase |
| SGSH | NM_000199 | N-sulfoglucosamine sulfohydrolase (sulfamidase) |
| SH3GL1 | NM_003025 | SH3-domain GRB2-like 1 |
| SIRT1 | NM_012238 | sirtuin 1 |
| SLC29A1 | NM_004955 | solute carrier family 29 (nucleoside |
| SLC6A6 | NM_003043 | solute carrier family 6 (neurotransmitter |
| SMAD3 | NM_005902 | MAD, mothers against decapentaplegic homolog 3 |
| SPARC | NM_003118 | secreted protein, acidic, cysteine-rich |
| SPFH1 | NM_006459 | SPFH domain family, member 1 |
| STC1 | NM_003155 | stanniocalcin 1 precursor |
| SVIL | NM_003174 | supervillin isoform 1 |
| SWAP70 | NM_015055 | SWAP-70 protein |
| SYT1 | NM_005639 | synaptotagmin I |
| TGFBR2 | NM_001024847 | TGF-beta type II receptor isoform A precursor |
| THBD | NM_000361 | thrombomodulin precursor |
| TIMM13 | NM_012458 | translocase of inner mitochondrial membrane 13 |
| TK1 | NM_003258 | thymidine kinase 1, soluble |
| TM4SF4 | NM_004617 | transmembrane 4 superfamily member 4 |
| TMEM48 | NM_018087 | transmembrane protein 48 |
| TNFRSF9 | NM_001561 | tumor necrosis factor receptor superfamily, |
| TPD52 | NM_001025252 | tumor protein D52 isoform 1 |
| TPI1 | NM_000365 | triosephosphate isomerase 1 |
| TRIM14 | NM_014788 | tripartite motif protein TRIM14 isoform alpha |
| TRIM22 | NM_006074 | tripartite motif-containing 22 |
| TRIO | NM_007118 | triple functional domain (PTPRF interacting) |
| TSN | NM_004622 | Translin |
| TUBB | NM_178014 | tubulin, beta polypeptide |
| UBE2L3 | NM_003347 | ubiquitin-conjugating enzyme E2L 3 isoform 1 |
| UROS | NM_000375 | uroporphyrinogen III synthase |
| VPS4A | NM_013245 | vacuolar protein sorting factor 4A |
| WHSC1 | NM_007331 | Wolf-Hirschhorn syndrome candidate 1 protein |
| XBP1 | NM_005080 | X-box binding protein 1 |
| YKT6 | NM_006555 | YKT6 v-SNARE protein |
| ZNF238 | NM_006352 | zinc finger protein 238 isoform 2 |
| ZNF281 | NM_012482 | zinc finger protein 281 |
| ZNF551 | NM_138347 | zinc finger protein 551 |
| ZNF580 | NM_016202 | zinc finger protein 580 |
| ZNF652 | NM_014897 | zinc finger protein 652 |

Predicted gene targets are shown in Table 3. Target genes whose mRNA expression levels are affected by hsa-miR-34 represent particularly useful candidates for cancer therapy and therapy of other diseases or conditions through manipulation of their expression levels.

Certain embodiments of the invention include determining expression of one or more marker, gene, or nucleic acid segment representative of one or more genes, by using an amplification assay, a hybridization assay, or protein assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The nucleic acids from a sample may be labeled from the sample and/or hybridizing the labeled nucleic acid to one or more nucleic acid probes. Nucleic acids, mRNA, and/or nucleic acid probes may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art. Proteins are typically assayed by immunoblotting, chromatography, or mass spectrometry or other methods known to those of ordinary skill in the art.

The present invention also concerns kits containing compositions of the invention or compositions to implement methods of the invention. In some embodiments, kits can be used to evaluate one or more marker molecules, and/or express one or more miRNA or miRNA inhibitor. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 100, 150, 200 or more probes, recombinant nucleic acid, or synthetic nucleic acid molecules related to the markers to be assessed or an miRNA or miRNA inhibitor to be expressed or modulated, and may include any range or combination derivable therein. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more. Kits for using probes, synthetic nucleic acids, recombinant nucleic acids, or non-synthetic nucleic acids of the invention for therapeutic, prognostic, or diagnostic applications are included as part of the invention. Specifically contemplated are any such molecules corresponding to any miRNA reported to influence biological activity or expression of one or more marker gene or gene pathway described herein. In certain aspects, negative and/or positive controls are included in some kit embodiments. The control molecules can be used to verify transfection efficiency and/or control for transfection-induced changes in cells.

Certain embodiments are directed to a kit for assessment of a pathological condition or the risk of developing a pathological condition in a patient by nucleic acid profiling of a sample comprising, in suitable container means, two or more nucleic acid hybridization or amplification reagents. The kit can comprise reagents for labeling nucleic acids in a sample and/or nucleic acid hybridization reagents. The hybridization reagents typically comprise hybridization probes. Amplification reagents include, but are not limited to amplification primers, reagents, and enzymes.

In some embodiments of the invention, an expression profile is generated by steps that include: (a) labeling nucleic acid in the sample; (b) hybridizing the nucleic acid to a number of probes, or amplifying a number of nucleic acids, and (c) determining and/or quantitating nucleic acid hybridization to the probes or detecting and quantitating amplification products, wherein an expression profile is generated. See U.S. Provisional Patent Application 60/575,743 and the U.S. Provisional Patent Application 60/649,584, and U.S. patent application Ser. No. 11/141,707 and U.S. patent application Ser. No. 11/273,640, all of which are hereby incorporated by reference.

Methods of the invention involve diagnosing and/or assessing the prognosis of a patient based on a miRNA and/or a marker nucleic acid expression profile. In certain embodiments, the elevation or reduction in the level of expression of a particular gene or genetic pathway or set of nucleic acids in a cell is correlated with a disease state or pathological condition compared to the expression level of the same in a normal or non-pathologic cell or tissue sample. This correlation allows for diagnostic and/or prognostic methods to be carried out when the expression level of one or more nucleic acid is measured in a biological sample being assessed and then compared to the expression level of a normal or non-pathologic cell or tissue sample. It is specifically contemplated that expression profiles for patients, particularly those suspected of having or having a propensity for a particular disease or condition such as cancer, can be generated by evaluating any of or sets of the miRNAs and/or nucleic acids discussed in this application. The expression profile that is generated from the patient will be one that provides information regarding the particular disease or condition. In many embodiments, the profile is generated using nucleic acid hybridization or amplification, (e.g., array hybridization or RT-PCR). In certain aspects, an expression profile can be used in conjunction with other diagnostic and/or prognostic tests, such as histology, protein profiles in the serum and/or cytogenetic assessment.

TABLE 5
Tumor associated mRNAs altered by hsa-miR-34a having prognostic or therapeutic value for the treatment
of various malignancies.

| Gene | | Cellular | | |
|---|---|---|---|---|
| Symbol | Gene Title | Process | Cancer Type | Reference |
| AKAP12 | Akap-12/ | signal | CRC, PC, LC, GC, AML, | (Xia et al., 2001; Wikman et al., 2002; Boultwood et al., |
| | SSeCKS/Gravin | transduction | CML | 2004; Choi et al., 2004; Mori et al., 2006) |
| ANG | Angiogenin | angiogenesis | BC, OC, M, PaC, UC, CeC | (Barton et al., 1997; Montero et al., 1998; Hartmann et al., |
| | | | | 1999; Miyake et al., 1999; Shimoyama et al., 1999; Bodner- |
| | | | | Adler et al., 2001) |
| AREG | Amphiregulin | signal | HCC, NSCLC, MM, PC, | (Kitadai et al., 1993; Ebert et al., 1994; Solic and Davies, |
| | | transduction | OC, CRC, PaC, GC | 1997; D'Antonio et al., 2002; Bostwick et al., 2004; Ishikawa |
| | | | | et al., 2005; Mahtouk et al., 2005; Castillo et al., 2006) |
| AURKB/ | aurora kinase B | chromosomal | PC, NSCLC, BC, CRC | (Keen and Taylor, 2004; Smith et al., 2005; Chieffi et al., |
| STK12 | | stability | | 2006) |
| BCL10 | BCL-10 | signal | MALT BCL | (Thome, 2004) |
| | | transduction | | |
| BRCA1 | BRCA-1 | chromosomal | BC, OC | (Wooster and Weber, 2003) |
| | | stability | | |
| BRCA2 | BRCA-2 | chromosomal | BC, OC | (Wooster and Weber, 2003) |
| | | stability | | |
| BUB1 | BUB1 | chromosomal | AML, SGT, ALL, HL, L, | (Cahill et al., 1998; Qian et al., 2002; Ru et al., 2002; |
| | | stability | CRC, GC | Grabsch et al., 2003; Shigeishi et al., 2006) |
| CCNA2 | cyclin A2 | cell cycle | AML | (Qian et al., 2002) |
| CCND1 | cyclin D1 | cell cycle | MCL, BC, SCCHN, OepC, | (Donnellan and Chetty, 1998) |
| | | | HCC, CRC, BldC, EC, OC, | |
| | | | M, AC, GB, GC, PaC | |
| CCND3 | cyclin D3 | cell cycle | EC, TC, BldC, CRC, LSCC, | (Florenes et al., 2000; Ito et al., 2001; Filipits et al., 2002; Bai |
| | | | BCL, PaC, M | et al., 2003; Pruneri et al., 2005; Tanami et al., 2005; Lopez- |
| | | | | Beltran et al., 2006; Troncone et al., 2007; Wu et al., 2006) |
| CDK4 | CDK-4 | cell cycle | G, GB, BC, LC, GC, EC, L, | (Malumbres and Barbacid, 2001) |
| | | | OS, OC, TT, HCC, CHN | |
| CDKN2C | CDK inhibitor 2C | cell cycle | HB, MB, HCC, HL, MM | (Iolascon et al., 1998; Kulkami et al., 2002; Morishita et al., |
| | | | | 2004; Sanchez-Aguilera et al., 2004) |
| CTGF | CTGF/IGFBP-8 | cell adhesion, | BC, GB, OepC, RMS, CRC, | (Hishikawa et al., 1999; Shimo et al., 2001; Koliopanos et al., |
| | | migration | PC, | 2002; Pan et al., 2002; Croci et al., 2004; Lin et al., 2005; |
| | | | | Yang et al., 2005) |
| EIF3S3 | eIF-3 subunit 3g | translation | BC, PC, HCC | (Nupponen et at., 1999; Nupponen et al., 2000; Okamoto et |
| | | | | al., 2003) |
| FAS | Fas | apoptosis | NSCLC, G, L, CRC, OepC | (Moller et al., 1994; Gratas et al., 1998; Martinez-Lorenzo et |
| | | | | al., 1998; Shinoura et al., 2000; Viard-Leveugle et al., 2003) |
| FOXM1 | forkhead box M1 | transcription | GB, LC, PC | (Kalin et al., 2006; Kim et al., 2006; Liu et al., 2006) |
| GMNN | Geminin | DNA | CRC, BC, CeC | (Wohlschlegel et al., 2002; Bravou et al., 2005; Shetty et al., |
| | | replication | | 2005) |
| HDAC1 | HDAC-1 | transcription | BC, PC | (Kawai et al., 2003; Halkidou et al., 2004) |
| IL8 | IL-8 | signal | BC, CRC, PaC, NSCLC, PC, | (Akiba et al., 2001; Sparmann and Bar-Sagi, 2004) |
| | | transduction | HCC | |
| JUN | c-Jun | transcription | HL, HCC | (Eferl et al., 2003; Weiss and Bohmann, 2004) |
| LMO4 | Lmo-4 | transcription | BC, SCCHN, SCLC | (Visvader et al., 2001; Mizunuma et al., 2003; Taniwaki et |
| | | | | al., 2006) |
| MCAM | MCAM | cell adhesion | M, AS, KS, LMS | (Boccaccia and Comoglio, 2006) |
| MCL1 | Mcl-1 | apoptosis | HCC, MM, TT, CLL, | (Krajewska et al., 1996; Kitada et al., 1998; Cho-Vega et al., |
| | | | ALCL, BCL, PC | 2004; Rust et al., 2005; Sano et al., 2005; Wuilleme-Toumi et |
| | | | | al., 2005; Fleischer et al., 2006; Sieghart et al., 2006) |
| MET | c-Met | signal | SPRC, HCC, GC, SCCHN, | (Boccaccio and Comoglio, 2006) |
| | | transduction | OS, RMS, GB, BC, M, | |
| | | | CRC, GI, PaC, PC, OC | |
| MLF1 | myeloid leukemia | cell cycle | AML | (Matsumoto et al., 2000) |
| | factor 1 | | | |
| MYBL2 | Myb L2 | transcription | BC, NSCLC, PC, OC | (Tanner et al., 2000; Bar-Shira et al., 2002; Borczuk et al., |
| | | | | 2003; Ginestier et al., 2006) |
| NF1 | NF-1 | signal | G, AC, NF, PCC, ML | (Rubin and Gutmann, 2005) |
| | | transduction | | |
| NF2 | Merlin/NF-2 | cell adhesion | Schw, TC, HCC, MG, MT | (McClatchey and Giovannini, 2005) |
| | | | of lung | |
| PBX1 | PBX-1 | transcription | ALL | (Aspland et al., 2001) |
| PIK3CD | PI 3-kinase IA | signal | AML, MSS, GI | (Vogt et al., 2006) |
| | delta (p110 delta) | transduction | | |
| PLK1 | polo-like kinase 1 | chromosomal | NSCLC, OrpC, OepC, GC, | (Strebhardt and Ullrich, 2006) |
| | | stability | M, BC, OC, EC, CRC, GB, | |
| | | | PapC, PaC, PC, HB, NHL | |
| RASSF2 | RASSF2 | signal | GC, CRC, OC | (Akino et al., 2005; Endoh et al., 2005; Lambros et al., 2005) |
| | | transduction | | |
| RBL1 | p107 | cell cycle | BCL, PC, CRC, TC | (Takimoto et al., 1998; Claudio et al., 2002; Wu et al., 2002; |
| | | signal | | Ito et al., 2003) |
| RRAS | R-RAS | transduction | CeC, BC | (Yu and Feig, 2002; Rincon-Arano et al., 2003) |
| SMAD3 | SMAD-3 | signal | GC, CRC, HCC, BC, ALL | (Zhu et al., 1998; Han et al., 2004; Liu and Matsuura, 2005; |
| | | transduction | | Yamagata et al., 2005; Yang et al., 2006) |
| TACSTD1 | tumor-associated | cell adhesion, | NSCLC, CRC | (Xi et al., 2006a; Xi et al., 2006b) |
| | calcium signal | vesicle | | |
| | transducer 1 | trafficking | | |
| TGFB2 | TGF beta-2 | signal | PaC, CRC, BC, M | (Krasagakis et al., 1998; Jonson et al., 2001; Nakagawa et al., |
| | | transduction | | 2004; Beisner et al., 2006) |
| TGFBR2 | TGF beta receptor | signal | BC, CRC | |
| | type II | transduction | | (Markowitz, 2000; Lucke et al., 2001; Biswas et al., 2004) |
| TPD52 | tumor protein D52 | signal | BC, LC, PC, OC, EC, HCC | (Boutros et al., 2004) |
| | | transduction | | |
| TXN | thioredoxin (trx) | thioredoxin | LC, PaC, CeC, HCC | (Marks, 2006) |
| | | redox system | | |
| WNT7B | Wnt-7b | signal | BC, BldC | (Huguet et al., 1994; Bui et al., 1998) |
| | | transduction | | |

Abbreviations:
AC, astrocytoma;
ALCL, anaplastic large cell lymphoma;
ALL, acute lymphoblastic leukemia;
AML, acute myeloid leukemia;
AS, angiosarcoma;
BC, breast carcinoma;
BCL, B-cell lymphoma;
BldC, bladder carcinoma;
CeC, cervical carcinoma;
CHN, carcinoma of the head and neck;
CLL, chronic lymphocytic leukemia;
CML, chronic myeloid leukemia;
CRC, colorectal carcinoma;
EC, endometrial carcinoma;
G, glioma;
GB, glioblastoma;
GC, gastric carcinoma;
GI, gastrinoma;
HB, hepatoblastoma;
HCC, hepatocellular carcinoma;
HL, Hodgkin lymphoma;
KS, Kaposi's sarcoma;
L, leukemia;
LC, lung carcinoma;
LMS, leiomyosarcoma;
LSCC, laryngeal squamous cell carcinoma;
M, melanoma;
MALT BCL, mucosa-associated lymphoid tissue B-cell lymphoma;
MB, medulloblastoma;
MCL, mantle cell lymphoma;
MG, meningioma;
ML, myeloid leukemia;
MM, multiple myeloma;
MSS, high-risk myelodysplastic syndrome;
MT, mesothelioma;
NF, neurofibroma;
NHL, non-Hodgkin lymphoma;
NSCLC, non-small cell lung carcinoma;
OC, ovarian carcinoma;
OepC, esophageal carcinoma;
OrpC, oropharyngeal carcinoma;
OS, osteosarcoma;
PaC, pancreatic carcinoma;
PapC, papillary carcinoma;
PC, prostate carcinoma;
PCC, pheochromocytoma;
RMS, rhabdomyosarcoma;
SCCHN, squamous cell carcinoma of the head and neck;
Schw, schwannoma;
SCLC, small cell lung cancer;
SGT, salivary gland tumor;
SPRC, sporadic papillary renal carcinoma;
TC, thyroid carcinoma;
TT, testicular tumor;
UC, urothelial carcinoma

The methods can further comprise one or more of the steps including: (a) obtaining a sample from the patient, (b) isolating nucleic acids from the sample, (c) labeling the nucleic acids isolated from the sample, and (d) hybridizing the labeled nucleic acids to one or more probes. Nucleic acids of the invention include one or more nucleic acid comprising at least one segment having a sequence or complementary sequence of to a nucleic acid representative of one or more of genes or markers in Table 1, 3, 4, and/or 5.

It is contemplated that any method or composition described herein can be implemented with respect to any other method or composition described herein and that different embodiments may be combined. It is specifically contemplated that any methods and compositions discussed herein with respect to miRNA molecules, miRNA, genes, and Certain embodiments of the invention include determining expression of one or more marker, gene, or nucleic acid representative thereof, by using an amplification assay, a hybridization assay, or protein assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The nucleic acids from a sample may be labeled from the sample and/or hybridizing the labeled nucleic acid to one or more nucleic acid probes. Nucleic acids, mRNA, and/or nucleic acid probes may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art. Protein are typically assayed by immunoblotting, chromatography, or mass spectrometry or other methods known to those of ordinary skill in the art.

The present invention also concerns kits containing compositions of the invention or compositions to implement methods of the invention. In some embodiments, kits can be used to evaluate one or more marker molecules, and/or express one or more miRNA. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 100, 150, 200 or more probes, recombinant nucleic acid, or synthetic nucleic acid molecules related to the markers to be assessed or an miRNA to be expressed or modulated, and may include any range or combination derivable therein. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more. Kits for using probes, synthetic nucleic acids, recombinant nucleic acids, or non-synthetic nucleic acids of the invention for therapeutic, prognostic, or diagnostic applications are included as part of the invention. Specifically contemplated are any such molecules corresponding to any miRNA reported to influence biological activity or expression of one or more marker gene or gene pathway described herein. In certain aspects, negative and/or positive controls are included in some kit embodiments. The control molecules can be used to verify transfection efficiency and/or control for transfection-induced changes in cells.

Certain embodiments are directed to a kit for assessment of a pathological condition or the risk of developing a pathological condition in a patient by nucleic acid profiling of a sample comprising, in suitable container means, two or more nucleic acid hybridization or amplification reagents. The kit can comprise reagents for labeling nucleic acids in a sample and/or nucleic acid hybridization reagents. The hybridization reagents typically comprise hybridization probes. Amplification reagents include, but are not limited to amplification primers, reagents, and enzymes.

In some embodiments of the invention, an expression profile is generated by steps that include: (a) labeling nucleic acid in the sample; (b) hybridizing the nucleic acid to a number of probes, or amplifying a number of nucleic acids, and (c) determining and/or quantitating nucleic acid hybridization to the probes or detecting and quantitating amplification products, wherein an expression profile is generated. See U.S. Provisional Patent Application 60/575,743 and the U.S. Provisional Patent Application 60/649,584, and U.S. patent application Ser. No. 11/141,707 and U.S. patent application Ser. No. 11/273,640, all of which are hereby incorporated by reference.

Methods of the invention involve diagnosing and/or assessing the prognosis of a patient based on a miRNA and/or a marker nucleic acid expression profile. In certain embodiments, the elevation or reduction in the level of expression of a particular gene or genetic pathway or set of nucleic acids in a cell is correlated with a disease state or pathological condition compared to the expression level of the same in a normal or non-pathologic cell or tissue sample. This correlation allows for diagnostic and/or prognostic methods to be carried out when the expression level of one or more nucleic acid is measured in a biological sample being assessed and then compared to the expression level of a normal or non-pathologic cell or tissue sample. It is specifically contemplated that expression profiles for patients, particularly those suspected of having or having a propensity for a particular disease or condition such as cancer, can be generated by evaluating any of or sets of the miRNAs and/or nucleic acids discussed in this application. The expression profile that is generated from the patient will be one that provides information regarding the particular disease or condition. In many embodiments, the profile is generated using nucleic acid hybridization or amplification, (e.g., array hybridization or RT-PCR). In certain aspects, an expression profile can be used in conjunction with other diagnostic and/or prognostic tests, such as histology, protein profiles in the serum and/or cytogenetic assessment.

The methods can further comprise one or more of the steps including: (a) obtaining a sample from the patient, (b) isolating nucleic acids from the sample, (c) labeling the nucleic acids isolated from the sample, and (d) hybridizing the labeled nucleic acids to one or more probes. Nucleic acids of the invention include one or more nucleic acid comprising at least one segment having a sequence or complementary sequence of to a nucleic acid representative of one or more of genes or markers in Table 1, 3, 4, and/or 5.

It is contemplated that any method or composition described herein can be implemented with respect to any other method or composition described herein and that different embodiments may be combined. It is specifically contemplated that any methods and compositions discussed herein with respect to miRNA molecules, miRNA, genes and nucleic acids representative of genes may be implemented with respect to synthetic nucleic acids. In some embodiments the synthetic nucleic acid is exposed to the proper conditions to allow it to become a processed or mature nucleic acid, such as a miRNA under physiological circumstances. The claims originally filed are contemplated to cover claims that are multiply dependent on any filed claim or combination of filed claims.

Also, any embodiment of the invention involving specific genes (including representative fragments there of), mRNA, or miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence of the specified miRNA.

It will be further understood that shorthand notations are employed such that a generic description of a gene or marker thereof, or of a miRNA refers to any of its gene family members (distinguished by a number) or representative fragments thereof, unless otherwise indicated. It is understood by those of skill in the art that a “gene family” refers to a group of genes having the same coding sequence or miRNA coding sequence. Typically, miRNA members of a gene family are identified by a number following the initial designation. For example, miR-16-1 and miR-16-2 are members of the miR-16 gene family and “mir-7” refers to miR-7-1, miR-7-2 and miR-7-3. Moreover, unless otherwise indicated, a shorthand notation refers to related miRNAs (distinguished by a letter). Exceptions to these shorthand notations will be otherwise identified.

Other embodiments of the invention are discussed throughout this application. Any embodiment discussed with respect to one aspect of the invention applies to other aspects of the invention as well and vice versa. The embodiments in the Example and Detailed Description section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Percent (%) proliferation of eight human lung cancer cell lines treated with hsa-miR-34a and other compounds, relative to cells treated with negative control miRNA (100%). Abbreviations: miR-34a, hsa-miR-34a; siEg5, siRNA against the motor protein kinesin 11 (Eg5); Etopo, etoposide; NC, negative control miRNA. Standard deviations are indicated in the graph.

FIG. 2. Long-term effects of hsa-miR-34a on cultured human H226 lung cancer cell numbers. Equal numbers of H226 cells were electroporated with 1.6 μM hsa-miR-34a (white squares) or negative control miRNA (NC, black diamonds), seeded and propagated in regular growth medium. When the control cells reached confluence (days 6, 17 and 25), cells were harvested, counted and electroporated again with the respective miRNAs. The population doubling and cumulative cell counts was calculated and plotted on a linear scale. Arrows represent electroporation days. Abbreviation: miR-34a, hsa-miR-34a; NC, negative control miRNA.

FIG. 3. Percent (%) proliferation of H460 lung cancer cells following administration of various combinations of microRNAs. A positive sign under each bar in the graph indicates that the miRNA was present in the administered combination. Standard deviations are shown in the graph. Abbreviations: miR-34a, hsa-miR-34a; miR-124a, hsa-miR-124a; miR-126, hsa-miR-126; miR-147, hsa-miR-147; let-7b, hsa-let-7b; let-7c, hsa-let-7c; let-7g, hsa-let-7g; Etopo, etoposide; NC, negative control miRNA.

FIG. 4. Average tumor volumes in groups of six (n=6) mice carrying human H460 lung cancer xenografts. Palpable tumors were treated with hsa-miR-34a (white squares) or with a negative control miRNA (NC, black diamonds) on days 11, 14, and 17 (arrows). Standard deviations are shown in the graph. Data points with p values<0.1, <0.05 and <0.01 are indicated by a cross, an asterisk or circles, respectively. Abbreviation: miR-34a, hsa-miR-34a; NC, negative control miRNA.

FIG. 5. Percent (%) proliferation of hsa-miR-34a treated human prostate cancer cells relative to cells treated with negative control miRNA (100%). Abbreviations: miR-34a, hsa-miR-34a; siEg5, siRNA against the motor protein kinesin 11 (Eg5); NC, negative control miRNA. Standard deviations are indicated in the graph.

FIG. 6. Long-term effects of hsa-miR-34a on cultured human PPC-1, PC3 and Du145 prostate cancer cells. Equal numbers cells were electroporated with 1.6 μM hsa-miR-34a (white squares) or negative control miRNA (NC, black diamonds), seeded and propagated in regular growth medium. When the control cells reached confluence (days 4 and 11 for PPC-1, days 7 and 14 for PC3 and Du145), cells were harvested, counted and electroporated again with the respective miRNAs. The population doubling and cumulative cell counts was calculated and plotted on a linear scale. Arrows represent electroporation days. Experiments with PC3 and Du145 cells were carried out in triplicates. Standard deviations are shown in the graphs. Abbreviation: miR-34a, hsa-miR-34a; NC, negative control miRNA.

FIG. 7. Average tumor volumes in groups of seven (n=7) mice carrying human PPC-1 prostate cancer xenografts. Human PPC-1 prostate tumor cells were treated with hsa-miR-34a (white squares) or with a negative control miRNA (NC, black diamonds) on days 0, 7, 13, 20, and 25 (arrows). Tumor growth was determined by caliper measurements for 32 days. Standard deviations are shown in the graph. All data points yielded p values <0.01. The p value obtained from data on day 22 is indicated by a circle. Abbreviation: miR-34a, hsa-miR-34a; NC, negative control miRNA.

FIG. 8. Histology of tumors that developed from PPC-1 prostate cancer cells treated with negative control miRNA (right) or hsa-miR-34a (left). Images show tumors stained with hematoxylin and eosin. The arrow indicates a pocket with seemingly viable cells. Abbreviation: miR-34a, hsa-miR-34a; NC, negative control miRNA.

FIG. 9. Immunohistochemistry of PPC-1 tumors treated with negative control miRNA (top panels) or hsa-miR-34a (bottom panels). For hsa-miR-34a-treated tumors, the analysis is limited to areas with seemingly viable cells as shown in FIG. 8. Left images show tumor cells stained with hematoxylin and eosin (H&E); center images show an immunohistochemistry analysis using antibodies against the Ki-67 antigen (dark spotted areas); right images show an immunohistochemistry analysis using antibodies against caspase 3. Areas with increased apoptotic activity are exemplarily denoted by arrows. Abbreviation: miR-34a, hsa-miR-34a; NC, negative control miRNA.

### DETAILED DESCRIPTION OF THE INVENTION

The present invention is directed to compositions and methods relating to the identification and characterization of genes and biological pathways related to these genes as represented by the expression of the identified genes, as well as use of miRNAs related to such, for therapeutic, prognostic, and diagnostic applications, particularly those methods and compositions related to assessing and/or identifying pathological conditions directly or indirectly related to miR-34 expression or the aberrant expression thereof.

In certain aspects, the invention is directed to methods for the assessment, analysis, and/or therapy of a cell or subject where certain genes have a reduced or increased expression (relative to normal) as a result of an increased or decreased expression of any one or a combination of miR-34 family members (including, but not limited to SEQ ID NO:1 to SEQ ID NO:71) and/or genes with an increased expression (relative to normal) as a result of an increased or decreased expression of one or a combination of miR-34 family members. The expression profile and/or response to miR-34 expression or inhibition may be indicative of a disease or an individual with a condition, e.g., cancer.

Prognostic assays featuring any one or combination of the miRNAs listed or the markers listed (including nucleic acids representative thereof) could be used in assessment of a patient to determine what if any treatment regimen is justified. As with the diagnostic assays mentioned above, the absolute values that define low expression will depend on the platform used to measure the miRNA(s). The same methods described for the diagnostic assays could be used for prognostic assays.

### I. Therapeutic Methods

Embodiments of the invention concern nucleic acids that perform the activities of or inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA.

The present invention concerns, in some embodiments, short nucleic acid molecules that function as miRNAs or as inhibitors of miRNA in a cell. The term “short” refers to a length of a single polynucleotide that is 15, 16, 17, 18, 19, 20, 21, 22, 23, 24, 25, 50, 100, or 150 nucleotides or fewer, including all integers or ranges derivable there between. The nucleic acid molecules are typically synthetic. The term “synthetic” refers to nucleic acid molecule that is isolated and not produced naturally in a cell. In certain aspects the sequence (the entire sequence) and/or chemical structure deviates from a naturally-occurring nucleic acid molecule, such as an endogenous precursor miRNA or miRNA molecule or complement thereof. While in some embodiments, nucleic acids of the invention do not have an entire sequence that is identical or complementary to a sequence of a naturally-occurring nucleic acid, such molecules may encompass all or part of a naturally-occurring sequence or a complement thereof. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA or an inhibitor thereof. The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together. In certain aspects, synthetic miRNA of the invention are RNA or RNA analogs. miRNA inhibitors may be DNA or RNA, or analogs thereof. miRNA and miRNA inhibitors of the invention are collectively referred to as “synthetic nucleic acids.”

In some embodiments, there is a miRNA or a synthetic miRNA having a length of between 17 and 130 residues. The present invention concerns miRNA or synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range there between.

In certain embodiments, synthetic miRNA have (a) a “miRNA region” whose sequence or binding region from 5′ to 3′ is identical or complementary to all or a segment of a mature miRNA sequence, and (b) a “complementary region” whose sequence from 5′ to 3′ is between 60% and 100% complementary to the miRNA sequence in (a). In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence or a complement thereof. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA or complement thereof.

The term “complementary region” or “complement” refers to a region of a nucleic acid or mimetic that is or is at least 60% complementary to the mature, naturally occurring miRNA sequence. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, an miRNA inhibitor may have a sequence (from 5′ to 3′) that is or is at least 70, 75, 80, 85, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the miRNA sequence that is complementary to the sequence of a mature miRNA as the sequence for a miRNA inhibitor. Moreover, that portion of the nucleic acid sequence can be altered so that it is still comprises the appropriate percentage of complementarity to the sequence of a mature miRNA.

In some embodiments, of the invention, a synthetic miRNA or inhibitor contains one or more design element(s). These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region. A variety of design modifications are known in the art, see below.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an aminohexyl phosphate group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there are one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification, a 2′F modification, a 2′H modification, a 2′amino modification, a 4′thioribose modification or a phosphorothioate modification on the carboxy group linked to the carbon at position 6′. In further embodiments, there are one or more sugar modifications in the first or last 2 to 4 residues of the complementary region or the first or last 4 to 6 residues of the complementary region. This design element can also be used with a miRNA inhibitor. Thus, a miRNA inhibitor can have this design element and/or a replacement group on the nucleotide at the 5′ terminus, as discussed above.

In other embodiments of the invention, there is a synthetic miRNA or inhibitor in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA or inhibitor region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

Methods of the invention include reducing or eliminating activity of one or more miRNAs in a cell comprising introducing into a cell a miRNA inhibitor (which may be described generally herein as an miRNA, so that a description of miRNA, where appropriate, also will refer to a miRNA inhibitor); or supplying or enhancing the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or the miRNA inhibitor are synthetic, as discussed above.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced or inhibited miRNA function. It is contemplated, however, that the miRNA molecule introduced into a cell is not a mature miRNA but is capable of becoming or functioning as a mature miRNA under the appropriate physiological conditions. In cases in which a particular corresponding miRNA is being inhibited by a miRNA inhibitor, the particular miRNA will be referred to as the “targeted miRNA.” It is contemplated that multiple corresponding miRNAs may be involved. In particular embodiments, more than one miRNA molecule is introduced into a cell. Moreover, in other embodiments, more than one miRNA inhibitor is introduced into a cell. Furthermore, a combination of miRNA molecule(s) and miRNA inhibitor(s) may be introduced into a cell. The inventors contemplate that a combination of miRNA may act at one or more points in cellular pathways of cells with aberrant phenotypes and that such combination may have increased efficacy on the target cell while not adversely effecting normal cells. Thus, a combination of miRNA may have a minimal adverse effect on a subject or patient while supplying a sufficient therapeutic effect, such as amelioration of a condition, growth inhibition of a cell, death of a targeted cell, alteration of cell phenotype or physiology, slowing of cellular growth, sensitization to a second therapy, sensitization to a particular therapy, and the like.

Methods include identifying a cell or patient in need of inducing those cellular characteristics. Also, it will be understood that an amount of a synthetic nucleic acid that is provided to a cell or organism is an “effective amount,” which refers to an amount needed (or a sufficient amount) to achieve a desired goal, such as inducing a particular cellular characteristic(s).

In certain embodiments of the methods include providing or introducing to a cell a nucleic acid molecule corresponding to a mature miRNA in the cell in an amount effective to achieve a desired physiological result.

Moreover, methods can involve providing synthetic or nonsynthetic miRNA molecules. It is contemplated that in these embodiments, that methods may or may not be limited to providing only one or more synthetic miRNA molecules or only one or more nonsynthetic miRNA molecules. Thus, in certain embodiments, methods may involve providing both synthetic and nonsynthetic miRNA molecules. In this situation, a cell or cells are most likely provided a synthetic miRNA molecule corresponding to a particular miRNA and a nonsynthetic miRNA molecule corresponding to a different miRNA. Furthermore, any method articulated using a list of miRNAs using Markush group language may be articulated without the Markush group language and a disjunctive article (i.e., or) instead, and vice versa.

In some embodiments, there is a method for reducing or inhibiting cell proliferation in a cell comprising introducing into or providing to the cell an effective amount of (i) an miRNA inhibitor molecule or (ii) a synthetic or nonsynthetic miRNA molecule that corresponds to a miRNA sequence. In certain embodiments the methods involves introducing into the cell an effective amount of (i) a miRNA inhibitor molecule having a 5′ to 3′ sequence that is at least 90% complementary to the 5′ to 3′ sequence of one or more mature miRNA.

Certain embodiments of the invention include methods of treating a pathologic condition, in particular cancer, e.g., lung or liver cancer. In one aspect, the method comprises contacting a target cell with one or more nucleic acid, synthetic miRNA, or miRNA comprising at least one nucleic acid segment having all or a portion of a miRNA sequence. The segment may be 5, 6, 7, 8, 9, 10, 11, 12, 13, 14, 15, 16, 17, 18, 19, 20, 21, 22, 23, 24, 25, 30 or more nucleotides or nucleotide analog, including all integers there between. An aspect of the invention includes the modulation of gene expression, miRNA expression or function or mRNA expression or function within a target cell, such as a cancer cell.

Typically, an endogenous gene, miRNA or mRNA is modulated in the cell. In particular embodiments, the nucleic acid sequence comprises at least one segment that is at least 70, 75, 80, 85, 90, 95, or 100% identical in nucleic acid sequence to one or more miRNA or gene sequence. Modulation of the expression or processing of an endogenous gene, miRNA, or mRNA can be through modulation of the processing of a mRNA, such processing including transcription, transportation and/or translation with in a cell. Modulation may also be effected by the inhibition or enhancement of miRNA activity with a cell, tissue, or organ. Such processing may affect the expression of an encoded product or the stability of the mRNA. In still other embodiments, a nucleic acid sequence can comprise a modified nucleic acid sequence. In certain aspects, one or more miRNA sequence may include or comprise a modified nucleobase or nucleic acid sequence.

It will be understood in methods of the invention that a cell or other biological matter such as an organism (including patients) can be provided a miRNA or miRNA molecule corresponding to a particular miRNA by administering to the cell or organism a nucleic acid molecule that functions as the corresponding miRNA once inside the cell. The form of the molecule provided to the cell may not be the form that acts a miRNA once inside the cell. Thus, it is contemplated that in some embodiments, a synthetic miRNA or a nonsynthetic miRNA is provided such that it becomes processed into a mature and active miRNA once it has access to the cell's miRNA processing machinery. In certain embodiments, it is specifically contemplated that the miRNA molecule provided is not a mature miRNA molecule but a nucleic acid molecule that can be processed into the mature miRNA once it is accessible to miRNA processing machinery. The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa. It will be understand that the term “providing” an agent is used to include “administering” the agent to a patient.

In certain embodiments, methods also include targeting a miRNA to modulate in a cell or organism. The term “targeting a miRNA to modulate” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with a miRNA inhibitor, which effectively inhibits the targeted miRNA in the cell or organism (negative modulation).

In some embodiments, the miRNA targeted to be modulated is a miRNA that affects a disease, condition, or pathway. In certain embodiments, the miRNA is targeted because a treatment can be provided by negative modulation of the targeted miRNA. In other embodiments, the miRNA is targeted because a treatment can be provided by positive modulation of the targeted miRNA or its targets.

In certain methods of the invention, there is a further step of administering the selected miRNA modulator to a cell, tissue, organ, or organism (collectively “biological matter”) in need of treatment related to modulation of the targeted miRNA or in need of the physiological or biological results discussed herein (such as with respect to a particular cellular pathway or result like decrease in cell viability). Consequently, in some methods of the invention there is a step of identifying a patient in need of treatment that can be provided by the miRNA modulator(s). It is contemplated that an effective amount of a miRNA modulator can be administered in some embodiments. In particular embodiments, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a symptom. For example, with respect to cancer, it is contemplated that a therapeutic benefit can be inhibition of tumor growth, prevention of metastasis, reduction in number of metastases, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, and/or delay of death directly or indirectly related to cancer.

Furthermore, it is contemplated that the miRNA compositions may be provided as part of a therapy to a patient, in conjunction with traditional therapies or preventative agents. Moreover, it is contemplated that any method discussed in the context of therapy may be applied as preventatively, particularly in a patient identified to be potentially in need of the therapy or at risk of the condition or disease for which a therapy is needed.

In addition, methods of the invention concern employing one or more nucleic acids corresponding to a miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating cancer in a patient comprising administering to the patient the cancer therapeutic and an effective amount of at least one miRNA molecule that improves the efficacy of the cancer therapeutic or protects non-cancer cells. Cancer therapies also include a variety of combination therapies with both chemical and radiation based treatments. Combination chemotherapies include but are not limited to, for example, 5-fluorouracil, alemtuzumab, amrubicin, bevacizumab, bleomycin, bortezomib, busulfan, camptothecin, capecitabine, cisplatin (CDDP), carboplatin, cetuximab, chlorambucil, cisplatin (CDDP), cyclophosphamide, camptothecin, COX-2 inhibitors (e.g., celecoxib), cyclophosphamide, cytarabine, dactinomycin, dasatinib, daunorubicin, dexamethasone, docetaxel, doxorubicin (adriamycin), EGFR inhibitors (gefitinib and cetuximab), erlotinib, estrogen receptor binding agents, etoposide (VP16), everolimus, farnesyl-protein transferase inhibitors, gefitinib, gemcitabine, gemtuzumab, ibritumomab, ifosfamide, imatinib mesylate, larotaxel, lapatinib, lonafarnib, mechlorethamine, melphalan, methotrexate, mitomycin, navelbine, nitrosurea, nocodazole, oxaliplatin, paclitaxel, plicomycin, procarbazine, raloxifene, rituximab, sirolimus, sorafenib, sunitinib, tamoxifen, taxol, taxotere, temsirolimus, tipifarnib, tositumomab, transplatinum, trastuzumab, vinblastin, vincristin, or vinorelbine or any analog or derivative variant of the foregoing.

Generally, inhibitors of miRNAs can be given to decrease the activity of an endogenous miRNA. Similarly, nucleic acid molecules corresponding to the mature miRNA can be given to achieve the opposite effect as compared to when inhibitors of the miRNA are given. For example, inhibitors of miRNA molecules that increase cell proliferation can be provided to cells to increase proliferation or decrease cell proliferation. The present invention contemplates these embodiments in the context of the different physiological effects observed with the different miRNA molecules and miRNA inhibitors disclosed herein. These include, but are not limited to, the following physiological effects: increase and decreasing cell proliferation, increasing or decreasing apoptosis, increasing transformation, increasing or decreasing cell viability, activating or inhibiting a kinase (e.g., Erk), activating/inducing or inhibiting hTert, inhibit stimulation of growth promoting pathway (e.g., Stat 3 signaling), reduce or increase viable cell number, and increase or decrease number of cells at a particular phase of the cell cycle. Methods of the invention are generally contemplated to include providing or introducing one or more different nucleic acid molecules corresponding to one or more different miRNA molecules. It is contemplated that the following, at least the following, or at most the following number of different nucleic acid or miRNA molecules may be provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein. This also applies to the number of different miRNA molecules that can be provided or introduced into a cell.

### II. Pharmaceutical Formulations and Delivery

Methods of the present invention include the delivery of an effective amount of a miRNA or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease.

A. Administration

In certain embodiments, it is desired to kill cells, inhibit cell growth, inhibit metastasis, decrease tumor or tissue size, and/or reverse or reduce the malignant or disease phenotype of cells. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation. Direct injection, intratumoral injection, or injection into tumor vasculature is specifically contemplated for discrete, solid, accessible tumors, or other accessible target areas. Local, regional, or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as a single dose comprise about 0.1 to about 0.5 ml volumes. Compositions of the invention may be administered in multiple injections to a tumor or a targeted site. In certain aspects, injections may be spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a miRNA or combinations thereof. Administration may be continued post-resection, for example, by leaving a catheter implanted at the site of the surgery. Periodic post-surgical treatment also is envisioned. Continuous perfusion of an expression construct or a viral construct also is contemplated.

Continuous administration also may be applied where appropriate, for example, where a tumor or other undesired affected area is excised and the tumor bed or targeted site is treated to eliminate residual, microscopic disease. Delivery via syringe or catherization is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well and often depend on tumor type, tumor location, immune condition, target site, disease progression, and health and age of the patient. Certain tumor types will require more aggressive treatment. The clinician will be best suited to make such decisions based on the known efficacy and toxicity (if any) of the therapeutic formulations.

In certain embodiments, the tumor or affected area being treated may not, at least initially, be resectable. Treatments with compositions of the invention may increase the resectability of the tumor due to shrinkage at the margins or by elimination of certain particularly invasive portions. Following treatments, resection may be possible. Additional treatments subsequent to resection may serve to eliminate microscopic residual disease at the tumor or targeted site.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. With respect to a viral component of the present invention, a unit dose may conveniently be described in terms of μg or mg of miRNA or miRNA mimetic. Alternatively, the amount specified may be the amount administered as the average daily, average weekly, or average monthly dose.

miRNA can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μg or mg, or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

B. Injectable Compositions and Formulations

In some embodiments, the method for the delivery of a miRNA or an expression construct encoding such or combinations thereof is via systemic administration. However, the pharmaceutical compositions disclosed herein may also be administered parenterally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of nucleic acids may be delivered by syringe or any other method used for injection of a solution, as long as the nucleic acid and any associated components can pass through the particular gauge of needle required for injection. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising a tumor suppressor protein or a nucleic acid encoding the same is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral, intralesional, and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human.

The nucleic acid(s) are administered in a manner compatible with the dosage formulation, and in such amount as will be therapeutically effective. The quantity to be administered depends on the subject to be treated, including, e.g., the aggressiveness of the disease or cancer, the size of any tumor(s) or lesions, the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

Various methods for nucleic acid delivery are described, for example in Sambrook et al., 1989 and Ausubel et al., 1994. Such nucleic acid delivery systems comprise the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. By way of example, and not by limitation, the nucleic acid delivery system can be provided to the cell by endocytosis; receptor targeting; coupling with native or synthetic cell membrane fragments; physical means such as electroporation; combining the nucleic acid delivery system with a polymeric carrier, such as a controlled release film or nanoparticle or microparticle or biocompatible molecules or biodegradable molecules; with vector. The nucleic acid delivery system can be injected into a tissue or fluid surrounding the cell, or administered by diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

C. Combination Treatments

In certain embodiments, the compositions and methods of the present invention involve a miRNA, or expression construct encoding such. These miRNA composition can be used in combination with a second therapy to enhance the effect of the miRNA therapy, or increase the therapeutic effect of another therapy being employed. These compositions would be provided in a combined amount effective to achieve the desired effect, such as the killing of a cancer cell and/or the inhibition of cellular hyperproliferation. This process may involve contacting the cells with the miRNA or second therapy at the same or different time. This may be achieved by contacting the cell with one or more compositions or pharmacological formulation that includes or more of the agents, or by contacting the cell with two or more distinct compositions or formulations, wherein one composition provides (1) miRNA; and/or (2) a second therapy. A second composition or method may be administered that includes a chemotherapy, radiotherapy, surgical therapy, immunotherapy or gene therapy.

It is contemplated that one may provide a patient with the miRNA therapy and the second therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example miRNA therapy is “A” and a second therapy is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a second therapy, such as chemotherapy, radiotherapy, immunotherapy, surgical therapy or other gene therapy, is employed in combination with the miRNA therapy, as described herein.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents.

b. Antimetabolites

Antimetabolites disrupt DNA and RNA synthesis. Unlike alkylating agents, they specifically influence the cell cycle during S phase. They have been used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

c. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

d. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

e. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. Radiotherapy may comprise the use of radiolabeled antibodies to deliver doses of radiation directly to the cancer site (radioimmunotherapy). Once injected into the body, the antibodies actively seek out the cancer cells, which are destroyed by the cell-killing (cytotoxic) action of the radiation. This approach can minimize the risk of radiation damage to healthy cells.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment a specially made metal frame is attached to the head. Then, several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

J. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor or disease cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Table 6 is a non-limiting list of several known anti-cancer immunotherapeutic agents and their targets. It is contemplated that one or more of these therapies may be employed with the miRNA therapies described herein.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

TABLE 6
Cancer immunotherapeutics and their targets.

| Generic Name | Target |
|---|---|
| Cetuximab | EGFR |
| Panitumumab | EGFR |
| Trastuzumab | erbB2 receptor |
| Bevacizumab | VEGF |
| Alemtuzumab | CD52 |
| Gemtuzumab ozogamicin | CD33 |
| Rituximab | CD20 |
| Tositumomab | CD20 |
| Matuzumab | EGFR |
| Ibritumomab tiuxetan | CD20 |
| Tositumomab | CD20 |
| HuPAM4 | MUC1 |
| MORAb-009 | Mesothelin |
| G250 | carbonic anhydrase IX |
| mAb 8H9 | 8H9 antigen |
| M195 | CD33 |
| Ipilimumab | CTLA4 |
| HuLuc63 | CS1 |
| Alemtuzumab | CD53 |
| Epratuzumab | CD22 |
| BC8 | CD45 |
| HuJ591 | Prostate specific membrane antigen |
| hA20 | CD20 |
| Lexatumumab | TRAIL receptor-2 |
| Pertuzumab | HER-2 receptor |
| Mik-beta-1 | IL-2R |
| RAV12 | RAAG12 |
| SGN-30 | CD30 |
| AME-133v | CD20 |
| HeFi-1 | CD30 |
| BMS-663513 | CD137 |
| Volociximab | anti-α5β1 integrin |
| GC1008 | TGFβ |
| HCD122 | CD40 |
| Siplizumab | CD2 |
| MORAb-003 | Folate receptor alpha |
| CNTO 328 | IL-6 |
| MDX-060 | CD30 |
| Ofatumumab | CD20 |
| SGN-33 | CD33 |

4. Gene Therapy

In yet another embodiment, a combination treatment involves gene therapy in which a therapeutic polynucleotide is administered before, after, or at the same time as one or more therapeutic miRNA. Delivery of a therapeutic polypeptide or encoding nucleic acid in conjunction with a miRNA may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

This application incorporates U.S. application Ser. No. 11/349,727 filed on Feb. 8, 2006 claiming priority to U.S. Provisional Application Ser. No. 60/650,807 filed Feb. 8, 2005 herein by references in its entirety.

### III. Mirna Molecules

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene or its gene product. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

A. Array Preparation

Certain embodiments of the present invention concerns the preparation and use of mRNA or nucleic acid arrays, miRNA or nucleic acid arrays, and/or miRNA or nucleic acid probe arrays, which are macroarrays or microarrays of nucleic acid molecules (probes) that are fully or nearly complementary (over the length of the prove) or identical (over the length of the prove) to a plurality of nucleic acid, mRNA or miRNA molecules, precursor miRNA molecules, or nucleic acids derived from the various genes and gene pathways modulated by miR-34 miRNAs and that are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of marker RNA and/or miRNA-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect miRNA, or genes or nucleic acid representative of genes; consequently, methods and compositions may be used with a variety of different types of nucleic acid arrays.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 2, 20, 25, 50, 80, 100 or more different probes. It is contemplated that they may contain 1000, 16,000, 65,000, 250,000 or 1,000,000 or more different probes. The probes can be directed to mRNA and/or miRNA targets in one or more different organisms or cell types. The oligonucleotide probes range from 5 to 50, 5 to 45, 10 to 40, 9 to 34, or 15 to 40 nucleotides in length in some embodiments. In certain embodiments, the oligonucleotide probes are 5, 10, 15, 20 to 20, 25, 30, 35, 40 nucleotides in length including all integers and ranges there between.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

B. Sample Preparation

It is contemplated that the RNA and/or miRNA of a wide variety of samples can be analyzed using the arrays, index of probes, or array technology of the invention. While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from biopsy, fine needle aspirates, exfoliates, blood, tissue, organs, semen, saliva, tears, other bodily fluid, hair follicles, skin, or any sample containing or constituting biological cells, particularly cancer or hyperproliferative cells. In certain embodiments, samples may be, but are not limited to, biopsy, or cells purified or enriched to some extent from a biopsy or other bodily fluids or tissues. Alternatively, the sample may not be a biological sample, but be a chemical mixture, such as a cell-free reaction mixture (which may contain one or more biological enzymes).

C. Hybridization

After an array or a set of probes is prepared and/or the nucleic acid in the sample or probe is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a tumor tissue sample labeled with Cy3, and normal tissue sample labeled with Cy5. Differences between the samples for particular miRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

D. Differential Expression Analyses

Arrays of the invention can be used to detect differences between two samples. Specifically contemplated applications include identifying and/or quantifying differences between miRNA or gene expression from a sample that is normal and from a sample that is not normal, between a disease or condition and a cell not exhibiting such a disease or condition, or between two differently treated samples. Also, miRNA or gene expression may be compared between a sample believed to be susceptible to a particular disease or condition and one believed to be not susceptible or resistant to that disease or condition. A sample that is not normal is one exhibiting phenotypic or genotypic trait(s) of a disease or condition, or one believed to be not normal with respect to that disease or condition. It may be compared to a cell that is normal with respect to that disease or condition. Phenotypic traits include symptoms of, or susceptibility to, a disease or condition of which a component is or may or may not be genetic, or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., (1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics, which is incorporated by reference in its entirety for all purposes.

Particularly, arrays can be used to evaluate samples with respect to pathological condition such as cancer and related conditions. It is specifically contemplated that the invention can be used to evaluate differences between stages or sub-classifications of disease, such as between benign, cancerous, and metastatic tissues or tumors.

Phenotypic traits to be assessed include characteristics such as longevity, morbidity, expected survival, susceptibility or receptivity to particular drugs or therapeutic treatments (drug efficacy), and risk of drug toxicity. Samples that differ in these phenotypic traits may also be evaluated using the compositions and methods described.

In certain embodiments, miRNA and/or expression profiles may be generated to evaluate and correlate those profiles with pharmacokinetics or therapies. For example, these profiles may be created and evaluated for patient tumor and blood samples prior to the patient's being treated or during treatment to determine if there are miRNA or genes whose expression correlates with the outcome of the patient's treatment. Identification of differential miRNAs or genes can lead to a diagnostic assay for evaluation of tumor and/or blood samples to determine what drug regimen the patient should be provided. In addition, it can be used to identify or select patients suitable for a particular clinical trial. If an expression profile is determined to be correlated with drug efficacy or drug toxicity, that profile is relevant to whether that patient is an appropriate patient for receiving a drug, for receiving a combination of drugs, or for a particular dosage of the drug.

In addition to the above prognostic assay, samples from patients with a variety of diseases can be evaluated to determine if different diseases can be identified based on miRNA and/or related gene expression levels. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005 in the names of David Brown, Lance Ford, Angie Cheng and Rich Jarvis, which is hereby incorporated by reference in its entirety.

E. Other Assays

In addition to the use of arrays and microarrays, it is contemplated that a number of different assays could be employed to analyze miRNAs or related genes, their activities, and their effects. Such assays include, but are not limited to, nucleic acid amplification, polymerase chain reaction, quantitative PCR, RT-PCR, in situ hybridization, Northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### IV. Nucleic Acids

The present invention concerns nucleic acids, modified or mimetic nucleic acids, miRNAs, mRNAs, genes, and representative fragments thereof that can be labeled, used in array analysis, or employed in diagnostic, therapeutic, or prognostic applications, particularly those related to pathological conditions such as cancer. The molecules may have been endogenously produced by a cell, or been synthesized or produced chemically or recombinantly. They may be isolated and/or purified. Each of the miRNAs described herein and includes the corresponding SEQ ID NO and accession numbers for these miRNA sequences. The name of a miRNA is often abbreviated and referred to without a “hsa-” prefix and will be understood as such, depending on the context. Unless otherwise indicated, miRNAs referred to in the application are human sequences identified as miR-X or let-X, where X is a number and/or letter.

In certain aspects, a miRNA probe designated by a suffix “5P” or “3P” can be used. “5P” indicates that the mature miRNA derives from the 5′ end of the precursor and a corresponding “3P” indicates that it derives from the 3′ end of the precursor, as described on the world wide web at sanger.ac.uk. Moreover, in some embodiments, a miRNA probe is used that does not correspond to a known human miRNA. It is contemplated that these non-human miRNA probes may be used in embodiments of the invention or that there may exist a human miRNA that is homologous to the non-human miRNA. In other embodiments, any mammalian cell, biological sample, or preparation thereof may be employed.

In some embodiments of the invention, methods and compositions involving miRNA may concern miRNA, markers (mRNAs), and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, mRNA, mRNA probes, control nucleic acids, and other probes and primers.

In many embodiments, miRNA are 19-24 nucleotides in length, while miRNA probes are 19-35 nucleotides in length, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides. It is further understood that the length of complementarity within a precursor miRNA or other nucleic acid or between a miRNA probe and a miRNA or a miRNA gene are such lengths. Moreover, the complementarity may be expressed as a percentage, meaning that the complementarity between a probe and its target is 90% or greater over the length of the probe. In some embodiments, complementarity is or is at least 90%, 95% or 100%. In particular, such lengths may be applied to any nucleic acid comprising a nucleic acid sequence identified in any of SEQ ID NOs described herein, accession number, or any other sequence disclosed herein. Typically, the commonly used name of the miRNA is given (with its identifying source in the prefix, for example, “hsa” for human sequences) and the processed miRNA sequence. Unless otherwise indicated, a miRNA without a prefix will be understood to refer to a human miRNA. Moreover, a lowercase letter in a miRNA name may or may not be lowercase; for example, hsa-mir-130b can also be referred to as miR-130B. The term “miRNA probe” refers to a nucleic acid probe that can identify a particular miRNA or structurally related miRNAs.

It is understood that some nucleic acids are derived from genomic sequences or a gene. In this respect, the term “gene” is used for simplicity to refer to the genomic sequence encoding the precursor nucleic acid or miRNA for a given miRNA or gene. However, embodiments of the invention may involve genomic sequences of a miRNA that are involved in its expression, such as a promoter or other regulatory sequences.

The term “recombinant” may be used and this generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids of the invention may encompass a molecule that comprises one or more complementary or self-complementary strand(s) or “complement(s)” of a particular sequence. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

It is understood that a “synthetic nucleic acid” of the invention means that the nucleic acid does not have all or part of a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

While embodiments of the invention may involve synthetic miRNAs or synthetic nucleic acids, in some embodiments of the invention, the nucleic acid molecule(s) need not be “synthetic.” In certain embodiments, a non-synthetic nucleic acid or miRNA employed in methods and compositions of the invention may have the entire sequence and structure of a naturally occurring mRNA or miRNA precursor or the mature mRNA or miRNA. For example, non-synthetic miRNAs used in methods and compositions of the invention may not have one or more modified nucleotides or nucleotide analogs. In these embodiments, the non-synthetic miRNA may or may not be recombinantly produced. In particular embodiments, the nucleic acid in methods and/or compositions of the invention is specifically a synthetic miRNA and not a non-synthetic miRNA (that is, not a miRNA that qualifies as “synthetic”); though in other embodiments, the invention specifically involves a non-synthetic miRNA and not a synthetic miRNA. Any embodiments discussed with respect to the use of synthetic miRNAs can be applied with respect to non-synthetic miRNAs, and vice versa.

It will be understood that the term “naturally occurring” refers to something found in an organism without any intervention by a person; it could refer to a naturally-occurring wildtype or mutant molecule. In some embodiments a synthetic miRNA molecule does not have the sequence of a naturally occurring miRNA molecule. In other embodiments, a synthetic miRNA molecule may have the sequence of a naturally occurring miRNA molecule, but the chemical structure of the molecule, particularly in the part unrelated specifically to the precise sequence (non-sequence chemical structure) differs from chemical structure of the naturally occurring miRNA molecule with that sequence. In some cases, the synthetic miRNA has both a sequence and non-sequence chemical structure that are not found in a naturally-occurring miRNA. Moreover, the sequence of the synthetic molecules will identify which miRNA is effectively being provided or inhibited; the endogenous miRNA will be referred to as the “corresponding miRNA.” Corresponding miRNA sequences that can be used in the context of the invention include, but are not limited to, all or a portion of those sequences in the SEQ IDs provided herein, as well as any other miRNA sequence, miRNA precursor sequence, or any sequence complementary thereof. In some embodiments, the sequence is or is derived from or contains all or part of a sequence identified herein to target a particular miRNA (or set of miRNAs) that can be used with that sequence. Any 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130 140, 150, 160, 170, 180, 190, 200, 210, 220, 230, 240, 250, 260 or any number or range of sequences there between may be selected to the exclusion of all non-selected sequences.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

A. Nucleobase, Nucleoside, Nucleotide, and Modified Nucleotides

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring. Different types of covalent attachment(s) of a nucleobase to a nucleobase linker moiety are known in the art (Kornberg and Baker, 1992).

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids include those in: U.S. Pat. Nos. 5,681,947, 5,652,099 and 5,763,167, 5,614,617, 5,670,663, 5,872,232, 5,859,221, 5,446,137, 5,886,165, 5,714,606, 5,672,697, 5,466,786, 5,792,847, 5,223,618, 5,470,967, 5,378,825, 5,777,092, 5,623,070, 5,610,289, 5,602,240, 5,858,988, 5,214,136, 5,700,922, 5,708,154, 5,728,525, 5,637,683, 6,251,666, 5,480,980, and 5,728,525, each of which is incorporated herein by reference in its entirety.

Labeling methods and kits of the invention specifically contemplate the use of nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the invention are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and the other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in many embodiments is alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, Biosearch Technologies and NEN. Functional groups may be prepared according to ways known to those of skill in the art, including the representative information found in U.S. Pat. Nos. 4,404,289; 4,405,711; 4,337,063 and 5,268,486, and U.K. Patent 1,529,202, which are all incorporated by reference.

Amine-modified nucleotides are used in several embodiments of the invention. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N6-(4-amino)butyl-dATP, N6-(6-amino)butyl-dATP, N4-[2,2-oxy-bis-(ethylamine)]-dCTP; N6-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

B. Preparation of Nucleic Acids

A nucleic acid may be made by any technique known to one of ordinary skill in the art, such as for example, chemical synthesis, enzymatic production, or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

Alternatively, nucleic acid synthesis is performed according to standard methods. See, for example, Itakura and Riggs (1980) and U.S. Pat. Nos. 4,704,362, 5,221,619, and 5,583,013, each of which is incorporated herein by reference. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. See also Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

C. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for forming a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried and resuspended in a liquid and volume appropriate for subsequent manipulation.

### V. Labels and Labeling Techniques

In some embodiments, the present invention concerns miRNA that are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

A. Labeling Techniques

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. Enzymes capable of adding such nucleotides include, but are not limited to, poly(A)polymerase, terminal transferase, and polynucleotide phosphorylase. In specific embodiments of the invention, a ligase is contemplated as not being the enzyme used to add the label, and instead, a non-ligase enzyme is employed. Terminal transferase catalyzes the addition of nucleotides to the 3′ terminus of a nucleic acid. Polynucleotide phosphorylase can polymerize nucleotide diphosphates without the need for a primer.

B. Labels

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phycoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

C. Visualization Techniques

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### VI. Kits

Any of the compositions described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from blood samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: (1) poly(A) polymerase; (2) unmodified nucleotides (G, A, T, C, and/or U); (3) a modified nucleotide (labeled or unlabeled); (4) poly(A) polymerase buffer; and, (5) at least one microfilter; (6) label that can be attached to a nucleotide; (7) at least one miRNA probe; (8) reaction buffer; (9) a miRNA array or components for making such an array; (10) acetic acid; (11) alcohol; (12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (acute myeloid leukemia), (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ IDs described herein. In certain embodiments, a kit or array of the invention can contain one or more probes for the miRNAs identified by the SEQ IDs described herein. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

Such kits may also include components that facilitate isolation of the labeled miRNA. It may also include components that preserve or maintain the miRNA or that protect against its degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Gene Expression Analysis Following Transfection with hsa-miR-34a

miRNAs are believed to regulate gene expression by binding to target mRNA transcripts and (1) initiating transcript degradation or (2) altering protein translation from the transcript. Translational regulation leading to an up or down change in protein expression may lead to changes in activity and expression of downstream gene products and genes that are in turn regulated by those proteins. These numerous regulatory effects may be revealed as changes in the global mRNA expression profile. Microarray gene expression analyses were performed to identify genes that are mis-regulated by hsa-miR-34a expression.

Synthetic pre-miR-34a (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Alogrithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1.

Manipulation of the expression levels of the genes listed in Table 1 represents a potentially useful therapy for cancer and other diseases in which increased or reduced expression of hsa-miR-34a has a role in the disease.

### Example 2

### Cellular Pathways Affected by hsa-miR-34a

The mis-regulation of gene expression by hsa-miR-34a (Table 1) affects many cellular pathways that represent potential therapeutic targets for the control of cancer and other diseases and disorders. The inventors determined the identity and nature of the cellular genetic pathways affected by the regulatory cascade induced by hsa-miR-34a expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following over-expression of hsa-miR-34a in A549 cells are shown in Table 2.

These data demonstrate that hsa-miR-34a directly or indirectly affects the expression of numerous cancer-, cellular proliferation-, cellular development-, cell signaling-, and cell cycle-related genes and thus primarily affects functional pathways related to cancer, cellular growth, development, and proliferation. Those cellular processes all have integral roles in the development and progression of various cancers. Manipulation of the expression levels of genes in the cellular pathways shown in Table 2 represents a potentially useful therapy for cancer and other diseases in which increased or reduced expression of hsa-miR-34a has a role in the disease.

### Example 3

### Predicted Gene Targets of hsa-miR-34a

Gene targets for binding of and regulation by hsa-miR-34a were predicted using the proprietary algorithm miRNATarget™ (Asuragen), which is an implementation of the method proposed by Krek et al. (2005). Predicted target genes are shown in Table 3.

The predicted gene targets that exhibited altered mRNA expression levels in human cancer cells, following transfection with pre-miR hsa-miR-34a, are shown in Table 4.

The predicted gene targets of hsa-miR-34a whose mRNA expression levels are affected by hsa-miR-34a represent particularly useful candidates for cancer therapy and therapy of other diseases through manipulation of their expression levels.

### Example 4

### Cancer Related Gene Expression Altered by hsa-miR-34a

Cell proliferation and survival pathways are commonly altered in tumors (Hanahan and Weinberg, 2000). The inventors have shown that hsa-miR-34a directly or indirectly regulates the transcripts of proteins that are critical in the regulation of these pathways. A detailed list of hsa-miR-34a targets that are associated with various cancer types are shown in Table 5. Hsa-miR-34a targets of particular interest are genes and their products that function in the regulation of intracellular signal transduction, cell cycle, chromosomal maintenance, cell adhesion and migration, mRNA translation, DNA replication, transcription, apoptosis and the thioredoxin redox system. Many of these targets have inherent oncogenic or tumor suppressor activity and, when deregulated, contribute to the malignant phenotype in vitro and in vivo. Hsa-miR-34a affects intracellular signaling at various layers and controls the expression of secretory growth factors, transmembrane growth factor receptors as well as cytoplasmic signaling molecules. Examples of secreted proteins regulated by hsa-miR-34a are amphiregulin (AREG), connective tissue growth factor (CTGF), tumor growth factor β-2 (TGFB2) and the inflammatory chemokine interleukin 8 (IL8). IL-8 is frequently upregulated in various cancers and correlates with tumor vascularization, metastasis and poor prognosis (Rosenkilde and Schwartz, 2004; Sparmann and Bar-Sagi, 2004). Amphiregulin functions as a ligand to epidermal growth factor receptor (EGFR) and activates EGFR dependent signaling (Hynes and Lane, 2005). Amphiregulin is frequently expressed in ovarian, gastric and pancreatic carcinoma as well as hepatocellular carcinoma tissues and cell lines (Kitadai et al., 1993; Ebert et al., 1994; D'Antonio et al., 2002; Castillo et al., 2006). Amphiregulin acts as a mitogenic and anti-apoptotic growth factor in hepatocarcinoma cells and contributes to the transformed phenotype of liver cancer cells. Inhibition of amphiregulin function by small interfering RNA (siRNA) or neutralizing antibodies diminishes the amphiregulin-mediated autocrine loop and oncogenic properties of hepatocarcinoma cells (Castillo et al., 2006). Amphiregulin expression also progressively increases from benign to malignant stages of prostate cancer and is indicative for poor response to treatment with the FDA-approved drug Iressa (gefitinib) in patients with non-small cell lung cancer (NSCLC) (Bostwick et al., 2004; Ishikawa et al., 2005).

CTGF (also referred to as insulin-like growth factor binding protein 8; IGFBP8) was originally described as a mitogen produced by umbilical vein endothelial cells (Bradham et al., 1991). CTGF functions as a modulator of growth factor activity and is overexpressed in various tumors (Hishikawa et al., 1999; Shimo et al., 2001; Lin et al., 2005; Yang et al., 2005). CTGF is induced by hypoxia and enhances angiogenesis as well as the growth of tumor xenografts (Shimo et al., 2001; Yang et al., 2005). However, a coherent role for CTGF in cancer remains elusive and may depend on the cellular context (Hishikawa et al., 1999; Lin et al., 2005). TGF-β2 is the corresponding ligand to TGF-β receptors (TGFBR), a class of receptors that may function as tumor suppressors. Among these is TGFBR-2 which is also regulated by hsa-miR-34a. TGFBR-2 forms a functional complex with TGFBR-1 and is the primary receptor for TGF-β (Massague et al., 2000). Central role of TGF-β is inhibition of cellular growth of numerous cell types, such as epithelial, endothelial, hematopoietic neural and mesenchymal cells. Many mammary and colorectal carcinomas with microsatellite instability harbor inactivating mutations of TGFBR-2 and therefore escape the growth-inhibitory function of TGF-β (Markowitz et al., 1995; Lucke et al., 2001).

Other transmembrane growth factor receptors regulated by hsa-miR-34a include Met and the Ras association domain family protein 2 (RASSF2). RASSF2 is a tumor suppressor candidate that is frequently downregulated in lung tumor cell lines (Vos et al., 2003). RASSF2 interacts with K-Ras and promotes cell cycle arrest and apoptosis. Met acts as the receptor for hepatocyte growth factor (HGF) and was originally isolated as an oncogene from a chemically transformed human cell line (Cooper et al., 1984; Dean et al., 1985). Met activating mutations are found in sporadic papillary renal cancer, childhood hepatocellular carcinoma and gastric cancer (Danilkovitch-Miagkova and Zbar, 2002). These somatic mutations are associated with increased aggressiveness and extensive metastases in various carcinomas. In several other cancer types, autocrine and paracrine mechanisms lead to an activation of Met signaling. The most frequent mechanism of Met activation, however, is overexpression which occurs in colorectal cancer, hepatocellular carcinoma, gastrinomas as well as carcinomas of the stomach, pancreas, prostate, ovary and breast (Boccaccio and Comoglio, 2006). Met overexpression correlates with a metastatic tumor phenotype and poor prognosis (Birchmeier et al., 2003). Cytoplasmic signaling molecules regulated by hsa-miR-34a include PIK3CD, neurofibromin 1 and 2 (NF1, NF2) and AKAP12. AKAP12, also referred to as gravin or SSeCKS (Src suppressed C kinase substrate), functions as a kinase scaffold protein that tethers the enzyme-substrate interaction (Nauert et al., 1997). Expression of AKAP12 interferes with oncogenic cell transformation induced by the Src or Jun oncoproteins in vitro and is lost or reduced in numerous cancers, such as leukemia and carcinomas of the rectum, lung and stomach (Lin and Gelman, 1997; Cohen et al., 2001; Xia et al., 2001; Wikman et al., 2002; Boultwood et al., 2004; Choi et al., 2004; Mori et al., 2006). An apparent anti-oncogenic activity of AKAP12 in prostate and gastric cancers marks this protein as a putative tumor suppressor (Xia et al., 2001; Choi et al., 2004). PIK3CD encodes p110δ, the delta catalytic subunit of class IA phosphoinositide 3-kinases (PI3K). Similar to the well characterized p110α isoform, p110δ activates the Akt signaling pathway in response to most upstream receptor tyrosine kinases (Vanhaesebroeck et al., 1997). PIK3CD is consistently expressed at high levels in blasts cells from patients with acute myeloid leukemia (AML); inhibition of PIK3CD activity specifically blocks AML cell proliferation (Sujobert et al., 2005; Billottet et al., 2006). NF1 and NF2 are bona fide tumor suppressors which—when either of them is lost or mutated—are the cause of neurofibromatosis, one of the most commonly inherited tumor-predisposition syndromes (Rubin and Gutmann, 2005). Loss of NF1 or NF2 function occurs also in other malignancies, such as astrocytomas, gliomas and leukemia for NF1 and hepatocellular and thyroid carcinomas for NF2 (McClatchey and Giovannini, 2005; Rubin and Gutmann, 2005). The tumor suppressor function of NF1 can be explained by the fact that NF1 acts as a GTPase activating protein (GAP) towards the inherently oncogenic RAS protein, inactivating RAS by catalyzing the RAS-associated GTP into GDP. In contrast, the tumor suppressor role for NF2 is less well defined. NF2, also known as merlin or schwannomin, associates with the cellular membrane as well as the cytoskeleton and regulates membrane organization. Overexpression or constitutive activation of NF2 can block cell proliferation and oncogenic transformation (Tikoo et al., 1994; Lutchman and Rouleau, 1995; Jin et al., 2006).

Another class of genes and their corresponding proteins that are regulated by hsa-miR-34a, functions in the progression of the cell cycle. Some of these proteins play pivotal roles in the transition through G1 and S phases, such as retinoblastoma-like 1 (RBL1), cyclins D1, D3, A2 (CCND1, CCND3, CCNA2), cyclin dependent kinase 4 (CDK4) and CDK inhibitor 2c (CDKN2C). Others are required for proper segregation of sister chromatids during mitosis to maintain chromosomal stability. These include aurora kinase B (AURKB, STK12), breast cancer 1 and 2 (BRCA1; BRCA2), budding uninhibited by benzimidazoles 1 (BUB1), polo-like kinase 1 (PLK1) and cell division cycle 23 (CDC23, anaphase promoting complex subunit 8). BRCA1, BRCA2 and aurora kinase B show deregulated expression in a various solid tumors, e.g., carcinomas of the breast, ovary, thyroid gland, lung, prostate and colorectum (Wooster and Weber, 2003; Keen and Taylor, 2004; Turner et al., 2004; Smith et al., 2005; Chieffi et al., 2006; Ulisse et al., 2006). PLK1 (also referred to as serine-threonine protein kinase 13; STPK13) is a protein kinase that regulates mitotic spindle function to maintain chromosomal stability (Strebhardt and Ullrich, 2006). PLK1 expression is tightly regulated during the cell cycle and peaks in M phase. PLK1 is inherently oncogenic and directly inhibits the tumor suppressor function of p53 (Ando et al., 2004). Overexpression of PLK1 induces a polynucleated phenotype and cellular transformation of NIH3T3 cells (Mundt et al., 1997; Smith et al., 1997). Likewise, PLK1 shows increased expression levels in most solid tumors, including carcinomas of the breast, colon, lung, stomach and prostate (Table 5). PLK1 overexpression is associated with disease progression and—when depleted—induces apoptosis in cancer cells (Liu and Erikson, 2003; Strebhardt and Ullrich, 2006). Currently, PLK1 is being tested as a target of various small molecule inhibitors for future therapeutic intervention (Strebhardt and Ullrich, 2006).

RBL1, also known as p107, is a member of the retinoblastoma tumor suppressor protein family that includes the pocket proteins p107, p130 and pRb. Similar to the pRb prototype, RBL1 interacts with the E2F family of transcription factors and blocks cell cycle progression and DNA replication (Sherr and McCormick, 2002). Accordingly, a subset of cancers show deregulated expression of RBL1 (Takimoto et al., 1998; Claudio et al., 2002; Wu et al., 2002; Ito et al., 2003). Cyclins are co-factors of cyclin-dependent kinases (CDKs) (Malumbres and Barbacid, 2001). The expression of cyclins is tightly controlled during the cell cycle to govern the activity of individual CDKs. Cyclin A2 associates with CDK2 during S phase; cyclin D1 is the predominant co-factor of CDK4/6 in G1 phase. Since many cyclins are promoters of cell growth, cyclins—such as cyclin D1—are frequently expressed at high levels in various tumor types (Donnellan and Chetty, 1998). CDK4 forms active complexes with D-type cyclins, including D1, D2 and D3. Primary function of CDK4 is to inactivate members of the retinoblastoma protein family. CDK4 is overexpressed in numerous cancers and is currently being explored as a potential cancer drug target (Malumbres and Barbacid, 2001).

Transcription factors regulated by hsa-miR-34a include the winged/helix forkhead protein FoxM1, histone deacetylase 1 (HDAC1), Jun and the zinc finger protein LIM domain only 4 (LMO4). LMO-4 is inherently oncogenic and inactivates the BRCA-1 tumor suppressor protein (Sum et al., 2002; Sum et al., 2005). LMO-4 is frequently overexpressed in multiple cancer types and predicts poor outcome in breast cancer (Visvader et al., 2001; Mizunuma et al., 2003; Sum et al., 2005; Taniwaki et al., 2006). Accordingly, RNAi directed against LMO-4 leads to reduced breast cancer cell growth and migration (Sum et al., 2005). Similar to LMO4, FoxM1 also controls the expression of cell cycle genes, such as cyclins B and D (Wang et al., 2001). FoxM1 is expressed at high levels in human glioblastomas and shows tumorigenic activity in various model systems (Kalin et al., 2006; Kim et al., 2006; Liu et al., 2006). Mice deficient in FoxM1 fail to develop chemically induced hepatocellular carcinomas (Kalinichenko et al., 2004). Jun belongs to the basic region/leucine zipper (bZIP) class of transcription factors and is the cellular homolog of the avian oncoprotein v-Jun that induces tumor formation in birds (Maki et al., 1987). HDAC1 acts as a general inhibitor of transcription and cooperates with the retinoblastoma tumor suppressor protein (Rb) to decrease cell growth and proliferation (Wade, 2001).

Hsa-miR-34a also governs the expression of Fas and MCL1, both of which are functionally linked to the apoptotic pathway. MCL1 is a member of the anti-apoptotic BCL-2 (B cell lymphoma 2) gene family that give rise to two alternatively spliced gene products with opposing functions (Bae et al., 2000). High levels of MCL1 are correlated with poor prognosis of patients with ovarian carcinoma and is indicative for leukemic relapse (Kaufmann et al., 1998; Shigemasa et al., 2002). RNA interference against MCL1 induces a therapeutic response in gastric and hepatocellular carcinoma cells (Schulze-Bergkamen et al., 2006; Zangemeister-Wittke and Huwiler, 2006). Fas, also known as CD95 or APO-1, is a transmembrane cell surface receptor that functions in the transduction of apoptotic signals in response to its ligand FasL (Houston and O'Connell, 2004). Reduced Fas expression is a common mechanism of cells to decrease the sensitivity to FasL-mediated cell death. Similarly, many different cancer types show lost or decreased Fas expression levels (Table 5). In colorectal carcinoma, Fas expression is progressively reduced in the transformation of normal epithelium to benign neoplasm, adenocarcinomas and metastases (Moller et al., 1994). Thus, despite expression of FasL, tumor cells may escape the FasL induced apoptotic signal. Transient transfection of hsa-miR-34a results in an increase of Fas transcripts and therefore may restore sensitivity to FasL in cancer cells.

Further growth-related genes regulated by hsa-miR-34a include thioredoxin (TXN), a 12-kDa thiol reductase targeting various proteins and multiple pathways. Thioredoxin modulates the activity of transcription factors, induces the expression of angiogenic Hif-1α (hypoxia induced factor 1α) as well as VEGF (vascular endothelial growth factor) and can act as a proliferative and anti-apoptotic agent (Marks, 2006). In accord, carcinomas of the lung, pancreas, cervix and liver show increased levels of thioredoxin. Thioredoxin expression is also correlated with aggressive tumor growth, poor prognosis and chemoresistance (Marks, 2006).

In summary, hsa-miR-34a governs the activity of proteins that are critical regulators of cell proliferation and survival. These targets are frequently deregulated in human cancer. Based on this review of the genes and related pathways that are regulated by miR-34a, introduction of hsa-miR-34a or an anti-hsa-miR-34a into a variety of cancer cell types would likely result in a therapeutic response.

### Example 5

### Synthetic hsa-miR-34a Inhibits Proliferation of Human Lung Cancer Cells

The inventors have previously demonstrated that hsa-miR-34 is involved in the regulation of numerous cell activities that represent intervention points for cancer therapy and for therapy of other diseases and disorders (U.S. patent application Ser. No. 11/141,707 filed May 31, 2005 and Ser. No. 11/273,640 filed Nov. 14, 2005, each of which is incorporated by reference). For example, overexpression of hsa-miR-34 decreases the proliferation and/or viability of certain normal or cancerous cell lines.

The development of effective therapeutic regimens requires evidence that demonstrates efficacy and utility of the therapeutic in various cancer models and multiple cancer cell lines that represent the same disease. The inventors assessed the therapeutic effect of hsa-miR-34a for lung cancer by using eight individual lung cancer cell lines. To measure cellular proliferation of lung cancer cells, the following non-small cell lung cancer (NSCLC) cells were used: cells derived from lung adenocarcinoma (A549, H522, Calu-3, HCC2935), cells derived from lung squamous cell carcinoma (H226), cell derived from lung adenosquamous cell carcinoma (H596), cells derived from lung bronchioalveolar carcinoma (H1650), and cells derived from lung large cell carcinoma (H460). Synthetic hsa-miR-34a (Pre-miR™-hsa-miR-34a, Ambion cat. no. AM17100) or negative control (NC) miRNA (Pre-miR™ microRNA Precursor Molecule-Negative Control #2; Ambion cat. no. AM17111) was delivered via lipid-based transfection into A549, H522, H596, Calu-3, HCC2935, H1650, H460 cells and via electroporation into H226 cells.

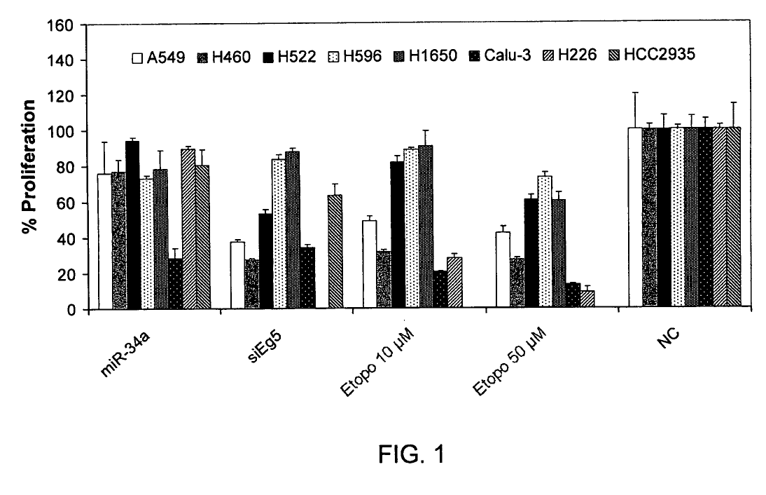

Lipid-based reverse transfections were carried out in triplicate according to a published protocol (Ovcharenko et al., 2005) and the following parameters: cells (5,000-12,000 per 96 well), 0.1-0.2 μl Lipofectamine™ 2000 (cat. no. 11668-019, Invitrogen Corp., Carlsbad, Calif., USA) in 20 μl OptiMEM (Invitrogen), 30 nM final concentration of miRNA in 100 μl. Electroporation of H226 cells was carried out using the BioRad Gene Pulser Xcell™ instrument (BioRad Laboratories Inc., Hercules, Calif., USA) with the following settings: 5×10⁶ cells with 5 μg miRNA in 200 μl OptiMEM (1.6 μM miRNA), square wave pulse at 250 V for 5 ms. Electroporated H226 cells were seeded at 7,000 cells per 96-well in a total volume of 100 μl. All cells except for Calu-3 cells were harvested 72 hours post transfection or electroporation for assessment of cellular proliferation. Calu-3 cells were harvested 10 days post transfection. Proliferation assays were performed using Alamar Blue (Invitrogen) following the manufacturer's instructions. As a control for inhibition of cellular proliferation, siRNA against the motor protein kinesin 11, also known as Eg5, was used. Eg5 is essential for cellular survival of most eukaryotic cells and a lack thereof leads to reduced cell proliferation and cell death (Weil et al., 2002). siEg5 was used in lipid-based transfection following the same experimental parameters that apply to miRNA. The inventors also used a DNA topoisomerase II inhibitor, etoposide, at a final concentration of 10 μM and 50 μM as an internal standard for the potency of miRNAs. Etoposide is an FDA-approved DNA topoisomerase II inhibitor in the treatment of lung cancer. IC50 values for various lung cancer cells have been reported to range between <1-25 μM for SCLC and NSCLC cells (Tsai et al., 1993; Ohsaki et al., 1992). Percent (%) proliferation values from the Alamar Blue assay were normalized to values from cells treated with negative control miRNA. Percent proliferation of hsa-miR-34a treated cells relative to cells treated with negative control miRNA (100%) is shown in Table 7 and in FIG. 1.

TABLE 7
Percent (%) proliferation of lung cancer cell lines treated with hsa-miR-34a, Eg5-
specific siRNA (siEg5), etoposide, or negative control miRNA (NC). Values are normalized to
values obtained from cells transfected with negative control miRNA (100% proliferation).

| | hsa-miR-34a | | | | | | | | | |
|---|---|---|---|---|---|---|---|---|---|---|
| | (30 nM) | | siEg5 (30 nM) | | etoposide (10 μM) | | etoposide (50 μM) | | NC (30 nM) | |
| | % | | % | | % | | % | | % | |
| Cells | proliferation | % SD | proliferation | % SD | proliferation | % SD | proliferation | % SD | proliferation | % SD |
| A549 | 75.95 | 17.72 | 37.84 | 1.06 | 49.13 | 2.55 | 42.18 | 3.57 | 100.00 | 19.53 |
| H460 | 77.46 | 6.20 | 27.97 | 0.33 | 32.13 | 1.14 | 27.82 | 0.58 | 100.00 | 2.52 |
| H522 | 94.41 | 1.79 | 53.45 | 2.35 | 82.13 | 3.14 | 61.08 | 2.65 | 100.00 | 7.48 |
| H596 | 73.19 | 1.62 | 83.48 | 2.82 | 88.75 | 1.11 | 73.39 | 2.67 | 100.00 | 1.89 |
| H1650 | 78.37 | 10.42 | 87.96 | 1.73 | 90.98 | 8.44 | 60.31 | 4.59 | 100.00 | 7.21 |
| Calu-3 | 28.51 | 5.65 | 34.59 | 1.33 | 20.81 | 0.19 | 13.53 | 0.64 | 100.00 | 5.54 |
| H226 | 89.50 | 1.67 | n.d. | n.d. | 28.17 | 2.32 | 9.33 | 2.70 | 100.00 | 2.43 |
| HCC2935 | 80.19 | 8.97 | 63.61 | 6.12 | n.d. | n.d. | n.d. | n.d. | 100.00 | 13.92 |

NC, negative control miRNA;
siEg5, Eg5-specific siRNA;
SD, standard deviation;
n.d., not determined.

Delivery of hsa-miR-34a inhibits cellular proliferation of lung cancer cells A549, H522, H596, Calu-3, HCC2935, H1650, H460, and H226 (Table 7 and FIG. 1). On average, hsa-miR-34a inhibits cellular proliferation by 25.30% (Table 7 and FIG. 1). hsa-miR-34a has maximal inhibitory activity in Calu-3 cells, reducing proliferation by 71.49%. The growth-inhibitory activity of hsa-miR-34a is comparable to that of etoposide at concentrations ≧10 μM. Since hsa-miR-34a induces a therapeutic response in all lung cancer cells tested, hsa-miR-34a may provide therapeutic benefit to a broad range of patients with lung cancer and other malignancies.

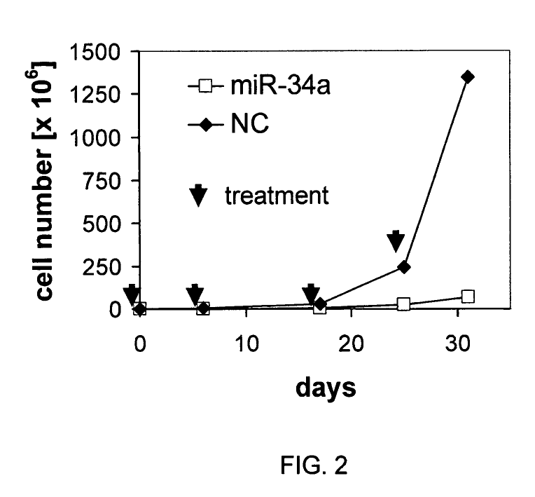

To evaluate the therapeutic activity of hsa-miR-34a over an extended period of time, the inventors conducted growth curve experiments in the presence of miRNA for up to 31 days in H226 lung cancer cells. Since in vitro transfections of naked interfering RNAs, such as synthetic miRNA, are transient by nature and compromised by the dilution of the oligo during ongoing cell divisions, miRNA was administered at multiple time points (Bartlett et al., 2006; Bartlett et al., 2007). To accommodate miRNA delivery into a large quantity of cells, hsa-miR-34a or negative control miRNA were delivered by the electroporation method. Briefly, 1×10⁶H226 were electroporated in triplicate with 1.6 μM hsa-miR-34a or negative control using the BioRad Gene Pulser Xcell™ instrument (BioRad Laboratories Inc., Hercules, Calif., USA), seeded and propagated in regular growth medium. When the control cells reached confluence (days 6, 17 and 25), cells were harvested, counted and electroporated again with the respective miRNAs. To ensure similar treatment of both conditions as well as to accommodate exponential growth, the cell numbers used for the second and third electroporation were titrated down to the lowest count. The population doubling was calculated from these electroporation events using the formula PD=ln(Nf/N0)/ln 2 and adjusting for the fact that approximately 72% of newly seeded cells adhere to the plate. Cell counts were extrapolated and plotted on a linear scale (FIG. 2). Arrows represent electroporation days. Standard deviations are included in the graphs.

Repeated administration of hsa-miR-34a robustly inhibited proliferation of human lung cancer cells (FIG. 2). In contrast, cells treated with negative control miRNA showed normal exponential growth. hsa-miR-34a treatment resulted in 94.9% inhibition of H226 cell growth on day 31 (5.1% remaining cells) relative to the proliferation of control cells (100%).

The data indicate that hsa-miR-34a provides a useful therapeutic tool in the treatment of human lung cancer cells.

### Example 6

### hsa-miR-34a, in Combination with Specific Human Micro-RNAs, Synergistically Inhibits Proliferation of Human Lung Cancer Cell Lines

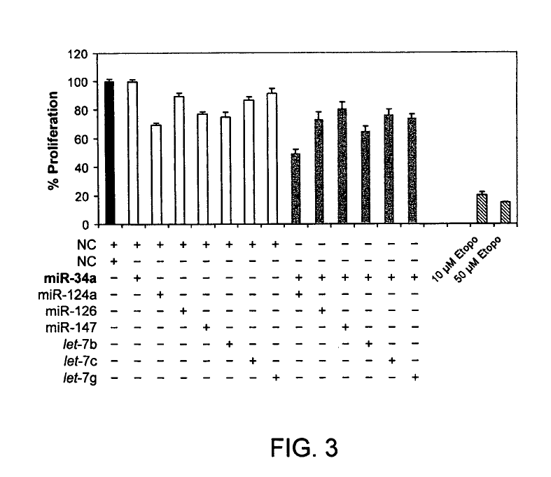

miRNAs function in multiple pathways controlling multiple cellular processes. Cancer cells frequently show aberrations in several different pathways, which determine their oncogenic properties. Therefore, administration of multiple miRNAs to cancer patients may result in a superior therapeutic benefit over administration of a single miRNA. The inventors assessed the efficacy of pair-wise miRNA combinations, administering hsa-miR-34a concurrently with either hsa-miR-124a, hsa-miR-126, hsa-miR-147, hsa-let-7b, hsa-let-7c or hsa-let-7g (Pre-miR™ miRNA, Ambion cat. no. AM17100). H460 lung cancer cells were transiently reverse-transfected in triplicate with each miRNA at a final concentration of 300 pM, resulting in 600 pM of total oligonucleotide. For negative controls, 600 pM of Pre-miR™ microRNA Precursor Molecule-Negative Control #2 (Ambion cat. no. AM17111) were used. To correlate the effect of various combinations with the effect of the sole miRNA, each miRNA at 300 pM was also combined with 300 pM negative control miRNA. Reverse transfection was carried out using the following parameters: 7,000 cells per 96 well, 0.15 μl Lipofectamine™ 2000 (Invitrogen) in 20 μl OptiMEM (Invitrogen), 100 μl total transfection volume. As an internal control for the potency of miRNA, etoposide was added at 10 pM and 50 pM to mock-transfected cells, 24 hours after transfection for the following 48 hours. Cells were harvested 72 hours after transfection and subjected to Alamar Blue assays (Invitrogen). Percent proliferation values from the Alamar Blue assays were normalized to those obtained from cells treated with 600 pM negative control miRNA. Data are expressed as % proliferation relative to negative control miRNA-treated cells (Table 8, FIG. 3).

Transfection of 300 pM hsa-miR-34a in combination with 300 pM negative control miRNA reduces proliferation of H460 cells by 0.42% (99.58% proliferation relative to cells treated with 600 pM negative control miRNA; Table 8 and FIG. 3). Additive activity of pair-wise combinations (e.g., hsa-miR-34a plus hsa-miR-147) is defined as an activity that is greater than the sole activity of each miRNA (e.g., the activity of hsa-miR-34a plus hsa-miR-147 is greater than that observed for hsa-miR-34a plus NC and the activity of hsa-miR-34a plus hsa-miR-147 is greater than that observed for hsa-miR-147 plus NC). Synergistic activity of pair-wise combinations is defined as an activity that is greater than the sum of the sole activity of each miRNA (e.g., the activity of hsa-miR-34a plus hsa-let-7g is greater than that observed for the sum of the activity of hsa-miR-34a plus NC and the activity of hsa-let-7g plus NC). The data indicate that hsa-miR-34a combined with hsa-miR-124a, hsa-miR-126, hsa-miR-147, hsa-let-7b, hsa-let-7c, or hsa-let-7g results in additive or synergistic activity (Table 8 and FIG. 3). Therefore, administering combinations of hsa-miR-34a with other miRNAs to cancer patients may induce a superior therapeutic response in the treatment of lung cancer. The combinatorial use of miRNAs represents a potentially useful therapy for cancer and other diseases.

TABLE 8
Cellular proliferation of H460 lung cancer cells in the presence
of pair-wise miR-34a miRNA combinations. Values are normalized to
values obtained from cells transfected with 600 pM negative
control (NC) miRNA.

| miRNA [300 pM] + | % Prolif- | | |
|---|---|---|---|
| miRNA [300 pM] | eration | % SD | Effect |
| NC + NC | 100.00 | 1.45 | |
| NC + miR-34a | 99.58 | 1.66 | |
| NC + miR-124a | 69.43 | 1.38 | |
| NC + miR-126 | 89.46 | 2.27 | |
| NC + miR-147 | 76.97 | 1.46 | |
| NC + let-7b | 74.92 | 3.38 | |
| NC + let-7c | 86.74 | 2.28 | |
| NC + let-7g | 91.41 | 3.26 | |
| miR-34a + miR-124a | 49.12 | 3.13 | S |
| miR-34a + miR-126 | 73.06 | 5.16 | S |
| miR-34a + miR-147 | 80.94 | 4.18 | A |
| miR-34a + let-7b | 64.85 | 3.50 | S |
| miR-34a + let-7c | 76.41 | 3.81 | S |
| miR-34a + let-7g | 73.83 | 2.85 | S |
| Etoposide (10 μM) | 20.19 | 1.89 | |
| Etoposide (50 μM) | 14.94 | 0.31 | |

SD, standard deviation;
S, synergistic effect;
A, additive effect

### Example 7

### Synthetic hsa-miR-34a Inhibits Tumor Growth of Human Lung Cancer Xenografts in Mice

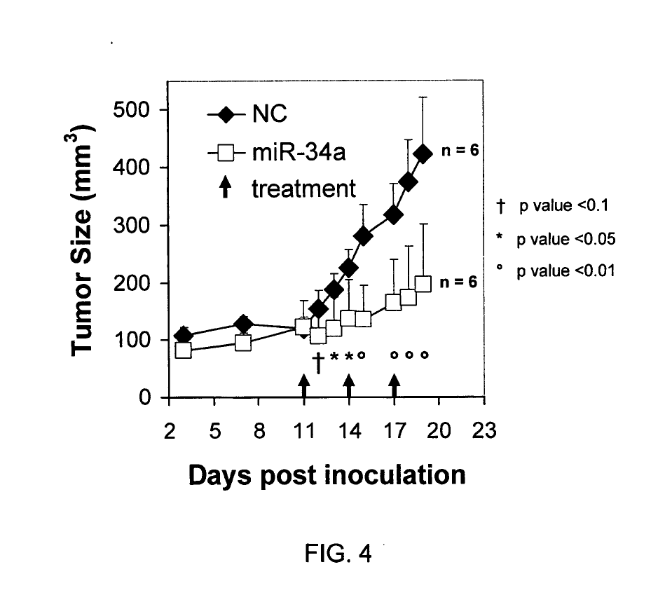

The inventors assessed the growth-inhibitory activity of hsa-miR-34a in human lung cancer xenografts grown in immunodeficient mice. Each 3×10⁶ human H460 non-small cell lung cancer cells were mixed with BD Matrigel™, (BD Biosciences; San Jose, Calif., USA; cat. no. 356237) in a 1:1 ratio and subcutaneously injected into the lower back of 23 NOD/SCID mice (Charles River Laboratories, Inc.; Wilmington, Mass., USA). Once animals developed palpable tumors (day 11 post xenograft implantation), a group of six animals received intratumoral injections of each 6.25 μg hsa-miR-34a (Dharmacon, Lafayette, Colo.) formulated with the lipid-based siPORT™ amine delivery agent (Ambion, Austin, Tex.; cat. no. AM4502) on days 11, 14, and 17. A control group of six animals received intratumoral injections of each 6.25 μg negative control miRNA (NC; Dharmacon, Lafayette, Colo.), following the same injection schedule that was used for hsa-miR-34a. Given an average mouse weight of 20 g, this dose equals 0.3125 mg/kg. In addition, a group of six H460 tumor-bearing mice received intratumoral injections of the siPORT™ amine delivery formulation lacking any oligonucleotide, and a group of five animals received intratumoral injections of phosphate-buffered saline (PBS). Caliper measurements were taken every 1-2 days, and tumor volumes were calculated using the formula, Volume=length×width×width/2, in which the length is greater than the width. Average tumor volumes, standard deviations and p-values were calculated and plotted over time (FIG. 4).

As shown in FIG. 4, three doses of hsa-miR-34a robustly inhibited growth of established H460 lung tumors (white squares). On day 19, the average volume of tumors treated with hsa-miR-34a was 196 mm³. In contrast, tumors treated with negative control miRNA (black diamonds) grew at a steady pace and yielded tumors with an average size of 421 mm³ on day 19. Negative control tumors developed as quickly as tumors treated with either PBS or the siPORT amine only control, indicating that the therapeutic activity of hsa-miR-34a is specific.

The data indicate that hsa-miR-34a represents a particularly useful candidate in the treatment of patients with lung cancer. The therapeutic activity of hsa-miR-34a is highlighted by the fact that hsa-miR-34a inhibits tumor growth of tumors that had developed prior to treatment.

In addition, the data demonstrate the therapeutic utility of hsa-miR-34a in a lipid-based formulation.

### Example 8

### Synthetic hsa-miR-34a Inhibits Proliferation of Human Prostate Cancer Cells

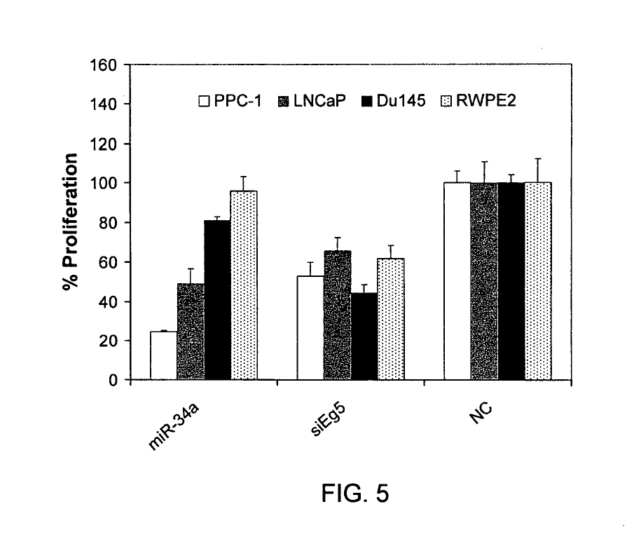

The inventors assessed the therapeutic effect of hsa-miR-34a for prostate cancer by using four individual human prostate cancer cell lines. To measure cellular proliferation of prostate cancer cells, the following prostate cancer cell lines were used: PPC-1, derived from a bone metastasis; Du145, derived from a brain metastasis; RWPE2, derived from prostate cells immortalized by human papillomavirus 18 and transformed by the K-RAS oncogene; and LNCaP, derived from a lymph node metastasis (Bello et al., 1997; Pretlow et al., 1993; Stone et al., 1978; Brothman et al., 1991; Horoszewicz et al., 1980). PPC-1 and Du145 cells lack expression of the prostate-specific antigen (PSA) and are independent of androgen receptor (AR) signaling. In contrast, RWPE2 and LNCaP cells test positive for PSA and AR. Cells were transfected with synthetic hsa-miR-34a (Pre-miR™-hsa-miR-34a, Ambion cat. no. AM17100) or negative control miRNA (NC; Pre-miR™ microRNA Precursor Molecule-Negative Control #2; Ambion cat. no. AM17111) in a 96-well format using a lipid-based transfection reagent. Lipid-based reverse transfections were carried out in triplicate according to a published protocol (Ovcharenko et al., 2005) and the following parameters: cells (6,000-7,000 per 96 well), 0.1-0.2 μl Lipofectamine™ 2000 (cat. no. 11668-019, Invitrogen Corp., Carlsbad, Calif., USA) in 20 μl OptiMEM (Invitrogen), 30 nM final concentration of miRNA in 100 μl. Proliferation was assessed 4-7 days post-transfection using Alamar Blue™ (Invitrogen) following the manufacturer's instructions. As a control for inhibition of cellular proliferation, siRNA against the motor protein kinesin 11, also known as Eg5, was used. Eg5 is essential for cellular survival of most eukaryotic cells and a lack thereof leads to reduced cell proliferation and cell death (Weil et al., 2002). siEg5 was used in lipid-based transfection following the same experimental parameters that apply to miRNA. Fluorescent light units (FLU) were measured after 3 hours, normalized to the control, and plotted as percent change in proliferation. Percent proliferation of hsa-miR-34a treated cells relative to cells treated with negative control miRNA (100%) is shown in Table 9 and in FIG. 5.

TABLE 9
Percent (%) proliferation of human prostate cancer cell lines
treated with hsa-miR-34a, Eg5-specific siRNA (siEg5), or negative
control miRNA (NC). Values are normalized to values
obtained from cells transfected with negative control miRNA
(100% proliferation).

| | hsa-miR-34a (30 nM) | | siEg5 (30 nM) | | NC (30 nM) | |
|---|---|---|---|---|---|---|
| | % | | % | | % | |
| Cells | proliferation | % SD | proliferation | % SD | proliferation | % SD |
| PPC-1 | 24.65 | 0.62 | 52.90 | 6.97 | 100.00 | 5.82 |
| LNCaP | 49.40 | 7.10 | 66.01 | 6.26 | 100.00 | 10.73 |
| Du145 | 81.26 | 1.80 | 44.47 | 4.23 | 100.00 | 4.12 |
| RWPE2 | 95.96 | 7.05 | 61.87 | 6.56 | 100.00 | 12.28 |

NC, negative control miRNA;
siEg5, Eg5-specific siRNA;
SD, standard deviation.

Delivery of hsa-miR-34a inhibits cellular proliferation of human prostate cancer cells PPC-1, Du145, LNCaP and RWPE2 (Table 9 and FIG. 5). On average, hsa-miR-34a inhibits cellular proliferation by 37.18%. The growth-inhibitory activity of hsa-miR-34a is comparable to that of Eg5-directed siRNA. Since hsa-miR-34a induces a therapeutic response in all prostate cancer cells tested, hsa-miR-34a may provide therapeutic benefit to a broad range of patients with prostate cancer and other malignancies.

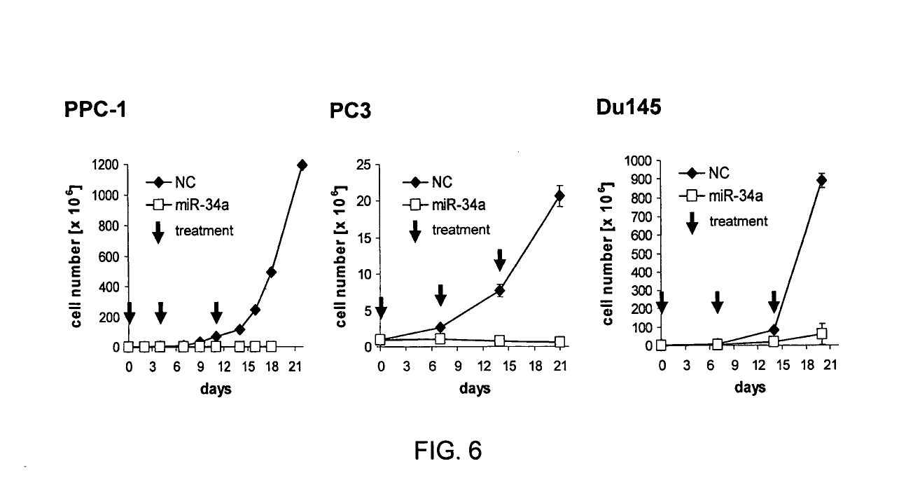

To evaluate the therapeutic activity of hsa-miR-34a over an extended period of time, we conducted growth curve experiments in the presence of miRNA for up to 22 days. Since in vitro transfections of naked interfering RNAs, such as synthetic miRNA, are transient by nature and compromised by the dilution of the oligo during ongoing cell divisions, miRNA was administered at multiple time points (Bartlett et al. 2006; Bartlett et al. 2007). To accommodate miRNA delivery into a large quantity of cells, the inventors employed the electroporation method to deliver hsa-miR-34a or negative control miRNA into PPC-1, PC3, and Du145 human prostate cancer cells. Briefly, 1×10⁶ PPC-1 or PC3 cells, or 0.5×10⁶ Du145 cells were electroporated with 1.6 μM hsa-miR-34a or negative control using the BioRad Gene Pulser Xcell™ instrument (BioRad Laboratories Inc., Hercules, Calif., USA), seeded and propagated in regular growth medium. Experiments with PC3 and Du145 cells were carried out in triplicates. When the control cells reached confluence (days 4 and 11 for PPC-1; days 7 and 14 for PC3 and Du145), cells were harvested, counted and electroporated again with the respective miRNAs. To ensure similar treatment of both conditions as well as to accommodate exponential growth, the cell numbers used for the second and third electroporation were titrated down to the lowest count. The population doubling was calculated from these electroporation events using the formula PD=ln(Nf/N0)/ln 2 and adjusting for the fact that approximately 72% of newly seeded cells adhere to the plate. Cell counts were extrapolated and plotted on a linear scale (FIG. 6). Arrows represent electroporation days. Standard deviations are included in the graphs.

Repeated administration of hsa-miR-34a robustly inhibited proliferation of human prostate cancer cells (FIG. 6, white squares). In contrast, cells treated with negative control miRNA showed normal exponential growth (FIG. 6, black diamonds). hsa-miR-34a treatment resulted in 97.2% inhibition of PC3 cell growth on day 21 (2.8% cells relative to cells electroporated with negative control miRNA), and 93.1% inhibition of Du145 cell growth on day 19 (6.9% cells relative to cells electroporated with negative control miRNA) relative to the proliferation of control cells (100%). All PPC-1 cells electroporated with hsa-miR34a were eliminated by day 22.

The data indicate that hsa-miR-34a provides a useful therapeutic tool in the treatment of human prostate cancer cells.

### Example 9

### Synthetic hsa-miR-34a Inhibits Tumor Growth of Human Prostate Cancer Xenografts in Mice

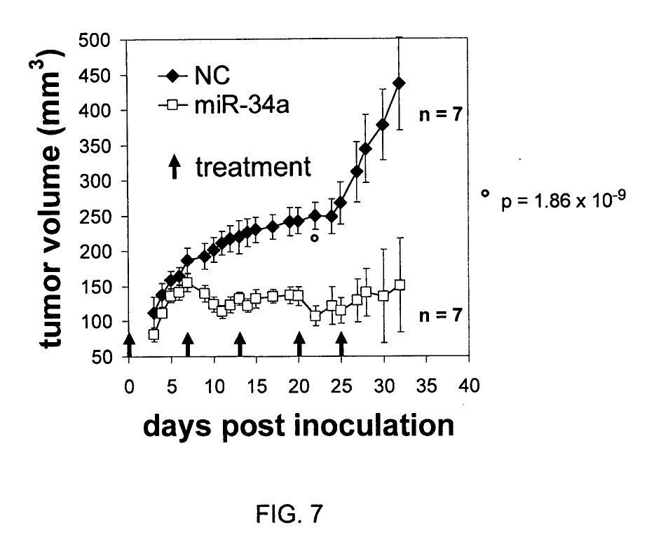

The in vitro studies demonstrate the therapeutic activity of hsa-miR-34a in cultured human prostate cancer cells. Therefore, hsa-miR-34a is likely to interfere with prostate tumor growth in the animal. To explore this possibility, the therapeutic potential of synthetic hsa-miR-34a miRNA was evaluated in the animal using the PPC-1 human prostate cancer xenograft. 5×10⁶ PPC-1 cells per animal were electroporated with 1.6 μM synthetic hsa-miR-34a or negative control miRNA (Pre-miR™-hsa-miR-34a, Ambion cat. no. AM17100; NC, Pre-miR™ microRNA Precursor Molecule-Negative Control #2, Ambion cat. no. AM17111), mixed with BD Matrigel™, (BD Biosciences; San Jose, Calif., USA; cat. no. 356237) in a 1:1 ratio and implanted subcutaneously into the lower back of NOD/SCID mice (Charles River Laboratories, Inc.; Wilmington, Mass., USA). A group of 7 mice was injected with hsa-miR-34a treated PPC-1 cells, and a group of 7 animals was injected with PPC-1 cells treated with negative control miRNA. To maintain steady levels of miRNA, 6.25 μg of each hsa-miR-34a or negative control miRNA conjugated with the lipid-based siPORT™ amine delivery agent (Ambion, Austin, Tex.; cat. no. AM4502) were repeatedly administered on days 7, 13, 20, and 25 via intra-tumoral injections. Given an average mouse weight of 20 g, this dose equals 0.3125 mg/kg. Tumor growth was monitored by taking caliper measurements every 1-2 days for 32 days. Tumor volumes were calculated using the formula, Volume=length×width×width/2, in which the length is greater than the width, and plotted over time (FIG. 7). Standard deviations are shown in the graph. All data points had p values<0.01. p values were as low as 1.86×10⁻⁹ for data obtained on day 22, indicating statistical significance.

Repeated dosing with hsa-miR-34a blocked tumor growth of the human PPC-1 prostate cancer xenograft (FIG. 7, white squares). The average volume of tumors that received hsa-miR-34a was 151 mm³ on day 32. Volumes of newly implanted tumors ranged between 111 and 155 mm³ (days 4-7) and thus, PPC-1 tumors failed to develop in response to hsa-miR-34a treatment. In contrast, tumors locally treated with negative control miRNA were unaffected and continued to grow at a steady pace (FIG. 7, black diamonds). The average volume of tumors treated with negative control miRNA was 437 mm³ on day 32. Of note, each single administration with hsa-miR-34a resulted in an acute regression of tumor volumes. This effect was not induced with negative control miRNA, indicating that the anti-tumor activity of hsa-miR-34a is specific.

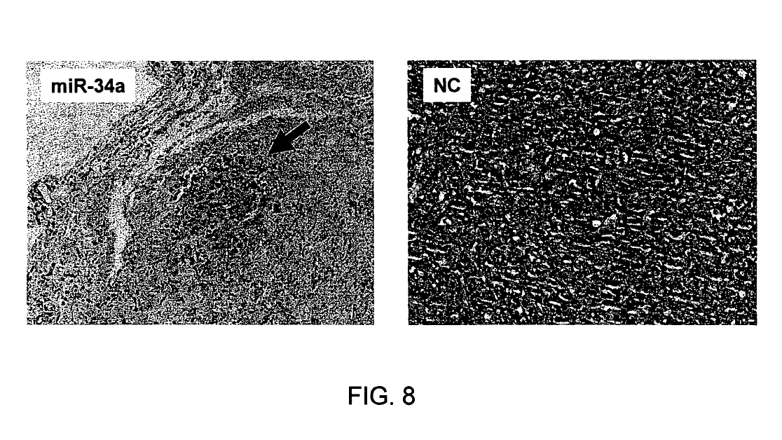

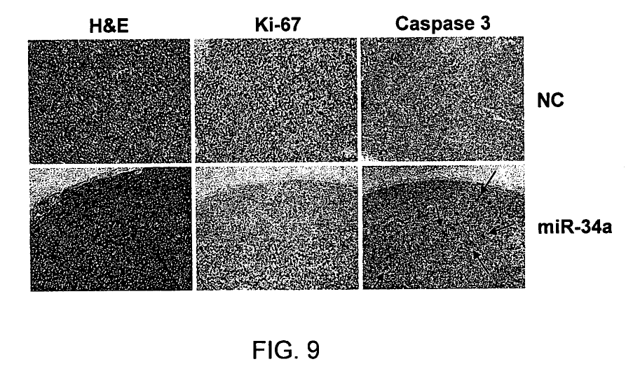

A histological analysis revealed that PPC-1 tumors treated with negative control miRNA were densely packed with healthy, viable prostate cancer cells (FIG. 8). In contrast, hsa-miR-34a-treated tumors consisted mostly of matrigel with cellular debris and sparsely distributed cells, as well as occasional pockets with seemingly viable cells (FIG. 8, arrow). To gain further insight into the biological status of these cells, immunohistochemistry analyses specific for the proliferation marker Ki-67, as well as caspase 3, an indicator of apoptosis were performed. As illustrated in FIG. 9, areas with viable cells in hsa-miR-34a-treated tumors showed reduced levels of Ki-67 and increased levels of caspase 3. The data indicate that hsa-miR-34a inhibits tumor growth by an anti-proliferative and pro-apoptotic mechanism.

The data indicate that hsa-miR-34a provides a powerful therapeutic tool in the treatment of patients with prostate cancer.

In addition, the data demonstrate the therapeutic utility of hsa-miR-34a in a lipid-based formulation.

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,870,287
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,399,363
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,468
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,525,464
U.S. Pat. No. 5,527,681
U.S. Pat. No. 5,529,756
U.S. Pat. No. 5,532,128
U.S. Pat. No. 5,543,158
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,289
U.S. Pat. No. 5,610,287
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,641,515
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,677,195
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,153
U.S. Pat. No. 5,708,154
U.S. Pat. No. 5,714,606
U.S. Pat. No. 5,728,525
U.S. Pat. No. 5,739,169
U.S. Pat. No. 5,744,305
U.S. Pat. No. 5,760,395
U.S. Pat. No. 5,763,167
U.S. Pat. No. 5,770,358
U.S. Pat. No. 5,777,092
U.S. Pat. No. 5,789,162
U.S. Pat. No. 5,792,847
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,846,225
U.S. Pat. No. 5,846,945
U.S. Pat. No. 5,847,219
U.S. Pat. No. 5,856,174
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 5,922,591
U.S. Pat. No. 6,004,755
U.S. Pat. No. 6,040,193
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. application Ser. No. 09/545,207
U.S. application Ser. No. 10/667,126
U.S. application Ser. No. 11/141,707
U.S. application Ser. No. 11/273,640
U.S. application Ser. No. 11/349,727
U.S. Prov. Appln. Ser. 60/575,743
U.S. Prov. Appln. Ser. 60/649,584
U.S. Prov. Appln. Ser. 60/650,807
Akiba et al., Int. J. Oncol., 18(2):257-264, 2001.
Akino et al., Gastroenterology, 129(1):156-169, 2005.
Ando et al., J. Biol. Chem., 279(24):25549-25561, 2004.
Arap et al., Cancer Res., 55(6):1351-1354, 1995.
Aspland et al., Oncogene, 20(40):5708-5717, 2001.
Austin-Ward and Villaseca, Revista Medica de Chile, 126(7):838-845, 1998.
Ausubel et al., In: Current Protocols in Molecular Biology, John, Wiley & Sons, Inc, NY, 1994.
Bae et al., J. Biol. Chem., 275(33):25255-25261, 2000.
Bagga et al., Cell, 122(4):553-563, 2005.
Bai et al., Histol. Histopathol., 18(2):449-457, 2003.
Bar-Shira et al., Cancer Res., 62(23):6803-6807, 2002.
Bartlett et al., Biotechnol Bioeng, 97(4):909-21, 2007.
Bartlett et al., Nucleic Acids Res, 34(1):322-33, 2006.
Barton et al., Clin. Cancer Res., 3(9):1579-1586, 1997.
Beisner et al., Cancer Res., 66(15):7554-7561, 2006.
Bello et al., Carcinogenesis, 18(6):1215-23, 1997.
Billottet et al., Oncogene, 25(50):6648-6659, 2006.
Birchmeier et al., Nat. Rev. Mol. Cell Biol., 4(12):915-925, 2003.
Biswas et al., Cancer Res., 64(14):4687-4692, 2004.
Boccaccio and Comoglio, Nat. Rev. Cancer, 6(8):637-645, 2006.
Bodner-Adler et al., Anticancer Res., 21(1B):809-812, 2001.
Borczuk et al., Am. J. Pathol., 163(5):1949-1960, 2003.
Bostwick et al., Prostate, 58(2):164-168, 2004.
Boultwood et al., Br. J. Haematol., 126(4):508-511, 2004.
Boutros et al., Biochem. Biophys. Res. Commun., 325(4):1115-1121, 2004.
Bradham et al., J. Cell Biol., 114(6):1285-1294, 1991.
Bravou et al., Int. J. Oncol., 27(6):1511-1518, 2005.
Brothman et al., J. Urol., 145(5):1088-91, 1991.
Bui et al., Br. J. Cancer, 77(2):319-324, 1998.
Bukowski et al., Clinical Cancer Res., 4(10):2337-2347, 1998.
Cahill et al., Nature, 392(6673):300-303, 1998.
Caldas et al., Cancer Res., 54:3568-3573, 1994.
Calin and Croce, Nat. Rev. Cancer, 6(11):857-866, 2006.
Carrington and Ambros, Science, 301(5631):336-338, 2003.
Castillo et al., Cancer Res., 66(12):6129-6138, 2006.
Cheng et al., Cancer Res., 54(21):5547-5551, 1994.
Chieffi et al., Prostate, 66(3):326-333, 2006.
Choi et al., Oncogene, 23(42):7095-7103, 2004.
Cho-Vega et al., Hum. Pathol., 35(9):1095-1100, 2004.
Christodoulides et al., Microbiology, 144(Pt 11):3027-3037, 1998.
Claudio et al., Clin. Cancer Res., 8(6):1808-1815, 2002.
Cohen et al., Oncogene, 20(2):141-146, 2001.
Cooper et al., Nature, 311(5981):29-33, 1984.
Croci et al., Cancer Res., 64(5):1730-1736, 2004.
Cummins et al., In: IRT: Nucleosides and nucleosides, La Jolla Calif., 72, 1996.
Danilkovitch-Miagkova and Zbar, J. Clin. Invest., 109(7):863-867, 2002.
D'Antonio et al., Int. J. Oncol., 21(5):941-948, 2002.
Davidson et al., J. Immunother., 21(5):389-398, 1998.
Dean et al., Nature, 318(6044):385-388, 1985.
Denli et al., Trends Biochem. Sci., 28:196, 2003.
Didenko, Biotechniques, 31(5):1106-1116, 1118, 1120-1121, 2001.
Dillman, Cancer Biother. Radiopharm., 14(1):5-10, 1999.
Donnellan and Chetty, Mol. Pathol., 51(1):1-7, 1998.
Ebert et al., Cancer Res., 54(15):3959-3962, 1994.
Eferl et al., Cell, 112(2):181-192, 2003.
Emptage et al., Neuron, 29(1):197-208, 2001.
Endoh et al., Br. J. Cancer, 93(12):1395-1399, 2005.
EP 266,032
EP 373 203
EP 785 280
EP 799 897
Esquela-Kerscher and Slack, Nat. Rev. Cancer, 6(4):259-269, 2006.
Filipits et al., Clin. Cancer Res., 8(3):729-733, 2002.
Fisher, J. Royal Statistical. Soc., 85(1):87-94, 1922.
Fleischer et al., Int. J. Oncol., 28(1):25-32, 2006.
Florenes et al., Clin. Cancer Res., 6(9):3614-3620, 2000.
Fodor et al., Biochemistry, 30(33):8102-8108, 1991.
Froehler et al., Nucleic Acids Res., 14(13):5399-5407, 1986.
Ginestier et al., Clin. Cancer Res., 12(15):4533-4544, 2006.
Grabsch et al., J. Pathol., 200(1):16-22, 2003.
Gratas et al., Cancer Res., 58(10):2057-2062, 1998.
Griffey et al., J Mass Spectrom., 32(3):305-13, 1997.
Halkidou et al., Prostate, 59(2):177-189, 2004.
Han et al., Oncogene, 23(7):1333-1341, 2004.
Hanahan and Weinberg, Cell, 100:57-70, 2000.
Hanibuchi et al., Int. J. Cancer, 78(4):480-485, 1998.
Hartmann et al., Cancer Res., 59(7):1578-1583, 1999.
Hellstrand et al., Acta Oncologica, 37(4):347-353, 1998.
Hishikawa et al., J. Biol. Chem., 274(52):37461-37466, 1999.
Horoszewicz et al., Prog Clin Biol Res, 37(115-32, 1980.
Houston and O'Connell, Curr. Opin. Pharmacol., 4(4):321-326, 2004.
Huguet et al., Cancer Res., 54(10):2615-2621, 1994.
Hui and Hashimoto, Infection Immun., 66(11):5329-5336, 1998.
Hussussian et al., Nat. Genet., 8(1):15-21, 1994.
Hynes and Lane, Nat. Rev. Cancer, 5(5):341-354, 2005.

Iolascon et al., Hepatology, 27(4):989-995, 1998.
Ishikawa et al., Cancer Res., 65(20):9176-9184, 2005.
Itakura and Riggs, Science, 209:1401-1405, 1980.
Ito et al., Anticancer Res., 21(2A): 1043-1048, 2001.
Ito et al., Anticancer Res., 23(5A):3819-3824, 2003.
Jin et al., Nature, 442(7102):576-579, 2006.
Jonson et al., Int. J. Oncol., 19(1):71-81, 2001.
Ju et al., Gene Ther., 7(19):1672-1679, 2000.
Kalin et al., Cancer Res., 66(3):1712-1720, 2006.
Kalinichenko et al., Genes Dev., 18(7):830-850, 2004.
Kamb et al., Science, 2674:436-440, 1994.
Kaufmann et al., Blood, 91(3):991-1000, 1998.
Kawai et al., Int. J. Cancer, 107(3):353-358, 2003.
Keen and Taylor, Nat. Rev. Cancer, 4(12):927-936, 2004.
Kim et al., Cancer Res., 66(4):2153-2161, 2006.
Kitada et al., Blood, 91(9):3379-3389, 1998.
Kitadai et al., Jpn. J. Cancer Res., 84(8):879-884, 1993.
Klostermeier and Millar, Biopolymers, 61(3):159-79, 2001-2002.
Koliopanos et al., World J. Surg., 26(4):420-427, 2002.
Komberg and Baker, DNA Replication, 2nd Ed., Freeman, San Francisco, 1992.
Krajewska et al., Am. J. Pathol., 148(5):1567-1576, 1996.
Krasagakis et al., Br. J. Cancer, 77(9):1492-1494, 1998.
Krek et al., Nature Genet., 37:495-500, 2005.
Kulkarni et al., Leukemia, 16(1):127-134, 2002.
Lagos-Quintana et al., Science, 294(5543):853-858, 2001.
Lambros et al., J. Pathol., 205(1):29-40, 2005.
Lau et al., Science, 294(5543):858-862, 2001.
Lee and Ambros, Science, 294(5543):862-864, 2001.
Lim et al., Nature, 433(7027):769-773, 2005.
Lin and Gelman, Cancer Res., 57(11):2304-2312, 1997.
Lin et al., Gastroenterology, 128(1):9-23, 2005.
Liu and Erikson, Proc. Natl. Acad. Sci. USA, 100(10):5789-5794, 2003.
Liu and Matsuura, Cell Cycle, 4(1):63-66, 2005.
Liu et al., Cancer Res., 66(2):653-658, 2006.
Liu et al., Cancer Res., 66(7):3593-3602, 2006.
Lopez-Beltran et al., J. Pathol., 209(1):106-113, 2006.
Lucke et al., Cancer Res., 61(2):482-485, 2001.
Lutchman and Rouleau, Cancer Res., 55(11):2270-2274, 1995.
Mahtouk et al., Oncogene, 24(21):3512-3524, 2005.
Maki et al., Proc. Natl. Acad. Sci. USA, 84(9):2848-2852, 1987.
Malumbres and Barbacid, Nat. Rev. Cancer, 1(3):222-231, 2001.
Markowitz et al., Science, 268(5215):1336-1338, 1995.
Markowitz, Biochim. Biophys. Acta, 1470(1):M13-20, 2000.
Marks, Semin. Cancer Biol., 16(6):436-443, 2006.
Marsters et al., Recent Prog. Horm. Res., 54:225-234, 1999.
Martinez-Lorenzo et al., Int. J. Cancer, 75(3):473-481, 1998.
Massague et al., Cell, 103(2):295-309, 2000.
Matsumoto et al., Leukemia, 14(10):1757-1765, 2000.
McClatchey and Giovannini, Genes Dev., 19(19):2265-2277, 2005.
Meng et al., Gastroenterology, 130:2113-2129, 2006.
Miyake et al., Cancer, 86(2):316-324, 1999.
Mizunuma et al., Br. J. Cancer, 88(10):1543-1548, 2003.
Moller et al., Int. J. Cancer, 57(3):371-377, 1994.
Montero et al., Clin. Cancer Res., 4(9):2161-2168, 1998.
Mori et al., Cancer Res., 54(13):3396-3397, 1994.
Mori et al., Gastroenterology, 131(3):797-808, 2006.
Morishita et al., Hepatology, 40(3):677-686, 2004.
Mundt et al., Biochem. Biophys. Res. Commun., 239(2):377-385, 1997.
Nakagawa et al., Oncogene, 23(44):7366-7377, 2004.
Nauert et al., Curr. Biol., 7(1):52-62, 1997.
Nobri et al., Nature (London), 368:753-756, 1995.
Nupponen et al., Am. J. Pathol., 154(6): 1777-1783, 1999.
Nupponen et al., Genes Chromosomes Cancer, 28(2):203-210, 2000.
Ohsaki et al., Cancer Res, 52(13):3534-8, 1992.
Okamoto et al., Hepatology, 38(5):1242-1249, 2003.
Okamoto et al., Proc. Natl. Acad. Sci. USA, 91(23):11045-11049, 1994.
Olsen et al., Dev. Biol., 216:671, 1999.
Orlow et al., Cancer Res, 54(11):2848-2851, 1994.
Ovcharenko et al., Rna, 11(6):985-93, 2005.
Pan et al., Neurol. Res., 24(7):677-683, 2002.
PCT Appln. WO 0138580
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
Pietras et al., Oncogene, 17(17):2235-2249, 1998.
Pretlow et al., J Natl Cancer Inst, 85(5):394-8, 1993.
Pruitt et al., Nucleic Acids Res., 33(1):D501-D504, 2005.
Pruneri et al., Clin. Cancer Res., 11(1):242-248, 2005.
Qian et al., Proc. Natl. Acad. Sci. USA, 99(23):14925-14930, 2002.
Qin et al., Proc. Natl. Acad. Sci. USA, 95(24):14411-14416, 1998.
Remington's Pharmaceutical Sciences” 15th Edition, pages 1035-1038 and 1570-1580, 1990.
Rincon-Arano et al., Cancer, 97(3):575-585, 2003.
Rosenkilde and Schwartz, Apmis, 112(7-8):481-495, 2004.
Ru et al., Oncogene, 21(30):4673-4679, 2002.
Rubin and Gutmann, Nat. Rev. Cancer, 5(7):557-564, 2005.
Rust et al., J. Clin. Pathol., 58(5):520-524, 2005.
Sambrook and Russell, Molecular Cloning: A Laboratory Manual 3^{rd} Ed., Cold Spring Harbor Laboratory Press, 1989.
Sambrook and Russell, Molecular Cloning: A Laboratory Manual 3^{rd} Ed., Cold Spring Harbor Laboratory Press, 2001.
Sambrook et al., In. DNA microaarays: a molecular cloning manual, Cold Spring Harbor Laboratory Press, Cold Spring Harbor, N.Y., 2003.
Sanchez-Aguilera et al., Blood, 103(6):2351-2357, 2004.
Sano et al., Histopathology, 46(5):532-539, 2005.
Scheit, In: Synthesis and Biological Function, Wiley-Interscience, New York, 171-172, 1980.
Schulze-Bergkamen et al., BMC Cancer, 6:232, 2006.
Seggerson et al., Dev. Biol., 243:215, 2002.
Serrano et al., Nature, 366:704-707, 1993.
Serrano et al., Science, 267(5195):249-252, 1995.
Sherr and McCormick, Cancer Cell, 2(2):103-112, 2002.
Shetty et al., Br. J. Cancer, 93(11):1295-1300, 2005.
Shigeishi et al., Oncol. Rep., 15(4):933-938, 2006.
Shigemasa et al., Jpn. J. Cancer Res, 93(5):542-550, 2002.
Shimo et al., Cancer Lett., 174(1):57-64, 2001.
Shimoyama et al., Clin. Cancer Res., 5(5):1125-1130, 1999.
Shinoura et al., Cancer Gene Ther., 7(2):224-232, 2000.
Sieghart et al., J. Hepatol., 44(1):151-157, 2006.
Smith et al., Biochem. Biophys. Res. Commun., 234(2):397-405, 1997.
Smith et al., Br. J. Cancer, 93(6):719-729, 2005.
Solic and Davies, Exp. Cell Res., 234(2):465-476, 1997.
Sparmann and Bar-Sagi, Cancer Cell, 6(5):447-458, 2004.
Stone et al., Int J Cancer, 21(3):274-81, 1978.
Strebhardt and Ullrich, Nat. Rev. Cancer, 6(4):321-330, 2006.
Sujobert et al., Blood, 106(3):1063-1066, 2005.
Sum et al., J. Biol. Chem., 277(10):7849-7856, 2002.
Sum et al., Proc. Natl. Acad. Sci. USA, 102(21):7659-7664, 2005.
Takimoto et al., Biochem. Biophys. Res. Commun., 251(1):264-268, 1998.
Tanami et al., Lab. Invest., 85(9):1118-1129, 2005.
Taniwaki et al., Int. J. Oncol., 29(3):567-575, 2006.
Tanner et al., Clin. Cancer Res., 6(5):1833-1839, 2000.
Thome, Nat. Rev. Immunol., 4(5):348-359, 2004.
Tikoo et al., J. Biol. Chem., 269(38):23387-23390, 1994.
Troncone et al., J. Clin. Pathol., 60(4):377-381, 2007.
Tsai et al., J Natl Cancer Inst, 85(11):897-901, 1993.
Turner et al., Nat. Rev. Cancer, 4(10):814-819, 2004.
UK Appln. 8 803 000
UK Patent 1,529,202
Ulisse et al., Int. J. Cancer, 119(2):275-282, 2006.
Vanhaesebroeck et al., Trends Biochem. Sci., 22(7):267-272, 1997.
Viard-Leveugle et al., J. Pathol., 201(2):268-277, 2003.
Visvader et al., Proc. Natl. Acad. Sci. USA, 98(25):14452-14457, 2001.
Vogt et al., Cell Cycle, 5(9):946-949, 2006.
Vos et al., J. Biol. Chem., 278(30):28045-28051, 2003.
Wade, Hum. Mol. Genet., 10(7):693-698, 2001.
Wang et al., Proc. Natl. Acad. Sci. USA, 98(20):11468-11473, 2001.
Weil et al., Biotechniques, 33(6):1244-8, 2002.
Weiss and Bohmann, Cell Cycle, 3(2):111-113, 2004.
Wikman et al., Oncogene, 21(37):5804-5813, 2002.
Wohlschlegel et al., Am. J. Pathol., 161(1):267-273, 2002.
Wooster and Weber, N. Engl. J. Med., 348(23):2339-2347, 2003.
Wu et al., Eur. J. Cancer, 38(14):1838-1848, 2002.
Wu et al., Eur. J. Cancer, 42(4):557-565, 2006.
Wu et al., Int. J. Gynecol. Cancer, 16(4):1668-1672, 2006.
Wuilleme-Toumi et al., Leukemia, 19(7):1248-1252, 2005.
Xi et al., Clin. Cancer Res., 12(8):2484-2491, 2006a.
Xi et al., Clin. Chem., 52(3):520-523, 2006b.
Xia et al., Cancer Res., 61(14):5644-5651, 2001.
Yamagata et al., Cancer Res., 65(1):157-165, 2005.
Yang et al., Cancer Cell, 9(6):445-457, 2006.
Yang et al., Cancer Res., 65(19):8887-8895, 2005.
Yu and Feig, Oncogene, 21(49):7557-7568, 2002.
Zangemeister-Wittke and Huwiler, Cancer Biol. Ther., 5(10):1355-1356, 2006.
Zhu et al., Cell, 94(6):703-714, 1998.

1. A method of modulating gene expression in a cell comprising administering to the cell an amount of an isolated nucleic acid comprising a miR-34 nucleic acid sequence in an amount sufficient to modulate the expression of one or more genes identified in Table 1, 3, 4, or 5.

2. The method of claim 1, wherein the cell is in a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous condition.

3. (canceled)

4. The method of claim 2, wherein the cancerous condition is astrocytoma; anaplastic large cell lymphoma; acute lymphoblastic leukemia; acute myeloid leukemia; angiosarcoma; breast carcinoma; B-cell lymphoma; bladder carcinoma; cervical carcinoma; carcinoma of the head and neck; chronic lymphocytic leukemia; chronic myeloid leukemia; colorectal carcinoma; endometrial carcinoma; glioma; glioblastoma; gastric carcinoma; gastrinoma; hepatoblastoma; hepatocellular carcinoma; Hodgkin lymphoma; Kaposi's sarcoma; leukemia; lung carcinoma; leiomyosarcoma; laryngeal squamous cell carcinoma; melanoma; mucosa-associated lymphoid tissue B-cell lymphoma; medulloblastoma; mantle cell lymphoma; meningioma; myeloid leukemia; multiple myeloma; high-risk myelodysplastic syndrome; mesothelioma; neurofibroma; non-Hodgkin lymphoma; non-small cell lung carcinoma; ovarian carcinoma; esophageal carcinoma; oropharyngeal carcinoma; osteosarcoma; pancreatic carcinoma; papillary carcinoma; prostate carcinoma; pheochromocytoma; rhabdomyosarcoma; squamous cell carcinoma of the head and neck; schwannoma; small cell lung cancer; salivary gland tumor; sporadic papillary renal carcinoma; thyroid carcinoma; testicular tumor; urothelial carcinoma wherein the modulation of one or more gene is sufficient for a therapeutic response.

5. The method of claim 4, wherein the cancerous condition is lung carcinoma

6. The method of claim 5, wherein lung carcinoma is non-small cell lung carcinoma.

7. (canceled)

8. The method of claim 4, wherein the cancerous condition is prostate carcinoma.

9. The method of claim 8, wherein prostate carcinoma is associated with detectable prostate-specific antigen (PSA).

10. The method of claim 8, wherein prostate carcinoma is androgen independent.

11. The method of claim 1, wherein the expression of a gene is down-regulated.

12. The method of claim 1, wherein the expression of a gene is up-regulated.

13-16. (canceled)

17. The method of claim 1, wherein the isolated miR-34 nucleic acid is a recombinant nucleic acid.

18-22. (canceled)

23. The method of claim 1, wherein the miR-34 nucleic acid is a synthetic nucleic acid.

24. The method of claim 23, wherein the nucleic acid is administered at a dose of 0.01 mg/kg of body weight to 10 mg/kg of body weight.

25. (canceled)

26. The method of claim 1, wherein the miR-34 is miR-34a, miR-34b, or miR-34c.

27. The method of claim 1, wherein the nucleic acid is administered enterally or parenterally.

28. (canceled)

29. (canceled)

30. The method of claim 1, wherein the nucleic acid is comprised in a pharmaceutical formulation.

31. The method of claim 30, wherein the pharmaceutical formulation is a lipid composition or a nanoparticle composition.

32. (canceled)

33. The method of claim 30, wherein the pharmaceutical formulation consists of biocompatible and/or biodegradable molecules.

34-52. (canceled)

53. A method of selecting a miRNA to be administered to a subject with, suspected of having, or having a propensity for developing a pathological condition or disease comprising:
(a) determining an expression profile of one or more genes selected from Table 1, 3, 4, or 5;
(b) assessing the sensitivity of the subject to miRNA therapy based on the expression profile; and
(c) selecting one or more miRNA based on the assessed sensitivity.

54-56. (canceled)

57. A method of assessing a cell, tissue, or subject comprising assessing expression of miR-34 in combination with assessing expression of one or more gene from Table 1, 3, 4, or 5 in at least one sample.

58. (canceled)
